(19) United States
(12) Patent Application Publication
(10) Pub. No.: US 2007/0160574 A1
(43) Pub. Date: Jul. 12, 2007

(54) Design of CXC chemokine analogs for the treatment of human diseases

(76) Inventors: Ahmed Merzouk, Richmond (CA); Hassan Salari, Vancouver (CA)

Correspondence Address: PERKINS COIE LLP, P.O. BOX 2168, MENLO PARK, CA, 94026, US

(21) Appl. No.: 11/649,928

(22) Filed: Jan. 4, 2007

Related U.S. Application Data
(63) Continuation-in-part of application No. 11/590,210, filed on Oct. 30, 2006.
Application No. 11/590,210 is a continuation-in-part of application No. 11/494,232, filed on Jul. 26, 2006.
Application No. 11/494,232 is a division of application No. 10/243,795, filed on Sep. 13, 2002, now Pat. No. 7,091,310.
(63) Continuation-in-part of application No. 11/393,769, filed on Mar. 29, 2006.
Application No. 11/393,769 is a division of application No. 10/222,703, filed on Aug. 16, 2002, now abandoned.
Application No. 10/222,703 is a continuation-in-part of application No. 10/086,177, filed on Feb. 26, 2002.
Application No. 10/086,177 is a continuation-in-part of application No. 09/835,107, filed on Apr. 12, 2001, now abandoned.
(63) Continuation-in-part of application No. 11/388,542, filed on Mar. 24, 2006.
(63) Continuation-in-part of application No. 10/945,674, filed on Sep. 20, 2004.
Application No. 10/945,674 is a continuation of application No. 09/852,424, filed on May 9, 2001, now abandoned.
(63) Continuation-in-part of application No. 10/932,208, filed on Aug. 31, 2004.
Application No. 10/932,208 is a continuation-in-part of application No. 10/243,795, filed on Sep. 13, 2002, now Pat. No. 7,091,310.
(60) Provisional application No. 60/232,425, filed on Sep. 14, 2000.
(60) Provisional application No. 60/373,629, filed on Apr. 17, 2002.
(60) Provisional application No. 60/373,628, filed on Apr. 17, 2002.
(60) Provisional application No. 60/205,467, filed on May 19, 2000.
(60) Provisional application No. 60/755,859, filed on Jan. 4, 2006.

(30) Foreign Application Priority Data
Feb. 23, 2001 (CA) 2,335,109
May 9, 2000 (CA) 2,305,787
Apr. 12, 2000 (CA) 2,305,036
Nov. 10, 2006 (CA) PCT/CA06/01848

(51) Int. Cl. A61K 38/19; A61K 39/395; C07K 14/52; C07K 16/24

(52) U.S. Cl. 424/85.1; 424/145.1; 530/351; 530/388.23

(57) ABSTRACT

The present invention generally relates to the design, preparation, derivation, and use of mimetics of CXC chemokines (CXCL1-CXCL17) in the prevention, treatment, and ameliorization of a wide variety of diseases and disorders. Generally speaking, this invention is directed to the design, synthesis, and use of chemokine analogs which bind to CXC chemokine receptors CXCR1-CXCR7, such that the analogs can be designed to affect the activity of the receptor, either as an agonist or an antagonist. The analogs can be useful for treating a wide variety of diseases and disorders, and can also serve as an adjunct to the treatment of a variety of diseases and disorders.

### CROSS-REFERENCE

This application is:

a continuation-in-part of U.S. patent application Ser. No. 11/590,210, filed Oct. 30, 2006, which is a continuation-in-part of U.S. patent application Ser. No. 11/494,232, filed Jul. 26, 2006, which is a divisional of U.S. patent application Ser. No. 10/243,795, filed Sep. 13, 2002;

a continuation-in-part of U.S. patent application Ser. No. 11/393,769, filed Mar. 28, 2006, which is a divisional of U.S. patent application Ser. No. 10/222,703, filed Aug. 16, 2002, which is a continuation-in-part of U.S. patent application Ser. No. 10/086,177, filed Feb. 26, 2002, which is a continuation-in-part of U.S. patent application Ser. No. 09/835,107, filed Apr. 12, 2001, which claims the benefit of U.S. Provisional Application No. 60/232,425, filed Sep. 14, 2000, Canadian Application Nos. 2,335,109, filed Feb. 23, 2001, and 2,305,036, filed Apr. 12, 2000; wherein, U.S. patent application Ser. No. 10/222,703, filed Aug. 16, 2002, claims the benefit of U.S. Provisional Application Nos. 60/373,628, filed Apr. 17, 2002, and 60/373,629, filed Apr. 17, 2002;

a continuation-in-part of U.S. patent application Ser. No. 11/388,542, filed Mar. 24, 2006;

a continuation-in-part of U.S. patent application Ser. No. 10/945,674, filed Sep. 20, 2004, which is a continuation of Ser. No. 09/852,424, filed May 9, 2001, which claims the benefit of U.S. Provisional Application No. 60/205,467, filed May 19, 2000; wherein, U.S. patent application Ser. No. 10/945,674, filed Sep. 20, 2004, claims the benefit of Canadian Application No. 2305787, filed May 9, 2000;

a continuation-in-part of U.S. patent application Ser. No. 10/932,208, filed Aug. 31, 2004, which is a continuation-in-part of U.S. patent application Ser. No. 10/243,795, filed Sep. 13, 2002; and,

claims the benefit of U.S. Provisional Application No. 60/755,859, filed Jan. 4, 2006; and PCT Application No. PCT/CA2006/001848, filed Nov. 10, 2006, which claims the benefit of U.S. Provisional Application No. 60/735,186, filed Nov. 10, 2005.

wherein, each of the references listed above in this cross-reference are hereby incorporated herein by reference in its entirety.

### SEQUENCE LISTING

The instant application contains a sequence listing which has been submitted as a paper copy and a computer readable format that is hereby incorporated herein by reference in its entirety.

### BACKGROUND

1. Field of the Invention

This invention relates to the preparation, design, derivation, and use of peptide agonists and antagonists of CXC chemokines.

2. Description of the State-of-the-Art

Receptors are macromolecules involved in chemical signaling between and within cells; they may be located on the cell surface membrane or within the cytoplasm. Activated receptors directly or indirectly regulate cellular biochemical processes (e.g., ion conductance, protein phosphorylation, DNA transcription, etc.) Molecules that bind to a receptor are called ligands, and identification of molecules that can control receptor activity can lead to new and desirable drugs. A ligand may activate or inactivate a receptor; activation may either increase or decrease a particular cell function, and each ligand may interact with multiple receptor subtypes. Few if any drugs are absolutely specific for one receptor or subtype, but most have relative selectivity. Selectivity is the degree to which a drug acts on a given site relative to other sites and relates largely to the physicochemical binding of the drug to cellular receptors.

Chemokines, a family of small cytokines, or proteins secreted by cells, potential sources of drugs because they are ligands that bind to cellular receptors. Chemokines induce directed chemotaxis in nearby responsive cells, hence the name chemotactic cytokines. Some chemokines are considered pro-inflammatory and can be induced during an immune response while others are considered homeostatic. All chemokines have molecular masses of between 8 and 10 kDa and are approximately 20-50% identical in that they share about 20-50% gene sequence and amino acid sequence homology with each other and share common tertiary structures. Their receptors are all integral membrane proteins containing seven membrane-spanning helices which are coupled to G proteins. All chemokines possess a number of conserved cysteine residues involved in intramolecular disulfide bond formation.

Without intending to be bound by any theory or mechanism of action, chemokines have been recognized as chemotactic agents that recruit leukocytes to the sites of injuries and have been found to have a wide variety of potential therapeutic uses. Chemokines have been found to participate in increasing the hemocrit, mobilizing stem cells, or in assisting in vaccine production or otherwise stimulating the immune system to effectuate tumor destruction. For example, the CXC chemokines CXCL9 and CXCL11 have been shown to be natural antagonists for the receptor CCR3 (Loetscher et al., J. Bio. Chem 276:2986-91, 2001); useful in improving asthma symptoms following intravenous injection (Zimmermann et al., J. Allaergy Clin. Immunol. 111: 227-242, 2003); useful in mobilizing stem cells (Gazitt, Y., J. Hematother Stem Cell Res 10:229-36, 2001; Hattori et al., Blood 97:3354-59, 2001); and useful in enhancing anti-tumor immunity (Nomura et al., Int. J. Cancer 91:597-606, 2001; Mach and Dranoff, Curr. Opin. Immunol. 12:571-75, 2000). Other aspects and roles of modulating chemokine function are reviewed in Schwarz and Wells (Schwarz and Wells, Nat. Rev. Drug Discov. 1:347-58, 2002). Chemokines have also been proven useful in facilitating gene therapy. Glimm and colleagues, for example, reported that one chemokine, SDF-1, arrests hematopoietic stem cell cycling, allowing for a better transfection of these cells with gene constructs for the purpose of gene therapy (Glimm H. et al., “Ex vivo treatment of proliferating human cord blood stem cells with stroma-derived factor-1 enhances their ability to engraft NOD/SCID mice,” Blood 99(9):3454-57, 2002).

Inflammatory chemokines are released from a wide variety of cells in response to bacterial infection, viruses, and agents that cause physical damage such as, for example, silica or the urate crystals that occur in gout. They function mainly as chemoattractants for leukocytes, recruiting monocytes, neutrophils and other effector cells from the blood to sites of infection or damage. Chemokines can be released by many different cell types and serve to guide cells involved in innate immunity and also the lymphocytes of the adaptive immune system. The cells that are attracted by chemokines tend to follow a signal of increasing chemokine concentration to the site of infection or tissue injury. Some chemokines also have roles in the development of lymphocytes, migration and angiogenesis (the growth of new blood vessels).

Most chemokines have four characteristic cysteines (Cys), and members of the chemokine family are categorized into four groups: (1) the CC chemokines (β-chemokines) with two adjacent cysteines near the amino terminus of the protein, (2) the C chemokines (γ chemokines), (3) the CX₃C chemokines (δ chemokines), and (4) the CXC chemokines (α-chemokines) in which the cysteines are separated by an amino acid. The four groups of chemokines act on different receptors, and each class has a characteristic function. For example, the α-chemokines are potent chemoattractants and activators of leukocytes such as neutrophils, whereas the β-chemokines are also potent chemoattractants and activators of monocytes.

Although similar in structure, the α and β chemokines have a low sequence homology of about 30-35% and, as such, are distinctive in their functions—the α chemokines cannot activate monocytes and the β chemokines have no effect on neutrophils. Since two disulfide bonds are characteristically formed between the first and third cysteine and between the second and fourth cysteine, it has generally been assumed that the disulfide bridges among four cysteines were required. See Clark-Lewis et al., J. Biol. Chem. 269:16075-16081, (1994). However, exceptions have been reported. For example, lymphotactin has only two cysteine residues, allowing only one disulfide bond. Regardless, lymphotactin manages to retain a functional structure with only the single disulfide bond.

The CC chemokines (β-chemokines), CCL1-CCL28, bind to CC chemokine receptors, of which ten have been discovered to date and are designated CCR1-CCR10. These receptors are expressed on the surface of different cell types allowing their specific attraction by the chemokines. Using this mechanism, the CC chemokines, such as RANTES, MIP-1-alpha, MCP-1, generally function as chemoattractants for monocytes, basophils, eosinophils, and T-cells but not neutrophils. Moreover, the CC chemokines induce the migration of monocytes and other cell types such as NK cells and dendritic cells. An example of a CC chemokine is monocyte chemoattractant protein-1 (MCP-1) which induces monocytes to leave the bloodstream and enter the surrounding tissue, becoming tissue macrophages. CCL28 attracts T cells and B cells that express CCR10, and eosinophils that express CCR3. It has also been implicated in anti-microbial activity. CCR5, or chemokine (C—C motif) receptor 5, binds RANTES/CCL5.

The C chemokines (γ-chemokines) lymphotactin-α (CL-1) and lymphotactin-β (CL-2) are thought to attract T cell precursors to the thymus. The CX3C chemokine (δ-chemokine) fractalkine (CX₃CL1) is both secreted and tethered to the surface of the cell that expresses it, thereby serving as both a chemoattractant and as an adhesion molecule.

The CXC chemokines (α-chemokines) have tremendous therapeutic potential as agonists and antagonists of cellular response and, thus, are the subject of the present application. The CXC subfamily has been divided into two groups depending on the presence of the ELR motif (Glu-Leu-Arg) preceding the first cysteine: the ELR⁺-CXC chemokines and ELR⁻-CXC chemokines (see, e.g., Clark-Lewis, supra, and Belperio et al., “CXC Chemokines in Angiogenesis,” J. Leukoc. Biol. 68:1-8, 2000). The ELR⁺-CXC chemokines (also known as ELR-CXC chemokines because they contain the ELR motif) are known to attract and activate human neutrophils in vitro at low nanomolar concentrations and induce neutrophils recruitment in vivo, whereas the ELR⁻-CXC chemokines (also known as non-ELR-CXC chemokines because they do not contain the ELR motif) are not known to be neutrophil chemoattractants but rather a chemoattractant for lymphocytes.

The ELR-CXC chemokines, such as IL-8, are generally strong neutrophil chemoattractants while the non-ELR chemokines such as, for example, IP-10 and SDF-1, predominantly recruit lymphocytes. All ELR⁺ CXC chemokines, including growth-regulated oncogene (GRO-α, -γ and -γ/CXCL1-CXCL3), ENA-78 (CXCL5), granulocyte chemotactic protein (GCP-2/CXCL6), neutrophil-activating protein 2 (NAP-2/CXCL7) and IL-8, stimulate endothelial cell chemotaxis in vitro and angiogenesis in vivo. Moreover, it is accepted that different functionalities can exist from chemokine activity. For example, the ability of ELR⁺ chemokines to induce angiogenesis appears to be independent from their ability to recruit inflammatory cells.

As such, the class that a chemokine falls into does not provide complete predictability about the scope of the chemokine's activity. For example, one non-ELR-CXC chemokine actually that actually stimulates, rather than inhibits, angiogenesis is stromal-derived factor 1 (SDF-1/CXCL12). The SDF-1 receptor CXCR4 is expressed on endothelial cells, which undergo chemotaxis in response to SDF-1. Levels of mRNA for the SDF-1 receptor on human endothelial cells are upregulated in response to vascular endothelial growth factor (VEGF) and basic fibroblast growth factor (bFGF), which are non-chemokine angiogenic factors. SDF-1 has been shown to induce angiogenesis from cross-sections of leukocyte-free rat aorta in vitro, and the formation of capillary-like structures by endothelial cells in culture. Another difference between SDF-1 and the non-ELR-CXC chemokines that inhibit angiogenesis is that the non-ELR CXC chemokines are induced by IFN and SDF-1 is not.

The receptors for the CXC chemokines are G-protein coupled seven-transmembrane receptors. These receptors have been named CXCR1-CXCR7. The receptors are listed in Table 1, along with corresponding chemokine ligands.

TABLE 1

| CXC chemokine | CXC chemokine | |
|---|---|---|
| receptors | ligands | Cell of interest |
| CXCR1 | CXCL2, 3, 5, | PMNs, monocytes, |
| | 6, 7, 8 | astrocytes, endothelia, |
| | | mast cells |
| CXCR2 | CXCL1, 2, 3, | PMNs, monocytes, |
| | 5, 6, 7, 8 | eosinophils, endothelia, |
| | | mast cells |
| CXCR3 | CXCL9, 10, 11 | T cells, B cells, NK cells, |
| (CXCR3B) | (CXCL4, | mesangial cells, smooth |
| | CXCL10) | muscle cells, endothelia |
| CXCR4 | CXCL12 | Hematopoietic progenitors, |
| | | T cells, immature |
| | | DCs, monocytes, B cells, |
| | | PMNs, platelets, astrocyte, |
| | | endothelia |
| CXCR5 | CXCL13 | T cells, B cells, astrocytes |
| | CXCL14 | endothelial cells have low |
| | | affinity receptors for BRAK, |
| | | immature monocyte-derived |
| | | dendritic cells (iDCs) have |
| | | high affinity receptors for |
| | | BRAK |
| | CXCL15 | (Expressed by lung tissue) |
| CXCR6 | CXCL16 | Memory T-cells |
| CXCR7 | CXCL12 | Hematopoietic progenitors, |
| | | T cells, immature |
| | | DCs, monocytes, B cells, |
| | | PMNs, platelets, astrocyte, |
| | | endothelia |

The CXCL1-CXCL17 CXC Chemokines

The CXCL1 chemokine is also known as growth-related oncogene-alpha (GRO-α), or melanoma growth stimulatory activity-alpha (MGSA-α), or neutrophil activating protein-3 (NAP-3) was first identified in 1989 as a chemokine with the ability to specifically activate neutrophils. May play a role in inflammation and exerts its effects on endothelial cells in an autocrine fashion. In vitro, the processed forms GRO-α (4-73), GRO-α (5-73) and GRO-α (6-73) show a 30-fold higher chemotactic activity.

The CXCL2 chemokine is also known as growth-related oncogene-beta (GRO-β), or melanoma growth stimulatory activity-alpha (MGSA-β), or macrophage inflammatory protein 2-alpha (MIP2-α) was first identified in 1991. Produced by activated monocytes and neutrophils and expressed at sites of inflammation. Hematoregulatory chemokine, which, in vitro, suppresses hematopoietic progenitor cell proliferation. GRO-β (5-73) shows a highly enhanced hematopoietic activity. GRO-β (5-73) is available under the name Garnocestim as immunomodulator. It is used prior to hematopoietic transplantation for peripheral blood stem cell mobilization and reduction of incidence, duration, and/or severity of chemotherapy induced cytopenias.

The CXCL3 chemokine is also known as growth-related oncogene-gamma (GRO-γ), or melanoma growth stimulatory activity-gamma (MGSA-γ), or macrophage inflammatory protein 2-beta (MIP2-β) was first identified in 1991. Has chemotactic activity for neutrophils. May play a role in inflammation and exert its effects on endothelial cells in an autocrine fashion. In vitro, the processed form GRO-γ (5-73) shows a fivefold higher chemotactic activity for neutrophilic granulocytes. Among other applications, the CXC chemokines GRO-gamma/CXCL3 has profound angiogenic potential mediated through the CXCR2 receptor.

The CXCL4 chemokine is also known as platelet-factor-4 (PF-4). Platelet factor-4 is a 70-amino acid protein that is released from the alpha-granules of activated platelets and binds with high affinity to heparin. Its major physiologic role appears to be neutralization of heparin-like molecules on the endothelial surface of blood vessels, thereby inhibiting local antithrombin III activity and promoting coagulation. As a strong chemoattractant for neutrophils and fibroblasts, PF4 probably has a role in inflammation and wound repair. See Eisman, R., et al. Blood 76: 336-344 (1990).

The CXCL5 chemokine is also known as epithelial-derived neutrophil-activating protein 78 (ENA-78), or neutrophil-activating peptide. ENA-78 was first identified in 1995 and is known as a chemotactic for neutrophil granulocytes. N-terminal processed forms ENA-78(8-78) and ENA-78(9-78) are produced by proteolytic cleavage after secretion from peripheral blood monocytes. Among other applications, the CXC chemokine ENA-78/CXCL5 has profound angiogenic potential mediated through the CXCR2 receptor. Data suggest that CXCL5 production contributes to both enhanced proliferation and invasion of squamous cell carcinomas and that targeting of specific pathways that include CXCL5 may represent a potential therapeutic modality for these lesions. Research has also shown that granulocyte colony-stimulating factor (G-CSF) stimulated the production of ENA-78 by neutrophils and that ENA-78 might promote the accumulation of neutrophils that had migrated from the intravascular space into inflammatory tissues. CXCL5 expression in bone cells has implications for inflammatory bone diseases such as arthritis and periodontal disease. For example, ENA-78 has been shown to contribute to the angiogenic activity found in the inflamed RA joint.

The CXCL6 chemokine is also known as granulocyte chemotactic protein 2 (GCP-2), or chemokine alpha 3 (CKA-3) was first identified in 1993. It is a chemotactic factor for neutrophil granulocytes. GCP-2 binds the CXCR1 and CXCR2 receptors, along with IL-8, both of which are co-induced in microvascular endothelial cells after stimulation with pro-inflammatory stimuli. Moreover, GCP-2 is considered to be a proangiogenic and has been shown to have an adverse affect on the inflammatory process of asthma.

The CXCL7 chemokine is also known as neutrophil-activating peptide 2 (NAP-2), connective tissue-activating peptide III (CTAP-III), or beta-thromboglobulin (Beta-TG) and was first identified in 1986. NAP-2 timulates DNA synthesis, mitosis, glycolysis, intracellular cAMP accumulation, prostaglandin E2 secretion, and synthesis of hyaluronic acid and sulfated glycosaminoglycan. It also stimulates the formation and secretion of plasminogen activator by human synovial cells. NAP-2 is a ligand for CXCR1 and CXCR2, weakly competing with IL-8; and, NAP-2, NAP-2(73), NAP-2(74), NAP-2(1-66), and the more potent NAP-2(1-63) are chemoattractants and activators for neutrophils. NAP-2 also appears to play a role in atherosclerosis, having potential therapeutic applications in another widespread disease.

The CXCL8 chemokine is also known as interleukin-8 (IL-8) and has been shown to have many potential therapeutic applications. They have been shown to have both anti-tumor and anti-infective therapeutic activity. IL-8s have shown to be responsible for the recruitment and activation of leukocytes and a mediator of acute inflammatory response. They have an ability, for example, to stimulate T-cell chemotaxis. The IL-8s have shown a profound angiogenic potential that is mediated through the CXCR2 receptor and have demonstrated an ability to contribute to the angiogenic activity found in the inflamed RA joint.

The CXCL9 chemokine is also known as gamma interferon-induced monokine (MIG) was first identified in 1994. Cytokine that affects the growth, movement, or activation state of cells that participate in immune and inflammatory response. Chemotactive for activated T-cells. Binds to CXCR3. Induced by interferon gamma. The induction is enhanced by TNF-alpha in dermal fibroblasts and vein endothelial cells.

The CXCL10 chemokine is also known as interferon-inducible protein-10 (IP-10). Interferon-inducible protein-10 (IP-10 or CXCL10) is induced by interferon-gamma and TNF-alpha, and is produced by keratinocytes, endothelial cells, fibroblasts and monocytes. IP-10 is thought to play a role in recruiting activated T cells to sites of tissue inflammation (Dufour, et al., “IFN-gamma-inducible protein 10 (IP-10; CXCL10)-deficient mice reveal a role for IP-10 in effector T cell generation and trafficking,” J Immunol., 168:3195-204, 2002). In addition, IP-10 may play a role in hypersensitivity. It may also play a role in the genesis of inflammatory demyelinating neuropathies (Kieseier, et al., “Chemokines and chemokine receptors in inflammatory demyelinating neuropathies: a central role for IP-10,” Brain 125:823-34, 2002).

The CXCL11 chemokine is also known as interferon-inducible T-cell alpha chemoattractant (I-TAC), or interferon-gamma-inducible protein 9 (IP-9), or H174, or Beta-R1 was first identified in 2000. It is chemotactive for interleukin-activated T cells but not unstimulated T cells, neutrophils or monocytes; induces calcium release in activated T cells; binds to CXCR3; may play an important role in CNS diseases which involve T cell recruitment; and may play a role in skin immune responses. There are high levels present in peripheral blood leukocytes, pancreas and liver astrocytes; moderate levels present in thymus, spleen and lung; and low levels present in placenta, prostate and the small intestine. CXCL11 is also found in epidermal basal layer keratinocytes in skin disorders and is induced by interferon gamma and interferon beta, where induction by IFN-gamma is enhanced by TNF-alpha in monocytes, dermal fibroblasts and endothelial cells, and by IL-1 in astrocytes.

The CXCL12 chemokine is also known as stromal-derived factor one (SDF-1). SDF-1 demonstrates in vitro activity with lymphocytes and monocytes but not neutrophils and is a highly potent in vivo chemoattractant for mononuclear cells. SDF-1 has been shown to induce intracellular actin polymerization in lymphocytes, and to induce a transient elevation of cytoplasmic calcium in some cells. SDF-1 activates leukocytes and is often induced by proinflammatory stimuli such as lipopolysaccharide, TNF-α, or IL-1. SDF-1 can mobilize and increase the number of circulating neutrophils, for example, in patients undergoing chemotherapy to facilitate blood cell recovery. In this example, intravenous injection of the CXCR-agonist may facilitate the creation of an artificial chemotactic gradient, which may facilitate an immune response in the target tissue (in this case, blood)

The CXCL13 chemokine is also known as B cell-attracting chemokine 1 (BCA-1), or B lymphocyte chemoattractant (BLC), or ANGIE was first identified in 2000. BCA-1 is a chemotactive for B lymphocytes but not for T-lymphocytes, monocytes and neutrophils; does not induce calcium release in B lymphocytes; binds to BLR1/CXCR5; and has its highest levels in the liver, spleen, lymph node, appendix, and stomach. Low levels of BCA-1 are found in salivary glands, mammary glands, and fetal spleen.

The CXCL14 chemokine is also known as chemokine BRAK was first identified in 2000. BRAK/CXCL14 is expressed at the mRNA level in certain normal tissues, such as the heart, brain, placenta, lung, liver, skeletal muscle, kidney and pancreas; but it is absent from many established tumor cell lines and human cancers. BRAK expression in normal and tumor specimens from patients with squamous cell carcinoma (SCC) of the tongue and used recombinant BRAK (rBRAK) showed abundant expression of BRAK protein in suprabasal layers of normal tongue mucosa but an absence of such expression in tongue SCC. BRAK protein is also found to be expressed strongly by stromal cells adjacent to tumors and is recognized as a potent inhibitor of in vivo angiogenesis stimulated by multiple angiogenic factors, including interleukin 8, basic fibroblast growth factor, and vascular endothelial growth factor. As such, a loss of BRAK expression from tumors may facilitate neovascularization and possibly contribute to immunologic escape. In vitro, rBRAK has been shown to block endothelial cell chemotaxis at concentrations as low as 1 nmol/L, suggesting a strong potential therapeutic use of BRAK for angiogenesis inhibition. Although endothelial cells only have low affinity receptors for BRAK, human immature monocyte-derived dendritic cells (iDCs) have high affinity receptors for rBRAK (i.e., Kd, 2 nmol/L). Moreover, rBRAK is chemotactic for iDCs at concentrations ranging from 1 to 10 nmol/L.

The CXCL15 chemokine is a mouse CXC chemokine known as lungkine and was first identified in 2000. Lungkine is chemotactic for neutrophils and appears to be specifically expressed in the lung. See Rossi, et al. J. Immunol. 162:5490-5497 (1999). Expression of lungkine in fetal lungs has been found to exist at low levels, and increased levels can be induced by inflammation in the lung. As such, lungkine may be involved in lung-specific neutrophil trafficking during normal and inflammatory conditions, having expression that is restricted to the lung, produced by bronchoepithelial cells, and released into the airways.

The CXCL16 chemokine is also known as a scavenger receptor for phosphatidylserine and oxidized low density lipoprotein (SR-PSOX) was first identified in 2003. Acts as a scavenger receptor on macrophages, which specifically binds to OxLDL (oxidized low density lipoprotein), suggesting that it may be involved in pathophysiology such as atherogenesis. SRPSOX induces a strong chemotactic response, calcium mobilization, and binds to CXCR6/Bonzo. SRPSOX is expressed in T-cell areas, the spleen, lymph nodes, lung, kidney, small intestine, and thymus. It is expressed weakly in the heart and liver, and there is no expression in the brain and bone marrow.

The CXCL17 chemokine is also known as DMC (dendritic cell and monocyte chemokine-like protein), which attracts dendritic cells and monocytes. DMC is predicted to have an IL-8-like chemokine fold and to be structurally and functionally related to CXCL8 and CXCL14. DMC induces migration of monocytes and immature dendritic cells, and expression studies show that DMC is constitutively expressed in the lung, suggesting a potential role for DMC in recruiting monocytes and dendritic cells from blood into lung parenchyma.

Accordingly, CXC chemokines participate in many diseases that include, but are not limited to, inflammation and/or conditions associated with immune/autoimmune responses. They also play a very important role in normal homeostasis, including lymphoid development and migration, and the growth of bone. As a result, the CXC chemokines have important potential therapeutic applications and, as such, one of skill will appreciate analogs of CXC chemokines that can be readily designed and manufactured to serve as either agonists or antagonists of CXC chemokine receptors CXCR1-CXCR7, providing society with an additional source of potent new therapeutics.

### SUMMARY OF THE INVENTION

The inventions taught herein are generally directed to the design, preparation, derivation, and use of mimetics of CXC chemokines in the prevention, treatment, and ameliorization of diseases and disorders. The CXC chemokine analogs bind to CXC chemokine receptors and can be designed to affect the activity of the receptor, either as an agonist or an antagonist.

In some embodiments, the invention includes a composition comprising an analog of a native CXC chemokine selected from a group consisting of CXCL1, CXCL2, CXCL3, CXCL5, CXCL6, CXCL7, CXCL9, CXCL11, CXCL13, CXCL14, CXCL15, CXCL16, and CXCL17, wherein the analog has a length ranging from about 20 to about 37 amino acids. The analog comprises an N-terminal region comprising a first conserved sequence consisting of about 13 to 17 of the first 17 of the native CXC chemokine N-terminal residues, or conservatively modified variants thereof, or a sequence having at least 90% homology to the first conserved sequence and capable of binding to a cellular receptor that binds to the first conserved sequence. The analog also comprises a C-terminal region comprising a second conserved sequence consisting of about 6 to 16 of the last 16 of the native CXC chemokine C-terminal residues; or conservatively modified variants thereof, or a sequence having at least 90% homology to the second conserved sequence and capable of binding to a cellular receptor that binds to the second conserved sequence.

And, the analog further comprises a linker selected from a group consisting of from 1 to 4 natural or non-natural amino acids having the following structure: 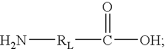
wherein, R_{L} is selected from a group consisting of saturated and unsaturated aliphatics and heteroaliphatics consisting of 20 or fewer carbon atoms that are optionally substituted with (i) a hydroxyl, carboxyl, amino, amido, or imino group, or (ii) an aromatic group having from 5 to 7 members in the ring; and —(CH₂)ₙ—, wherein n is an integer ranging from 1 to 20. The analog is optionally modified with a modifier selected from a group consisting of a poly(ethylene glycol) or derivative thereof, a glycosaminoglycan, a diagnostic label, a radioactive group, an acyl group, an acetyl group, a peptide, a modifier capable of reducing the ability of the analog to act as a substrate for aminopeptidases, and a modifier capable of reducing the ability of the analog to act as a substrate for carboxypeptidases.

In some embodiments, the analog is a non-ELR-CXC chemokine analog; wherein, the first conserved sequence consists of about 13 to 17 of the first 17 of the native CXC chemokine N-terminal residues, or conservatively modified variants thereof, or a sequence having at least 90% homology to the first conserved sequence and capable of binding to a cellular receptor that binds to the first conserved sequence, wherein the first conserved sequence does not include an ELR motif. In these embodiments, the second conserved sequence consists of about 6 to 16 of the last 16 of the native CXC chemokine C-terminal residues, or conservatively modified variants thereof, or a sequence having at least 90% homology to the second conserved sequence and capable of binding to a cellular receptor that binds to the second conserved sequence.

In some embodiments, the analog is an ELR-CXC chemokine analog, wherein the first conserved sequence consists of about 13 to 17 of the first 17 of the native CXC chemokine N-terminal residues, or conservatively modified variants thereof, or a sequence having at least 90% homology to the first conserved sequence and capable of binding to a cellular receptor that binds to the first conserved sequence, wherein the first conserved sequence includes an ELR motif. In these embodiments, the second conserved sequence consisting of about 6 to 16 of the last 16 of the native CXC chemokine C-terminal residues, or conservatively modified variants thereof, or a sequence having at least 90% homology to the second conserved sequence and capable of binding to a cellular receptor that binds to the second conserved sequence.

In some embodiments, the C-terminal region of the analog is cyclized. And, in some embodiments, the linker is 11-aminoundecanoic acid or a combination of 4 natural amino acids, wherein the linker optionally contains an amino acid having a side chain bearing positive charge.

In some embodiments, the invention is directed to a method of increasing the activity of a cell having a CXC receptor comprising binding the CXC receptor to an analog taught herein, wherein the increase is relative to the activity of the cell in the absence of the analog. In some embodiments, the invention is directed to a method of decreasing the activity of a cell having a CXC receptor comprising binding the CXC receptor to an analog taught herein, wherein the increase is relative to the activity of the cell in the absence of the analog. In some embodiments, the invention is directed to an antibody produced using an analog taught herein as the antigen.

### BRIEF DESCRIPTION OF THE FIGURES

FIG. 1 illustrates the induction of [Ca²+]i mobilization by select IP-10 analogs at a concentration of 100 μM according to some embodiments.

FIGS. 2A and 2B shows the incubation of SUP-T1 cells with SDF-1 according to some embodiments.

FIG. 3 shows a competitive dose response for binding to the SDF-1 receptor by native SDF-1 and the CXCR4 agonists (competing ligands) against ¹²⁵I-SDF-1 according to some embodiments.

FIG. 4 shows the CXCR2 receptor binding of the IL-8 mimetics as competing ligands according to some embodiments.

FIG. 5 shows the response of circulating neutrophil counts to the administration of varying doses of the test mimetic following one hour of treatment according to some embodiments.

FIG. 6 describes the kinetics of the rise in circulating neutrophil counts in response to the administration of the test mimetic according to some embodiments.

FIG. 7 shows the response of circulating haematopoietic progenitor/stem cells to the administration of varying doses of the test mimetic according to some embodiments.

FIG. 8 describes the kinetics of the rise in haematopoietic progenitor/stem cells in response to the administration of the test mimetic according to some embodiments.

FIGS. 9-11 illustrate the efficacy of the PF-4 analogs as agonists according to some embodiments.

### DETAILED DESCRIPTION OF THE INVENTION

The present invention generally relates to the design, preparation, derivation, and use of mimetics of CXC chemokines in the prevention, treatment, and ameliorization of diseases and disorders. Generally speaking, this invention is directed to the design, synthesis, and use of chemokine analogs which bind to CXC chemokine receptors, such that the analogs can be designed to affect the activity of the receptor, either as an agonist or an antagonist.

In one aspect, this invention is directed to the synthesis or use of CXC chemokine analogs which bind to receptors for any of the 17 CXC chemokines to modulate cellular activity. The term “modulates” refers to altering the function or activity of a chemokine receptor by contacting it with a chemokine or chemokine analog and thus increasing or decreasing the probability that a complex forms between the receptor and a natural binding partner. The chemokine analogs can be designed to increase or decrease the probability that such a complex forms between a chemokine receptor and a natural binding partner, for example, and the relative effect can, in some embodiments, depend on the concentration of the chemokine analog exposed to the receptor.

The term “CXC chemokine receptor” refers to a CXC chemokine receptor as the term is used by one skilled in the art, as well as any other chemical moiety, such as a peptide, capable of binding to a CXC chemokine analog.

The term “natural binding partner” refers to G proteins, polypeptides, lipids, small molecules, or nucleic acids that bind to CXC chemokine receptors in cells or in the extracellular environment. The term natural binding partner includes a substrate to be acted upon by the CXC chemokine receptor. A change in the interaction between a CXC chemokine receptor and a natural binding partner can result in a decreased or increased activity of the CXC chemokine receptor.

The terms “activate,” “activated,” “activating,” and “activation” can refer to an interaction between a CXC chemokine analog and a CXC chemokine receptor that increases the cellular or extracellular function of a CXC chemokine receptor. The CXC chemokine receptor function can be the interaction with a natural binding partner and can result in a catalytic activity. The term “inhibit,” “inhibited,” and “inhibiting” refers to decreasing the cellular or extracellular activity of the CXC chemokine receptor.

The terms “complex,” “complexed,” and “complexing” can refer to an assembly of at least two molecules bound to one another. A signal transduction complex often contains at least two protein molecules bound to one another. In some embodiments, a protein tyrosine receptor protein kinase, GRB2, SOS, RAF, and RAS assemble to form a signal transduction complex in response to a mitogenic ligand. In some embodiments, a CXC chemokine analog is bound to a CXC chemokine receptor. In other embodiments, a G protein bound to a CXC chemokine receptor.

The terms “contact,” “contacted,” and “contacting” can refer to combining a solution or a composition comprising the CXC chemokine or CXC chemokine analog with a liquid medium bathing the polypeptide or cells comprising a CXC chemokine receptor. The solution comprising the CXC chemokine or CXC chemokine analog may also comprise another component, such as dimethyl sulfoxide (DMSO), which can facilitate the uptake of the CXC chemokine or CXC chemokine analog into the cells of interest. The solution comprising the CXC chemokine or CXC chemokine analog may be added to the medium bathing the cells by utilizing a delivery apparatus, such as a pipette-based device or syringe-based device.

In most embodiments, however, the invention is directed to the synthesis, design, derivation, or use of CXC chemokin analogs of one or more of the 17 CXC chemokines. The native sequences of the 17 CXC chemokines are provided in the attached Sequence Listings as SEQ ID NOs:1-17:

The N-terminal region of CXC chemokines is involved in the binding and activating site of its receptor, as well as is the carboxy terminal region. The beta sheet structure that connects the two termini appears to play a role in the stabilization of the CXCR and assuring that the termini are in the proper conformation.

In most embodiments, the CXC chemokine analogs contain structures corresponding to various regions or portions of the native CXC chemokines, or conservatively modified variants thereof. In some embodiments, the CXC chemokine analogs comprise an N-terminal region and a C-terminal region joined together using a linker. In some embodiments, the amino acid residues of the CXC chemokine or chemokine analog can be cyclized, for example, by etherification of lysine and serine residues or by any other means described herein or known in the art. In some embodiments, the CXC chemokine analog comprises a sequence derived from the corresponding wild-type CXC chemokine but with one or more of the cysteines replaced with another amino acid, which can include any natural or non-natural amino acids. Some embodiments consist of linking from about 3 to about 17 amino acids of the wild-type N-terminal region to about 3 to about 17 amino acids of the wild-type C-terminal region directly with a linker. In most embodiments, the N-terminal can be acetylated and/or the C-terminal can be amidated.

In some embodiments, the regions selected from the N-terminal, internal and C-terminal regions may be 3, 4, 5, 6, 7, 8, 9, 10, 11, 12, 13, 14, 15, 16, 17, 20, 25, 30, 35, 40, 41, or 45 amino acids in length, and this length can be independent to the N-terminal region, C-terminal region, linker, or a combination thereof. In some embodiments, the CXC chemokine analogs range from about 12 to about 20, from about 20 to about 40, from about 20 to about 35, from about 21 to about 34, from about 21 to about 28, or any range therein, amino acids in length. In some embodiments, the analogs are a hybrid structure that includes a first region from one CXC chemokine and a second region from a different CXC chemokine, wherein the first and second regions are connected using a linker.

CXC Chemokine analogs of the invention are useful for treating or preventing inflammatory conditions, autoimmune disorders, cancer, graft rejection, bacterial infection, viral infection, vascular conditions (for example, atherosclerosis, restenosis, systemic lupus erythematosis, and ischemia-reperfusion), sepsis, tumorigenesis, and angiogenesis, gene therapy, stem cell mobilization, vaccine production, and blood cell recovery following chemotherapy. Inflammatory conditions can include acute and/or chronic inflammatory diseases. The CXC Chemokine analogs may assist in gene therapy, for example, by providing a means for arresting the cell cycle.

In some embodiments, the CXC chemokine analogs can be used for treatments that include, but are not limited to, treatment or management of arthritis, asthma, colitis/illeitis, psoriasis, atherosclerosis and the like; treatment or management of autoimmune conditions that include, but are not limited to, rheumatoid arthritis, multiple sclerosis and other autoimmunological diseases; treatment or management of cancers that include, but are not limited to, human malignancy/cancer cell metastasis and relapses; treatment or management in assisting with blood cell recovery that includes, but is not limited to, blood cell elevation after chemotherapy/radiotherapy and stem cell mobilization for transplantations; vaccine production that includes, but is not limited to, enhancement in humoral antibody production, increases in antigen presenting T-cells, increases in dendritic cells and immunological features known as vaccine induction; treatment or management of osteoporosis; or treatment or management of genetic disease through gene therapy.

In many embodiments, the therapeutic uses are effective because a CXC chemokine analog can be designed to act as an agonist or antagonist to a native CXC chemokine. The agonistic activity of the CXC chemokine analogs may include mimicking the biological activity normally induced by a native CXC chemokine. The antagonistic activity of the CXC chemokine analogs may include inhibiting the biological activity normally induced by a native CXC chemokine. In some embodiments, for example, the analog does not have to be an analog of the native chemokine in order to serve as an agonist or antagonist of a particular cellular function—an analog of a first native CXC chemokine can act as an agonist or antagonist with respect to the cellular activity normally induced by a second native CXC chemokine.

In some embodiments, the CXC chemokine analogs can be used to prepare vaccines, to enhance humoral antibody production, to increase antigen-presenting T-cells, to increase dendritic cells and immunological features known as vaccine induction, and combinations thereof. The term “antibody” can refer to any antibody-like molecule that has an antigen binding region, and includes antibody fragments such as Fab′, Fab, F(ab′)₂, single domain antibodies (DABs), Fv, scFv (single chain Fv), and the like. Techniques for preparing and using various antibody-based constructs and fragments are well known in the art as are techniques for preparing and characterizing antibodies. In some embodiments, the CXC chemokine analogs taught herein can be used as antigens to produce antibodies using methods well-known to those skilled in the art. In these embodiments, the antibody can be polyclonal or monoclonal. In some embodiments, the antibody is humanized.

CXC Chemokine Analogs

In this application, the products of the present invention can be referred to using various terms, including “analog,” “mimetic,” “peptide,” “polypeptide,” “chemokine analog,” “chemokine mimetic,” “chemokine derivative,” and the like. These terms, and others that would share the same meaning to one of skill, can be used interchangeably herein. The CXC chemokine analogs can comprise a sequence selected from any sequences taught herein and may comprise additional elements such as R-group substituents and a linker selected from the possibilities set forth herein. However, the analogs taught herein are not necessarily limited to the sequences taught herein, as they are taught by way of example. One of skill would still be operating within the scope of the invention by making obvious variations of the analogs using information known in the art to optimize the therapeutic effect, for example, by optimizing a result-effective variable, modifying an analog's structure for desired means of delivery to a subject, etc.

The term biological activity can refer to any physiological or biological response produced by a CXC chemokine or CXC chemokine analog, whether the response is manifested as a symptom in a subject, measurable using an in vivo laboratory test, traceable using a biomarker or the like, or measurable using an in vitro method. In some embodiments, the activity can refer to what is referred to in the scientific reports known in the art such as, for example, the activities referred to in Bruce, L. et al., “Radiolabeled Chemokine binding assays,” Methods in Molecular Biology (2000) vol. 138, pp 129-134; Raphaele, B. et al. “Calcium Mobilization,” Methods in Molecular Biology (2000) vol. 138, pp 143-148; and Paul D. Ponath et al., “Transwell Chemotaxis,” Methods in Molecular Biology (2000) vol. 138, pp 113-120. For example, a biological activity can include, but is not limited to, receptor binding, chemotaxis, calcium mobilization, cellular apoptosis, an increase or decrease in a symptom of a disease relative to the degree of the symptom present prior to administration of a chemokine analog, along with any other such ligand/receptor activities recognized by those skilled in the art as a physiological or biological response.

The amino acids are identified in the present application by the following conventional three-letter abbreviations shown in Table 2. The single letter identifier is provided for ease of reference. The three-letter abbreviations are generally accepted in the peptide art, recommended by the IUPAC-IUB commission in biochemical nomenclature, and are required by WIPO Standard ST.25:

TABLE 2

| | | |
|---|---|---|
| Alanine | A | Ala |
| Arginine | R | Arg |
| Asparagine | N | Asn |
| Aspartic acid | D | Asp |
| Cysteine | C | Cys |
| Glutamic acid | E | Glu |
| Glutamine | Q | Gln |
| Glycine | G | Gly |
| Histidine | H | His |
| Isoleucine | I | Ile |
| Ornithine | O | Orn |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| Norleucine | | NLeu |
| Other | | Xaa |

Furthermore, the peptide sequences are described using the generally accepted convention of placing the N-terminus on the left and the C-terminus on the right of the sequence listing as required by WIPO Standard ST.25. Amino acid substitutions are indicated using brackets and superscript numbers to indicate the position of the residue substituted. Cyclized regions are indicated using underlined residues to show the cyclic portion, as well as by using the term “cyclo” or the term “cyclic” to show the cyclized portion.

The following CXC chemokine analogs provide examples of analogs that can be used according to some embodiments of the present invention. In these embodiments, the analog can include a first conserved region and a second conserved region, wherein the first conserved region can include an N-terminal region, and the second conserved region can include a C-terminal region.

The N-terminal region can include a series of up to 17 of the first 17 amino acids of a native CXC chemokine, and the C-terminal region can include a series of up to 17 of the last 17 amino acids in the native chemokine. In some embodiments, the analog can comprise an N-terminal region having the first 15 residues of the native chemokine, and the C-terminal region can comprise the last 13 residues of the chemokine. In some embodiments, the first and second conserved regions can be linked using a linker:
(first conserved region)-[linker]-(second conserved region).

CXCL1, GRO-α, Compounds

In some embodiments, the CXCL1 (GRO-α) chemokine analogs include:

(SEQ ID NO:18)

R-X₀₁ X₀₂ X₀₃ X₀₄ X₀₅ X₀₆ X₀₇ X₀₈ X₀₉ X₁₀ X₁₁ X₁₂
X₁₃ X₁₄ X₁₅ X₁₆ [linker] Y₀₁ Y₀₂ Y₀₃ Y₀₄ Y₀₅ Y₀₆
Y₀₇ Y₀₈ Y₀₉ Y₁₀ Y₁₁ Y₁₂ Y₁₃ Y₁₄

(SEQ ID NO:19)

R-X₀₄ X₀₅ X₀₆ X₀₇ X₀₈ X₀₉ X₁₀ X₁₁ X₁₂ X₁₃ X₁₄ X₁₅
X₁₆ [linker] Y₀₁ Y₀₂ Y₀₃ Y₀₄ Y₀₅ Y₀₆ Y₀₇ Y₀₈ Y₀₉
Y₁₀ Y₁₁ Y₁₂ Y₁₃ Y₁₄

(SEQ ID NO:20)

R-X₀₅ X₀₆ X₀₇ X₀₈ X₀₉ X₁₀ X₁₁ X₁₂ X₁₃ X₁₄ X₁₅ X₁₆
[linker] Y₀₁ Y₀₂ Y₀₃ Y₀₄ Y₀₅ Y₀₆ Y₀₇ Y₀₈ Y₀₉ Y₁₀
Y₁₁ Y₁₂ Y₁₃ Y₁₄

(SEQ ID NO:21)

R-X₀₆ X₀₇ X₀₈ X₀₉ X₁₀ X₁₁ X₁₂ X₁₃ X₁₄ X₁₅ X₁₆
[linker] Y₀₁ Y₀₂ Y₀₃ Y₀₄ Y₀₅ Y₀₆ Y₀₇ Y₀₈ Y₀₉ Y₁₀
Y₁₁ Y₁₂ Y₁₃ Y₁₄

wherein,
X₀₁ is L- or D-Ala, Gly, L- or D-Phe, the preferred amino acid residues is L- or D-Ala;
X₀₂ is any natural or non natural amino acid different from L- or D-Cys, such as L- or D-Ser, L- or D-Thr, or L- or D-Tyr;
X₀₃ is L- or D-Val, L- or D-Leu, L- or D-Ile, L- or D-Pro, L- or D-Phe, L- or D-Tyr, L- or D-Ser, or L- or D-Thr;
X₀₄ is any natural or non natural amino acid residue different from L- or D-Cys residue, such as L- or D-Ala, Gly, or D-L-Phe;
X₀₅ is L- or D-Thr, L- or D-Ser, L- or D-Tyr, L- or D-Trp, or L- or D-His;
X₀₆ is any natural or non natural amino acid residue different from L- or D-Glx, L- or D-Asx, L- or D-Arg, L- or D-Lys, L- or D-His, L- or D-Tyr, L- or D-Ser, L- or D-Thr, such as L- or D-Glu, or L- or D-Asp;
X₀₇ is any natural or non natural amino acid residue different from L- or D-Cys residue, such as L- or D-Leu, L- or D-Ile, L- or D-L- or D-Val, or L- or D-Ala, L- or D-Phe, or L- or D-Tyr;
X₀₈ is any natural or non natural amino acid residue different from L- or D-Cys residue, such as L- or D-Arg, L- or D-Lys, L- or D-Orn- or D-Ala;
X₀₉ is L- or D-Cys, L- or D-Ala, L- or D-Phe, L- or D-His, L- or D-Trp, L- or D-Ser, L- or D-Thr, L- or D-Lys, L- or D-His, or L- or D-Tyr;
X₁₀ is any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, or L- or D-Trp;
X₁₁ is L- or D-Cys, L- or D-Ala, L- or D-Phe, L- or D-His, L- or D-Trp, L- or D-Ser, L- or D-Thr, L- or D-Lys, L- or D-His, or L- or D-Tyr;
X₁₂ is any natural or non natural amino acid residue different from L- or D-Cys residue, such as L- or D-Leu, L- or D-Ile, L- or D-L- or D-Val, or L- or D-Ala, L- or D-Phe, or L- or D-Tyr;
X₁₃ is any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, or L- or D-Trp;
X₁₄ is L- or D-Thr, L- or D-Ser, L- or D-Tyr, L- or D-Trp, or L- or D-His;
X₁₅ is any natural or non natural amino acid residue different from L- or D-Cys residue, such as L- or D-Leu, L- or D-Ile, L- or D-L- or D-Val, or L- or D-Ala, L- or D-Phe, or L- or D-Tyr;
X₁₆ is one or up to six natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, L- or D-Trp, L- or D-Lys, and L- or D-Gln, L- or D-(Gln-Gly), L- or D-(Gln-Gly-Ile), L- or D-(Gln-Gly-Ile-His), L- or D-(Gln-Gly-Ile-His-Pro), or L- or D-(Gln-Gly-Ile-His-Pro-Lys);

and wherein,
Y₀₁ is any natural or non natural amino acid residue different from L- or D-Cys residue, such as L- or D-Ile, L- or D-Ieu, L- or D-L- or D-Val, or L- or D-Ala, L- or D-Phe, L- or D-Tyr, or L- or D-Lys;
Y₀₂ is L- or D-Val, L- or D-Leu, L- or D-Ile, L- or D-Pro, L- or D-Phe, L- or D-Tyr, L- or D-Ser, L- or D-Thr, or L- or D-Lys;
Y₀₃ is any natural or non natural amino acid residue different from L- or D-Cys residue, such as L- or D-Lys, L- or D-Arg, L- or D-Orn- or D-Ala, or L- or D-Ile;
Y₀₄ is any natural or non natural amino acid residue different from L- or D-Cys residue, such as L- or D-Lys, L- or D-Arg, L- or D-Orn- or D-Ala, or L- or D-Ile;
Y₀₅ is any natural or non natural amino acid residue different from L- or D-Cys residue, such as L- or D-Ile, L- or D-Ieu, L- or D-L- or D-Val, or L- or D-Ala, L- or D-Phe, L- or D-Tyr, or L- or D-Glu;
Y₀₆ is any natural or non natural amino acid residue different from L- or D-Cys residue, such as L- or D-Ile, L- or D-Ieu, L- or D-L- or D-Val, or L- or D-Ala, L- or D-Phe, L- or D-Tyr, L- or D-Glu, or L- or D-Lys;
Y₀₇ is any natural or non natural amino acid residue different from L- or D-Glx, L- or D-Asx, L- or D-Arg, L- or D-Lys, L- or D-His, L- or D-Tyr, L- or D-Ser, L- or D-Thr, L- or D-Met, or L- or D-Nle;
Y₀₈ is any natural or non natural amino acid residue different from L- or D-Lys, L- or D-Met, L- or D-Arg, L- or D-Asx, L- or D-Glx, L- or D-His, L- or D-Tyr, L- or D-Ser, L- or D-Thr, L- or D-Leu, or L- or D-Ile;
Y₀₉ is L- or D-Met, L- or D-Ieu, L- or D-Asx, L- or D-Nle, L- or D-Ala, L- or D-Phe, L- or D-Glx, L- or D-Ile, or L- or D-Tyr;
Y₁₀ is any natural or non natural amino acid residue different from L- or D-Cys residue, such as L- or D-Leu, L- or D-Ile, L- or D-L- or D-Asx, or L- or D-Glx, L- or D-Ala, L- or D-Val, L- or D-Ser, L- or D-Thr, or L- or D-Tyr;
Y₁₁ is L- or D-Asx, L- or D-Glx, L- or D-Ser, L- or D-Thr, L- or D-Tyr, L- or D-Lys, or L or D-Arg;
Y₁₂ is any natural or non natural amino acid different from L- or D-Cys, such as L- or D-Ser, L- or D-Asx, L- or D-Glx, or L- or D-Lys;
Y₁₃ is L- or D-Glx, L- or D-Asx, L- or D-Lys, L- or D-Ser, L- or D-Thr, L- or D-Tyr, or L- or Arg; and,
Y₁₄ is one or up to three natural or non natural amino acid residue different from L- or D-Cys residue, such as L- or D-Lys, L- or D-Arg, L- or D-Ser, L- or D-Asn, L- or D-Orn-, or D-Ala, and L- or D-(Lys-Ser), L- or D-(Lys-Ser-Asn).

CXCL2, GRO-β, Compounds

In some embodiments, the CXCL2 (GRO-β) chemokine analogs include:

(SEQ ID NO:29)

R-X₀₁ X₀₂ X₀₃ X₀₄ X₀₅ X₀₆ X₀₇ X₀₈ X₀₉ X₁₀ X₁₁ X₁₂
X₁₃ X₁₄ X₁₅ X₁₆ [linker] Y₀₁ Y₀₂ Y₀₃ Y₀₄ Y₀₅ Y₀₆
Y₀₇ Y₀₈ Y₀₉ Y₁₀ Y₁₁ Y₁₂ Y₁₃ Y₁₄

wherein,
X₀₁ is L- or D-Thr, L- or D-Ser, L- or D-Tyr, L- or D-Trp, L- or D-His, L- or D-Glx, L- or D-Asx, L- or D-Leu, L- or D-Ile, or L- or D-Ala;
X₀₂ is any natural or non natural amino acid residue different from L- or D-Glx, L- or D-Asx, L- or D-Arg, L- or D-Lys, L- or D-His, L- or D-Arg, L- or D-Ser, or L- or D-Thr;
X₀₃ is any natural or non natural amino acid residue different from L- or D-Cys residue, such as L- or D-Leu, L- or D-Ile, L- or D-L- or D-Val, or L- or D-Ala, L- or D-Phe, or L- or D-Tyr;
X₀₄ is any natural or non natural amino acid residue different from L- or D-Cys residue, such as L- or D-Arg, L- or D-Lys, L- or D-Orn-, or D-Ala;
X₀₅ is L- or D-Cys, L- or D-Ala, L- or D-Phe, L- or D-His, L- or D-Trp, L- or D-Ser, L- or D-Thr, L- or D-Lys, L- or D-His, or L- or D-Tyr;
X₀₆ is any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, or L- or D-Trp;
X₀₇ is L- or D-Cys, L- or D-Ala, L- or D-Phe, L- or D-His, L- or D-Trp, L- or D-Ser, L- or D-Thr, L- or D-Lys, L- or D-His, or L- or D-Tyr;
X₀₈ is any natural or non natural amino acid residue different from L- or D-Cys residue, such as L- or D-Leu, L- or D-Ile, L- or D-L- or D-Val, or L- or D-Ala, L- or D-Phe, or L- or D-Tyr;
X₀₉ is any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, or L- or D-Trp;
X₁₀ is L- or D-Thr, L- or D-Ser, L- or D-Tyr, L- or D-Trp, or L- or D-His;
X₁₁ is any natural or non natural amino acid residue different from L- or D-Cys residue, such as L- or D-Leu, L- or D-Ile, L- or D-L- or D-Val, or L- or D-Ala, L- or D-Phe, or L- or D-Tyr;
X₁₂ is any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, or L- or D-Trp;
X₁₃ is Gly, L- or D-Ala, L- or D-Val, L- or D-Leu, or L- or D-Ile;
X₁₄ is any natural or non natural amino acid residue different from L- or D-Cys residue, such as L- or D-Ile, L- or D-Ieu, L- or D-L- or D-Val, or L- or D-Ala, L- or D-Phe, or L- or D-Tyr;
X₁₅ is L- or D-His, L- or D-Trp, L- or D-Tyr, L- or D-Arg, L- or D-Lys, or L- or D-Phe;
X₁₆ is any natural or non natural amino acid residue different from L- or D-Cys residue, such as L- or D-Leu, L- or D-Ile, L- or D-L- or D-Val, or L- or D-Ala, L- or D-Phe, or L- or D-Tyr;

and wherein,
Y₀₁ is L- or D-Val, L- or D-Leu, L- or D-Ile, L- or D-Pro, L- or D-Phe, L- or D-Tyr, L- or D-Ser, L- or D-Thr, L- or D-Met, L- or D-Lys, L- or D-Arg, or L- or D-Orn;
Y₀₂ is any natural or non natural amino acid residue different from L- or D-Cys residue, such as L- or D-Lys, L- or D-Arg, L- or D-Orn-, or D-Val;
Y₀₃ is any natural or non natural amino acid residue different from L- or D-Cys residue, such as L- or D-Lys, L- or D-Arg, L- or D-Ile, or L- or D-Leu;
Y₀₄ is any natural or non natural amino acid residue different from L- or D-Cys residue, such as L- or D-Ile, L- or D-Ieu, L- or D-L- or D-Lys, or L- or D-Ala, L- or D-Phe, or L- or D-Tyr;
Y₀₅ is any natural or non natural amino acid residue different from L- or D-Cys residue, such as L- or D-Ile, L- or D-Ieu, L- or D-L- or D-Glx, or L- or D-Asx, L- or D-Phe, or L- or D-Tyr;
Y₀₆ is any natural or non natural amino acid residue different from L- or D-Glx, L- or D-Asx, L- or D-Arg, L- or D-Lys, L- or D-His, L- or D-Tyr, L- or D-Ser, L- or D-Thr, or L- or D-Ile;
Y₀₇ is any natural or non natural amino acid residue different from L- or D-Cys residue, such as L- or D-Lys, L- or D-Arg, L- or D-Glu-, or D-Ile;
Y₀₈ is L- or D-Met, L- or D-Ile, L- or D-Nle, L- or D-Ala, L- or D-Phe, L- or D-Leu, L- or D-Lys, or L- or D-Arg;
Y₀₉ is any natural or non natural amino acid residue different from L- or D-Cys residue, such as L- or D-Leu, L- or D-Ile, L- or D-L- or D-Val, or L- or D-Ala, L- or D-Phe, L- or D-Lys, or L- or D-Arg;
Y₁₀ is any natural or non natural amino acid residue different from L- or D-Cys residue or D-Leu, L- or D-Ile, such as L- or D-Lys, L- or D-Arg, L- or D-Leu, L- or D-Asx, or L- or D-Glx;
Y₁₁ is L- or D-Asx, L- or D-Glx, L- or D-Arg, L- or D-Lys, L- or D-Ala, L- or D-Leu, or L or D-Ile;
Y₁₂ is Gly, L- or D-Asx, L- or D-Glx, L- or D-Leu, L- or D-Ile, or L- or D-Ala;
Y₁₃ is any natural or non natural amino acid residue different from L- or D-Cys residue, such as L- or D-Lys, L- or D-Arg, L- or D-Ser-, or L- or D-Gly; and,
Y₁₄ is one or up to three natural or non natural amino acid residue different from L- or D-Cys residue, such as L- or D-Lys, L- or D-Arg, L- or D-Ser, L- or D-Asn, L- or D-Orn-, or D-Ala, and L- or D-Lys, L- or D-(Lys Ser), or L- or D-(Lys-Ser-Asn).

CXCL3, GRO-γ, Compounds

In some embodiments, the CXCL3 (GRO-γ) chemokine analogs include:

(SEQ ID NO:35)

R-X₀₁ X₀₂ X₀₃ X₀₄ X₀₅ X₀₆ X₀₇ X₀₈ X₀₉ X₁₀ X₁₁ X₁₂
X₁₃ X₁₄ X₁₅ X₁₆ [linker] Y₀₁ Y₀₂ Y₀₃ Y₀₄ Y₀₅ Y₀₆
Y₀₇ Y₀₈ Y₀₉ Y₁₀ Y₁₁ Y₁₂ Y₁₃ Y₁₄

wherein,
X₀₁ is L- or D-Thr, L- or D-Ser, L- or D-Tyr, L- or D-Trp, L- or D-His, L- or D-Glx, L- or D-Asx, L- or D-Leu, L- or D-Ile, or L- or D-Ala;
X₀₂ is any natural or non natural amino acid residue different from L- or D-Glx, L- or D-Asx, L- or D-Arg, L- or D-Lys, L- or D-His, L- or D-Arg, L- or D-Ser, L- or D-Thr;
X₀₃ is any natural or non natural amino acid residue different from L- or D-Cys residue, such as L- or D-Leu, L- or D-Ile, L- or D-L- or D-Val, or L- or D-Ala, L- or D-Phe, L- or D-Tyr;
X₀₄ is any natural or non natural amino acid residue different from L- or D-Cys residue, such as L- or D-Arg, L- or D-Lys, L- or D-Orn- or D-Ala;
X₀₅ is L- or D-Cys, L- or D-Ala, L- or D-Phe, L- or D-His, L- or D-Trp, L- or D-Ser, L- or D-Thr, L- or D-Lys, L- or D-His, L- or D-Tyr;
X₀₆ is any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, L- or D-Trp;
X₀₇ is L- or D-Cys, L- or D-Ala, L- or D-Phe, L- or D-His, L- or D-Trp, L- or D-Ser, L- or D-Thr, L- or D-Lys, L- or D-His, L- or D-Tyr;
X₀₈ is any natural or non natural amino acid residue different from L- or D-Cys residue, such as L- or D-Leu, L- or D-Ile, L- or D-L- or D-Val, or L- or D-Ala, L- or D-Phe, L- or D-Tyr;
X₀₉ is any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, L- or D-Trp;
X₁₀ is L- or D-Thr, L- or D-Ser, L- or D-Tyr, L- or D-Trp, L- or D-His; L- or D-Ser, L- or D-Tyr;
X₁₁ is any natural or non natural amino acid residue different from L- or D-Cys residue, such as L- or D-Leu, L- or D-Ile, L- or D-L- or D-Val, or L- or D-Ala, L- or D-Phe, L- or D-Tyr;
X₁₂ is any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, L- or D-Trp;
X₁₃ is Gly, L- or D-Ala, L- or D-Val, L- or D-Leu, L- or D-Ile;
X₁₄ is any natural or non natural amino acid residue different from L- or D-Cys residue, such as L- or D-Ile, L- or D-Ieu, L- or D-L- or D-Val, or L- or D-Ala, L- or D-Phe, L- or D-Tyr;
X₁₅ is L- or D-His, L- or D-Trp, L- or D-Tyr, L- or D-Arg, L- or D-Lys, L- or D-Phe;
X₁₆ is any natural or non natural amino acid residue different from L- or D-Cys residue, such as L- or D-Leu, L- or D-Ile, L- or D-L- or D-Val, or L- or D-Ala, L- or D-Phe, L- or D-Tyr;

and wherein,
Y₀₁ is L- or D-Val, L- or D-Leu, L- or D-Ile, L- or D-Pro, L- or D-Phe, L- or D-Tyr, L- or D-Ser, L- or D-Thr, L- or D-Met;
Y₀₂ is any natural or non natural amino acid residue different from L- or D-Cys residue, such as Ls- or D-Lys, L- or D-Arg, L- or D-Orn- or D-Val;
Y₀₃ is any natural or non natural amino acid residue different from L- or D-Cys residue, such as L- or D-Glx, L- or D-Asx, L- or D-Lys, L- or D-Ile;
Y₀₄ is any natural or non natural amino acid residue different from L- or D-Cys residue, such as L- or D-Ile, L- or D-Ieu, L- or D-L- or D-Lys, or L- or D-Ala, L- or D-Phe, L- or D-Tyr;
Y₀₅ is any natural or non natural amino acid residue different from L- or D-Cys residue, such as L- or D-Ile, L- or D-Ieu, L- or D-L- or D-Glx, or L- or D-Ala, L- or D-Phe, L- or D-Tyr;
Y₀₆ is any natural or non natural amino acid residue different from L- or D-Glx, L- or D-Asx, L- or D-Arg, L- or D-Lys, L- or D-His, L- or D-Tyr, L- or D-Ser, L- or D-Thr, L- or D-Ile; L- or D-Glu, L- or D-Lys;
Y₀₇ is any natural or non natural amino acid residue different from L- or D-Cys residue, such as L- or D-Lys, L- or D-Arg, L- or D-Glu- or D-Ile;
Y₀₈ is L- or D-Lys, L- or D-Ile, L- or D-Leu, L- or D-Ala, L- or D-Phe, L- or D-Tyr, L- or D-Ser, L- or D-Arg, L- or D-Orn;
Y₀₉ is any natural or non natural amino acid residue different from L- or D-Cys residue, such as L- or D-Leu, L- or D-Ile, L- or D-L- or D-Asx, or L- or D-Glx, L- or D-Phe, L- or D-Tyr, L- or D-Nle;
Y₁₀ is any natural or non natural amino acid residue different from L- or D-Cys residue or D-Leu, L- or D-Ile, such as L- or D-Lys, L- or D-Arg, L- or D-Leu- or D-Asx, L- or D-Glx;
Y₁₁ is L- or D-Asx, L- or D-Glx, L- or D-Arg, L- or D-Lys, L- or D-Ala, L- or D-Orn, Gly;
Y₁₂ is any natural or non natural amino acid residue different from L- or D-Cys residue or Gly, L- or D-Asx, L- or D-Glx, L- or D-Ser, L- or D-Tyr, L- or D-Thr;
Y₁₃ is any natural or non natural amino acid residue different from L- or D-Cys residue, such as Gly, L- or D-Ser, L- or D-Thr;
Y₁₄ is one or up to three natural or non natural amino acid different from L- or D-Cys, such as L- or D-Asx, L- or D-Glx, L- or D-Ser, L- or D-Thr, L- or D-Tyr, L- or D-Lys, and L- or D-Ser, L- or D-(Ser-Thr), L- or D-(Ser-Thr-Asn);

CXCL4, PF-4, Compounds

In some embodiments, the CXCL4 (PF-4) chemokine analogs include:

(SEQ ID NO:38)

R-X₀₁ X₀₂ X₀₃ X₀₄ X₀₅ X₀₆ X₀₇ X₀₈ X₀₉ X₁₀ X₁₁ X₁₂
X₁₃ X₁₄ X₁₅ X₁₆ X₁₇ [linker] Y₀₁ Y₀₂ Y₀₃ Y₀₄ Y₀₅
Y₀₆ Y₀₇ Y₀₈ Y₀₉ Y₁₀ Y₁₁ Y₁₂ Y₁₃ Y₁₄

wherein,
X₀₁ is an optional natural or non natural amino acid residue different from L- or D-Cys residue, such as L- or D-Glx, L- or D-Asx;
X₀₂ is any natural or non natural amino acid residue different from L- or D-Cys, L- or D-Ala, L- or D-Val, L- or D-Ile, L- or D-Leu;
X₀₃ is L- or D-Glx, L- or D-Asx, L- or D-Ala, L- or D-Pro, L- or D-Phe, L- or D-Tyr, L- or D-Ser, L- or D-Thr;
X₀₄ is any natural or non natural amino acid residue different from L- or D-Cys residue, such as L- or D-Glx, L- or D-Asx, L- or D,L- or D-Ala, L- or D-Val, L- or D-Phe;
X₀₅ is L- or D-Asx, L- or D-Glx, L- or D-Ala, L- or D-Phe, L- or D-Tyr;
X₀₆ is any natural or non-natural amino acid residue different from L- or D-Cys, such as L- or D-Ala, L- or D-Val, L- or D-Leu, L- or D-Ile, L- or D-Phe, L- or D-Tyr, L- or D-Glx;
X₀₇ is L- or D-Asx, L- or D-Glx, L- or D-Ala, L- or D-Phe, L- or D-Tyr;
X₀₈ is any natural or non-natural amino acid residue different from L- or D-Cys, such as L- or D-Leu, L- or D-Ala, L- or D,L- or D-Val, L- or D-Leu, L- or D-Ile, L- or D-Phe, L- or D-Tyr;
X₀₉ is L- or D-Glx, L- or D-Asx, L- or D-Arg, L- or D-Lys, L- or D-His, L- or D-Tyr, L- or D-Ser, L- or D-Thr;
X₁₀ is L- or D-Cys, L- or D-Ala, L- or D-Phe, L- or D-His, L- or D-Trp, L- or D-Ser, L- or D-Thr;
X₁₁ is any natural or non-natural amino acid residue different from L- or D-Cys, such as L- or D-Glx, L- or D-Asx, L- or D-Gly, L- or D-Val, L- or D-Leu, L- or D-Ile, L- or D-Pro, L- or D-Phe, L- or D-Tyr, L- or D-His, L- or D-Trp;
X₁₂ is L- or D-Cys, L- or D-Ala, L- or D-Phe, L- or D-His, L- or D-Trp, L- or D-Ser, L- or D-Thr, L- or D-Lys, L- or D-His, L- or D-Tyr;
X₁₃ is L- or D-Val, L- or D-Leu, L- or D-Ile, L- or D-Ala, L- or D-Phe, L- or D-Tyr;
X₁₄ is optional, and may be any natural or non-natural amino acid residue different from L- or D-Cys, such as L- or D-Lys, L- or D-Arg, L- or D-His;
X₁₅ is optional and may be L- or D-Thr, L- or D-Ser, L- or D-Tyr, L- or D-Ala, L- or D-Phe;
X₁₆ is optional and may be any natural or non-natural amino acid residue different from L- or D-Cys, such as L- or D-Thr, L- or D-Ser, L- or D-Tyr, L- or D-Ala, L- or D-Phe;
X₁₇ is optional and may be any natural or non-natural amino acid residue different from L- or D-Cys, such as L- or D-Thr, L- or D-Ser, L- or D-Tyr, L- or D-Ala, L- or D-Phe;

and wherein,
Y₀₁ is any natural or non natural amino acid residue different from L- or D-Cys residue, such as L- or D-Ala, L- or D-Val, L- or D-Ile, L- or D-Leu;
Y₀₂ is L- or D-Pro, L- or D-Ala;
Y₀₃ is any natural or non natural amino acid residue different from L- or D-Cys, such as L- or D-Leu, L- or D-Ala, L- or D,L- or D-Val, L- or D-Leu, L- or D-Ile, L- or D-Phe, L- or D-Tyr, L- or D-Ser, L- or D-Thr, L- or D-Phe;
Y₀₄ is L- or D-Tyr, L- or D-Ser, L- or D-Thr, L- or D-Phe;
Y₀₅ is any natural or non natural amino acid residue different from L- or D-Cys, such as L- or D-Lys, L- or D-Arg, L- or D-His;
Y₀₆ is any natural or non natural amino acid residue different from L- or D-Cys, such as L- or D-Lys, L- or D-Arg, L- or D-His;
Y₀₇ is any natural or non natural amino acid residue different from L- or D-Cys, such as L- or D-Glx, L- or D-Asx, L- or D-Gly, L- or D-Val, L- or D-Leu, L- or D-Ile, L- or D-Pro, L- or D-Phe, L- or D-Tyr, L- or D-His, L- or D-Trp;
Y₀₈ is any natural or non natural amino acid residue different from L- or D-Cys, such as L- or D-Glx, L- or D-Asx, L- or D-Gly, L- or D-Val, L- or D-Leu, L- or D-Ile, L- or D-Pro, L- or D-Phe, L- or D-Tyr, L- or D-His, L- or D-Trp;
Y₀₉ is any natural or non natural amino acid residue different from L- or D-Cys, such as L- or D-Lys, L- or D-Arg, L- or D-His;
Y₁₀ is any natural or non natural amino acid residue different from L- or D-Cys, such as L- or D-Lys, L- or D-Arg, L- or D-His;
Y₁₁ is any natural or non natural amino acid residue different from L- or D-Cys, such as L- or D-Leu, L- or D-Ala, L- or D,L- or D-Val, L- or D-Leu, L- or D-Ile, L- or D-Phe, L- or D-Tyr;
Y₁₂ is any natural or non natural amino acid residue different from L- or D-Cys, such as L- or D-Leu, L- or D-Ala, L- or D,L- or D-Val, L- or D-Leu, L- or D-Ile, L- or D-Phe, L- or D-Tyr;
Y₁₃ is L- or D-Asx, L- or D-Glx, L- or D-Ala; and
Y₁₄ is any natural or non natural amino acid residue different from L- or D-Cys, such as L- or D-Thr, L- or D-Ser, L- or D-Tyr, L- or D-Ala, L- or D-Phe.

CXCL5, ENA-78, Compounds

In some embodiments, the CXCL5 (ENA-78) chemokine analogs include:

(SEQ ID NO:44)

R-X₀₁ X₀₂ X₀₃ X₀₄ X₀₅ X₀₆ X₀₇ X₀₈ X₀₉ X₁₀ X₁₁ X₁₂
X₁₃ X₁₄ X₁₅ X₁₆ [linker] Y₀₁ Y₀₂ Y₀₃ Y₀₄ Y₀₅ Y₀₆
Y₀₇ Y₀₈ Y₀₉ Y₁₀ Y₁₁ Y₁₂ Y₁₃ Y₁₄

(SEQ ID NO:45)

R-X₀₂ X₀₃ X₀₄ X₀₅ X₀₆ X₀₇ X₀₈ X₀₉ X₁₀ X₁₁ X₁₂ X₁₃
X₁₄ X₁₅ X₁₆ [linker] Y₀₁ Y₀₂ Y₀₃ Y₀₄ Y₀₅ Y₀₆ Y₀₇
Y₀₈ Y₀₉ Y₁₀ Y₁₁ Y₁₂ Y₁₃ Y₁₄

wherein,
X₀₁ is any natural or non natural amino acid residue different from L- or D-Cys residue, such as L- or D-Leu, L- or D-Ile, L- or D-L- or D-Arg, or L- or D-Lys, L- or D-Orn, L- or D-Ala;
X₀₂ is any natural or non natural amino acid residue different from L- or D-Cys, such as L- or D-Arg, L- or D-Lys, L- or D-Orn- or L- or D-Glx, L- or D-Asx, L- or D-Thr, L- or D-Tyr;
X₀₃ is any natural or non natural amino acid residue different from L- or D-Cys, such as L- or D-Glx, L- or D-Asx, L- or D-Leu, L- or D-Ile, L- or D-Arg, L- or D-Ser, L- or D-Thr;
X₀₄ is any natural or non natural amino acid residue different from L- or D-Cys residue, such as L- or D-Leu, L- or D-Arg, or L- or D-Ile, L- or D-Lys;
X₀₅ is L- or D-Arg, L- or D-Cys, L- or D-Lys, L- or D-Orn, L- or D-Ala residue, such as L- or D-Arg, L- or D-Cys, L- or D-Orn- or D-Ala, L- or D-Lys;
X₀₆ is L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn;
X₀₇ is any natural or non natural amino acid residue different from L- or D-Cys residue, such as L- or D-Val, L- or D-Ile, L- or D-Ala, L- or D-Phe, L- or D-Tyr;
X₀₈ is L- or D-Cys, L- or D-Leu, L- or D-Phe, L- or D-His, L- or D-Trp, L- or D-Ser, L- or D-Thr, L- or D-Lys, L- or D-His, L- or D-Tyr, L- or D-Ile, L- or D-Orn;
X₀₉ is any natural or non natural amino acid residue different from L- or D-Cys residue, such as L- or D-Leu, L- or D-Glx, or L- or D-Asx, L- or D-Lys, L-Ile;
X₁₀ is any natural or non natural amino acid residue different from L- or D-Cys residue, such as L- or D-Glx, L- or D-Asx, L- or D-Thr, L- or D-Ser, L- or D-Leu, L- or D-Ile, L- or D-Pro, L- or D-Phe, L- or D-Tyr, L- or D-His, L- or D-Trp;
X₁₁ is L- or D-Thr, L- or D-Ser, L- or D-Tyr, L- or D-Trp, L- or D-His;
X₁₂ is any natural or non natural amino acid residue different from L- or D-Cys residue, such as L- or D-Thr, L- or D-Glx, L- or D-L- or D-Asx, L- or D-Tyr;
X₁₃ is any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, L- or D-Trp;
X₁₄ is any natural or non natural amino acid residue different from L- or D-Cys residue, such as Gly, L- or D-Val, L- or D-Ala;
X₁₅ is any natural or non natural amino acid residue different from L- or D-Cys residue, such as L- or D-Val, L- or D-His, L- or D-Orn- or D-Arg;
X₁₆ is one or up to two natural or non natural amino acid residue different from L- or D-Cys residue, such as L- or D-His, L- or D-Pro, Gly, L- or D-Val, L- or D-Leu, L- or D-Ile, L- or D-Ala, L- or D-Phe, L- or D-Tyr;

and wherein,
Y₀₁ is any natural or non natural amino acid residue different from L- or D-Cys residue, such as L- or D-Phe, L- or D-Ieu, L- or D-Lys, L- or D-Tyr, or L- or D-His, L- or D-Trp;
Y₀₂ is L- or D-Leu, L- or D-Ile, L- or D-Lys, L- or D-Phe, L- or D-Tyr, L- or D-Ser, L- or D-Thr, L- or D-Orn, L- or D-Arg, L- or D-Val;
Y₀₃ is any natural or non natural amino acid residue different from L- or D-Cys residue, such as L- or D-Lys, L- or D-Arg, L- or D-Orn- or D-Val, L- or D-Ile, L- or D-Leu;
Y₀₄ is any natural or non natural amino acid residue different from L- or D-Cys residue, such as L- or D-Lys, L- or D-Arg, L- or D-Glx, L- or D-Asx, L- or D-Orn- or D-Ala, IL- or D-Ile, L- or D-Val;
Y₀₅ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
Y₀₆ is any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys;
Y₀₇ is any natural or non natural amino acid residue different from L- or D-Cys, such as L- or D-Glx, L- or D-Asx, L- or D-Lys, L- or D-Ile, L- or D-Leu, L- or D-Ala;
Y₀₈ is any natural or non natural amino acid residue different from L- or D-Cys, such as L- or D-Lys, L- or D-Ile, L- or D-Leu, L- or D-Asx, L- or D-Glx, L- or D-His, L- or D-Tyr, L- or D-Ser, L- or D-Thr, L- or D-Leu, L- or D-Ile;
Y₀₉ is any natural or non natural amino acid residue different from L- or D-Cys residue, such as L- or D-Ile, L- or D-Glx, L- or D-Asx, L- or D-Leu, Gly, L- or D-Phe, L- or D-Tyr, L- or D-Glu, L- or D-Lys;
Y₁₀ is any natural or non natural amino acid residue different from L- or D-Cys residue, such as L- or D-Leu, L- or D-Glx, or L- or D-Asx, Gly;
Y₁₁ is L- or D-Asx, L- or D-Glx, L- or D-Ser, L- or D-Thr, L- or D-Tyr, L- or D-Lys, L or D-Arg, Gly;
Y₁₂ is any natural or non natural amino acid different from L- or D-Cys, such as Gly, L- or D-Asx, L- or D-Glx, L- or D-Lys, L- or D-Arg;
Y₁₃ is any natural or non natural amino acid different from L- or D-Cys, such as Gly, L- or D-Asx, L- or D-Glx, L- or D-Lys, L- or D-Arg; L- or D-Glu; and,
Y₁₄ is one or up to four natural or non natural amino acid residue different from L- or D-Cys residue, such as L- or D-Asx, L- or D-Glx, L- or D-Lys, L- or D-Orn- or D-Ala, the preferred amino acid residues are: L- or D-Asn, L- or D-(Asn-Lys), L- or D-(Asn-Lys-Glu), L- or D-(Asn-Lys-Glu-Asn).

CXCL6, GCP-2, Compounds

In some embodiments, the CXCL6 (GCP-2) chemokine analogs include:

(SEQ ID NO:62)

R-X₀₁ X₀₂ X₀₃ X₀₄ X₀₅ X₀₆ X₀₇ X₀₈ X₀₉ X₁₀ X₁₁ X₁₂
X₁₃ X₁₄ X₁₅ X₁₆ [linker] Y₀₁ Y₀₂ Y₀₃ Y₀₄ Y₀₅ Y₀₆
Y₀₇ Y₀₈ Y₀₉ Y₁₀ Y₁₁ Y₁₂ Y₁₃ Y₁₄

(SEQ ID NO:63)

R-X₀₆ X₀₇ X₀₈ X₀₉ X₁₀ X₁₁ X₁₂ X₁₃ X₁₄ X₁₅ X₁₆
[linker] Y₀₁ Y₀₂ Y₀₃ Y₀₄ Y₀₅ Y₀₆ Y₀₇ Y₀₈ Y₀₉ Y₁₀
Y₁₁ Y₁₂ Y₁₃ Y₁₄

(SEQ ID NO:64)

R-X₀₃ X₀₄ X₀₅ X₀₆ X₀₇ X₀₈ X₀₉ X₁₀ X₁₁ X₁₂ X₁₃ X₁₄
X₁₅ X₁₆ [linker] Y₀₁ Y₀₂ Y₀₃ Y₀₄ Y₀₅ Y₀₆ Y₀₇ Y₀₈
Y₀₉ Y₁₀ Y₁₁ Y₁₂ Y₁₃ Y₁₄

(SEQ ID NO:65)

R-X₀₉ X₁₀ X₁₁ X₁₂ X₁₃ X₁₄ X₁₅ X₁₆ [linker] Y₀₁ Y₀₂
Y₀₃ Y₀₄ Y₀₅ Y₀₆ Y₀₇ Y₀₈ Y₀₉ Y₁₀ Y₁₁ Y₁₂ Y₁₃ Y₁₄

wherein,
X₀₁ is any natural or non natural amino acid residue different from L- or D-Cys residue, such as Gly, L- or D-Pro, L- or D-L- or D-Val, or L- or D-Ala;
X₀₂ is any natural or non natural amino acid residue different from L- or D-Cys, such as L- or D-Pro, L- or D-Ala, L- or D-Leu- or L- or D-Ile, L- or D-Phe, L- or D-Thr, L- or D-Tyr;
X₀₃ is any natural or non natural amino acid residue different from L- or D-Cys residue, such as L- or D-Val, L- or D-Ile, L- or D-Ala, L- or D-Phe, L- or D-Tyr;
X₀₄ is any natural or non natural amino acid different from L- or D-Cys, such as L- or D-Asx, L- or D-Glx, L- or D-Ser, L- or D-Thr, L- or D-Tyr, L- or D-Lys;
X₀₅ is any natural or non natural amino acid residue different from L- or D-Cys residue, such as L- or D-Ala, Gly, or D-L-Phe;
X₀₆ is L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn;
X₀₇ is any natural or non natural amino acid residue different from L- or D-Cys residue, such as L- or D-Leu, L- or D-Arg, or L- or D-Ile, L- or D-Lys;
X₀₈ is L- or D-Thr, L- or D-Ser, L- or D-Tyr, L- or D-Trp, L- or D-His;
X₀₉ is any natural or non natural amino acid residue different from L- or D-Cys, such as L- or D-Glx, L- or D-Asx, L- or D-Leu, L- or D-Ile, L- or D-Arg, L- or D-Ser, L- or D-Thr;
X₁₀ is any natural or non natural amino acid residue different from L- or D-Cys residue, such as L- or D-Leu, L- or D-Arg, or L- or D-Ile, L- or D-Lys;
X₁₁ is L- or D-Arg, L- or D-Lys, L- or D-Orn- or L- or D-Glx, L- or D-Asx, L- or D-Thr, L- or D-Tyr;
X₁₂ is L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe;
X₁₃ is any natural or non natural amino acid residue different from L- or D-Cys residue, such as L- or D-Thr, L- or D-Glx, L- or D-L- or D-Asx, L- or D-Tyr;
X₁₄ is L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe;
X₁₅ is any natural or non natural amino acid residue different from L- or D-Cys residue, such as L- or D-Leu, L- or D-Arg, or L- or D-Ile, L- or D-Lys;
X₁₆ is one up to ten natural or non natural amino acid residues different from L- or D-Cys residue, such as L- or D-Arg, L- or D-Val, L- or D-Thr, L- or D-Leu, L- or D-Ile, L- or D-Asx, L- or D-Pro, L- or D-Lys, and L- or D-Leu, L- or D-(Leu-Arg-Val), L- or D-(Leu-Arg-Val-Thr-Leu-Arg), L- or D-(Leu-Arg-Val-Thr-Leu-Arg-Val-Asn-Pro-Lys);

and wherein,
Y₀₁ is any natural or non natural amino acid residue different from L- or D-Cys residue, such as L- or D-Phe, L- or D-Ieu, L- or D-Lys, L- or D-Tyr, or L- or D-His, L- or D-Trp;
Y₀₂ is L- or D-Leu, L- or D-Ile, L- or D-Lys, L- or D-Phe, L- or D-Tyr, L- or D-Ser, L- or D-Thr, L- or D-Orn, L- or D-Arg, L- or D-Val;
Y₀₃ is any natural or non natural amino acid residue different from L- or D-Cys residue, such as L- or D-Lys, L- or D-Arg, L- or D-Orn- or D-Val, L- or D-Ile, L- or D-Leu;
Y₀₄ is any natural or non natural amino acid residue different from L- or D-Cys residue, such as L- or D-Lys, L- or D-Arg, L- or D-Orn- or D-Val, L- or D-Ile, L- or D-Leu;
Y₀₅ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
Y₀₆ is any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys;
Y₀₇ is any natural or non natural amino acid residue different from L- or D-Cys, such as L- or D-Glx, L- or D-Asx, L- or D-Lys, L- or D-Ile, L- or D-Leu, L- or D-Ala;
Y₀₈ is any natural or non natural amino acid residue different from L- or D-Cys residue, such as L- or D-Lys, L- or D-Arg, L- or D-Orn- or D-Val, L- or D-Ile, L- or D-Leu;
Y₀₉ is any natural or non natural amino acid residue different from L- or D-Cys residue, such as L- or D-Ile, L- or D-Glx, L- or D-Asx, L- or D-Leu, Gly, L- or D-Phe, L- or D-Tyr, L- or D-Glu, L- or D-Lys; L- or D-Leu, L- or D-Asp, Gly,
Y₁₀ is any natural or non natural amino acid residue different from L- or D-Cys, such as L- or D-Glx, L- or D-Asx, L- or D-Lys, L- or D-Ile, L- or D-Leu, L- or D-Ala;
Y₁₁ is L- or D-Asx, L- or D-Glx, L- or D-Ser, L- or D-Thr, L- or D-Tyr, L- or D-Lys, L or D-Arg, Gly;
Y₁₂ is any natural or non natural amino acid different from L- or D-Cys, such as L- or D-Asx, L- or D-Glx, L- or D-Ser, L- or D-Thr, L- or D-Tyr, L- or D-Lys;
Y₁₃ is any natural or non natural amino acid residue different from L- or D-Cys residue, such as Gly, L- or D-Pro, L- or D-L- or D-Val, or L- or D-Ala;
Y₁₄ is one or up to four natural or non natural amino acid residue different from L- or D-Cys residue, such as L- or D-Asx, L- or D-Glx, L- or D-Lys, L- or D-Orn- or D-Ala, and L- or D-Asn, L- or D-(Asn-Lys), L- or D-(Asn-Lys-Lys), L- or D-(Asn-Lys-Lys-Asn);

CXCL7, NAP-2, Compounds

In some embodiments, the CXCL7 (NAP-2) chemokine analogs include:

(SEQ ID NO:74)

R-X₀₁ X₀₂ X₀₃ X₀₄ X₀₅ X₀₆ X₀₇ X₀₈ X₀₉ X₁₀ X₁₁ X₁₂
X₁₃ X₁₄ X₁₅ X₁₆ [linker] Y₀₁ Y₀₂ Y₀₃ Y₀₄ Y₀₅ Y₀₆
Y₀₇ Y₀₈ Y₀₉ Y₁₀ Y₁₁ Y₁₂ Y₁₃ Y₁₄

(SEQ ID NO:75)

R-X₀₂ X₀₃ X₀₄ X₀₅ X₀₆ X₀₇ X₀₈ X₀₉ X₁₀ X₁₁ X₁₂ X₁₃
X₁₄ X₁₅ X₁₆ [linker] Y₀₁ Y₀₂ Y₀₃ Y₀₄ Y₀₅ Y₀₆ Y₀₇
Y₀₈ Y₀₉ Y₁₀ Y₁₁ Y₁₂ Y₁₃ Y₁₄

wherein,
X₀₁ is any natural or non natural amino acid residue different from L- or D-Cys residue, such as L- or D-Ala, Gly, or D-L-Phe;
X₀₂ is any natural or non natural amino acid residue different from L- or D-Cys, such as L- or D-Glx, L- or D-Asx, L- or D-Leu, L- or D-Ile, L- or D-Arg, L- or D-Ser, L- or D-Thr;
X₀₃ is any natural or non natural amino acid residue different from L- or D-Cys, such as L- or D-Glx, L- or D-Asx, L- or D-Lys, L- or D-Ile, L- or D-Leu, L- or D-Ala;
X₀₄ is L- or D-Arg, L- or D-Lys, L- or D-Orn- or L- or D-Glx, L- or D-Asx, L- or D-Thr, L- or D-Tyr;
X₀₅ is L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe;
X₀₆ is L- or D-Met, L- or D-Ieu, L- or D-Asx, L- or D-Nle, L- or D-Ala, L- or D-Phe, L- or D-Glx, L- or D-Ile, L- or D-Tyr;
X₀₇ is L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe;
X₀₈ is any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys;
X₀₉ is any natural or non natural amino acid residue different from L- or D-Cys residue, such as L- or D-Lys, L- or D-Arg, L- or D-Orn- or D-Val, L- or D-Ile, L- or D-Leu;
X₁₀ is any natural or non natural amino acid residue different from L- or D-Cys residue, such as L- or D-Thr, L- or D-Glx, L- or D-L- or D-Asx, L- or D-Tyr;
X₁₁ is any natural or non natural amino acid residue different from L- or D-Cys residue, such as L- or D-Thr, L- or D-Glx, L- or D-L- or D-Asx, L- or D-Tyr;
X₁₂ is any natural or non natural amino acid different from L- or D-Cys, such as L- or D-Asx, L- or D-Glx, L- or D-Ser, L- or D-Thr, L- or D-Tyr, L- or D-Lys;
X₁₃ is any natural or non natural amino acid residue different from L- or D-Cys residue, such as Gly, L- or D-Pro, L- or D-L- or D-Val, or L- or D-Ala;
X₁₄ is any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys;
X₁₅ is L- or D-His, L- or D-Lys, L- or D-Arg, L- or D-Orn, L- or D-Trp, L- or D-Ala, L- or D-Phe, L- or D-Pro;
X₁₆ is any natural or non natural amino acid residue different from L- or D-Cys, such as L- or D-Pro, L- or D-Ala, L- or D-Leu- or L- or D-Ile, L- or D-Phe, L- or D-Thr, L- or D-Tyr;

and wherein,
Y₀₁ is one or up to five natural or non natural amino acid residue different from L- or D-Cys residue, such as L- or D-Pro, L- or D-Asx, L- or D-Ala, L- or D-Arg, or L- or D-Phe, L- or D-Tyr, and L- or D-(Pro-Asp-Ala-Pro-Arg), L- or D-Arg;
Y₀₂ is L- or D-Leu, L- or D-Ile, L- or D-Lys, L- or D-Phe, L- or D-Tyr, L- or D-Ser, L- or D-Thr, L- or D-Orn, L- or D-Arg, L- or D-Val;
Y₀₃ is any natural or non natural amino acid residue different from L- or D-Cys residue, such as L- or D-Lys, L- or D-Arg, L- or D-Orn- or D-Val, L- or D-Ile, L- or D-Leu;
Y₀₄ is any natural or non natural amino acid residue different from L- or D-Cys residue, such as L- or D-Lys, L- or D-Arg, L- or D-Orn- or D-Val, L- or D-Ile, L- or D-Leu;
Y₀₅ is L- or D-Leu, L- or D-Ile, L- or D-Lys, L- or D-Phe, L- or D-Tyr, L- or D-Ser, L- or D-Thr, L- or D-Orn, L- or D-Arg, L- or D-Val;
Y₀₆ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
Y₀₇ is any natural or non natural amino acid residue different from L- or D-Cys, such as L- or D-Glx, L- or D-Asx, L- or D-Lys, L- or D-Ile, L- or D-Leu, L- or D-Ala;
Y₀₈ is any natural or non natural amino acid residue different from L- or D-Cys residue, such as L- or D-Lys, L- or D-Arg, L- or D-Orn- or D-Val, L- or D-Ile, L- or D-Leu;
Y₀₉ is any natural or non natural amino acid residue different from L- or D-Cys residue, such as L- or D-Lys, L- or D-Arg, L- or D-Orn- or D-Val, L- or D-Ile, L- or D-Leu;
Y₁₀ no residue, or is any natural or non natural amino acid residue different from L- or D-Cys, such as L- or D-Glx, L- or D-Asx, L- or D-Lys, L- or D-Ile, L- or D-Leu, L- or D-Ala;
Y₁₁ no residue, or is any natural or non natural amino acid residue different from L- or D-Cys residue, such as L- or D-Ala, Gly, or D-L-Phe;
Y₁₂ no residue, or is any natural or non natural amino acid residue different from L- or D-Cys residue, such as Gly, L- or D-Pro, L- or D-L- or D-Val, or L- or D-Ala;
Y₁₃ no residue, or is L- or D-Asx, L- or D-Glx, L- or D-Ser, L- or D-Thr, L- or D-Tyr, L- or D-Lys, L or D-Arg, Gly;
Y₁₄ no residue, or is one to four natural or non natural amino acid residues different from L- or D-Cys, such as L- or D-Glx, L- or D-Asx, L- or D-Leu, L- or D-Ile, L- or D-Arg, L- or D-Ser, L- or D-Thr; L- or D-(Glu-Ser), L- or D-(Glu-Ser-Ala), L- or D-(Glu-Ser-Ala-Asp),

CXCL8, IL-8, Compounds

In some embodiments, the invention includes mimetics of human chemokine interleukin-8 (IL-8). In these embodiments, the analog can include a first conserved region and a second conserved region, wherein the first conserved region can include an N-terminal region, and the second conserved region can include a C-terminal region. The N-terminal region can include a series of up to 17 of the first 17 amino acids of a native IL-8 chemokine, and the C-terminal region can include a series of up to 17 of the last 17 amino acids in the native IL-8 chemokine. In some embodiments, these conserved regions can be linked using a linker.

In some embodiments, for example, the analog can comprise an N-terminal region having the first 15 residues of the native IL-8 chemokine, and the C-terminal region can comprise residues 56-71 of the native IL-8 chemokine:
(1-15)-[linker]-(56-71)

Possibilities for IL-8 analogs are taught in detail in U.S. patent application Ser. Nos. 10/932,208 and 10/243,795, each of which is hereby incorporated by reference herein in its entirety. Possible configurations for the CXC chemokine analogs can include those shown in Table 3, where the linker can be 11-aminoundecanoic acid (UDA) or four glycine residues (Gly₄), substituted residues are indicated using brackets and superscripts, the N-terminus can be acetylated, and the C-terminal can be amidated.

TABLE 3

(1-15)-[Gly]₄-(56-71)
(1-15)-[Gly]₄-(56-71)-cyclo-(Glu⁶³-Lys⁶⁷)
(1-15)-[UDA]-(56-71)
[Ala⁷,Phe⁹]-(1-15)-[UDA]-(56-71)
[Ser⁷,Ser⁹]-(1-15)-[UDA]-(56-71)
[Ala⁷,Tyr⁹]-(1-15)-[UDA]-(56-71)
Ac-[Ala⁷,Tyr⁹]-(1-15)-[UDA]-(56-71)
[Tyr⁷,Phe⁹] (1-15)-[UDA]-1(56-71)
Ac-[Tyr⁷,Phe⁹]-(1-15)-[linker]-(56-71)
[Tyr⁷,Ala⁹]-(1-15)-[UDA]-(56-71)-NH₂
Ac-[Tyr⁷,Ala⁹]-(1-15)-[UDA]-(56-71)
[Tyr⁷,Tyr⁹]-IL-8-1 (1-15)-[UDA]-IL-8-1(56-71)-NH₂
Ac-[Tyr⁷,Tyr⁹]-(1-15)-[UDA]-(56-71)
Ac-[Tyr⁷,Phe⁹,Arg¹¹]-(1-15)-[UDA]-(56-71)
[Ala⁷,Phe⁹](1-15)-[UDA]-(56-71)-cyclo-(Glu⁶³-Lys⁶⁷)
Ac-[Ala⁷,Phe⁹]-(1-15)-[UDA]-(56-71)-cyclo-(Glu⁶³-Lys⁶⁷)
Ac-[Ala⁷,Tyr⁹]-(1-15)-[UDA]-(56-71)-cyclo-(Glu⁶³-Lys⁶⁷)
Ac-[Tyr⁷,Phe⁹]-(1-15)-[UDA]-(56-71)-cyclo-(Glu⁶³-Lys⁶⁷)
Ac-[Tyr⁷,Tyr⁹]-(1-15)-[UDA]-(56-71)-cyclo-(Glu⁶³-Lys⁶⁷)
Ac-[His⁷,Tyr⁹]-(1-15)-[UDA]-(56-71)
Cyclic 63/67 (Glu⁶³-Lys⁶⁷)
Ac-[Tyr⁷,Trp⁹]-(1-15)-[UDA]-(56-71)
Cyclic 63/67 (Glu⁶³-Lys⁶⁷)
Ac-[Ala⁷,Phe⁹,Arg¹¹]-(1-15)-[UDA]-1 (56-71)-cyclo-(Glu⁶³-
Lys⁶⁷)
Ac-[Tyr⁷,Trp⁹,Arg¹¹]-(1-15)-[UDA]-(56-71)-cyclo-(Glu⁶³-Lys⁶⁷)
Ac-[Tyr⁷,Trp⁹,Arg¹¹,Arg¹⁵]-(1-15)-[UDA]-(56-71)-cyclo-
(Glu⁶³-Lys⁶⁷)
Ac-[Trp⁷,Trp⁹,Arg¹¹,Arg¹⁵]-(1-15)-[UDA]-(56-71)-cyclo-
(Glu⁶³-Lys⁶⁷)
Ac-[His⁷,Trp⁹,Arg¹¹,Arg¹⁵]-(1-15)-[UDA]-1(56-71)-cyclo-
(Glu⁶³-Lys⁶⁷)
Wherein, UDA (11-aminoundecanoic acid) and [Gly]₄ are
linkers and can also be any linker taught herein.

CXCL9, MIG, Compounds

In some embodiments, the CXCL9 (MIG) chemokine analogs include:

(SEQ ID NO:112)

R-X₀₁ X₀₂ X₀₃ X₀₄ X₀₅ X₀₆ X₀₇ X₀₈ X₀₉ X₁₀ X₁₁ X₁₂
X₁₃ X₁₄ X₁₅ X₁₆ [linker] Y₀₁ Y₀₂ Y₀₃ Y₀₄ Y₀₅ Y₀₆
Y₀₇ Y₀₈ Y₀₉ Y₁₀ Y₁₁ Y₁₂ Y₁₃ Y₁₄

wherein,
X₀₁ is any natural or non natural amino acid residue different from L- or D-Cys residue, such as L- or D-Thr, L- or D-Glx, L- or D-L- or D-Asx, L- or D-Tyr;
X₀₂ is any natural or non natural amino acid residue different from L- or D-Cys, such as L- or D-Pro, L- or D-Ala, L- or D-Leu- or L- or D-Ile, L- or D-Phe, L- or D-Thr, L- or D-Tyr;
X₀₃ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
X₀₄ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
X₀₅ is L- or D-Arg, L- or D-Lys, L- or D-Orn- or L- or D-Glx, L- or D-Asx, L- or D-Thr, L- or D-Tyr;
X₀₆ is any natural or non natural amino acid residue different from L- or D-Cys residue, such as L- or D-Lys, L- or D-Arg, L- or D-Orn- or D-Val, L- or D-Ile, L- or D-Leu;
X₀₇ is any natural or non natural amino acid residue different from L- or D-Cys residue, such as Gly, L- or D-Pro, L- or D-L- or D-Val, or L- or D-Ala;
X₀₈ is L- or D-Arg, L- or D-Lys, L- or D-Orn- or L- or D-Glx, L- or D-Asx, L- or D-Thr, L- or D-Tyr;
X₀₉ is L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe;
X₁₀ is any natural or non natural amino acid different from L- or D-Cys, such as L- or D-Asx, L- or D-Glx, L- or D-Ser, L- or D-Thr, L- or D-Tyr, L- or D-Lys;
X₁₁ is L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe;
X₁₂ is any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys;
X₁₃ is any natural or non natural amino acid different from L- or D-Cys, such as L- or D-Asx, L- or D-Glx, L- or D-Ser, L- or D-Thr, L- or D-Tyr, L- or D-Lys;
X₁₄ is any natural or non natural amino acid residue different from L- or D-Cys residue, such as L- or D-Thr, L- or D-Glx, L- or D-L- or D-Asx, L- or D-Tyr;
X₁₅ is L- or D-Asx, L- or D-Glx, L- or D-Arg, L- or D-Lys, L- or D-Ala, L- or D-Orn, Gly;
X₁₆ is any natural or non natural amino acid residue different from L- or D-Cys, such as L- or D-Glx, L- or D-Asx, L- or D-Lys, L- or D-Ile, L- or D-Leu, L- or D-Ala;

and wherein,
Y₀₁ is any natural or non natural amino acid residue different from L- or D-Cys residue, such as L- or D-Glx, L- or D-Asx, L- or D-Ala, L- or D-Arg, or L- or D-Phe, L- or D-Tyr;
Y₀₂ is L- or D-Ser, L- or D-Thr, L- or D-Lys, L- or D-Phe, L- or D-Tyr, L- or D-Arg, L- or D-Ala, L- or D-His, L- or D-Trp, L- or D-Val;
Y₀₃ is L- or D-Ser, L- or D-Thr, L- or D-Lys, L- or D-Phe, L- or D-Tyr, L- or D-Arg, L- or D-Ala, L- or D-His, L- or D-Trp, L- or D-Val;
Y₀₄ is any natural or non natural amino acid residue different from L- or D-Cys residue, such as L- or D-Glx, L- or D-Asx, L- or D-Lys, L- or D-Arg, or L- or D-Phe, L- or D-Tyr;
Y₀₅ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
Y₀₆ is L- or D-Ser, L- or D-Thr, L- or D-Lys, L- or D-Arg, L- or D-Tyr, L- or D-Leu, L- or D-Ile, L- or D-Nle, L- or D-Ala, L- or D-Val;
Y₀₇ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val; L- or D-Lys,
Y₀₈ is L- or D-Leu, L- or D-Thr, L- or D-Lys, L- or D-Arg, L- or D-Tyr, L- or D-Ser, L- or D-Ile, L- or D-Nle, L- or D-Ala, L- or D-Val;
Y₀₉ is any natural or non natural amino acid residue different from L- or D-Cys residue, such as L- or D-Lys, L- or D-Arg, L- or D-Orn- or D-Val, L- or D-Ile, L- or D-Leu;
Y₁₀ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
Y₁₁ is any natural or non natural amino acid residue different from L- or D-Cys residue, such as L- or D-Glx, L- or D-Asx, L- or D-Lys, L- or D-Arg, or L- or D-Ser, L- or D-Tyr, L- or D-Thr;
Y₁₂ is any natural or non natural amino acid residue different from L- or D-Cys residue, such as L- or D-Glx, L- or D-Asx, L- or D-Trp, L- or D-His, or L- or D-Phe, L- or D-Tyr, L- or D-Thr, L- or D-Ser;
Y₁₃ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val; L- or D-Arg,
Y₁₄ is one or up to seven natural or non natural amino acid residues different from L- or D-Cys, such as L- or D-Glx, L- or D-Asx, L- or D-Lys, L- or D-Ser, L- or D-Arg, L- or D-Ala, L- or D-Thr, L- or D-Tyr, and L- or D-Lys, L- or D-Ser, L- or D-(Lys-Gln), L- or D-(Ser-Arg), L- or D-(Ser-Arg-Gln), L- or D-(Ser-Arg-Gln-Lys), L- or D-(Ser-Arg-Gln-Lys-Lys), L- or D-(Ser-Arg-Gln-Lys-Lys-Thr), L- or D-(Ser-Arg-Gln-Lys-Lys-Thr-Thr);

CXCL10, IP-10, Compounds

In some embodiments, the invention includes mimetics of human chemokine IP-10. In these embodiments, the analog can include a first conserved region and a second conserved region, wherein the first conserved region can include an N-terminal region, and the second conserved region can include a C-terminal region. The N-terminal region can include a series of up to 17 of the first 17 amino acids of a native IP-10 chemokine, and the C-terminal region can include a series of up to 17 of the last 17 amino acids in the native IP-10 chemokine. In some embodiments, these conserved regions can be linked using a linker.

In some embodiments, for example, the analog can comprise an N-terminal region having the first 14 residues of the native IP-10 chemokine, and the C-terminal region can comprise residues 55-67, 58-71, 59-72, or 66-78, for example, of the native IP-10 chemokine:
(1-14)-[linker]-(59-72)

Possibilities for IP-10 analogs are taught in detail in U.S. patent application Ser. Nos. 11/590,210 and 10/243,795, each of which is hereby incorporated by reference herein in its entirety. Possible configurations for the IP-10 mimetics can include those shown in Table 4. The N-terminus can be acetylated, and the C-terminal can be amidated.

TABLE 4

(1-14)-linker-(66-78)
(1-14)-linker-(55-67)
(1-14)-linker-(59-72)
(1-17)-linker-(66-78)
(1-17)-linker-(55-67)
(1-17)-linker-(59-72)
(1-15)-linker-(58-71)
(1-16)-linker-(66-78)
Wherein the linker can be any linker taught herein.

The IP-10s can be cyclized in their C-terminal region using the methods taught herein.

CXCL11, I-TAC, Compounds

In some embodiments, the CXCL11 (I-TAC) chemokine analogs include:

(SEQ ID NO:128)

R-X₀₁ X₀₂ X₀₃ X₀₄ X₀₅ X₀₆ X₀₇ X₀₈ X₀₉ X₁₀ X₁₁ X₁₂
X₁₃ X₁₄ X₁₅ X₁₆ [linker] Y₀₁ Y₀₂ Y₀₃ Y₀₄ Y₀₅ Y₀₆
Y₀₇ Y₀₈ Y₀₉ Y₁₀ Y₁₁ Y₁₂ Y₁₃ Y₁₄

wherein,
X₀₁ is any natural or non natural amino acid residue different from L- or D-Cys residue, such as L- or D-Phe, L- or D-His, L- or D-L- or D-Trp, L- or D-Tyr;
X₀₂ is any natural or non natural amino acid residue different from L- or D-Cys, such as L- or D-Pro, L- or D-Ala, L- or D-Leu- or L- or D-Ile, L- or D-Phe, L- or D-Thr, L- or D-Tyr;
X₀₃ is any natural or non natural amino acid residue different from L- or D-Cys residue, such as L- or D-Met, L- or D-Nle, L- or D-Leu, L- or D-Glx, L- or D-Asx, L- or D-Orn- or D-Ala, IL- or D-Ile, L- or D-Val;
X₀₄ is any natural or non natural amino acid residue different from L- or D-Cys residue, such as L- or D-Phe, L- or D-His, L- or D-L- or D-Trp, L- or D-Tyr;
X₀₅ is any natural or non natural amino acid residue different from L- or D-Cys residue, such as L- or D-Lys, L- or D-Arg, L- or D-Orn- or D-Val, L- or D-Ile, L- or D-Leu;
X₀₆ is L- or D-Arg, L- or D-Lys, L- or D-Orn- or L- or D-Glx, L- or D-Asx, L- or D-Thr, L- or D-Tyr;
X₀₇ is any natural or non natural amino acid residue different from L- or D-Cys residue, such as Gly, L- or D-Pro, L- or D-L- or D-Val, or L- or D-Ala;
X₀₈ is L- or D-Arg, L- or D-Lys, L- or D-Orn- or L- or D-Glx, L- or D-Asx, L- or D-Thr, L- or D-Tyr;
X₀₉ is L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe;
X₁₀ is any natural or non natural amino acid different from L- or D-Cys, such as L- or D-Leu, L- or D-Ile, L- or D-Ala, L- or D-Thr, L- or D-Tyr, L- or D-Nle, L- or D-Ser;
X₁₁ is L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe;
X₁₂ is any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys;
X₁₃ is any natural or non natural amino acid residue different from L- or D-Cys residue, such as Gly, L- or D-Pro, L- or D-L- or D-Val, or L- or D-Ala;
X₁₄ is any natural or non natural amino acid residue different from L- or D-Cys, such as L- or D-Pro, L- or D-Ala, L- or D-Leu- or L- or D-Ile, L- or D-Phe, L- or D-Thr, L- or D-Tyr;
X₁₅ is any natural or non natural amino acid residue different from L- or D-Cys residue, such as Gly, L- or D-Pro, L- or D-L- or D-Val, or L- or D-Ala;
X₁₆ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;

and wherein,
Y₀₁ is one or four natural or non natural amino acid residue different from L- or D-Cys residue, such as L- or D-Ser, L- or D-Thr, L- or D-Lys, L- or D-Phe, L- or D-Tyr, L- or D-Arg, L- or D-Ala, L- or D-Glx, L- or D-Asx, L- or D-Val, and L- or D-Lys, L- or D-(Asn-Arg-Ala-Ser);
Y₀₂ is L- or D-Ser, L- or D-Thr, L- or D-Lys, L- or D-Phe, L- or D-Tyr, L- or D-Arg, L- or D-Ala, L- or D-His, L- or D-Trp, L- or D-Val;
Y₀₃ is L- or D-Ser, L- or D-Thr, L- or D-Lys, L- or D-Phe, L- or D-Tyr, L- or D-Arg, L- or D-Ala, L- or D-His, L- or D-Trp, L- or D-Val;
Y₀₄ is any natural or non natural amino acid residue different from L- or D-Cys residue, such as L- or D-Glx, L- or D-Asx, L- or D-Lys, L- or D-Arg, or L- or D-Phe, L- or D-Tyr;
Y₀₅ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
Y₀₆ is any natural or non natural amino acid residue different from L- or D-Cys residue, such as L- or D-Ser, L- or D-Thr, L- or D-Lys, L- or D-Arg, L- or D-Tyr, L- or D-Leu, L- or D-Ile, L- or D-Nle, L- or D-Ala, L- or D-Val, L- or D-Orn;
Y₀₇ is any natural or non natural amino acid residue different from L- or D-Cys residue, such as L- or D-Leu, L- or D-Ile, L- or D-Ala, L- or D-Nle, L- or D-Val;
Y₀₈ is any natural or non natural amino acid residue different from L- or D-Cys residue, such as L- or D-Leu, L- or D-Ile, L- or D-Ala, L- or D-Nle, L- or D-Val;
Y₀₉ is any natural or non natural amino acid residue different from L- or D-Cys residue, such as L- or D-Leu, L- or D-Ile, L- or D-Ala, L- or D-Nle, L- or D-Val;
Y₁₀ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
Y₁₁ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
Y₁₂ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val; L- or D-Ala;
Y₁₃ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val; and,

Y₁₄ is one or up to four natural or non natural amino acid residues different from L- or D-Cys, such as L- or D-Arg, L- or D-Lys, L- or D-Orn, L- or D-Glx, L- or D-Asx, L- or D-Phe, L- or D-Thr, L- or D-Tyr, L- or D-Ser, and L- or D-Arg, L- or D-(Arg-Lys), L- or D-(Arg-Lys-Asn-), L- or D-(Arg-Lys-Asn-Phe).

CXCL12, SDF-1 Compounds

In some embodiments, the invention includes mimetics of human chemokine SDF-1. In these embodiments, the analog can include a first conserved region and a second conserved region, wherein the first conserved region can include an N-terminal region, and the second conserved region can include a C-terminal region. The N-terminal region can include a series of up to 17 of the first 17 amino acids of a native SDF-1 chemokine, and the C-terminal region can include a series of up to 17 of the last 17 amino acids in the native SDF-1 chemokine. In some embodiments, these conserved regions can be linked using a linker.

In some embodiments, for example, the analog can comprise an N-terminal region having the first 14 residues of the native SDF-1 chemokine, and the C-terminal region can comprise residues 55-67 of the native SDF-1 chemokine:
(1-14)-[linker]-(55-67)

Possibilities for SDF-1 analogs are taught in detail in U.S. patent application Ser. Nos. 11/393,769, 11/388,542, 10/945,674, 10/086,177, 09/852,424, and 09/835,107, each of which is hereby incorporated herein by reference in its entirety. Possible configurations for the SDF-1 mimetics can include those shown in Table 5. The N-terminus can be acetylated, and the C-terminal can be amidated.

TABLE 5

(1-14)-linker-(55-67)
(1-17)-linker-(55-67)
(1-14)-linker-(55-67)-cyclo-56/60
(1-14)-linker-(55-67)-cyclo-60/64
(1-17)-linker-(55-67)-cyclo-56/60
(1-17)-linker-(55-67)-cyclo-60/64
Wherein, the linker can be any linker taught herein.

In some embodiments, the SDF-1 mimetics can be cyclized in their C-terminal region using the methods taught herein.

CXCL13, BCA-1, Compounds

In some embodiments, the CXCL13 (BCA-1) chemokine analogs include:

(SEQ ID NO:169)

R-X₀₁ X₀₂ X₀₃ X₀₄ X₀₅ X₀₆ X₀₇ X₀₈ X₀₉ X₁₀ X₁₁ X₁₂
X₁₃ X₁₄ X₁₅ X₁₆ [linker] Y₀₁ Y₀₂ Y₀₃ Y₀₄ Y₀₅ Y₀₆
Y₀₇ Y₀₈ Y₀₉ Y₁₀ Y₁₁ Y₁₂ Y₁₃ Y₁₄

wherein,
X₀₁ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
X₀₂ is any natural or non natural amino acid residue different from L- or D-Cys residue, such as L- or D-Leu, L- or D-Ile, L- or D-Ala, L- or D-Nle, L- or D-Val;
X₀₃ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
X₀₄ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
X₀₅ is L- or D-Tyr, L- or D-Phe, L- or D-His- or L- or D-Glx, L- or D-Asx, L- or D-Thr, L- or D-Trp, L- or D-Ser;
X₀₆ is L- or D-Tyr, L- or D-Phe, L- or D-His- or L- or D-Glx, L- or D-Asx, L- or D-Thr, L- or D-Trp, L- or D-Ser;
X₀₇ is any natural or non natural amino acid residue different from L- or D-Cys residue, such as L- or D-Thr, L- or D-Ser, L- or D-Tyr, or L- or D-Ala;
X₀₈ is any natural or non natural amino acid residue different from L- or D-Cys residue, such as L- or D-Thr, L- or D-Ser, L- or D-Tyr, or L- or D-Ala;
X₀₉ is any natural or non natural amino acid different from L- or D-Cys, such as L- or D-Leu, L- or D-Ile, L- or D-Ala, L- or D-Thr, L- or D-Tyr, L- or D-Nle, L- or D-Ser;
X₁₀ is L- or D-Arg, L- or D-Lys, L- or D-Orn- or L- or D-Glx, L- or D-Asx, L- or D-Thr, L- or D-Tyr;
X₁₁ is L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe;
X₁₂ is L- or D-Arg, L- or D-Lys, L- or D-Orn- or L- or D-Glx, L- or D-Asx, L- or D-Thr, L- or D-Tyr;
X₁₃ is L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe;
X₁₄ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
X₁₅ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
X₁₆ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;

and wherein,
Y₀₁ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val; L- or D-Gln;
Y₀₂ is L- or D-Val, L- or D-Thr, L- or D-Lys, L- or D-Phe, L- or D-Tyr, L- or D-Arg, L- or D-Ala, L- or D-Ser, L- or D-Trp, L- or D-Leu, L- or D-Ile;
Y₀₃ is any natural or non natural amino acid different from L- or D-Cys, such as L- or D-Leu, L- or D-Ile, L- or D-Ala, L- or D-Thr, L- or D-Tyr, L- or D-Nle, L- or D-Ser, L- or D-Glx, L- or D-Asx;
Y₀₄ is L- or D-Arg, L- or D-Lys, L- or D-Orn- or L- or D-Glx, L- or D-Asx, L- or D-Thr, L- or D-Tyr, L- or D-Trp, L- or D-His;
Y₀₅ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
Y₀₆ is any natural or non natural amino acid residue different from L- or D-Cys residue, such as L- or D-Ser, L- or D-Thr, L- or D-Lys, L- or D-Arg, L- or D-Tyr, L- or D-Leu, L- or D-Ile, L- or D-Nle, L- or D-Ala, L- or D-Glx, L- or D-Asx;
Y₀₇ is any natural or non natural amino acid residue different from L- or D-Cys residue, such as L- or D-Thr, L- or D-Ser, L- or D-Tyr, or L- or D-Ala, L- or D-Arg;
Y₀₈ is any natural or non natural amino acid residue different from L- or D-Cys residue, such as L- or D-Thr, L- or D-Ser, L- or D-Tyr, or L- or D-Ala, L- or D-Met, L- or D-Nle;
Y₀₉ is any natural or non natural amino acid residue different from L- or D-Cys residue, such as L- or D-Thr, L- or D-Ser, L- or D-Tyr, or L- or D-Ala, L- or D-Met, L- or D-Nle;
Y₁₀ is any natural or non natural amino acid residue different from L- or D-Cys residue, such as L- or D-Thr, L- or D-Ser, L- or D-Tyr, or L- or D-Ala, L- or D-Glx, L- or D-Asx; L- or D-Glu;
Y₁₁ is any natural or non natural amino acid different from L- or D-Cys, such as L- or D-Leu, L- or D-Ile, L- or D-Ala, L- or D-Thr, L- or D-Tyr, L- or D-Nle, L- or D-Ser, L- or D-Val;
Y₁₂ is any natural or non natural amino acid residue different from L- or D-Cys residue, such as L- or D-Pro, L- or D-Ala, L- or D-Leu, L- or D-Glx, L- or D-Asx, L- or D-Orn- or, L- or D-Val;
Y₁₃ is L- or D-Val, L- or D-Thr, L- or D-Lys, L- or D-Phe, L- or D-Tyr, L- or D-Arg, L- or D-Ala, L- or D-Ser, L- or D-Trp, L- or D-Leu, L- or D-Ile;
Y₁₄ is one or up to eight natural or non natural amino acid residues different from L- or D-Cys, such as L- or D-Pro, L- or D-Val, L- or D-Phe, L- or D-Glx, L- or D-Asx, L- or D-Lys, L- or D-Arg, L- or D-Ile, L- or D-Ser, and L- or D-Pro, L- or D-Lys, L- or D-(Pro-Val), L- or D-(Pro-Val-Phe), L- or D-(Pro-Val-Phe-Lys), L- or D-(Pro-Val-Phe-Lys-Arg), L- or D-(Pro-Val-Phe-Lys-Arg-Lys), L- or D-(Pro-Val-Phe-Lys-Arg-Lys-Ile), L- or D-(Pro-Val-Phe-Lys-Arg-Lys-Ile-Pro),

CXCL14, BRAK, Compounds

In some embodiments, the CXCL14 (BRAK) chemokine analogs include:

(SEQ ID NO:180)

R-X₀₁ X₀₂ X₀₃ X₀₄ X₀₅ X₀₆ X₀₇ X₀₈ X₀₉ X₁₀ X₁₁ X₁₂
X₁₃ X₁₄ X₁₅ X₁₆ [linker] Y₀₁ Y₀₂ Y₀₃ Y₀₄ Y₀₅ Y₀₆
Y₀₇ Y₀₈ Y₀₉ Y₁₀ Y₁₁ Y₁₂ Y₁₃ Y₁₄

wherein,
X₀₁ is any natural or non natural amino acid residue different from L- or D-Cys residue, such as L- or D-Thr, L- or D-Ser, L- or D-Tyr, or L- or D-Ala;
X₀₂ is any natural or non natural amino acid residue different from L- or D-Cys residue, such as L- or D-Lys, L- or D-Arg, L- or D-Orn- or D-Val, L- or D-Ile, L- or D-Leu;
X₀₃ is L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe;
X₀₄ is any natural or non natural amino acid residue different from L- or D-Cys residue, such as L- or D-Lys, L- or D-Arg, L- or D-Orn- or D-Val, L- or D-Ile, L- or D-Leu;
X₀₅ is L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe;
X₀₆ is any natural or non natural amino acid residue different from L- or D-Cys residue, such as L- or D-Thr, L- or D-Ser, L- or D-Tyr, or L- or D-Ala;
X₀₇ is L- or D-Arg, L- or D-Lys, L- or D-Orn- or L- or D-Glx, L- or D-Asx, L- or D-Thr, L- or D-Tyr;
X₀₈ is any natural or non natural amino acid residue different from L- or D-Cys residue, such as L- or D-Lys, L- or D-Arg, L- or D-Orn- or D-Val, L- or D-Ile, L- or D-Leu;
X₀₉ is any natural or non natural amino acid residue different from L- or D-Cys residue, such as Gly, L- or D-Pro, L- or D-L- or D-Val, or L- or D-Ala;
X₁₀ is any natural or non natural amino acid residues different from L- or D-Cys, such as L- or D-Pro, L- or D-Val, L- or D-Phe, L- or D-Glx, L- or D-Asx, L- or D-Lys, L- or D-Arg, L- or D-Ile, L- or D-Ser;
X₁₁ is any natural or non natural amino acid residue different from L- or D-Cys residue, such as L- or D-Lys, L- or D-Arg, L- or D-Orn- or D-Val, L- or D-Ile, L- or D-Leu;
X₁₂ is any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys;
X₁₃ is L- or D-Arg, L- or D-Lys, L- or D-Orn- or L- or D-Glx, L- or D-Asx, L- or D-Thr, L- or D-Tyr;
X₁₄ is L- or D-Tyr, L- or D-Phe, L- or D-His- or L- or D-Glx, L- or D-Asx, L- or D-Thr, L- or D-Trp, L- or D-Ser;
X₁₅ is any natural or non natural amino acid residue different from L- or D-Cys residue, such as L- or D-Thr, L- or D-Ser, L- or D-Tyr, or L- or D-Ala;
X₁₆ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;

and wherein,
Y₀₁ is any natural or non natural amino acid residue different from L- or D-Cys residue, such as L- or D-Lys, L- or D-Trp, L- or D-Arg, L- or D-Phe, L- or D-Tyr, L- or D-His, L- or D-Ala, L- or D-Glx, L- or D-Asx, L- or D-Val;
Y₀₂ is L- or D-Ser, L- or D-Thr, L- or D-Lys, L- or D-Phe, L- or D-Tyr, L- or D-Arg, L- or D-Ala, L- or D-His, L- or D-Trp, L- or D-Val, L- or D-Leu, L- or D-Ile;
Y₀₃ is any natural or non natural amino acid residue different from L- or D-Cys residue, such as L- or D-Glx, L- or D-Asx, L- or D-Lys, L- or D-Arg, or L- or D-Phe, L- or D-Tyr;
Y₀₄ is any natural or non natural amino acid residue different from L- or D-Cys residue, such as L- or D-Val, L- or D-Lys, L- or D-Arg, L- or D-Glx, L- or D-Asx, L- or D-Ser- or D-Ala, IL- or D-Thr, L- or D-Tyr;
Y₀₅ is any natural or non natural amino acid residue different from L- or D-Cys residue, such as L- or D-Thr, L- or D-Ser, L- or D-Tyr, or L- or D-Trp;
Y₀₆ is any natural or non natural amino acid residue different from L- or D-Cys residue, such as L- or D-Lys, L- or D-Trp, L- or D-Arg, L- or D-Phe, L- or D-Tyr, L- or D-His, L- or D-Ala, L- or D-Glx, L- or D-Asx, L- or D-Val;
Y₀₇ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
Y₀₈ is any natural or non natural amino acid residue different from L- or D-Cys residue, such as L- or D-Phe, L- or D-Lys, L- or D-Ala, L- or D-Arg;
Y₀₉ is any natural or non natural amino acid residue different from L- or D-Cys residue, such as L- or D-Leu, L- or D-Ile, L- or D-Ala, L- or D-Nle, L- or D-Arg, L- or D-Lys; L- or D-Arg;
Y₁₀ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
Y₁₁ is any natural or non natural amino acid residue different from L- or D-Cys residue, such as L- or D-Val, L- or D-Lys, L- or D-Arg, L- or D-Glx, L- or D-Asx, L- or D-Trp- or D-Ala, IL- or D-Phe, L- or D-His;
Y₁₂ is L- or D-Tyr, L- or D-Phe, L- or D-His- or L- or D-Glx, L- or D-Asx, L- or D-Thr, L- or D-Trp, L- or D-Ser;
Y₁₃ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
Y₁₄ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;

CXCL15, Lungkine, Compounds

In some embodiments, the CXCL15 (Lungkine) chemokine analogs include:

(SEQ ID NO:194)

R-X₀₁ X₀₂ X₀₃ X₀₄ X₀₅ X₀₆ X₀₇ X₀₈ X₀₉ X₁₀ X₁₁ X₁₂
X₁₃ X₁₄ X₁₅ X₁₆ [linker] Y₀₁ Y₀₂ Y₀₃ Y₀₄ Y₀₅ Y₀₆
Y₀₇ Y₀₈ Y₀₉ Y₁₀ Y₁₁ Y₁₂ Y₁₃ Y₁₄

wherein,
X₀₁ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile;
X₀₂ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile;
X₀₃ is any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys;
X₀₄ is L- or D-Arg, L- or D-Lys, L- or D-Orn- or L- or D-Glx, L- or D-Asx, L- or D-Thr, L- or D-Tyr;
X₀₅ is L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe;
X₀₆ is any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys;
X₀₇ is L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe;
X₀₈ is any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys;
X₀₉ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile;
X₁₀ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile;
X₁₁ is L- or D-His, L- or D-Lys, L- or D-Orn- or L- or D-Glx, L- or D-Asx, L- or D-Trp, L- or D-Phe, L- or D-Tyr;
X₁₂ is any natural or non natural amino acid residue different from L- or D-Cys residue, such as L- or D-Thr, L- or D-Ser, L- or D-Tyr, or L- or D-Ala;
X₁₃ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile;
X₁₄ is any natural or non natural amino acid residue different from L- or D-Cys residue, such as L- or D-Phe, L- or D-Lys, L- or D-Ala, L- or D-Arg;
X₁₅ is any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys;
X₁₆ is any natural or non natural amino acid residues different from L- or D-Cys, such as L- or D-Pro, L- or D-Val, L- or D-Phe, L- or D-Glx, L- or D-Asx, L- or D-Lys, L- or D-Arg, L- or D-Ile, L- or D-Ser;

and wherein,
Y₀₁ is any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys; L- or D-Asp;
Y₀₂ is L- or D-Arg, L- or D-Lys, L- or D-Orn- or L- or D-Glx, L- or D-Asx, L- or D-Thr, L- or D-Tyr;
Y₀₃ is any natural or non natural amino acid residue different from L- or D-Cys residue, such as L- or D-Glx, L- or D-Asx, L- or D-Lys, L- or D-Arg, or L- or D-Phe, L- or D-Tyr; L- or D-Asn;
Y₀₄ is any natural or non natural amino acid residue different from L- or D-Cys residue, such as L- or D-Phe, L- or D-Tyr, L- or D-His, L- or D-Glx, L- or D-Asx, L- or D-Ser- or D-Ala, IL- or D-Thr, L- or D-Trp; L- or D-Phe;
Y₀₅ is L- or D-Ser, L- or D-Leu, L- or D-Ile- or L- or D-Glx, L- or D-Asx, L- or D-Thr, L- or D-Tyr, L- or Ala, L- or D-Phe, L- or D-His;
Y₀₆ is L- or D-Arg, L- or D-Lys, L- or D-Orn- or L- or D-Glx, L- or D-Asx, L- or D-Thr, L- or D-Tyr;
Y₀₇ is any natural or non natural amino acid residue different from L- or D-Cys residue, such as L- or D-His, L- or D-Arg, L- or D-Glx, L- or D-Asx, L- or D-Orn- or D-Phe, L- or D-Ile, L- or D-Trp;
Y₀₈ is any natural or non natural amino acid residue different from L- or D-Cys residue, such as L- or D-Phe, L- or D-Ser, L- or D-Thr, L- or D-Tyr;
Y₀₉ is any natural or non natural amino acid residue different from L- or D-Cys residue, such as L- or D-Ser, L- or D-Thr, L- or D-Ala, L- or D-Tyr, L- or D-Arg, L- or D-Lys;
Y₁₀ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
Y₁₁ is any natural or non natural amino acid residue different from L- or D-Cys residue, such as L- or D-Val, L- or D-Leu, L- or D-Ile, L- or D-Glx, L- or D-Asx, L- or D-Trp- or D-Ala, IL- or D-Phe, L- or D-His;
Y₁₂ is any natural or non natural amino acid residue different from L- or D-Cys residue, such as L- or D-Ser, L- or D-Thr, L- or D-Ala, L- or D-Tyr, L- or D-Arg, L- or D-Lys;
Y₁₃ is any natural or non natural amino acid residue different from L- or D-Cys residue, such as L- or D-His, L- or D-Leu, L- or D-Ile, L- or D-Glx, L- or D-Asx, L- or D-Trp- or D-Ala, L- or D-Phe;
Y₁₄ is one or up to five natural or non natural amino acid residue different from L- or D-Cys residue, such as L- or D-Thr, L- or D-Ser, L- or D-Arg, L- or D-Glx, L- or D-Asx, L- or D-Orn- or D-Ala, IL- or D-Ile, Gly, and L- or D-Asn, L- or D-Thr, L- or D-(Thr-Gly), L- or D-(Thr-Gly-Ser), (Thr-Gly-Ser-Asp), L- or D-(Thr-Gly-Ser-Asp-Ala),

CXCL16, SRPSOX, Compounds

In some embodiments, the CXCL16 (SRPSOX) chemokine analogs include:

(SEQ ID NO:204)

R-X₀₁ X₀₂ X₀₃ X₀₄ X₀₅ X₀₆ X₀₇ X₀₈ X₀₉ X₁₀ X₁₁ X₁₂
X₁₃ X₁₄ X₁₅ X₁₆ [linker] Y₀₁ Y₀₂ Y₀₃ Y₀₄ Y₀₅ Y₀₆
Y₀₇ Y₀₈ Y₀₉ Y₁₀ Y₁₁ Y₁₂ Y₁₃ Y₁₄

wherein,
X₀₁ is any natural or non natural amino acid residue different from L- or D-Cys residue, such as Gly, L- or D-Pro, L- or D-L- or D-Val, or L- or D-Ala;
X₀₂ is any natural or non natural amino acid residue different from L- or D-Cys residue, such as L- or D-Thr, L- or D-Ser, L- or D-Tyr, or L- or D-Ala;
X₀₃ is any natural or non natural amino acid residue different from L- or D-Cys residue, such as L- or D-Val, L- or D-Ala, L- or D-Leu- or D-Ile;
X₀₄ is any natural or non natural amino acid residue different from L- or D-Cys residue, such as L- or D-Thr, L- or D-Ser, L- or D-Tyr, or L- or D-Ala;
X₀₅ is any natural or non natural amino acid residue different from L- or D-Cys residue, such as Gly, L- or D-Pro, L- or D-L- or D-Val, or L- or D-Ala;
X₀₆ is any natural or non natural amino acid residue different from L- or D-Cys residue, such as L- or D-Thr, L- or D-Ser, L- or D-Tyr, or L- or D-Ala;
X₀₇ is L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe;
X₀₈ is any natural or non natural amino acid residue different from L- or D-Cys residue, such as L- or D-Tyr, L- or D-Phe, L- or D-His- or L- or D-Glx, L- or D-Asx, L- or D-Thr, L- or D-Trp, L- or D-Ser;
X₀₉ is L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe;
X₁₀ is any natural or non natural amino acid residue different from L- or D-Cys residue, such as Gly, L- or D-Pro, L- or D-L- or D-Val, or L- or D-Ala;
X₁₁ is any natural or non natural amino acid residue different from L- or D-Cys residue, such as L- or D-Lys, L- or D-Arg, L- or D-Orn- or D-Val, L- or D-Ile, L- or D-Leu;
X₁₂ is L- or D-Arg, L- or D-Lys, L- or D-Orn- or L- or D-Glx, L- or D-Asx, L- or D-Thr, L- or D-Tyr;
X₁₃ is any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys;
X₁₄ is any natural or non natural amino acid residue different from L- or D-Cys residue, such as L- or D-Thr, L- or D-Ser, L- or D-Tyr, or L- or D-Ala;
X₁₅ is any natural or non natural amino acid residue different from L- or D-Cys residue, such as L- or D-Thr, L- or D-Ser, L- or D-Tyr, or L- or D-Ala;
X₁₆ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;

and wherein,
Y₀₁ is any natural or non natural amino acid residue different from L- or D-Cys residue, such as L- or D-Trp, L- or D-Arg, L- or D-Phe, L- or D-Tyr, L- or D-His, L- or D-Ala, L- or D-Glx, L- or D-Asx, L- or D-Val;
Y₀₂ is any natural or non natural amino acid residue different from L- or D-Cys residue, such as L- or D-Val, L- or D-Ala, L- or D-Leu- or D-Ile;
Y₀₃ is any natural or non natural amino acid residue different from L- or D-Cys residue, such as L- or D-Glx, L- or D-Asx, L- or D-Lys, L- or D-Arg, or L- or D-Phe, L- or D-Tyr;
Y₀₄ is any natural or non natural amino acid residue different from L- or D-Cys residue, such as L- or D-Val, L- or D-Lys, L- or D-Arg, L- or D-Glx, L- or D-Asx, L- or D-Ser- or D-Ala, IL- or D-Thr, L- or D-Tyr;
Y₀₅ is any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys;
Y₀₆ is L- or D-Met, L- or D-Nle, L- or D-His- or L- or D-Glx, L- or D-Asx, L- or D-Thr, L- or D-Trp, L- or D-Ser, L- or D-Tyr, L- or D-Lys, L- or D-Orn;
Y₀₇ is any natural or non natural amino acid residue different from L- or D-Cys residue, such as L- or D-Thr, L- or D-Ser, L- or D-Tyr, or L- or D-Ala;
Y₀₈ is L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe, L- or D-Nle;
Y₀₉ is any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys;
Y₁₀ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val;
Y₁₁ is any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys;
Y₁₂ is any natural or non natural amino acid residue different from L- or D-Cys residue, such as L- or D-Lys, L- or D-Arg, L- or D-Orn- or D-Val, L- or D-Ile, L- or D-Leu;
Y₁₃ is any natural or non natural amino acid residue different from L- or D-Cys residue, such as L- or D-Val, L- or D-Lys, L- or D-Arg, L- or D-Glx, L- or D-Asx, L- or D-Orn- or D-Ala, IL- or D-Ile, L- or D-Val; and
Y₁₄ is one or up to six natural or non natural amino acid residues, such as L- or D-Cys, Gly, L- or D-His, L- or D-Ala, L- or D-Tyr, L- or D-Ser, L- or D-Phe, IL- or D-Nle, L- or D-Tyr, and L- or D-Cys, L- or D-(Cys-Gly), L- or D-(Cys-Gly-His), L- or D-(Cys-Gly-His-Ala), L- or D-(Cys-Gly-His-Ala-Tyr), L- or D-(Cys-Gly-His-Ala-Tyr-Ser).

CXCL17, DMC, Compounds

In some embodiments, the invention includes mimetics of human chemokine DMC. In these embodiments, the analog can include a first conserved region and a second conserved region, wherein the first conserved region can include an N-terminal region, and the second conserved region can include a C-terminal region. The N-terminal region can include a series of up to 17 of the first 17 amino acids of a native DMC chemokine, and the C-terminal region can include a series of up to 17 of the last 17 amino acids in the native DMC chemokine. In some embodiments, these conserved regions can be linked using a linker.

In some embodiments, for example, the analog can comprise an N-terminal region having the first 14 residues of the native DMC chemokine, and the C-terminal region can comprise residues 55-67 of the native DMC chemokine:
(1-15)-[linker]-(105-119)

Possible configurations for the DMC mimetics can include those shown in Table 6. The N-terminus can be acetylated, and the C-terminal can be amidated.

TABLE 6

(1-15)-linker-(105-119)
(1-17)-linker-(105-119)
(1-14)-linker-(102-119)-cyclo
(1-14)-linker-(103-119)-cyclo
(1-17)-linker-(104-119)-cyclo
(1-17)-linker-(105-119)-cyclo
Wherein, the linker can be any linker taught herein.

In some embodiments, the DMC mimetics can be cyclized in their C-terminal region using the methods taught herein.

### Synthesis

CXC chemokine analog compounds of the invention may be prepared by standard techniques known in the art. A peptide or polypeptide component of a CXC chemokine analog may comprise, at least in part, a peptide synthesized using standard techniques (such as those described by Clark-Lewis, I., Dewald, B., Loetscher, M., Moser, B., and Baggiolini, M., (1994) J. Biol. Chem., 269, 16075-16081). Automated peptide synthesizers are commercially available (e.g., Advanced ChemTech Model 396; Milligen/Biosearch 9600, Appliedbiosystems/Pioneer).

Peptides and polypeptides may be assayed for CXC chemokine receptor agonist or antagonist activity in accordance with standard methods. Peptides and polypeptides may be purified by HPLC and analyzed by mass spectrometry. Peptides and polypeptides may be dimerized. In some embodiments, the peptides are dimerized via a disulfide bridge formed by gentle oxidation of the cysteines using 10% DMSO in water. Following HPLC purification, dimer formation may be verified, by mass spectrometry. One or more modifying groups may be attached to a peptidic component by standard methods, for example, using methods for reaction through an amino group (e.g., the alpha-amino group at the amino-terminus of a peptide), a carboxyl group (e.g., at the carboxy terminus of a peptide), a hydroxyl group (e.g., on a tyrosine, serine or threonine residue) or other suitable reactive group on an amino acid side chain.

In some embodiments, analogs derived from the C-terminal and N-terminal joined by a linker could be cyclized in their C-terminal moiety using side-chain to side-chain; side-chain to scaffold or, scaffold to scaffold cyclization. In some embodiments, lactamization, etherification, or RCM (Ring Closing Methatesis) are used to carry out this reaction. The CXC chemokine analogs may be cyclized using a lactam formation procedure by joining the γ-carboxy side chain or the α-carboxy moiety of glutamate (Glu) residue to the ε-amino side chain of lysine (Lys) residue, as indicated in the following sequences by underlining of linked residues. Lactams may for example be formed between glutamic acid and lysine (Lys) in the C-terminal portion of the polypeptide (which does not correspond necessarily with the numbering of that residue in the native sequence). In further alternatives, a lysine (Lys) may be substituted by ornithine (Orn) or any other (L or D) natural or (L or D) non-natural amino acid having an amino group on its side chain. Similarly, glutamate (Glu) may for example be substituted with aspartate (Asp), denoted by nomenclature such as (Glu->Asp) indicating a substitution in a given position in the peptide wherein aspartate replaces glutamate.

The CXC chemokine analogs include sequences wherein one or more of the amino acids have been replaced by a conservative amino acid substitution. The term “conservative amino acid substitution” refers to a polypeptide chain in which one of the amino acid residues is replaced with an amino acid residue having a side chain with similar properties. Families of amino acid residues having side chains with similar properties are well known in the art. These families include amino acids with acidic side chains (e.g., aspartic acid, glutamic acid), basic side chains (e.g., lysine, arginine, histidine), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, an amino acid residue in a chemokine is replaced with another amino acid residue from the same side chain family.

### Recombinant Synthesis

CXC chemokines, CXC chemokine fragments, or CXC chemokine analogs may also be synthesized, in whole or in part, by recombinant methods using expression vectors encoding all or part of a CXC chemokine. Vectors, or preferably expression vectors, may contain a gene encoding a polypeptide of the invention, a functional derivative thereof, or another useful polypeptide. These vectors may be employed to express the encoded polypeptide in either prokaryotic or eukaryotic cells.

The term “vector” in this application refers to a DNA molecule into which another DNA of interest can be inserted by incorporation into the DNA of the vector. One skilled in the art is familiar with the term. Examples of classes of vectors can be plasmids, cosmids, viruses, and bacteriophage. Typically, vectors are designed to accept a wide variety of inserted DNA molecules and then used to transfer or transmit the DNA of interest into a host cell (e.g., bacterium, yeast, higher eukaryotic cell). A vector may be chosen based on the size of the DNA molecule to be inserted, as well as based on the intended use. For transcription into RNA or transcription followed by translation to produce an encoded polypeptide, an expression vector would be chosen. For the preservation or identification of a specific DNA sequence (e.g., one DNA sequence in a cDNA library) or for producing a large number of copies of the specific DNA sequence, a cloning vector would be chosen. If the vector is a virus or bacteriophage, the term vector may include the viral/bacteriophage coat.

Following entry into a cell, all or part of the vector DNA, including the insert DNA, may be incorporated into the host cell chromosome, or the vector may be maintained extrachromosomally. Those vectors that are maintained extrachromosomally are frequently capable of autonomous replication in a host cell into which they are introduced (e.g., many plasmids having a bacterial origin of replication). Other vectors are integrated into the genome of a host cell upon introduction into the host cell, and thereby are replicated along with the host genome.

The term “expression vector” refers to a DNA construct which allows one to place a gene encoding a gene product of interest, usually a protein, into a specific location in a vector from which the selected gene product can be expressed by the machinery of the host cell, or alternately, by in vitro expression system. This type of vector is frequently a plasmid, but other forms of expression vectors, such as bacteriophage vectors and viral vectors (e.g., adenoviruses, replication defective retroviruses, and adeno-associated viruses), may be employed. The selection of expression vectors, control sequences, transformation methods, and the like, are dependent on the type of host cell used to express the gene.

Prokaryotic hosts are, in generally, very efficient and convenient for the production of recombinant polypeptides and are, therefore, one type of preferred expression system. Prokaryotes most frequently are represented by various strains of E. coli, but other microbial strains may be used, including other bacterial strains. Recognized prokaryotic hosts include bacteria such as E. coli, Bacillus, Streptomyces, Pseudomonas, Salmonella, Serratia, and the like. However, under such conditions, recombinantly-produced polypeptides will not be glycosylated. Other plant cells may also be utilized as hosts, and control sequences compatible with plant cells are available, such as the cauliflower mosaic virus 35S and 19S promoters, and nopaline synthase promoter and polyadenylation signal sequences. Furthermore, the protein of interest may be expressed in plants which have incorporated the expression vector into their germ line.

In prokaryotic systems, vectors that contain replication sites and control sequences derived from a species compatible with the host may be used. Preferred prokaryotic vectors include plasmids such as those capable of replication in E. coli (such as, for example, pBR322, ColEl, pSC101, pACYC 184, pVX, pUC118, pUC119 and the like). Suitable phage or bacteriophage vectors may include λgt10, λgt11, vectors derived from filamentous bacteriophage such as m13, and the like. Suitable Streptomyces plasmids include p1J101, and streptomyces bacteriophages such as fC31. Bacillus plasmids include pC194, pC221, pT127, and the like. Suitable Pseudomonas plasmids have been reviewed by Izaki (Jpn. J. Bacteriol. 33:729-742, 1978) and John et al. (Rev. Infect. Dis. 8:693-704, 1986).

For expression of a protein in a prokaryotic cell, it is necessary to operably link the sequence encoding the protease of the invention to a functional prokaryotic promoter. Such promoters are either constitutive or inducible promoters, but commonly inducible promoters are used. Examples of constitutive promoters include the int promoter of bacteriophage λ, the bla promoter of the β-lactamase gene sequence of pBR322, and the cat promoter of the chloramphenicol acetyl transferase gene sequence of pPR325, and the like. Examples of inducible prokaryotic promoters include the major right and left promoters of bacteriophage λ (PL and PR), the trp, recA, lacZ, lac, and gal promoters of E. coli, the α-amylase and the V-28-specific promoters of B. subtilis, the promoters of the bacteriophages of Bacillus, and Streptomyces promoters. Prokaryotic promoters are reviewed by Glick (Ind. Microbiot. 1:277-282, 1987), Cenatiempo (Biochimie 68:505-516, 1986), and Gottesman (Ann. Rev. Genet. 18:415-442, 1984). Additionally, proper expression in a prokaryotic cell also requires the presence of a ribosome-binding site upstream of the encoding sequence.

Recombinant protein expression in E. coli can be increased by expressing the protein or fusion protein in a host bacteria with an impaired proteolytic system so as to reduce the post-synthesis degradation of the recombinant protein (Gottesman, S., Gene Expression Technology: Methods in Enzymology 185, Academic Press, San Diego, Calif. (1990) 119-128). Another strategy is to alter the mix of codons used in the coding sequence to reflect the usage of the individual codons for each amino acid in the host (e.g., E. coli (Wada et al., (1992) Nucleic Acids Res. 20:2111-2118)). Such alteration of nucleic acid sequences of the invention can be carried out by standard DNA synthesis techniques and may prove useful for a variety of prokaryotic and eukaryotic expression systems.

Suitable hosts may also include eukaryotic cells. Preferred eukaryotic hosts include, for example, yeast, fungi, insect cells, and mammalian cells both in vivo and in tissue culture. Useful mammalian cell hosts include HeLa cells, cells of fibroblast origin such as VERO or CHO-K1, and cells of lymphoid origin and their derivatives. Preferred mammalian host cells include SP2/0 and J558L, as well as neuroblastoma cell lines such as IMR 332, which may provide better capacities for correct post-translational processing. In general, eukaryotic organisms such as yeast provide substantial advantages in that they can also carry out post-translational modifications. 35:365-404, 1981).

A large number of yeast expression systems may be potentially utilized which incorporate promoter and termination elements from the actively expressed sequences coding for glycolytic enzymes. These expression systems produce large quantities of proteins when yeast are grown in mediums rich in glucose. Known glycolytic gene sequences can also provide very efficient transcriptional control signals. A number of recombinant DNA strategies utilize strong promoter sequences and high copy number plasmids which can be utilized for production of the desired proteins in yeast. Examples of vectors suitable for expression in S. cerivisae include pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), pYES2 (InVitrogen Corporation, San Diego, Calif.), and picZ (Invitrogen Corp, San Diego, Calif.).

In another embodiment, the protein of interest may be expressed in insect cells for example the Drosophila larvae. Using insect cells as hosts, the Drosophila alcohol dehydrogenase promoter may be used (Rubin, Science 240:1453-1459, 1988). Additionally, baculovirus vectors can be engineered to express large amounts of the protein of interest in cultured insect cells (e.g., Sf 9 cells) (Jasny, Science 238:1653, 1987; Miller et al., in: Genetic Engineering, Vol. 8, Plenum, Setlow et al., eds., pp. 277-297, 1986). Vectors which may be used include the pAc series (Smith et al. (1983) Mol. Cell. Biol. 3:2156-2165) and the pVL series (Lucklow and Summers (1989) Virology 170:31-39).

Possibilities and techniques for expression in mammalian cells has recently been summarized (Colosimo, et al., “Transfer and expression of foreign genes in mammalian cells,” Biotechniques 29(2):314-8, 320-2, 324 passim, 2000; which is hereby incorporated by reference in its entirety including any drawings, tables, and figures.). Examples of mammalian expression vectors include pCDM8 (Seed, B. (1987) Nature 329:840) and pMT2PC (Kaufman et al. (1987) EMBO J. 6:187-195). For use in mammalian cells, the regulatory sequences of the expression vector are often derived from viral regulatory elements. For example, commonly used promoters are derived from Simian Virus 40 (SV40), polyoma, Adenovirus 2, and cytomegalovirus (CMV) viruses. Preferred eukaryotic promoters include, for example, the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-31, 1981); and the yeast gal4 gene sequence promoter (Johnston et al., Proc. Natl. Acad. Sci. (USA) 79:6971-6975, 1982; Silver et al., Proc. Natl. Acad. Sci. (USA) 81:5951-5955, 1984). Alternatively, promoters from mammalian expression products, such as actin, collagen, myosin, and the like, may be employed. Regulatory elements may also be derived from adenovirus, bovine papilloma virus, cytomegalovirus, simian virus, or the like.

Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or are subject to chemical (such as metabolite) regulation. Expression of proteins of interest in eukaryotic hosts requires the use of eukaryotic regulatory regions. Such regions will, in general, include a promoter region sufficient to direct the initiation of RNA synthesis.

A recombinant mammalian expression vector may also be designed to be capable of directing expression of the nucleic acid preferentially in a particular cell type (i.e., tissue-specific regulatory elements are used to control the expression). Such tissue-specific promoters include the liver-specific albumin promoter (Pinkert et al. (1987) Genes Dev. 1:268-277); lymphoid-specific promoters (e.g., Calame and Eaton (1988) Adv. Immunol. 43:235-275), and in particular promoters of immunoglobulins and T cell receptors (Winoto and Baltimore (1989) EMBO J. 8:729-733, Banerji et al. (1983) Cell 33:729-740; Queen and Baltimore (1983) Cell 33:741-748); mammary gland-specific promoters (e.g., milk whey promoter; U.S. Pat. No. 4,873,316 and European Application Publication No. 264,166); and pancreas-specific promoters (Edlund et al. (1985) Science 230:912-916). Developmentally-regulated promoters may also be utilized, for example, the α-fetoprotein promoter (Campes and Tilghman (1989) Genes Dev. 3:537-546), and the murine hox promoters (Kessel and Gruss (1990) Science 249:374-379).

Preferred eukaryotic plasmids include, for example, SV40, BPV, pMAM-neo, pKRC, vaccinia, 2-micron circle, and the like, or their derivatives. Such plasmids are well known in the art (Botstein et al., Miami Wntr. Symp. 19:265-274, 1982; Broach, In: “The Molecular Biology of the Yeast Saccharomyces: Life Cycle and Inheritance,” Cold Spring Harbor Laboratory, Cold Spring Harbor, N.Y., p. 445-470, 1981; Broach, Cell 28:203-204, 1982; Bollon et al., J. Clin. Hematol. Oncol. 10:39-48, 1980; Maniatis, In: Cell Biology: A Comprehensive Treatise, Vol. 3, Gene Sequence Expression, Academic Press, NY, pp. 563-608, 1980).

Once the vector or nucleic acid molecule containing the construct(s) has been prepared for expression, the DNA construct(s) may be introduced into an appropriate host cell by any of a variety of suitable means, i.e., transformation, transfection, conjugation, protoplast fusion, electroporation, particle gun technology, DEAE-dextran-mediated transfection, lipofection, calcium phosphate-precipitation, direct microinjection, and the like. Suitable methods for transforming or transfecting host cells can be found in Sambrook, et al. (2001). After the introduction of the vector, recipient cells are grown in a selective medium, which selects for the growth of vector-containing cells. Expression of the cloned gene(s) results in the production of a protein of interest, or fragments thereof.

For transformation of eukaryotic cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin, neomycin, methotrexate, glyphosate, and bialophos. Nucleic acid encoding a selectable marker can be introduced into a host cell on the same vector as that encoding the protein of interest or can be introduced on a separate vector. Cells stably transformed with the introduced nucleic acid can be identified by drug selection (e.g., cells that have incorporated the selectable marker gene will survive, while the other cells die).

Proteins may be expressed as fusion proteins. Genes for proteins expressed as fusion proteins ligated into expression vectors that add a number of amino acids to a protein encoded and expressed, usually to the amino terminus of the recombinant protein. Such a strategy of producing fusion proteins is usually adopted for three purposes: (1) to assist in the purification by acting as a ligand in affinity purification, (2) to increase the solubility of the product, and (3) to increase the expression of the product. Often, expression vectors for use in fusion protein production, a proteolytic cleavage site is included at the junction of the fusion region and the protein of interest to enable purification of the recombinant protein away from the fusion region following affinity purification of the fusion protein. Such enzymes, and their cognate recognition sequences, include Factor Xa, thrombin and enterokinase, and may also include trypsin or chymotrypsin. Typical fusion expression vectors include pGEX (Pharmacia Biotech Inc; Smith, D. B. and Johnson, K. S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

For a variety of suitable expression systems for both prokaryotic and eukaryotic cells see Sambrook, et al., “Molecular Cloning: A Laboratory Manual,” 3rd ed., Cold Spring Harbor Laboratory, Cold Spring Harbor Laboratory Press, Cold Spring Harbor, N.Y., 2001, which is hereby incorporated by reference in its entirety, including any drawings, figures, and tables.

As described herein, the CXC chemokine analogs can be modified in a variety of ways. In many embodiments, the R-group consists of a hydrogen or is an N-terminal modifier comprising a component selected from a group consisting of a poly(ethylene glycol) or derivative thereof, a glycosaminoglycan, a diagnostic label, a radioactive group, an acyl group, an acetyl group, a peptide, and a modifier capable of reducing the ability of the analog to act as a substrate for aminopeptidases. In many embodiments, the C-terminus of the analogs can be amidated.

In many embodiments, a side chain to side chain cyclization can be produced between amino acid residues in the C-terminal region and can include lactamization, etherification, thioetherification, or cyclization generated by Mitsunubo or Ring Closing Methathesis (RCM) type of reactions. In many embodiments, the C-terminal region included in these peptides can form a stable α-helix moiety;

In many of the embodiments taught herein, the linker can consist of up to four amino acids, -Xaa₁-Xaa₂-Xaa₃-Xaa₄-, wherein Xaa₁, Xaa₂, Xaa₃, and Xaa₄ are each independently selected from a group consisting of (a) any natural amino acid, and (b) any non-natural amino acid having the following structure:

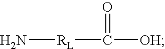

wherein, R_{L} is selected from a group consisting of saturated and unsaturated aliphatics and heteroaliphatics consisting of 20 or fewer carbon atoms that are optionally substituted with (i) a hydroxyl, carboxyl, amino, amido, or imino group; or (ii) an aromatic group having from 5 to 7 members in the ring. In some embodiments, the R_{L} group can have from 0 to 10 carbon atoms and bear a positive charge. In some embodiments, the linker can comprise at least one amino acid having a side chain bearing positive charge. In some embodiments, the natural amino acid is not L- or D-Cys.

The amino acids used in the present invention may be organic compounds comprising an amino group and a carboxyl group, and the amino group may be primary or secondary. Examples of amino acids include, but are not limited to, glycine, alanine, valine, leucine, isoleucine, methionine, phenylalanine, tyrosine, aspartic acid, glutamic acid, lysine, arginine, serine, threonine, cysteine, asparagine, proline, tryptophan, histidine and combinations thereof. In some embodiments, R_{L} may be a substituted, unsubstituted, hetero-, straight-chained, branched, cyclic, saturated or unsaturated aliphatic radical; or a substituted, unsubstituted, or hetero-aromatic radical. In some embodiments, R_{L} can be substituted, unsubstituted, or hetero-forms of methyl, iso-propyl, sec-butyl, iso-butyl, benzyl, or a combination thereof.

In embodiments where R_{L} is substituted, examples of substitutents include, but are not limited to, hydroxyl, carboxyl, amino, imino groups and combinations thereof. In embodiments where R_{L} is heteroaliphatic, examples of heteroatoms include, but are not limited to, sulfur, phosphorous, oxygen, nitrogen and combinations thereof. In some embodiments, R_{L} can comprise substituted or unsubstituted poly(alkylene glycols), which include, but are not limited to, PEG and PEG derivatives functionalized to link to specific chemical groups (available from Nektar Therapeutics, San Carlos, Calif.), poly(ethylene oxide), PPG, poly(tetramethylene glycol), poly(ethylene oxide-co-propylene oxide), or copolymers and combinations thereof.

In some embodiments, the amino acids may be bifunctional or trifunctional amino acids. In some embodiments, the amino acids may be limited to diamines, triamines, monocarboxylics, dicarboxylics, aliphatics, aromatics, amides, or a combination thereof. In some embodiments, the amino acids may not include any amino acid or group of amino acids, such as, for example lysine and its conservative substitutions. It is to be appreciated that one skilled in the art should recognize that some of the groups, subgroups, and individual amino acids may not be used in some embodiments of the present invention.

In some embodiments, R_{L} can be a substituted or unsubstituted alkylene comprising Cₙ carbons in the alkylene backbone, wherein n is an integer ranging from 1 to about 20; from about 2 to about 16; from about 3 to about 12; from about 4 to about 10; from about 3 to about 8, and any range therein. In these embodiments, the linker can be, for example, 11-aminoundecanoic acid.

In some embodiments, the linker can include any combination of natural or non-natural amino acids, wherein the number of amino acids can range from 1 to about 20; from about 2 to about 20; from about 3 to about 15, from about 4 to about 12, or any range therein. In some embodiments, the number of amino acids can range from 1 to 4. In some embodiments, the linker comprises any combination of four natural or non-natural amino acids such as, for example, -(Gly)₄- (SEQ ID NO:212). In some embodiments, the linker is not -(Gly)₄- (SEQ ID NO:212).

In some embodiments, the linker consists of four amino acids, -Xaa₁-Xaa₂-Xaa₃-Xaa₄- (SEQ ID NO:213), wherein Xaa₁, Xaa₂, Xaa₃, and Xaa₄ are each independently selected from a group consisting of (a) any natural amino acid, and (b) any non-natural amino acid. Examples of natural amino acids include, but are not limited to, glycine, alanine, valine, leucine, isoleucine, methionine, phenylalanine, tyrosine, aspartic acid, glutamic acid, lysine, arginine, serine, threonine, cysteine, asparagine, proline, tryptophan, histidine and combinations thereof. In some embodiments, provided that the natural amino acid is not L- or D-Cys.

In many embodiments, the linker comprises at least one amino acid having a side chain bearing positive charge. Examples of such amino acids include Lys, Arg, His, and Orn. In some embodiments, Xaa₁, Xaa₂, Xaa₃, and Xaa₄ can each be independently selected from a group consisting of Gly, L- or D-Lys, L- or D-Arg, L- or D-His, and L- or D-Orn. In some embodiments, the linker can contain any combination of Gly and Lys, Gly and Arg, Gly and Orn, or Gly and His. In some embodiments, the linker can contain all Lys, all Arg, all His, or all Orn.

In some embodiments, there is no linker. The mimetics can include portions of the CXC chemokines connected directly to each other through amide bonds; or disulfide bonds, such as the disulfide bonds that can form between Cys residues.

As discussed above, it should to be appreciated that a wide variety of amino acid substitutions may also be made in the polypeptide sequences. Examples of such substitutions include, but are not limited to, substituting lysine for glutamic acid, lysine for aspartic acid, ornithine for glutamic acid, and ornithine for aspartic acid.

Any of the analogs taught herein can have 75, 80, 85, 90, 95, 97, 99%, or any range therein, homology to the corresponding regions of the native chemokine sequence, so long as the function of the respective analog is preserved. Percent homology can be determined using any method known to one of skill, such as the NCBI BLAST tool and techniques, for example, which is available at www.ncbi.nlm.nih.gov.

The CXC chemokine analogs may include at least one modifying group connected either directly or indirectly somewhere on the analog structure. The term “modifying group” refers to any chemical moiety that was either absent from the corresponding native chemokine or comprises an isolated sequence of less than five amino acids. Such sequences are “isolated” in that they are positioned differently in the CXC chemokine analog than they were positioned in the native chemokine. A linker can also comprise a modifying group. The CXC chemokine analog modifications can include, but are not limited to, modifications of an N-terminus; modifications of a C-terminus; modifications of an internal region; modifications of an N-terminal region containing a sequence Glu-Leu-Arg; modifications of an internal region containing three anti-parallel β-sheets in the structure; modifications of a C-terminal region containing an α-helical structure; modifications of a combination of N-terminal and C-terminal regions; combinations of these modifications linked together either directly or through a linker; combinations of N-terminal and internal regions and modifications thereof; combinations of internal and C-terminal regions and modifications thereof; combinations of N-terminal, internal and C-terminal regions and modifications thereof; and combinations thereof. In some embodiments, the N-terminal region of each sequence must include a sub-sequence of Glu-Leu-Arg. In some embodiments, the N-terminal region does not include a sub-sequence Glu-Leu-Arg.

A modifying group can be connected, for example, to the N-terminus or C-terminus of a peptide; to a peptidic or peptidomimetic region flanking the core domain; to a side chain of at least one amino acid residue such as, for example, an ε-amino group of a lysyl residue, a carboxyl group of an aspartic acid or glutamic acid residue, a hydroxy group of a tyrosyl, serine or threonine residue, or other suitable reactive group on an amino acid side chain; or in-chain as a linker. Examples of chemical connections used to attach the modifying groups can include, but are not limited to, ether, azide, amide, ester, anhydride, orthoester, alkylamine, sulphide, disulphide, carbamate, carbonate, urea bonds, and the like.

In general, a modifying group can include any of the functional groups described herein, such as a “biotinyl structure”, which includes biotinyl groups and analogues and derivatives thereof. Examples of biotinyl structures include, but are not limited to, iminiobiotinyl structures such as, for example, a 2-iminobiotinyl group.

In some embodiments, the modifications can control the pharmacokinetic or pharmacodynamic properties of a CXC chemokine analog without substantially reducing its bioactive function. In some embodiments, the modifications can alter in vivo stability, bioavailability, or half-life of a mimetic. In some embodiments, the modifications can provide a diagnostic capability such as, for example, by creating a means of detecting the presence or location of a mimetic in vivo or in vitro. Examples of detectable substances are described below.

The mimetics are constructed by connecting the components of the analogs (N-terminal region, C-terminal region, linker, modifying groups, etc.) through functional groups. The terms “radical,” “group,” “functional group” and “substituent” can be used interchangeably in some contexts to describe a chemical that has been added to another chemical to modify its structure. The term “substituted” is used to characterize a chemical structure that has been modified by the addition of at least one functional group to at least one position that can be in-chain, pendant, and/or terminal to the chemical structure. In some embodiments, the functional groups can include, but are not limited to, aliphatics, aromatics, and combinations thereof; alkyls, alkenes, alkynes, cyclic structures, heterocyclic structures, and combinations thereof.

In some embodiments, the functional groups themselves can serve as a modifying group. The functional groups of the present invention can be independently selected from substituted, unsubstituted, hetero-, straight-chained, branched, cyclic, saturated or unsaturated aliphatic radical; or a substituted, unsubstituted, or hetero-aromatic radicals. For example, a functional group can be selected from H; aliphatic hydrocarbon groups such as, for example, alkyl, alkenyl, and alkynyl groups; aromatic groups such as, for example, aryl, aralkyl, aralkenyl, and aralkynyl groups; and, various other groups as defined below.

In some embodiments, the functional groups may include biobeneficial, bioactive, and/or diagnostic agents. A “bioactive agent” is a functional group that can be connected to the CXC chemokine analog to provide a therapeutic effect, a prophylactic effect, both a therapeutic and a prophylactic effect, or other biologically active effect. A “biobeneficial agent” is a functional group that can also be connected to a CXC chemokine analog to provide a biological benefit within a subject. In one example, a biobeneficial agent can be non-inflammatory, such as, for example, by acting as a biomimic to passively avoid attracting monocytes and neutrophils, which leads to the cascade of events creating inflammation.

A “diagnostic agent” is a type of bioactive agent that can be used, for example, in diagnosing the presence, nature, or extent of a disease or medical condition in a subject. In one embodiment, a diagnostic agent can be any agent that may be used in connection with methods for imaging an internal region of a patient and/or diagnosing the presence or absence of a disease in a patient. Diagnostic agents include, for example, contrast agents for use in connection with ultrasound imaging, magnetic resonance imaging (MRI), nuclear magnetic resonance (NMR), computed tomography (CT), electron spin resonance (ESR), nuclear medical imaging, optical imaging, elastography, radiofrequency (RF) and microwave laser. Diagnostic agents may also include any other agents useful in facilitating diagnosis of a disease or other condition in a patient, whether or not imaging methodology is employed.

In some embodiments, the biobeneficial agents can have a reactive group that can be used to connect an agent to a CXC chemokine analog. Examples of such reactive groups include, but are not limited to, hydroxyl, carboxyl, and amino groups. In some embodiments, the biobeneficial agents can remain attached to the CXC chemokine analog or be controllably released from the CXC chemokine analog.

In some embodiments, the molecular weight of an agent connected to a CXC chemokine analog should be at or below about 40,000 Daltons, or any range therein, to ensure elimination of the agent from a subject. In one embodiment, the molecular weight of the agent ranges from about 300 Daltons to about 40,000 Daltons, from about 8,000 Daltons to about 30,000 Daltons, from about 10,000 Daltons to about 20,000 Daltons, or any range therein. It is to be appreciated that one skilled in the art should recognize that some of the groups, subgroups, and individual biobeneficial agents may not be used in some embodiments of the present invention.

In some embodiments of the present invention, the aliphatic radicals have from about 1 to about 50 carbon atoms, from about 2 to about 40 carbon atoms, from about 3 to about 30 carbon atoms, from about 4 to about 20 carbon atoms, from about 5 to about 15 carbon atoms, from about 6 to about 10 carbon atoms, and any range therein. In some embodiments, the aromatic radicals have from about 6 to about 180 carbon atoms, from about 12 to about 150 carbon atoms, from about 18 to about 120 carbon atoms, from about 24 to about 90 carbon atoms, from about 30 to about 60 carbon atoms, and any range therein.

The term “alkyl” can be used interchangeably with the term “alkylene” in some contexts and refers to a straight-chained or branched hydrocarbon chain. Examples of alkyl groups include lower alkyl groups such as, for example, methyl, ethyl, n-propyl, iso-propyl, n-butyl, iso-butyl, t-butyl or iso-hexyl; upper alkyl groups such as for example, n-heptyl, n-octyl, iso-octyl, nonyl, decyl, and the like; lower alkylene such as, for example, ethylene, propylene, butylenes, butadiene, pentene, n-hexene and iso-hexene; and upper alkylene such as, for example, n-heptene, n-octene, iso-octene, nonene, decene, and the like. Persons of ordinary skill in the art are familiar with numerous straight-chained and branched alkyl groups, which are within the scope of the present invention. In addition, such alkyl groups may also contain various substituents in which one or more hydrogen atoms can be replaced by a functional group, or the alkyl groups can contain an in-chain functional group.

The term “alkenyl” refers to a straight-chained or branched hydrocarbon chain where at least one of the carbon-carbon linkages is a carbon-carbon double bond. The term “alkynyl” refers to a straight-chained or branched hydrocarbon chain where at least one of the carbon-carbon linkages is a carbon-carbon triple bond.

The term “aryl” refers to a hydrocarbon ring bearing a system of conjugated double bonds often comprising at least six π (pi) electrons. Examples of aromatic groups include, but are not limited to, phenyl, pyrrolyl, furyl, thiophenyl, imidazolyl, oxazole, thiazolyl, triazolyl, pyrazolyl, pyridyl, pyrazinyl, pyridazinyl and pyrimidinyl, naphthyl, anysyl, toluyl, xylenyl, and the like. The term “aralkyl” refers to an alkyl group substituted with at least one aryl group. Examples of aralkyls include substituted benzyls such as, for example, phenylmethyl, 2-naphthylethyl, 2-(2-pyridyl) propyl, 5-dibenzosuberyl, and the like. The term “aralkenyl” refers to an alkenyl group substituted with at least one aryl group. Those aryl groups having heteroatoms in the ring structure may also be referred to as “aryl heterocycles” or “heteroaromatics.” The aromatics can be substituted at one or more ring positions and can also be part of a polycyclic group. For example, aryl groups can include fused aromatic moieties such as naphthyl, anthracenyl, quinolyl, indolyl, and the like.

The phrase “straight-chained or branched” includes any substituted or unsubstituted acyclic carbon-containing compounds including, but not limited to, alkanes, alkenes and alkynes. A radical is “straight-chained” when it has less than 0.1 mole percent of sidechains having 1 or more carbon atoms. In some embodiments, a radical is straight-chained if it has less than 0.01 mole percent of such sidechains. In some embodiments, a radical is straight-chained if it has less than 0.001 mole percent of such sidechains. A radical is “branched” when it has more than 0.1 mole percent of sidechains having 1 or more carbon atoms. In some embodiments, a radical is branched when it has more than 0.01 mole percent of such sidechains. In some embodiments, a radical is branched when it has more than 0.001 mole percent of such sidechains.

The terms “radical,” “group,” “functional group,” and “substituent” can be used interchangeably in some contexts and can be used together to further describe a chemical structure. For example, the term “functional group” can refer to a chemical “group” or “radical,” which is a chemical structure variable that is in-chain, pendant and/or terminal to the chemical structure. In some embodiments, a straight chain or branched alkyl has from about 1 to about 20 carbon atoms, from about 2 to about 18 carbon atoms, from about 3 to about 17 carbon atoms, from about 5 to about 15 carbon atoms, from about 2 to about 10 carbon atoms, or any range therein. In some embodiments, a cycloalkyl may have a ring structure containing from about 2 to about 12 carbon atoms, from about 3 to about 11 carbon atoms, from about 4 to about 10 carbon atoms, or any range therein.

A functional group may comprise a cyclic or polycyclic group. The term “cyclic group” refers to a ring structure that can be substituted, unsubstituted, hetero-, saturated or unsaturated and have from 3 to 24 carbon atoms, from 3 to 18 carbon atoms, from 3 to 12 carbon atoms, or any range therein. Examples of cyclic groups include, but are not limited to, cycloalkyls such as cyclopropyl, cyclobutyl, cyclopentyl, cyclohexyl, and cyclooctyl structures; cycloalkenes; and aromatics. The term “polycyclic group” refers to two or more substituted, unsubstituted, hetero-, saturated or unsaturated cyclic rings in which two or more ring carbons are common among two adjoining rings such that the rings are “fused rings.” The rings can also be “bridged rings” in that they are joined through atoms that are not common among the adjoining rings.

The term “heterocyclic group” includes cyclic saturated, unsaturated and aromatic groups having from 3 to 10; from 4 to 8; or 5, 6, or 7 carbon atoms, wherein the ring structure includes one or more heteroatoms, such as oxygen, nitrogen, sulfur, or combinations thereof. Heterocyclic groups include pyrrolidine, oxolane, thiolane, imidazole, oxazole, piperidine, piperazine, and morpholine. The heterocyclic ring may be substituted at one or more positions with such substituents as, for example, halogens, alkyls, cycloalkyls, alkenyls, alkynyls, aryls, arylalkyls, other heterocycles, hydroxyl, amino, nitro, thiol, amines, imines, amides, phosphonates, phosphines, carbonyls, carboxyls, silyls, ethers, thioethers, sulfonyls, selenoethers, ketones, aldehydes, esters, —CF₃, —CN. Heterocycles may also be bridged or fused to other cyclic groups. A linker may also link the heterocyclic group to such substituents as, for example, halogens, alkyls, cycloalkyls, alkenyls, alkynyls, aryls, arylalkyls, heterocycles, hydroxyls, aminos, nitros, thiols amines, imines, amides, phosphonates, phosphines, carbonyls, carboxyls, silyls, ethers, thioethers, sulfonyls, sulfonates, selenoethers, ketones, aldehydes, esters, —CF₃, —CN.

The term “alkylcarbonyl,” as used herein, refers to —C(O)-alkyl. Similarly, the term “arylcarbonyl” refers to —C(O)-aryl. The term “alkyloxycarbonyl,” as used herein, refers to the group —C(O)—O-alkyl, and the term “aryloxycarbonyl” refers to —C(O)—O-aryl. The term “acyloxy” refers to —O—C(O)—R₇, in which R₇ is alkyl, alkenyl, alkynyl, aryl, aralkyl or heterocyclyl.

The term “amino,” as used herein, refers to —N(R_{α})(R_{β}), in which R_{α} and R_{β} are each independently hydrogen, alkyl, alkyenyl, alkynyl, aralkyl, aryl, or in which R_{α} and R_{β} together with the nitrogen atom to which they are attached form a ring having 4-8 atoms. Thus, the term “amino,” as used herein, includes unsubstituted, monosubstituted (e.g., monoalkylamino or monoarylamino), and disubstituted (e.g., dialkylamino or alkylarylamino) amino groups. The term “amido” refers to —C(O)—N(R_{α})(R_{β}), in which R_{α} and R_{β} are as defined above. The term “acylamino” refers to —N(R′_{α})C(O)—R₇, in which R₇ is as defined above and R_{α} is alkyl. As used herein, the term “nitro” means —NO₂; the term “halogen” designates —F, —Cl, —Br or —I; the term “sulfhydryl” means —SH; and the term “hydroxyl” means —OH.

In some embodiments, the functional groups can include, but are not limited to, oxygen-containing groups such as, for example, alcohols, ethers, phenols, and derivatives thereof. Such oxygen-containing groups include, but are not limited to, acetonides, alcohols, alkoxides, bisphenols, carbinols, cresols, diols, enols, enolates, epoxides, ethers, glycols, hydroperoxides, peroxides, phenols, phenolates, phenoxides, pinacols, trioxides, and ynols.

In some embodiments, the functional groups can include, but are not limited to, oxygen-containing groups such as, for example, aldehydes, ketones, quinones and derivatives thereof. Such oxygen-containing groups include, but are not limited to, acetals, acyloins, aldehydes, carbonyl compounds, diosphenols, dypnones, hemiacetals, hemiketals, ketals, ketenes, keto compounds, ketones, quinhydrones, quinomethanes, quinines, and combinations thereof.

In some embodiments, the functional groups can include, but are not limited to, oxygen-containing groups such as, for example, carboxylic acids and derivatives thereof. Such oxygen-containing groups include, but are not limited to, carboxylic acids, oxoacids, sulfonic acids, acid anhydrides, acid thioanhydrides, acyl groups, acyl halides, acylals, anhydrides, carboxylic acids, cyclic acid anhydrides, cyclic anhydrides, esters, fulgides, lactides, lactols, lactones, macrolides, naphthenic acids, ortho acids, ortho esters, oxo carboxylic acids, peroxy acids, and combinations thereof,

In some embodiments, the functional groups can include, but are not limited to, nitrogen-containing groups containing one nitrogen such as, for example, aldimines, aldoximes, alkoxyamines, amic acids, amides, amines, amine oxides, amine ylides, carbamates, hemiaminals, carbonitriles, carboxamides, isocyanides, cyanates, isocyanates, diisocyanates, cyanides, cyanohydrins, diacylamines, enamines, fulminates, hemiaminals, hydroxamic acids, hydroximic acids, hydroxylamines, imides, imidic acids, imidines, imines, oximes, isoureas, ketenimines, ketimines, ketoximes, lactams, lactims, nitriles, nitro, nitroso, nitrosolic acids, oxime O-ethers, quaternary ammonium compounds, quinone imines, quinonoximes, azomethines, ureides, urethanes, and combinations thereof.

In some embodiments, the functional groups can include, but are not limited to, nitrogen-containing groups containing two or more nitrogens such as, for example, aldazines, amide hydrazones, amide oximes, amidines, amidrazones, aminals, amine imides, amine imines, isodiazenes, azans, azides, azo imides, azines, azo compounds, azomethine imides, azoxy compounds, carbodiimides, carboxamidines, diamidides, diazo compounds, diazoamino compounds, diazoates, diazooxides, formamidine disulfides, formazans, hydrazides, hydrazide hydrazones, hydrazide imides, hydrazidines, hydrazines, hydrazo compounds, hydrazones, ketazines, nitramines, nitrile imines, nitrimines, nitrolic acids, nitrosamides, nitrosamines, nitrosimines, ortho amides, semicarbazones, semioxamazones, triazanes, triazenes, and combinations thereof.

In some embodiments, the functional groups can include, but are not limited to, sulfur-containing groups such as thio, thiol, thioether, sulfonyl, sulfido, sulfinamides, sulfilimines, sulfimines, sulfimides, sulfinamidines, sulfines, sulfinic acids, sulfinic anhydrides, sulfinylamines, sulfonamides, sulfones, sulfonediimines, sulfonic acids, sulfonic anhydrides, sulfoxides, sulfoximides;

In some embodiments, the functional groups can include, but are not limited to, silyl groups, halogens, selenoethers, trifluoromethyls, thio-derivatives of urethanes where at least one oxygen atom is replaced by a sulfur atom; phosphoryls, phosphonates, phosphinates; and ethyleneically unsaturated groups such as, for example, allyl, acryloyl and methacrylol, and maleate and maleimido; and combinations thereof.

Examples of heteroatoms of the hetero-radicals include, but are not limited to, sulfur, phosphorous, oxygen, nitrogen and combinations thereof. Examples of heterocyclic groups include, but are not limited to, pyrrolidine, oxolane, thiolane, imidazole, oxazole, piperidine, piperazine, and morpholine. The heterocyclics may also be bridged or fused to other cyclic groups as described below.

In some embodiments, the modifying groups can include, but are not limited to, O-modified derivatives including, but not limited to, C-terminal hydroxymethyl benzyl ether, and other C-terminal hydroxymethyl derivatives; N-modified derivatives including, but not limited to, substituted amides such as alkylamides; hydrazides and compounds in which a C-terminal phenylalanine residue is replaced with a phenethylamide analogue such as, for example, by replacing Ser-Ile-Phe with Ser-Ile-phenethylamide.

In some embodiments, the functional group may include a fluorescein-containing group. Examples of fluorescein-containing groups include, but are not limited to, 5-(and 6-)-carboxyfluorescein succinimidyl ester and fluorescein isothiocyanate. In some embodiments, the modifying group may include a cholyl structure. An example of a cholyl derivative is 3-(O-aminoethyl-iso)-cholyl (Aic).

In some embodiments, the functional group may include N-acetylneuraminyl, trans-4-cotininecarboxyl, 2-imino-1-imidazolidineacetyl, (S)-(−)-indoline-2-carboxyl, 2-norbornaneacetyl, γ-oxo-5-acenaphthenebutyryl, (−)-2-oxo-4-thiazolidinecarboxyl group, tetrahydro-3-furoyl group, 4-morpholinecarbonyl group, 2-thiopheneacetyl group, 2-thiophenesulfonyl group, diethylene-triaminepentaacetyl group, (O)-methoxyacetyl group, N-acetylneuraminyl group, and combinations thereof. In some embodiments, the functional group may include light scattering groups, magnetic groups, nanogold, other proteins, a solid matrix, radiolabels, carbohydrates, and combinations thereof.

Examples of biobeneficial agents include, but are not limited to, many of the polymers listed above such as, for example, carboxymethylcellulose, poly(alkylene glycols), poly(N-vinyl pyrrolidone), poly(acrylamide methyl propane sulfonic acid), poly(styrene sulfonate), sulfonated dextran, polyphosphazenes, poly(orthoesters), poly(tyrosine carbonate), dermatan sulfate, hyaluronic acid, heparin, and any derivatives, analogs, homologues, congeners, salts, copolymers and combinations thereof.

Examples of heparin derivatives include, but are not limited to, earth metal salts of heparin such as, for example, sodium heparin, potassium heparin, lithium heparin, calcium heparin, magnesium heparin, and low molecular weight heparin. Other examples of heparin derivatives include, but are not limited to, heparin sulfate, heparinoids, heparin-based compounds and heparin derivatized with hydrophobic materials.

Examples of hyaluronic acid derivates include, but are not limited to, sulfated hyaluronic acid such as, for example, O-sulphated or N-sulphated derivatives; esters of hyaluronic acid wherein the esters can be aliphatic, aromatic, arylaliphatic, cycloaliphatic, heterocyclic or a combination thereof; crosslinked esters of hyaluronic acid wherein the crosslinks can be formed with hydroxyl groups of a polysaccharide chain; crosslinked esters of hyaluronic acid wherein the crosslinks can be formed with polyalcohols that are aliphatic, aromatic, arylaliphatic, cycloaliphatic, heterocyclic, or a combination thereof; hemiesters of succinic acid or heavy metal salts thereof; quaternary ammonium salts of hyaluronic acid or derivatives such as, for example, the O-sulphated or N-sulphated derivatives.

Examples of poly(alkylene glycols) and its derivatives include, but are not limited to, PEG, mPEG, poly(ethylene oxide), poly(propylene glycol)(PPG), poly(tetramethylene glycol), and any derivatives, analogs, homologues, congeners, salts, copolymers and combinations thereof. In some embodiments, the poly(alkylene glycol) is poly(ethylene glycol-co-hydroxybutyrate).

The copolymers that may be used as biobeneficial agents include, but are not limited to, any derivatives, analogs, homologues, congeners, salts, copolymers and combinations of the foregoing examples of agents. Examples of copolymers that may be used as biobeneficial agents in the present invention include, but are not limited to, dermatan sulfate, which is a copolymer of D-glucuronic acid or L-iduronic acid and N-acetyl-D-galactosamine; poly(ethylene oxide-co-propylene oxide); copolymers of PEG and hyaluronic acid; copolymers of PEG and heparin; copolymers of PEG and hirudin; graft copolymers of poly(L-lysine) and PEG; copolymers of PEG and a poly(hydroxyalkanoate) such as, for example, poly(ethylene glycol-co-hydroxybutyrate); and, any derivatives, analogs, congeners, salts, or combinations thereof. In some embodiments, the copolymer that may be used as a biobeneficial agent can be a copolymer of PEG and hyaluronic acid, a copolymer of PEG and hirudin, and any derivative, analog, congener, salt, copolymer or combination thereof. In some embodiments, the copolymer that may be used as a biobeneficial agent is a copolymer of PEG and a poly(hydroxyalkanoate) such as, for example, poly(hydroxybutyrate); and any derivative, analog, congener, salt, copolymer or combination thereof.

The bioactive agents can be any moiety capable of contributing to a therapeutic effect, a prophylactic effect, both a therapeutic and prophylactic effect, or other biologically active effect in a subject. A bioactive agent can also have diagnostic properties. The bioactive agents include, but are not limited to, small molecules, nucleotides, oligonucleotides, polynucleotides, amino acids, oligopeptides, polypeptides, and proteins. Bioactive agents include, but are not limited to, anti proliferatives, anti neoplastics, anti mitotics, anti-inflammatories, antiplatelets, anticoagulants, antifibrins, antithrombins, antibiotics, antiallergics, antioxidants, and any prodrugs, codrugs, metabolites, analogs, homologues, congeners, derivatives, salts and combinations thereof. It is to be appreciated that one skilled in the art should recognize that some of the groups, subgroups, and individual bioactive agents may not be used in some embodiments of the present invention.

Antiproliferatives include, for example, actinomycin D, actinomycin IV, actinomycin I1, actinomycin X1, actinomycin C1, and dactinomycin (Cosmegen®, Merck & Co., Inc.). Antineoplastics or antimitotics include, for example, paclitaxel (Taxol®, Bristol-Myers Squibb Co.), docetaxel (Taxotere®, Aventis S.A.), methotrexate, azathioprine, vincristine, vinblastine, fluorouracil, doxorubicin hydrochloride (Adriamycin®, Pfizer, Inc.) and mitomycin (Mutamycin®, Bristol-Myers Squibb Co.), and any prodrugs, codrugs, metabolites, analogs, homologues, congeners, derivatives, salts and combinations thereof. Antiplatelets, anticoagulants, antifibrin, and antithrombins include, for example, sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, and thrombin inhibitors (Angiomax®, Biogen, Inc.), and any prodrugs, codrugs, metabolites, analogs, homologues, congeners, derivatives, salts and combinations thereof.

Cytostatic or antiproliferative agents include, for example, angiopeptin, angiotensin converting enzyme inhibitors such as captopril (Capoten® and Capozide®, Bristol-Myers Squibb Co.), cilazapril or lisinopril (Prinivil®) and Prinzide®), Merck & Co., Inc.); calcium channel blockers such as nifedipine; colchicines; fibroblast growth factor (FGF) antagonists, fish oil (omega 3-fatty acid); histamine antagonists; lovastatin (Mevacor®, Merck & Co., Inc.); monoclonal antibodies including, but not limited to, antibodies specific for Platelet-Derived Growth Factor (PDGF) receptors; nitroprusside; phosphodiesterase inhibitors; prostaglandin inhibitors; suramin; serotonin blockers; steroids; thioprotease inhibitors; PDGF antagonists including, but not limited to, triazolopyrimidine; and nitric oxide, and any prodrugs, codrugs, metabolites, analogs, homologues, congeners, derivatives, salts and combinations thereof. Antiallergic agents include, but are not limited to, pemirolast potassium (Alamast®, Santen, Inc.), and any prodrugs, codrugs, metabolites, analogs, homologues, congeners, derivatives, salts and combinations thereof.

Other bioactive agents useful in the present invention include, but are not limited to, free radical scavengers; nitric oxide donors; rapamycin; everolimus; tacrolimus; 40-O-(2-hydroxy)ethyl-rapamycin; 40-O-(3-hydroxy)propyl-rapamycin; 40-O-[2-(2-hydroxy)ethoxy]ethyl-rapamycin; tetrazole containing rapamycin analogs such as those described in U.S. Pat. No. 6,329,386; estradiol; clobetasol; idoxifen; tazarotene; alpha-interferon; host cells such as epithelial cells; genetically engineered epithelial cells; dexamethasone; cytokines; chemokines, chemokine mimetics, chemokine receptor ligands, and, any prodrugs, codrugs, metabolites, analogs, homologues, congeners, derivatives, salts and combinations thereof.

Free radical scavengers include, but are not limited to, 2,2′,6,6′-tetramethyl-1-piperinyloxy, free radical (TEMPO); 4-amino-2,2′,6,6′-tetramethyl-1-piperinyloxy, free radical (4-amino-TEMPO); 4-hydroxy-2,2′,6,6′-tetramethyl-piperidene-1-oxy, free radical (TEMPOL), 2,2′,3,4,5,5′-hexamethyl-3-imidazolinium-1-yloxy methyl sulfate, free radical; 16-doxyl-stearic acid, free radical; superoxide dismutase mimic (SODm) and any analogs, homologues, congeners, derivatives, salts and combinations thereof. Nitric oxide donors include, but are not limited to, S-nitrosothiols, nitrites, N-oxo-N-nitrosamines, substrates of nitric oxide synthase, diazenium diolates such as spermine diazenium diolate and any analogs, homologues, congeners, derivatives, salts and combinations thereof.

Chemokines include, but are not limited to, IL-8, IP-10, PF-4, MIP-1α, RANTES, 1-309, MCP-1, CCL28, and SDF-1. Chemokine mimetics include, but are not limited to, those taught in U.S. Patent Application Publication Nos. 2002/0156034, 2002/0165123, and 2003/0148940; and U.S. patent application Ser. No. 10/243,795; each of which is incorporated by reference herein in its entirety. Chemokine receptor ligands include, but are not limited to, those taught in U.S. Pat. Nos. 6,515,001 and 6,693,134; and U.S. Patent Application Publication Nos. 2003/0004136, 2003/0045550, 2003/0092674, and 2003/0125380; each of which is incorporated by reference herein in its entirety.

Diagnostic agents include, but are not limited to, materials that are radiopaque, radioactive, paramagnetic, fluorescent, lumiscent, and detectable by ultrasound. In some embodiments, the radiopaque agents are materials comprising iodine or iodine-derivatives such as, for example, iohexal and iopamidol. In some embodiments, the radioactive materials are radioisotopes, which can be detected by tracing radioactive emissions. Examples of radioactive materials include, but are not limited to, ¹⁴C, ¹²³I, ¹²⁴I, ¹²⁵I, ¹³¹I, ^{99m}Tc, ³⁵S or ³H. In some embodiments, the paramagnetic agents include, but are not limited to, gadolinium chelated compounds. Examples of fluorescent agents include, but are not limited to, indocyanine green, umbelliferone, fluorescein, fluorescein isothiocyanate, rhodamine, dichlorotriazinylamine fluorescein, dansyl chloride or phycoerythrin. Examples of agents detectable by ultrasound include, but are not limited to, perflexane, Albunex® and Optison®. Examples of agents used in PET include, but are not limited to, fluorodeoxyglucose, sodium fluoride, methionine, choline, deoxyglucose, butanol, raclopride, spiperone, bromospiperone, carfentanil, and flumazenil. Other examples of detectable substances include, but are not limited to, various enzymes and prosthetic groups. Examples of suitable enzymes include, but are not limited to, horseradish peroxidase, alkaline phosphatase, β-galactosidase, or acetylcholinesterase. Examples of suitable prosthetic group complexes include, but are not limited to, streptavidin/biotin and avidin/biotin.

Labeled CXC chemokine analogs can be used to assess in vivo pharmacokinetics, as well as detect the progression of a disease or the propensity of a subject to develop a disease. For example, chemokine receptors for tissue distribution can be detected using a labeled CXC chemokine analog either in vivo or in an in vitro sample derived from a subject. In some embodiments, a CXC chemokine analog may be radioactively labeled with ¹⁴C, either by incorporation of ¹⁴C into the modifying group or one or more amino acid structures in the CXC chemokine analog.

A modifying group can be chosen to provide a chelation site for a diagnostic label. In some embodiments, the modifying group can be the Aic derivative of cholic acid, which provides a free amino group; a tyrosine residue within a CXC chemokine sequence may be substituted with radioactive iodotyrosyl; or a CXC chemokine analog may be labeled with radioactive technetium or iodine. In fact, any isotope of radioactive iodine may be incorporated to create a diagnostic agent. In some embodiments, ¹²³I has a half-life of 13.2 hours and can be used for whole body scintigraphy; ¹²⁴I has a half life of 4 days and can be used for PET; ¹²⁵I has a half life of 60 days and can be used for metabolic turnover studies; and, ¹³¹I has a half life of 8 days and can be used for whole body counting and delayed low resolution imaging studies.

In some embodiments, a modification may be introduced at the C-terminus of a peptide, the N-terminus of a peptide, in the region between the C-terminus and N-terminus, or a combination thereof. In some embodiments, a modification to the C-terminus may reduce the ability of a CXC chemokine analog to act as a substrate for carboxypeptidases. Examples of such C-terminal modifiers include, but are not limited to, an amide group, an ethylamide group and various non-natural amino acids such as, for example, D-amino acids and β-alanine. In another embodiment, a modification of a C-terminus may be accompanied by a modification to the N-terminus to reduce the ability of a CXC chemokine analog to act as a substrate for aminopeptidases. Examples of such N-terminus modifiers include, but are not limited to acyl, alkyl, aryl, arylalkyl, hydroxyalkyl, alkanoyl groups, alkanoics, diacids, and other modifiers having a carboxyl functional group. In another embodiment, the modification to an N-terminus can be deamidation.

Aminopeptidases and carboxypeptidases have been found to have important functions in biological activities such as, for example, diabetes, memory and learning, antigen formation, and angiogenesis. The term “aminopeptidase” refers to a multifunctional enzyme that cleaves proteins from the N-terminus. Aminopeptidases can be classified into a number families such as, for example, the zinc-containing (M1) aminopeptidase family which consists of nine aminopeptidases that include, but are not limited to, placental leucine aminopeptidase (P-LAP), adipocyte-derived leucine aminopeptidase (A-LAP) and leukocyte-derived arginine aminopeptidase (L-RAP). Modulation of aminopeptidase activity can have many therapeutic and prophylactic applications. In one example, control of the activity of P-LAP can control the inducement of uterine contractions and treat or prevent disorders such as premature delivery and spontaneous abortion, as well as other disorders associated with water resorption, memory and learning and glucose metabolism. In another example, control of the activity of A-LAP can treat disorders associated with antigen production, blood pressure and inflammation. In another example, control of the activity of L-RAP can treat disorders association with antigen formation.

Although both aminopeptidases and carboxypeptidases can terminate biological activity, the carboxypeptidases clearly predominate in such terminations. The term “carboxypeptidase” refers to a multifunctional enzyme that cleaves proteins from the C-terminus. Carboxypeptidases are derived from the zymogens, procarboxypeptidase A and B. Modulation of carboxypeptidase activity can have many therapeutic and prophylactic applications. In one example, control of the activity of the carboxypeptidases such as kininase II (angiotensin-converting enzyme), carboxypeptidase M, and carboxypeptidase N, can potentially control hypertensive disorders relating to cardiovascular and kidney disorders. These carboxypeptidases are efficient at cleaving the C-terminal arginine of kinins, which appear to be important regulators of cardiovascular function; and are likely participants in the actions of drugs that affect the heart, kidney, and circulation. The kinins also have some role in the regulation of local and systemic hemodynamics; vascular permeability; inflammatory response; activation of neuronal pathways; and movement of electrolytes, water, and metabolic substrates across epithelia and into other tissues. Accordingly, control of carboxypeptidase activity can control the activity of other chemicals such as, for example, kinins, and thus can have many therapeutic applications in the diagnosis and treatment of disease.

### Pharmaceutical Compositions

In most embodiments, the invention includes pharmaceutical compositions containing CXC chemokine receptor agonists or antagonists. The pharmaceutical compositions include a mimetic in an amount that is diagnostic, therapeutic and/or prophylactic in the diagnosis, prevention, treatment and amelioration of symptoms of disease.

In some embodiments, such compositions include a CXC chemokine analog compound to be used in treating diseases or disorders selected from the group consisting of autoimmune diseases, acute chronic inflammation, cancer, cardiovascular disease, infectious disease, and inflammatory disorders including rheumatoid arthritis, chronic inflammatory bowel disease, chronic inflammatory pelvic disease, multiple sclerosis, asthma, osteoarthritis, atherosclerosis, psoriasis, rhinitis, autoimmunity, and organ transplant rejection. In some embodiments, such compositions include a CXC chemokine analog compound in a therapeutically or prophylactically effective amount sufficient to be used to increase the hemocrit, assist in mobilizing and recovering stem cells, stimulate the production of blood cells, assist in vaccine production, or assist in gene therapy.

The amount of a mimetic used in the compositions can vary according to factors such as type of disease, age, sex, and weight of the subject. Dosage regimens may be adjusted to optimize a therapeutic response. In some embodiments, a single bolus may be administered; several divided doses may be administered over time; the dose may be proportionally reduced or increased; or any combination thereof, as indicated by the exigencies of the therapeutic situation and factors known one of skill in the art. It is to be noted that dosage values may vary with the severity of the condition to be alleviated. Dosage regimens may be adjusted over time according to the individual need and the professional judgment of the person administering or supervising the administration of the compositions, and the dosage ranges set forth herein are exemplary only and do not limit the dosage ranges that may be selected by medical practitioners.

The terms “administration” or “administering” refer to a method of incorporating a compound into the cells or tissues of a subject, either in vivo or ex vivo to diagnose, prevent, treat, or ameliorate a symptom of a disease. In one example, a compound can be administered to a subject in vivo parenterally. In another example, a compound can be administered to a subject by combining the compound with cell tissue from the subject ex vivo for purposes that include, but are not limited to, cell expansion and mobilization assays. When the compound is incorporated in the subject in combination with one or active agents, the terms “administration” or “administering” can include sequential or concurrent incorporation of the compound with the other agents such as, for example, any agent described above. A pharmaceutical composition of the invention is formulated to be compatible with its intended route of administration. Examples of routes of administration include, but are not limited to, parenteral such as, for example, intravenous, intradermal, intramuscular, and subcutaneous injection; oral; inhalation; intranasal; transdermal; transmucosal; and rectal administration.

An “effective amount” of a compound of the invention can be used to describe a therapeutically effective amount or a prophylactically effective amount. A “therapeutically effective amount” refers to an amount that is effective at the dosages and periods of time necessary to achieve a desired therapeutic result and may also refer to an amount of active compound, prodrug or pharmaceutical agent that elicits any biological or medicinal response in a tissue, system, or subject that is sought by a researcher, veterinarian, medical doctor or other clinician that may be part of a treatment plan leading to a desired effect.

The therapeutically effective amount may need to be administered in an amount sufficient to result in amelioration of one or more symptoms of a disorder, prevention of the advancement of a disorder, or regression of a disorder. In some embodiments, a therapeutically effective amount can refer to the amount of a therapeutic agent that improves a subject's condition by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. The term “therapeutic effect” refers to the inhibition or activation of factors causing or contributing to the abnormal condition (including a disease or disorder).

A therapeutic effect relieves or prevents to some extent one or more of the symptoms of the abnormal condition. A therapeutic effect can refer to one or more of the following: (a) an increase or decrease in the number of lymphocytic cells present at a specified location, (b) an increase or decrease in the ability of lymphocytic cells to migrate, (c) an increase or decrease in the response of lymphocytic cells to a stimulus, (d) an increase or decrease in the proliferation, growth, and/or differentiation of cells; (e) inhibition (i.e., slowing or stopping) or acceleration of cell death; (f) relieving, to some extent, one or more of the symptoms associated with an abnormal condition; (g) enhancing or inhibiting the function of the affected population of cells; (h) activating an enzyme activity present in cells associated with the abnormal condition; and (i) inhibiting an enzyme activity present in cells associated with the abnormal condition.

The term “abnormal condition” refers to a function in the cells or tissues of an organism that deviates from their normal functions in that organism and includes, but is not limited to, conditions commonly referred to as diseases or disorders. An abnormal condition can relate to cell proliferation, cell differentiation, cell survival, cell migration or movement, or the activities of enzymes within a cell. Diseases and disorders may include inflammatory disorders including rheumatoid arthritis, chronic inflammatory bowel disease, chronic inflammatory pelvic disease, multiple sclerosis, asthma, osteoarthritis, atherosclerosis, psoriasis, rhinitis, autoimmunity, organ transplant rejection, and genetic diseases.

A “prophylactically effective amount” refers to an amount that is effective at the dosages and periods of time necessary to achieve a desired prophylactic result. Typically, a prophylactic dose is used in a subject prior to the onset of a disease, or at an early stage of the onset of a disease, to prevent or inhibit onset of the disease or symptoms of the disease. A prophylactically effective amount may be less than, greater than, or equal to a therapeutically effective amount.

In some embodiments, the administration can be oral. In some embodiments, the administration can be subcutaneous injection. In some embodiments, the administration can be intravenous injection using a sterile isotonic aqueous buffer. In some embodiments, the administration can include a solubilizing agent and a local anesthetic such as lignocaine to ease discomfort at the site of injection. In some embodiments, the administrations may be parenteral to obtain, for example, ease and uniformity of administration.

The compounds can be administered in dosage units. The term “dosage unit” refers to discrete, predetermined quantities of a compound that can be administered as unitary dosages to a subject. A predetermined quantity of active compound can be selected to produce a desired therapeutic effect and can be administered with a pharmaceutically acceptable carrier. The predetermined quantity in each unit dosage can depend on factors that include, but are not limited to, (a) the unique characteristics of the active compound and the particular therapeutic effect to be achieved, and (b) the limitations inherent in the art of creating and administering such dosage units.

A “pharmaceutically acceptable carrier” is a diluent, adjuvant, excipient, or vehicle with which the mimetic is administered. A carrier is pharmaceutically acceptable after approval by a state or federal regulatory agency or listing in the U.S. Pharmacopeial Convention or other generally recognized sources for use in subjects. The pharmaceutical carriers include any and all physiologically compatible solvents, dispersion media, coatings, antibacterial and antifungal agents, isotonic and absorption delaying agents, and the like. Examples of pharmaceutical carriers include, but are not limited to, sterile liquids, such as water, oils and lipids such as, for example, phospholipids and glycolipids. These sterile liquids include, but are not limited to, those derived from petroleum, animal, vegetable or synthetic origin such as, for example, peanut oil, soybean oil, mineral oil, sesame oil, and the like. Water can be a preferred carrier for intravenous administration. Saline solutions, aqueous dextrose and glycerol solutions can also be liquid carriers, particularly for injectable solutions.

Suitable pharmaceutical excipients include, but are not limited to, starch, sugars, inert polymers, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain minor amounts of wetting agents, emulsifying agents, pH buffering agents, or a combination thereof. The compositions can take the form of solutions, suspensions, emulsion, tablets, pills, capsules, powders, sustained-release formulations and the like. Oral formulations can include standard carriers such as, for example, pharmaceutical grades mannitol, lactose, starch, magnesium stearate, sodium saccharine, cellulose, magnesium carbonate, and the like. See Martin, E. W. Remington's Pharmaceutical Sciences. Supplementary active compounds can also be incorporated into the compositions.

In some embodiments, the carrier is suitable for parenteral administration. In some embodiments, the carrier can be suitable for intravenous, intraperitoneal, intramuscular, sublingual or oral administration. In some embodiments, the pharmaceutically acceptable carrier may comprise pharmaceutically acceptable salts, such as acid addition salts. For purposes of the present invention, the term “salt” and “pharmaceutically acceptable salt” can be used interchangeably in most embodiments. Pharmaceutically acceptable salts are non-toxic at the concentration in which they are administered and include those salts containing sulfate, hydrochloride, phosphate, sulfonate, sulfamate, sulfate, acetate, citrate, lactate, tartrate, methanesulfonate, ethanesulfonate, benzenesulfonate, p-toluenesulfonate, cyclohexylsulfonate, cyclohexylsulfamate, and quinate. Pharmaceutically acceptable salts can be obtained from acids, such as hydrochloric acid, sulfuric acid, phosphoric acid, sulfonic acid, sulfonic acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfonic acid, cyclohexylsulfamic acid, and quinic acid. Such salts can be prepared, for example, by reacting the free acid or base form of the product with one or more equivalents of the desired base or acid in a solvent in which the salt is insoluble, or in water that is later removed using a vacuum. Ion exchange can also be used to prepare desired salts.

Pharmaceutical formulations for parenteral administration may include liposomes. Liposomes and emulsions are delivery vehicles or carriers that are especially useful for hydrophobic drugs. Depending on biological stability of the therapeutic reagent, additional strategies for protein stabilization may be employed. Furthermore, one may administer the drug in a targeted drug delivery system such as, for example, in a liposome coated with target-specific antibody. The liposomes will bind to the target protein and be taken up selectively by the cell expressing the target protein.

Therapeutic compositions typically must be sterile and stable under the conditions of manufacture and storage. The composition can be formulated as a solution, microemulsion, liposome, or other ordered structure suitable for a high drug concentration. In some embodiments, the carrier can be a solvent or dispersion medium including, but not limited to, water; ethanol; a polyol such as for example, glycerol, propylene glycol, liquid polyethylene glycol, and the like; and, combinations thereof. The proper fluidity can be maintained in a variety of ways such as, for example, using a coating such as lecithin, maintaining a required particle size in dispersions, and using surfactants.

In some embodiments, isotonic agents can be used such as, for example, sugars; polyalcohols that include, but are not limited to, mannitol, sorbitol, glycerol, and combinations thereof; and sodium chloride. Sustained absorption characteristics can be introduced into the compositions by including agents that delay absorption such as, for example, monostearate salts, gelatin, and slow release polymers. Carriers can be used to protect active compounds against rapid release, and such carriers include, but are not limited to, controlled release formulations in implants and microencapsulated delivery systems. Biodegradable and biocompatible polymers can be used such as, for example, ethylene vinyl acetate, polyanhydrides, polyglycolic acid, collagen, polyorthoesters, polylactic acid, polycaprolactone, polyglycolic copolymer (PLG), and the like. Such formulations can generally be prepared using methods known to one of skill in the art.

Local administration of the mimetics to a target tissue, particular in diseases that include ischemic tissue, can be used in the methods taught herein. In some embodiments, the mimetics are administered by injections that can include intramuscular, intravenous, intra-arterial, intracoronary, intramyocardial, intrapericardial, intraperitoneal, subcutaneous, intrathecal, or intracerebrovascular injections.

The compounds may be administered as suspensions such as, for example, oily suspensions for injection. Lipophilic solvents or vehicles include, but are not limited to, fatty oils such as, for example, sesame oil; synthetic fatty acid esters, such as ethyl oleate or triglycerides; and liposomes. Suspensions that can be used for injection may also contain substances that increase the viscosity of the suspension such as, for example, sodium carboxymethyl cellulose, sorbitol, or dextran. Optionally, a suspension may contain stabilizers or agents that increase the solubility of the compounds and allow for preparation of highly concentrated solutions.

In some embodiments, a sterile and injectable solution can be prepared by incorporating an effective amount of an active compound in a solvent with any one or any combination of desired additional ingredients described above, filtering, and then sterilizing the solution. In some embodiments, dispersions can be prepared by incorporating an active compound into a sterile vehicle containing a dispersion medium and any one or any combination of desired additional ingredients described above. Sterile powders can be prepared for use in sterile and injectable solutions by vacuum drying, freeze-drying, or a combination thereof, to yield a powder that can be comprised of the active ingredient and any desired additional ingredients. Moreover, the additional ingredients can be from a separately prepared sterile and filtered solution. In some embodiments, a mimetic may be prepared in combination with one or more additional compounds that enhance the solubility of the mimetic.

In some embodiments, the compounds can be administered by inhalation through an aerosol spray or a nebulizer that may include a suitable propellant such as, for example, dichlorodifluoromethane, trichlorofluoromethane, dichlorotetrafluoroethane, carbon dioxide, or a combination thereof. In some aspects, a dosage unit for a pressurized aerosol may be delivered through a metering valve. In some aspects, capsules and cartridges of gelatin, for example, may be used in an inhaler and can be formulated to contain a powderized mix of the compound with a suitable powder base such as, for example, starch or lactose.

In some embodiments, a therapeutically or prophylactically effective amount of a mimetic may range in concentration from about 0.001 nM to about 0.1 M; from about 0.001 nM to about 0.05 M; from about 0.01 nM to about 15 μM; from about 0.01 nM to about 10 μM, or any range therein. In some embodiments, the mimetics may be administered in an amount ranging from about 0.001 mg/kg to about 50 mg/kg; from about 0.005 mg/kg to about 40 mg/kg; from about 0.01 mg/kg to about 30 mg/kg; from about 0.01 mg/kg to about 25 mg/kg; from about 0.1 mg/kg to about 20 mg/kg; from about 0.2 mg/kg to about 15 mg/kg; from about 0.4 mg/kg to about 12 mg/kg; from about 0.15 mg/kg to about 10 mg/kg, or any range therein, wherein a human subject is assumed to average about 70 kg.

The mimetics can be administered as a diagnostic, therapeutic or prophylactic agent in a combination therapy with the administering of one or more other agents. The agents of the present invention can be administered concomitantly, sequentially, or cyclically to a subject. Cycling therapy involves the administering a first agent for a predetermined period of time, administering a second agent for a second predetermined period of time, and repeating this cycling for any desired purpose such as, for example, to enhance the efficacy of the treatment. The agents can also be administered concurrently. The term “concurrently” is not limited to the administration of agents at exactly the same time, but rather means that the agents can be administered in a sequence and time interval such that the agents can work together to provide additional benefit. Each agent can be administered separately or together in any appropriate form using any appropriate means of administering the agent or agents.

Each of the agents described herein can be administered to a subject in combination therapy. In some embodiments, the agents can be administered at points in time that vary by about 15 minutes, 30 minutes, 1 hour, 2 hours, 4 hours, 8 hours, 12 hours, 18 hours, 24 hours, 48 hours or 1 week in time. In some embodiments, at least one of the agents is an immunomodulatory agent. In some embodiments, the agents can include antiproliferatives, antineoplastics, antimitotics, anti-inflammatories, antiplatelets, anticoagulants, antifibrins, antithrombins, antibiotics, antiallergics, antioxidants, and any prodrugs, codrugs, metabolites, analogs, homologues, congeners, derivatives, salts and combinations thereof.

According to some embodiments, the invention includes sustained release formulations for the administration of one or more agents. The sustained release formulations can reduce the dosage and/or frequency of the administrations of such agents to a subject.

In some embodiments, a CXC chemokine analog may be prepared in a “prodrug” form, wherein the mimetic begins acting upon its metabolism in vivo, in which the mimetic can become, for example, an agonist or an antagonist. The prodrugs can have, for example, an alkyl group attached through a hydrolyzable linkage, such as an ester or anhydride linkage that must hydrolyze before the analog can be active. In some embodiments, the analog is a pharmaceutically acceptable salt form of the analog. A CXC chemokine analog can also be hydrolyzably connected to an additional agent and, thus, deliver the additional agent in vivo upon the hydrolysis of the analog from the additional agent; such a construct is known as a “codrug” form of the analog. Examples of such agents include the bioactive agents, biobeneficial agents, diagnostic agents, and additional CXC chemokine analogs. In some embodiments, the agent comprises a glycosaminoglycan such as for example, heparin, hirudin, hyaluronic acid, and any prodrugs, codrugs, metabolites, analogs, homologues, congeners, derivatives, salts and combinations thereof. In some embodiments, the agent comprises a phospholipid such as, for example, phosphatidylcholine (lecithin). In some embodiments, the phospholipids can be conjugated to any functional group on a CXC chemokine analog, wherein the phospholipid and/or the CXC chemokine analog can be modified as necessary. In these embodiments, the phospholipids can be connected to an amino functional group, such as for example the N-terminus of a CXC chemokine analog. It is to be appreciated that one skilled in the art should recognize that some of the groups, subgroups, and individual biobeneficial agents described herein may not be used in some embodiments of the present invention.

Phosphatidylcholine is a phospholipid that is a major constituent of cell membranes. Phosphatidylcholine may have hepatoprotective activity, is important for normal cellular membrane composition and repair, and is the major delivery form of the essential nutrient choline, which is a precursor in the synthesis of the neurotransmitter acetylcholine. Phosphatidylcholine's role in the maintenance of cell-membrane integrity is vital to all of the basic biological processes such as, for example, information flow that occurs within cells in the transcription of DNA to RNA; the translation of RNA to proteins; the formation of cellular energy; and intracellular communication or signal transduction. Phosphatidylcholine has a fluidizing effect on cellular membranes, which is important in that a decrease in cell-membrane fluidization, a breakdown of cell-membrane integrity, and an impairment of cell-membrane repair mechanisms are associated with a number of disorders, including, but not limited to liver disease, neurological diseases, various cancers, cell death.

In some embodiments, the CXC chemokine could be administered with phosphatidylcholine to treat a disease. In some embodiments, the disease can include or be associated with liver disease. The liver diseases may include, but are not limited to, alcoholic and non-alcoholic liver disorders such as, for example, fibrosis; cirrhosis; and hepatitis A, B, C and E. In some embodiments, the disease can be neurological disease. The neurological diseases include, but are not limited to, manic conditions; cognitive disorders such as old-age memory loss, short-term memory loss, and Alzheimer's Disease; and tardive dyskinesia. In some embodiments, the disease can be any cancer that is associated with a deficiency in choline and phosphatidylcholine such as, for example, liver cancer. In some embodiments, the disease can be a choline deficiency that results in apoptosis, atherosclerosis or a loss of memory. In some embodiments, an effective amount of phosphatidylcholine is a daily administration that ranges from about 10 mg/kg to about 1000 mg/kg, from about 20 mg/kg to about 800 mg/kg, from about 30 mg/kg to about 600 mg/kg, from about 40 mg/kg to about 400 mg/kg, from about 40 mg/kg to about 200 mg/kg, from about 50 mg/kg to about 100 mg/kg, or any range therein.

In some embodiments, a CXC chemokine analog compound of the invention may be co-administered with a second agent by administering the CXC chemokine before, at the same time, or after the administration of the second agent. The concurrent administration can be made using a co-drug form of the CXC chemokine and second agent, where the separate activities of the two drugs are not realized until the codrug is broken down in vivo, such that the separation of the linkage between the two compounds creates the two separate activities.

### EXAMPLES

The following examples illustrate, but do not limit, the present invention.

### Example 1

Peptides of the invention may be synthesized chemically from the C-terminus to the N-terminus (“reverse sequence”) using the Fmoc/tBu strategy either manually or automatically using a batchwise or continuous flow peptide synthesizer.

Reagents and Procedures

Main Solvent: a grade certified, ACS spectroanalyzed, N,N-dimethylformamide (DMF) (Fisher, D131-4). The DMF is treated with activated molecular sieves, type 4A (BDH, B54005) for at least two weeks and then tested with 2,4-dinitrofluorobenzene (FDNB) (Eastman). Equal volumes of an FDNB solution (1 mg/ml of FDNB in 95% EtOH) and DMF are mixed and allowed to stand for 30 minutes. The absorbance of the mixture is then taken at 381 nm over an FDNB blank solution (no DMF), and if the absorbance is approximately 0.2, then the DMF is suitable for the synthesis.

Deblocking Agent: 20% piperidine (Aldrich, 10,409-4) in DMF containing 0.5% (v/v) triton X100 (Sigma, T-9284).

Activating Agents: 2-(H-benzotriazol-lyl)-1,1,3,3-tetramethyl uronium tetrafluoroborate (TBTU) (Quantum RichelLeu, R0139); hydroxybenzotriazole (HOBt) (Quantum RichelLeu, R0166-100), each at a concentration of 0.52 M in DMF; and 4-methylmorpholine (NMM) (Aldrich, M5 655-7) at a concentration of 0.9 M in DMF. In the case of amino acids sensitive to racemization such as, for example, cysteine, a 2,4,6-collidine (Aldrich, 14,238-7) is used at a concentration of 0.78 M in a 1/1 (v/v) mixture of DMF/dichloromethane (DCM).

Support Resin: TentaGel® RAM (90 μm) beads are used with a 9-fluorenylmethoxycarbonyl (Fmoc) Rink-type linker (Peptides Int'l, RTS-9995-PI) in a column. The synthesis begins using 0.5 g of the resin with a degree of substitution of 0.21 mmol/g for 0.21 (0.5) or 0.101 mmol of peptide.

An Fmoc-L-amino derivative is prepared with protected side-chains. The side-chains are protected using t-butoxycarbonyl (Boc), t-butyl (tBu), and triphenylmethyl (Trt) groups in a 4 fold excess (Peptides Int'l; Bachem; Novabiochem; Chem-Impex, Inc). The residues to be cyclized, for example Glu⁶⁰ and Lys⁵⁶ in some embodiments, are Allyl-protected (Millipore/Perseptive Biosys.).

Initial Amino Loading and Peptide Synthesis Procedure

The synthesis starts from the C-terminus, and the residues are double coupled automatically at ambient temperature using a 4-fold excess of the residues and the coupling reagents, TBTU and HOBt in DMF, for each coupling. Double coupling is used to ensure a high yield of coupling and can be a second coupling step that follows single coupling.

The synthesis can be interrupted after select residues for cyclization, such as Leu⁵⁵, for lactamization of residues Glu⁶⁰ and Lys⁵⁶ away from the column. In this example, the peptide bound to the support is cyclized by first removing the lateral allyl groups from protected residues, such as Glu⁶⁰ and Lys⁵⁶, as described below. The peptide synthesis is then resumed.

Removal of the Allyl Groups

The support-bound peptide is removed from the column and a 3-fold solution (347 mg) of tetrakis(triphenylphosphine) palladium(0) (Pd(PPh₃)₄) (Sigma-Aldrich, 21,666-6) and 0.1 mmol of the peptide attached to the resin is dissolved in 5% acetic acid. The peptide is activated using 2.5% NMM in CHCl₃ at a concentration of 0.14 M under an argon purge. The solution is added to the support-bound peptide in a reaction vial containing a small magnetic bar for gentle stirring. The mixture is flushed with argon, sealed and stirred at room temperature for 6 hours. The support-bound peptide is transferred to a filter funnel and subject to a series of washes: (i) the first wash is with a 30 ml of a 0.5% (w/w) solution of sodium diethyldithiocarbonate in DMF; (ii) the second wash is with DCM alone; (iii) the third wash is with a 1/1 (v/v) mixture of DCM/DMF; and (iv) the fourth wash is with DMF alone. A positive Kaiser test indicated the deprotection of the amino side chained of the Lys⁵⁶.

Lactam Formation:

Activating Agent: 7-azabenztriazol-1-yloxytris(pyrrolindino)phosphonium-hexafluorophosphate (PyAOP) (PerSeptive Biosys. GmbH, GEN076531) is used at a concentration that is 1.4-fold over the 0.105 mmol peptide sample size (e.g., 0.105 mmol×1.4 fold×521.7 MW=76.6 mg PyAOP); and NMM is used at a concentration that is 1.5-fold over the PyAOP e.g., 0.105 mmol×1.4 fold×1.5 fold=0.23 mmol NMM; volume=0.23/0.9M NMM=263 μl).

The lactamization is a cyclization reaction that is carried out with the support-bound peptide in an amino acid vial at room temperature overnight (e.g., ˜16 hours) with gentle agitation. The support-bound peptide is poured back into the column, washed with DMF, and then allowed to continue through completion of the cyclization process, wherein a cyclic amide bridge is thereby introduced into the peptide. A negative Kaiser test is used to indicate the completion of the cyclization process.

Removal of the Final Product from the Support

The support-bound peptide is removed from the synthesizer, placed in a medium filter funnel, washed with DCM to replace the non-volatile DMF, and thoroughly dried under high vacuum for at least two hours, or preferably, overnight.

Cleavage Mixture (reagent K): 100 ml of a trifluoroacetic acid (TFA)/Phenol/Water/Thio-Anisol/EDT (82/5/5/5/2.5)(v/v) mixture is prepared. The support-bound peptide (0.5 g) is poured into 7.5 ml of reagent K with gentle agitation on a rocker, allowed to react for 4 hours at room temperature, filtered, and washed with neat TFA. The 7.5 ml of reagent K contains the following:

| | | |
|---|---|---|
| TFA | 6.15 | ml (Halocarbon) |
| Phenol | 0.375 | ml (Aldrich) |
| Water | 0.375 | ml (MillQ) |
| Thio-Anisol | 0.375 | ml (Aldrich) |
| EDT | 0.187 | ml (Aldrich) |
| Total | 7.5 | ml |

Precipitation of the Peptide

The cleaved (free) peptide solution is filtered through a filter funnel into a 50 ml round bottom flask. The support is rinsed twice with 4 ml TFA to release the free peptide. The solution of TFA and peptide is concentrated on a rotavap and added drop wise into cold diethyl ether previously treated with activated neutral aluminum oxide to make it free of peroxide. An excess of ether is used at approximately 10-fold the weight of the support. The support beads from which the peptide was cleaved were stored until the yield was determined and the peptide was characterized. The precipitate is collected at room temperature in a screw-capped 50 ml polypropylene vial after centrifugation for 4 minutes at 2000 rpm in a bench-top centrifuge. The pellets of free peptide were washed 3× with cold ether, centrifuged and dried under a flow of argon. The precipitate was dissolved in 20% acetonitrile with 0.1% TFA and lyophilized.

Crude Product Characterization

The product is purified and characterized using an analytical HPLC procedure. A Vydac 218TP54 column (C18 reversed-phase, 4.6 mm×150 mm inner column dimensions, and 5 μm particle size). A multisolvent mobile phase is used, and the eluants are a 0.1% TFA/H₂O (solvent A) and a 0.1% TFA/acetonitrile (solvent B).

Elution Conditions: A multisolvent delivery system is used and combines solvent A and solvent B to alter the polarity of the mobile phase during elution. The mobile phase is delivered at a flow rate of 1.0 ml/min and at a concentration of 20-50% B for 40 minutes; at a concentration of 60-90% B for 5 minutes; at a concentration of 90-20% B for 5 minutes; and at a concentration of 20% B for 10 minutes. The detector is set at 214 nm to read 0.5 absorbance units over a full scale.

Sample Preparation

An aliquot of the product is weighed and dissolved in a mixture of 20% acetonitrile/0.1% TFA (v/v) at a concentration of 2 mg/ml. The solution is microfuged and 20 μl is injected into the HPLC column. Samples corresponding to the main and major peaks are collected, SpeedVac dried, and characterized by molecular weights using mass spectroscopy.

Several sequences have been prepared and contemplated. A Listing of Sequences follows this example section and precedes the claims. The sequences include some useful CXC mimetics that can be prepared by the solid phase peptide synthesis. The underline residues represent a cyclic portion of the mimetic.

A representative number of species of the CXC chemokine analogs have been produced and tested to show that it is reasonable to expect that all CXC chemokines will have utility as agonists and antagonists when designed according to the general constructs taught herein. The experimental tests have included binding assays, calcium mobilization, neutrophil mobility, in vitro and in vivo tissue response, and the like, all of which are accepted by those in the art as indicators of utility as ligands and potential therapeutics.

### Example 2

Modification with an Agent

Agents can be attached as modifying groups that are pendant or in-chain with a CXC chemokine mimetic. A trifunctional amino acid, for example, can be incorporated into the CXC chemokine mimetic as a linker and the third functionality can be connected to an agent. Protecting groups can be used to selectively attach an agent to the trifunctional amino acid. Benzyl esters are one type of protecting group that can be used for a lysine carboxyl, for example, and t-butoxycarbonyl can be used for amino groups such as, for example, the amino group in glutamic acid.

Amino, hydroxyl and carboxyl groups can be used, for example, as a connecting site for agents. Carboxyl groups can be used as a connecting site for agents having, for example, amino, hydroxyl, or thiol groups. Coupling agents include, but are not limited to, 1-ethyl-3(3-dimethylaminopropyl)carbodiimide (EDC) and 1,3-dicyclohexylcarbodiimide (DCC).

An example of an amine functional compound is 4-amino-TEMPO, an antioxidant and antihypertensive that can be administered as a codrug in combination with a CXC chemokine mimetic. Such an amine functional compound may be connected to an amino acid sequence containing free carboxyls such as, for example, the lysine-derived carboxyls, by first activating the carboxyls and coupling the amine in a solvent under agitation. The carboxyls may be activated with, for example, N-hydroxysuccinimide (NHS) and DCC in a solvent such as, for example, THF or chloroform, which produces N-hydroxysuccinimidyl ester. Examples of the solvent that may be used to couple the amine to the carboxyls include, but are not limited to, THF and DMF. One of skill will appreciate that other linkages can be preselected and created in order to increase the rate of release of a desired agent from a CXC chemokine mimetic such as, for example, an ester or an anhydride linkage.

In some embodiments, the reaction can occur at a temperature ranging from about 5° C. to about 50° C., from about 15° C. to about 35° C., from about 20° C. to about 30° C., or any range therein. In some embodiments, the reaction time can range from about 0.5 hours to about 24 hours, from about 1 hour to about 18 hours, from about 4 hours to about 16 hours, from about 6 hours to about 12 hours, or any range therein.

A benzyl ester protecting group can be removed from a lysine carboxyl by hydrogenolysis with hydrogen gas over a catalyst such as, for example, palladium or platinum on carbon. Examples of suitable solvents include, but are not limited to, ethanol, methanol, isopropanol, and THF. The reaction may be conducted under about 1 atm of hydrogen for about 6 hours to about 24 hours, for about 8 hours to about 16 hours, for about 10 hours to about 14 hours, or any range therein.

Modification with a Glycosaminoglycan

A glycosaminoglycan can be connected to an amine functional group as an aldehyde-terminated heparin, for example, to provide additional control over the behavior of the CXC chemokine mimetic in vivo and/or to provide a codrug form of the mimetic. An example of an aldehyde-terminated heparin is represented by the following formula: 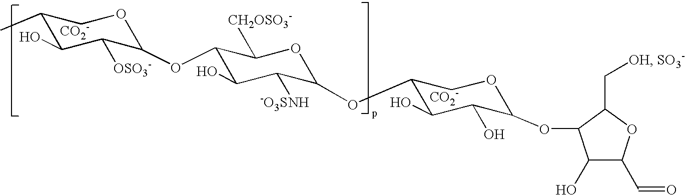
wherein p is an integer not equal to 0.

The aldehyde-terminated heparin can be combined with the amine functional group in a DMF/water solvent and subsequently reduced with NaCNBH₃ to produce a heparin linked to a CXC chemokine mimetic through an amide bond.

Modification with PEG

CXC chemokines and CXC chemokine analogs of the invention may be modified by the addition of polyethylene glycol (PEG). PEG modification may lead to improved circulation time, improved solubility, improved resistance to proteolysis, reduced antigenicity and immunogenicity, improved bioavailability, reduced toxicity, improved stability, and easier formulation (For a review see, Francis et al., International Journal of Hematology 68:1-18, 1998). PEGylation may also result in a substantial reduction in bioactivity.

There are a variety of available PEG sizes and derivatives that are commercially designed for specific applications such as, for example, attachment to a variety of different chemical functionalities including, but not limited to, amines, thiols, hydroxyls, sulfhydryls, and carboxyls. In one example, an amine group of a CXC chemokine mimetic can be combined with a carboxyl-terminated PEG (Nektar Corp.) in the presence of, for example, EDC or DCC to form a pegylated structure through formation of an amide bond between the CXC chemokine mimetic and the PEG. In another example, either a succinimidyl derivative of mPEG (Nektar Corp.) or an isocyanate-terminated mPEG (Nektar Corp.) can be combined with an CXC chemokine mimetic under conditions known to those of skill in the art. In another example, the carboxyl group of an CXC chemokine mimetic can be activated with, for example, EDC or DCC and combined with an amino-terminated mPEG (Nektar Corp.) In another example, an amine group of an CXC chemokine mimetic can be combined with a methacrylate-terminated mPEG (Nektar Corp.) in the presence of an initiator capable of undergoing thermal or photolytic free radical decomposition. Examples of suitable initiators include benzyl-N,N-diethyldithiocarbamate or p-xylene-N,N-diethyldithiocarbamate.

### Example 3

IP-10s

The IP-10 CXC chemokines are the subject of U.S. application Ser. No. 11/590,210, which is hereby incorporated herein by reference in its entirety. The cross-reference SEQ ID NOs from the source application are used in the explanation and in any associated table or figure, and the SEQ ID NOs used in the present application are provided to allow for location of the sequences in the formal sequence listing of the instant application

SEQ ID NOs: 1641-1645 were prepared for testing their ability to bind to an IP-10 receptor and their efficacy in mediating intracellular calcium mobilization ([Ca²⁺]ᵢ) at a variety of concentrations.

Binding and Calcium Mobilization: Suspensions of CXCR-3/300-19 cells were used to assess binding and intracellular calcium mobilization induced by IP-10 analogs. These are mouse pre-B lymphocytes transfected with the CXCR3 receptor, (Moser, et al). The cells were washed in RPMI media and resuspended in RPMI media supplemented with 10% FCS, then plated at 1.2×10⁵ cells per well of 96-well black wall/clear bottom plates coated with poly-D-lysine (Becton Dickinson) and loaded with 100 uL fluorescent calcium indicator FLIPR Calcium 3 assay kit component A (Molecular Probes) for 1 hr at 37° C. The cells on the plates were then spun at 1000 rpm for 15 minutes at room temperature.

Each of the sequences successfully bound to the cellular receptors. The intracellular calcium mobilization in response to 25 uL (0-100000 nM final concentrations) of the appropriate and various concentrations of analogue was measured at 37° C. by monitoring fluorescence as a function of time in all the wells using the Flexstation Fluorometric Imaging Plate Reader (Molecular Devices). All analogues were run simultaneously with rhIP-10 (R&D Systems) as the standard. Table 7 provides the dosage effect of the binding of each of the IP-10 analogs on the calcium mobilization activity of the cells.

TABLE 7

| Peptide Analogs | | | | | | |
|---|---|---|---|---|---|---|
| (SEQ ID NO:) | | | | | | |
| U.S. App. | | Dosage | | | | |
| No. | Present | (μM) | | | | |
| 11/590,210 | Application | 1.2 | 3.7 | 11.1 | 33.3 | 100 |
| 1641 | 123 | 12.6 | 12.7 | 14.1 | 20 | 52.9 |
| 1642 | 124 | 15.2 | 13.8 | 15.4 | 20 | 3.1 |
| 1643 | 125 | 16 | 14 | 14.4 | 18.6 | 55.1 |
| 1644 | 126 | 18.8 | 18.5 | 17.2 | 18.9 | 46.6 |
| 1645 | 127 | 17.4 | 15.4 | 12.5 | 13.3 | 3.4 |

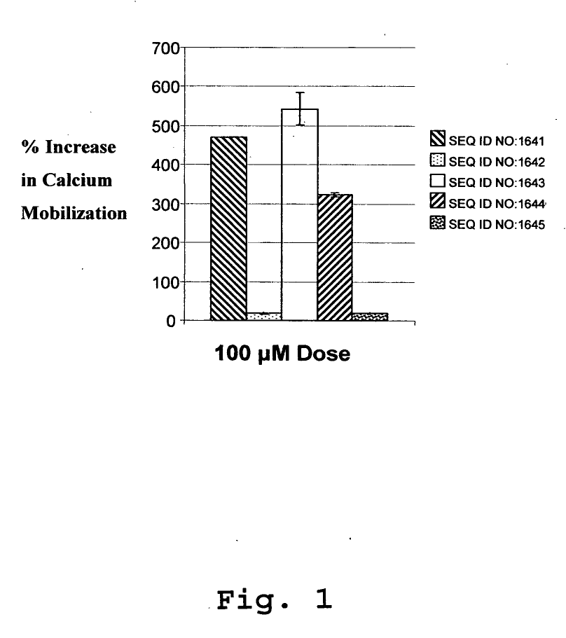

FIG. 1 illustrates the induction of [Ca²⁺]i mobilization by select IP-10 analogs at a concentration of 100 μM according to some embodiments. The results are resentative of three independent experiments. SEQ ID NOs: 1641-1645 all bound to the receptor and affected calcium mobilization. SEQ ID NOs: 1641, 1643, and 1644, however, increased calcium mobilization by 300 to over 500%. The results are compared to a recombinant human IP-10 chemokine, as described above.

The acetylated-IP-10-(1-16)-[linker]-IP-10-(66-78) analog represented be SEQ ID NO:1641 increased intracellular calcium mobilization by nearly 500%, but an [Ala⁹,Phe¹¹] amino acid substitution in the same type of analog decreased the calcium mobilization dramatically as shown by the effects of SEQ ID NO:1642.

In an IP-10-(1-15)-[linker]-IP-10-(58-71) analog, a [Pro⁷] amino acid substitution in SEQ ID NO:1643 resulted in an increase in intracellular calcium mobilization when compared to the results of SEQ ID NO:1641. A [Ser⁹,Ser¹¹,Glu⁶³] amino acid substitution of the same type of analog still provide a very substantial increase in intracellular calcium mobilization of over 300% using SEQ ID NO:1644. Interestingly, however, a [Glu⁶⁷] amino acid substitution decreased the effect on calcium mobilization dramatically as shown by the effect of SEQ ID NO:1645.

The results provided by this example show that IP-10 analogs having a total of about 30 amino acids and conserving N-terminal residues 1-15 and C-terminal residues 66-71 of the IP-10 chemokine are effective at binding and can increase the cellular activity induced by the binding to different degrees, depending on the dosage of the analog administered and the presence of amino acid substitutions. In particular, the results suggest that the Cys⁹ and Cys¹¹ residues can be substituted with Ser⁹ and Ser¹¹ in the conserved N-terminal 1-15 region with little effect, and Lys⁶³ can be substituted by Glu⁶³ with little effect, but substantial differences in results occur where Lys⁶⁷ is substituted with Glu⁶⁷, which is in the range of the conserved C-terminal region of 66-71.

Accordingly, an IP-10 analog that is supported by these results would range from about 21 to about 34 amino acids in length and comprise:

an N-terminal region comprising and conserving the IP-10 chemokine residues 1-15;

C-terminal region comprising and conserving the IP-10 chemokine residues 66-71, and conservatively modified variants thereof;

and an optional linker having up to 4 amino acids, wherein the linker is preferably 11-aminoundecanoic acid.

### Example 4

SDF-1s

The SDF-1 CXC chemokines are the subject, for example, of U.S. application Ser. Nos. 11/393,769, 11/388,542, and 10/945,674, each of which is hereby incorporated herein by reference in its entirety. The results provided in the instant application are by no means comprehensive and are provided to show the usefulness of SDF-1 mimetics in general. The cross-reference SEQ ID NOs from the source application are used in the explanation and in any associated table or figure, and the SEQ ID NOs used in the present application are provided to allow for location of the sequences in the formal sequence listing of the instant application.

Calcium Mobilization

his example illustrates the efficacy of SDF-1 and SDF-1 peptide analogs in mediating intracellular calcium mobilization ([Ca²⁺]ᵢ). To illustrate that the binding of SDF-1 and SDF-1 peptide analogs results in the agonistic activation of the CXCR4 receptor, [Ca²⁺]ᵢ mobilization assays were conducted

Fluo-4, AM loaded SUP-T1 cells (5×10⁶ cells/ml), a human lymphoid cell line, were stimulated with SDF-1 and Compound A (SEQ ID NO:809), Compound B (SEQ ID NO:810), Compound C (SEQ ID NO:811), Compound D (SEQ ID NO:812) and Compound E (SEQ ID NO:813) at the concentrations indicated. The values represent the mean+/−one S.D. of a representative experiment from three independent experiments.

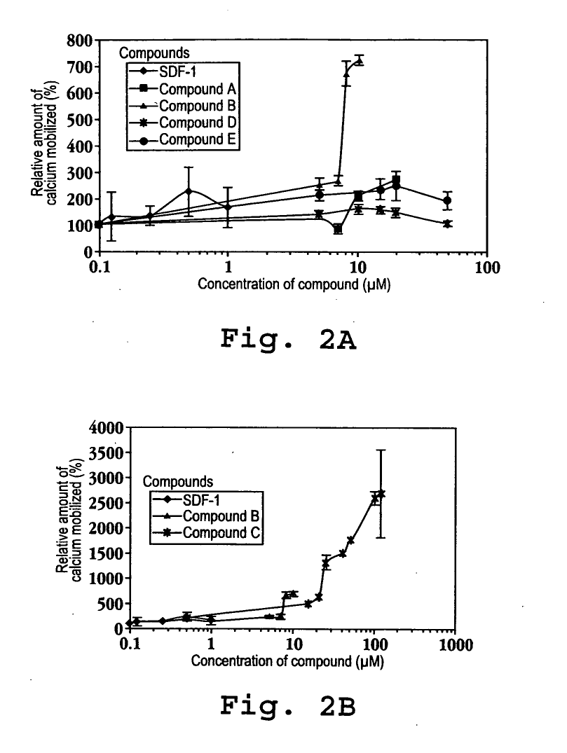

FIGS. 2A and 2B shows the incubation of SUP-T1 cells with SDF-1 according to some embodiments.rief The mimetics used include Compound A (SEQ ID NO:809), Compound B (SEQ ID NO:810), Compound C (SEQ ID NO:811), Compound D (SEQ ID NO:812) or Compound E (SEQ ID NO:813), and the results showed a receptor-mediated induction of [Ca²⁺]ᵢ mobilization. (The underlined residues in the structures depicted below were cyclized by a lactamization reaction between lysine and glutamic acid residues.)

SEQ ID NOs.:809-813 were prepared for testing their ability to bind and activate an SDF-1 receptor, for example, mediate intracellular calcium mobilization ([Ca²⁺]ᵢ) at a variety of concentrations, etc.

The [Ca²⁺]ᵢ mobilization assays were conducted as follows. Briefly, SUP-T1 cells (ATCC, Manassas, Va.), a human lymphoid cell line, were cultured in RPMI containing phenol red (Invitrogen, Burlington, Ontario, Canada) with 10% fetal bovine serum and antibiotics consisting of 100 U/ml penicillin G sodium and 100 μg/ml streptomycin sulfate (Invitrogen) at a density between 2×10⁵ and 8×10⁵ cells/ml. Cells were harvested and suspended in Tyrode's salt solution, consisting of 137 mM NaCl, 2.7 mM KCl, 1 mM MgCl₂, 1 mM CaCl₂, 0.2 mM NaH₂PO₄, 12 mM NaHCO₃, and 5.5 mM glucose, at 2×10⁶ cells/ml then labeled with 4 μM Fluo-4/AM (Molecular Probes, Eugene, Oreg.) for 45 min at 37° C.

Subsequently, cells were washed three times with Tyrode's salt solution, and resuspended at 5×10⁶ cells/ml. SDF-1, Compound A (SEQ ID NO:809), Compound B (SEQ ID NO:810), Compound C (SEQ ID NO:811), Compound D (SEQ ID NO:812) or Compound E (SEQ ID NO:813) at the concentrations indicated were injected into aliquots of 5×10⁵ cells. Changes in the level of cellular fluorescence were read in a Thermo Labsystems Fluorskan Acsent fluorescence plate reader (VWR, Mississauga, Ontario, Canada). Controls include cells treated with the recombinant chemokine or plain medium. Data is expressed with 100% being the level of fluorescence in plain medium. The values represent the mean+/−one S.D. of a representative experiment from three independent experiments.

Binding Assays

The efficacy of SDF-1 and SDF-1 peptide analogs as CXCR4 agonists was demonstrated through CXCR4 receptor binding assays. FIG. 3 shows a competitive dose response for binding to the SDF-1 receptor by native SDF-1 and the CXCR4 agonists (competing ligands) against ¹²⁵I-SDF-1 according to some embodiments.

Briefly, SUP-T1 cells were grown using methods known to those of skill in the art and described in U.S. application Ser. No. 11/393,769. Millipore MultiScreen plates with Durapore membrane (Millipore, Bedford, Mass.) were used for high throughput binding assays. The buffer used for the assay (binding buffer) consisted of 0.1% bovine albumin, 25 mM HEPES, 100 μg/ml chondroitin sulphate C, and 0.02% sodium azide in RPMI-1640. SUP-T1 cells were harvested, washed with plain RPMI and resuspended in binding buffer at 5×10⁶ cells/ml. The Durapore membrane of the Millipore MultiScreen plates was moistened with blocking buffer containing 0.5% BSA (Sigma), 50 mM HEPES, 150 mM NaCl, 5 mM MgCl₂, 1 mM CaCl₂ and 0.02% sodium azide for 40 min before use. To the wells were added binding buffer, antagonist, the appropriate radiolabeled chemokine, and the appropriate cells. Cells were preincubated with peptide analogs for 30 min then incubated with ¹²⁵I-SDF-1 for 2 h with shaking at 4° C. SDF-1 peptide analogs were used at concentrations indicated along with 0.5 nM radiolabeled SDF-1.

After three washes with cold PBS, plates were dried and radioactivity counted using a CliniGamma gamma counter (LKB Wallac, Gaithersburg, Md.). Controls include wells with only binding buffer and radiolabeled chemokine for background, and wells with binding buffer, unlabelled chemokine standard, radiolabeled chemokine and cells for standardization. The results are expressed as percentages of the maximal specific binding that was determined without competing ligand, and are the representative results from three independent experiments. A concentration-dependent inhibition of ¹²⁵I-SDF-1 is illustrated, indicating the affinity of SDF-1 and SDF-1 peptide analogs for the receptor. The inhibition of ¹²⁵I-SDF-1 binding by SDF-1 and the SDF-1 analogs is indicative of CXCR4 receptor binding.

Mobilizing Neutrophils

This example illustrates the efficacy of SDF-1 peptide analogs (as represented by Compound A (SEQ ID NO:809) and Compound B (SEQ ID NO:810)) in mobilizing circulating neutrophils in a mouse model. This study consisted of three groups of female Balb/c mice (Charles River, Wilmington, Mass.): an untreated control group of 6 mice and two 18-mouse test groups. Before the start of the study, 20-23 g mice were randomly grouped in appropriately labeled cages and identified by cage markings and shaved marks on the dorsal region. The two test groups were treated one time intravenously with SDF-1 analogs at a dose of 2.5 mg/kg in volumes approximating 200 μl. The evaluated end points included moribundity and complete blood counts with differentials.

Blood samples were obtained from 6 mice from each test group at t=30 minutes, 1 hour and 24 hours post analog administration. Prior to blood collection, mice were weighed and anesthetized. Blood was collected via a 1 cc syringe and 25 G needle (Becton Dickinson/VWR) by cardiac puncture. One fresh blood smear was produced. The remaining blood was expelled into a Becton Dickinson EDTA microtainer and mixed gently by 5 inversions. The smear and microtainer tubes were used for differential and CBC analysis on a CellDyn 3500 (Abbott Diagnostic Products, Mississauga, Ontario, Canada) and by veterinarians (Central Laboratory for Veterinarians, Langley, B.C, Canada). The differentials were used to evaluate the mobilization of neutrophils and were compared to the untreated control group.

The results are expressed as percentage of the count from untreated control animals and are representative of at least two experiments each with six animals per treatment. A time-dependent mobilization of neutrophils to the circulation is shown, indicating the rapid and potent activity of the peptide analogs. Compound B (represented by SEQ ID NO:810) exhibits an especially rapid and sustained action. Table 8 shows the percentage change in circulating neutrophils in Balb/c mice treated with 2.5 mg/kg of the designated compound compared to untreated control animals.

TABLE 8

| | Duration of treatment (hours) | | |
|---|---|---|---|
| | Percent change in circulating neutrophils | | |
| Compound | ½ | 1 | 24 |
| Compound A | 175% | 299% | 25% |
| Compound B | 348% | 304% | 113% |

SEQ ID NOs:3-32 have been prepared to use in the prevention, treatment, and ameliorization of diseases that can benefit from therapeutic angiogenesis.

Binding Assay

The efficacy of the SDF-1 mimetics of the invention to bind to mammalian cells and compete with SDF-1 was measured. The experiments include contacting an SDF-1 mimetic with a cell, and the experiments were performed using a human lymphoid cell line of SUP-T1 cells (American Type Culture Collection or ATCC) at a concentration of 5×10⁶ cells/ml. A DURAPORE membrane and Millipore MultiScreen 96-well plates were used in the binding assay, and the membrane was blocked with a PVP/Tween-based blocking buffer before use. An RPMI-based binding buffer, 0-400 nM of SDF-1 or 0-400 μM of an SDF-1 mimetic, a competitive dose of 0.02 nM ¹²⁵I-SDF-1 (Amersham), and SUP-T1 cells were added to the wells. The cells were incubated at 4° C. with shaking for 2 h, followed by triplicate washes with PBS. Bound ¹²⁵I-SDF-1 was counted using a CliniGamma gamma counter (LKB Wallac).

Experiments were performed in triplicate. Competition curves were fitted with Graphpad Prism v4.0 after subtracting non-specific binding to both filters and cells. The results are expressed as Ki values for the different SDF-1 mimetics and are shown in Table 9, where Ki is the binding affinity constant, SEM is the standard error of the measurement, and n is the number of samples.

TABLE 9

| Compound | | | | |
|---|---|---|---|---|
| Cross-ref. SEQ | | | | |
| ID NO. to | SEQ ID NO | | | |
| U.S. App. No. | in Present | | | |
| 11/388,542 | Application | Kᵢ (μM) | +/−SEM | N |
| Natural SDF-1 | 12 | 0.009 | 2.379 | 6 |
| SEQ ID NO: 1 | | | | |
| SEQ ID NO: 3 | 139 | 0.663 | 0.446 | 4 |
| SEQ ID NO: 4 | 140 | 0.586 | 0.224 | 3 |
| SEQ ID NO: 5 | 141 | 0.378 | 0.048 | 3 |
| SEQ ID NO: 6 | 142 | 0.306 | 0.022 | 3 |
| SEQ ID NO: 7 | 143 | 0.412 | 0.245 | 3 |
| SEQ ID NO: 8 | 144 | 0.137 | 0.006 | 3 |
| SEQ ID NO: 9 | 145 | 0.343 | 0.252 | 3 |
| SEQ ID NO: 10 | 146 | 0.493 | 0.097 | 3 |
| SEQ ID NO: 11 | 147 | 1.213 | 0.510 | 3 |
| SEQ ID NO: 12 | 148 | 0.877 | 0.568 | 3 |
| SEQ ID NO: 13 | 149 | 2.553 | 1.288 | 4 |
| SEQ ID NO: 14 | 150 | 1.173 | 0.645 | 4 |
| SEQ ID NO: 15 | 151 | 2.002 | 0.654 | 4 |
| SEQ ID NO: 16 | 152 | 2.115 | 1.074 | 4 |
| SEQ ID NO: 17 | 153 | 1.243 | 0.517 | 4 |
| SEQ ID NO: 18 | 154 | 2.308 | 0.056 | 4 |
| SEQ ID NO: 19 | 155 | 1.761 | 0.137 | 4 |
| SEQ ID NO: 20 | 156 | 3.351 | 0.992 | 3 |
| SEQ ID NO: 21 | 157 | 2.453 | 0.561 | 4 |
| SEQ ID NO: 22 | 158 | 0.744 | 0.143 | 4 |
| SEQ ID NO: 23 | 159 | 1.675 | 0.478 | 4 |
| SEQ ID NO: 24 | 160 | 1.780 | 0.921 | 4 |
| SEQ ID NO: 25 | 161 | 1.078 | 0.243 | 4 |
| SEQ ID NO: 26 | 162 | 1.265 | 0.730 | 4 |
| SEQ ID NO: 27 | 163 | 1.535 | 0.673 | 4 |
| SEQ ID NO: 28 | 164 | 0.741 | 0.360 | 4 |
| SEQ ID NO: 29 | 165 | 1.261 | 0.462 | 4 |
| SEQ ID NO: 30 | 166 | 1.112 | 0.323 | 4 |
| SEQ ID NO: 31 | 167 | 0.797 | 0.240 | 4 |
| SEQ ID NO: 32 | 168 | 0.833 | 0.268 | 4 |

Calcium Mobilization

The efficacy of the chemokine analogs of the invention to activate mammalian cell receptors is demonstrated by their ability to mobilize intracellular calcium in SUP-T1 cells. The experiments include contacting an SDF-1 mimetic with a cell. For the experiments, SUP-T1 cells (ATCC) were plated on the day of the experiment using 1.2×10⁵ cells per well in 96-well black-wall/clear-bottom plates coated with poly-D-lysine (BD Biosciences) and loaded using a fluorescent calcium indicator. The indicator used was from a FLIPR Calcium 3 assay kit, component A, (Molecular Probes) and was loaded in the cell for 1 hr at 37° C. The intracellular calcium mobilization in response to the appropriate analogue was measured at 37° C. by monitoring the fluorescence as a function of time simultaneously in all the wells using a Flexstation Fluorometric Imaging Plate Reader (Molecular Devices). The EC₅₀ values of the different SDF-1 mimetics of the present invention are summarized in Table 10.

TABLE 10

| Compound | | |
|---|---|---|
| Cross-ref. SEQ | | |
| ID NO. to | SEQ ID NO | |
| U.S. App. No. | in Present | |
| 11/388,542 | Application | EC₅₀ (μM) |
| SEQ ID NO: 3 | 139 | 0.346 |
| SEQ ID NO: 4 | 140 | 0.312 |
| SEQ ID NO: 5 | 141 | 0.211 |
| SEQ ID NO: 6 | 142 | 0.283 |
| SEQ ID NO: 7 | 143 | 0.281 |
| SEQ ID NO: 8 | 144 | 0.304 |
| SEQ ID NO: 9 | 145 | 0.225 |
| SEQ ID NO: 10 | 146 | 0.233 |
| SEQ ID NO: 11 | 147 | 0.228 |
| SEQ ID NO: 12 | 148 | 0.307 |
| SEQ ID NO: 13 | 149 | 0.137 |
| SEQ ID NO: 14 | 150 | 0.092 |
| SEQ ID NO: 15 | 151 | 0.157 |
| SEQ ID NO: 16 | 152 | 0.140 |
| SEQ ID NO: 17 | 153 | 0.316 |
| SEQ ID NO: 18 | 154 | 0.219 |
| SEQ ID NO: 19 | 155 | 0.253 |
| SEQ ID NO: 20 | 156 | 0.307 |
| SEQ ID NO: 21 | 157 | 0.361 |
| SEQ ID NO: 22 | 158 | 0.171 |
| SEQ ID NO: 23 | 159 | 0.202 |
| SEQ ID NO: 24 | 160 | 0.173 |
| SEQ ID NO: 25 | 161 | 0.132 |
| SEQ ID NO: 26 | 162 | 0.248 |
| SEQ ID NO: 27 | 163 | 4.315 |
| SEQ ID NO: 28 | 164 | 0.597 |
| SEQ ID NO: 29 | 165 | 1.873 |
| SEQ ID NO: 30 | 166 | 0.178 |
| SEQ ID NO: 31 | 167 | 0.709 |
| SEQ ID NO: 32 | 168 | 1.117 |

The SDF-1 mimetics were also shown to induce the survival of Human Umbilical Vein Endothelial Cells (HUVEC) in a serum free medium using an MTT assay to analyse cell viability after peptide treatment. The SDF-1 mimetics were shown to induce the differentiation of Human Vein Endothelial Cells using a matrigel tube formation assay, and they were also shown to induce neo-vessel formation in an aortic ring assay. Moreover, neovascularization was measured to show the effect on angiogenesis and the ability to induce a vascular supply to promote wound healing, and this was shown using a MATRIGEL plug assay.

### Example 5

IL-8s

The IL-8 CXC chemokines are the subject of U.S. application Ser. Nos. 10/932,208 and 10/243,795, each of which is hereby incorporated herein by reference in its entirety. The cross-reference SEQ ID NOs from the source application are used in the explanation and in any associated table or figure, and the SEQ ID NOs used in the present application are provided to allow for location of the sequences in the formal sequence listing of the instant application.

SEQ ID NOs 1642-1675 have been prepared to use in the prevention, treatment, and ameliorization of diseases.

Binding Assay

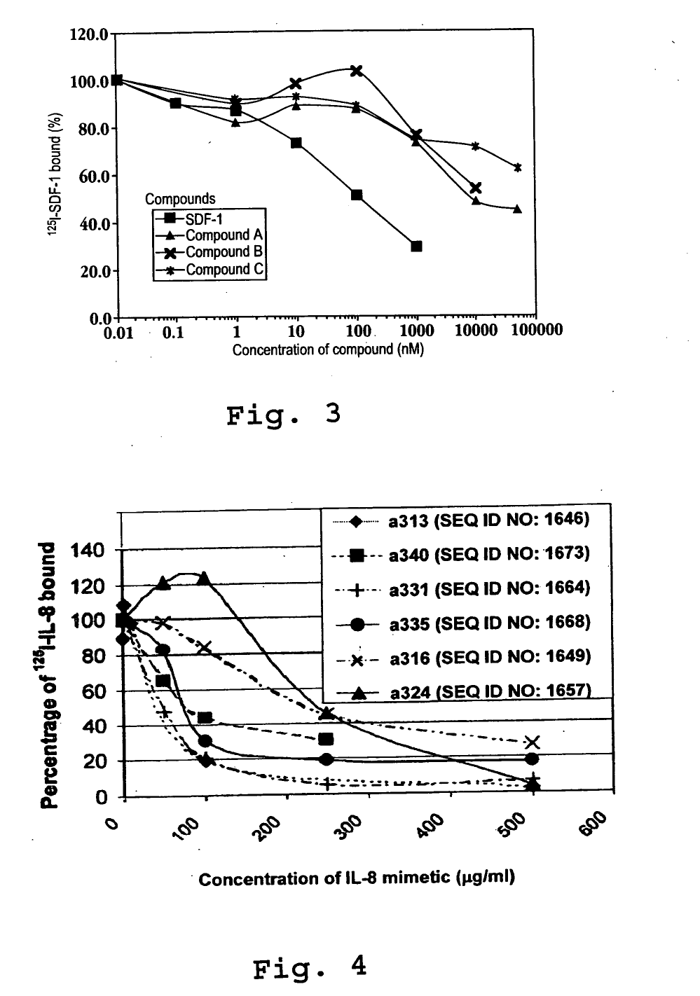

A competitive-dose-response binding assay was used to compare the ability of the native IL-8 to bind to the CXCR1/CXCR2 receptors with the ability of IL-8 agonists to bind to the CXCR1/CXCR2 receptors. An ¹²⁵I radiolabeled derivative of native IL-8 (“¹²⁵I-IL-8”) was used to measure the binding activity of native IL-8. The competitive dose response is shown in FIG. 4.

FIG. 4 shows the CXCR2 receptor binding of the IL-8 mimetics as competing ligands according to some embodiments. Differentiated HL-60 cells were assessed for ¹²⁵I-IL-8 binding following 2 hours of incubation with IL-8 or its agonist, and ¹²⁵I-IL-8. The ¹²⁵I-IL-8 was added at a concentration of 2 nM in the presence of native IL-8 and the IL-8 mimetics at their respective concentrations as shown. The results are expressed as percentages of the maximal specific binding that was determined without competing ligand and are representative of one independent experiment.

The procedure used HL-60 cells (American Type Culture Collection) that were grown in an RPMI culture medium containing phenol red, 10% fetal bovine serum, and antibiotics consisting of 100 U/ml penicillin G sodium and 100 μg/ml streptomycin sulfate. The cells were added at a density ranging from about 2×10⁵ to about 8×10⁵ cells/ml. The cells were then induced to differentiate and express CXCR2 by treating the cells for 3-7 days with 1.25% DMSO. Millipore MultiScreen plates and a Durapore® membrane (Millipore Corp.) were used for high throughput binding assays.

The binding buffer used for the assay consisted of 0.5% (w/v) bovine serum albumin (BSA) (e.g., 0.5 g BSA/100 ml buffer), 50 mM 4-(2-hydroxyethyl)-1-piperazineethanesulfonic acid (HEPES), 150 mM NaCl, 5 mM MgCl₂, 1 mM CaCl₂ and 0.02% sodium azide. The HL-60 cells were harvested, washed with plain RPMI, and resuspended in binding buffer at density of about 5×10⁶ cells/ml. The cells were preincubated with the IL-8 mimetics for 30 minutes. The binding buffer, the ¹²⁵I-IL-8, and the cells incubating with native IL-8 or IL-8 mimetic were then added to wells used to hold the cells in the assay. The cells were then incubated in the wells for another 2 hours with shaking. The IL-8 mimetics were used in concentrations indicated in FIG. 4 with a competitive dose of 2 nM of radiolabeled ¹²⁵I-IL-8.

After three washes with cold phosphate buffered saline (PBS), plates were dried and radioactivity counted using a CliniGamma gamma counter (LKB Wallac). Controls include (i) wells with only binding buffer and radiolabeled IL-8 chemokine for background, and (ii) wells with binding buffer, an unlabelled native IL-8 chemokine standard, radiolabeled chemokine and cells for standardization. A dose response curve is developed using a range of native IL-8 concentrations, and a concentration of 0 μg/ml is included.

Each data point is expressed as a percentage of the maximal specific binding that was determined using the radiolabeled native IL-8 without the competing IL-8 mimetics (“IL-8 agonist”) and represents measurements obtained from two or three wells in a representative experiment. A concentration-dependent inhibition of ¹²⁵I-IL-8 binding is shown in FIG. 4 and summarized in Table 11 and indicates the affinity of native IL-8 and IL-8 mimetics for the receptor.

TABLE 11

| Compound | | | |
|---|---|---|---|
| Cross-ref. | | | |
| SEQ ID NO. to | SEQ ID NO | | ¹²⁵I-IL-8 |
| U.S. App. No. | in Present | IC₅₀ | bound at maximal |
| 10/932,208 | Application | (μg/ml) | inhibition (%) |
| (Native IL-8) | 8 | 2.8 | 2.2 |
| 1646 | 88 | 47.5 | 2.9 |
| 1673 | 109 | 50.0 | 30.4 |
| 1664 | 101 | 56.7 | 7.0 |
| 1670 | 106 | 56.7 | 19.3 |
| 1674 | 110 | 60.0 | 34.5 |
| 1665 | 102 | 61.7 | 7.9 |
| 1667 | 104 | 61.7 | 15.8 |
| 1666 | 103 | 63.3 | 15.4 |
| 1671 | 107 | 65.0 | 16.6 |
| 1663 | 100 | 66.7 | 3.3 |
| 1672 | 108 | 70.0 | 22.2 |
| 1675 | 111 | 70.0 | 18.0 |
| 1654 | 93 | 78.3 | 8.9 |
| 1668 | 105 | 78.8 | 14.2 |
| 1661 | 99 | 91.7 | 13.1 |
| 1655 | 94 | 93.3 | 12.5 |
| 1642 | 86 | 105.0 | 51.5 |
| 1647 | 89 | 130.6 | 4.1 |
| 1658 | 97 | 136.7 | 22.4 |
| 1656 | 95 | 175.0 | 6.7 |
| 1659 | 98 | 193.8 | 21.0 |
| 1649 | 90 | 200.0 | 16.3 |
| 1652 | 91 | 200.0 | 4.2 |
| 1653 | 92 | 200.0 | 8.2 |
| 1657 | 96 | 225.0 | 16.0 |
| 1645 | 87 | 283.3 | 37.3 |

Table 11 provides (i) IC₅₀ values for a variety of IL-8 mimetics to show the concentration of a particular IL-8 mimetic that is necessary to provide 50% of the maximal inhibition of ¹²⁵I-IL-8 binding that can be obtained with a particular IL-8 mimetic; and (ii) the maximal inhibition of the percent of ¹²⁵I-IL-8 bound to CXCR2 receptors on differentiated HL-60 cells for both native IL-8 and IL-8 mimetics. The inhibition of ¹²⁵I-IL-8 binding by IL-8 mimetics is indicative of the relative ability of the analogs to bind to CXCR1/CXCR2 receptors.

Calcium Mobilization

The results of calcium mobilization assays can be used to show the agonistic activation of the IL-8 receptor by the native IL-8 and IL-8 mimetics. The HL-60 cells are cultured as described above, harvested and suspended in Tyrode's salt solution at a density of about 2×10⁶ cells/ml. The Tyrode's salt solution contains about 137 mM NaCl, 2.7 mM KCl, 1 mM MgCl₂, 1 mM CaCl₂, 0.2 mM NaH₂PO₄, 12 mM NaHCO₃, and 5.5 mM glucose.

The cells are labeled with 4 μM of Fluo-4/AM (Molecular Probes, Inc.) for 45 minutes at 37° C. to measure calcium mobilization from cells. The label is a dye that fluoresces when bound to calcium. The cells are labeled with the dye to obtain a measure of the amount of calcium released by the cells when the cells are treated with the IL-8 mimetic or native IL-8. An increase in fluorescence indicates an increase in calcium mobilization. The cells are washed three times with the Tyrode's salt solution after labeling and re-suspended at 5×10⁶ cells/ml.

The native IL-8 and IL-8 mimetics are injected to produce a final concentration of about 10 μg/ml to about 200 μg/ml in aliquots containing about 5×10⁵ cells. Changes in the level of cellular fluorescence are read in a Thermo Labsystems Fluorskan Acsent fluorescence plate reader (VWR Scientific Prod's). The controls include cells treated with either the native chemokine or the plain medium of Tyrode's Salt Solution. Data is expressed using 1.0 as the standard level of fluorescence in the plain medium. The reported values represent the mean of at least duplicate measurements from wells in one or more experiments.

Table 12 provides a summary of the average fold increase of calcium mobilization in differentiated HL-60 cells over the control wells for native IL-8 and IL-8 mimetics.

TABLE 12

| Compound | | |
|---|---|---|
| Cross-ref. | | |
| SEQ ID NO. to | SEQ ID NO | |
| U.S. App. No. | in Present | Average fold increase |
| 10/932,208 | Application | in calcium mobilization |
| (Native IL-8) | 8 | 2.5 |
| 1664 | 101 | 147.5 |
| 1666 | 103 | 143.8 |
| 1671 | 107 | 134.8 |
| 1663 | 100 | 112.9 |
| 1668 | 105 | 105.4 |
| 1665 | 102 | 103.6 |
| 1661 | 99 | 100.6 |
| 1656 | 95 | 86.8 |
| 1670 | 106 | 65.4 |
| 1647 | 89 | 57.3 |
| 1667 | 104 | 57.3 |
| 1655 | 94 | 41.8 |
| 1646 | 88 | 33.9 |
| 1649 | 90 | 28.2 |
| 1672 | 108 | 13.4 |
| 1675 | 111 | 12.2 |
| 1673 | 109 | 5.7 |
| 1674 | 110 | 1.4 |

The incubation of the HL-60 cells with the IL-8 mimetics enhanced the receptor-mediated calcium mobilization. Similarly, 10 μg/ml of native IL-8 was used as a positive control that induced a two to three fold increase in calcium mobilization.

Neutrophil Mobilization

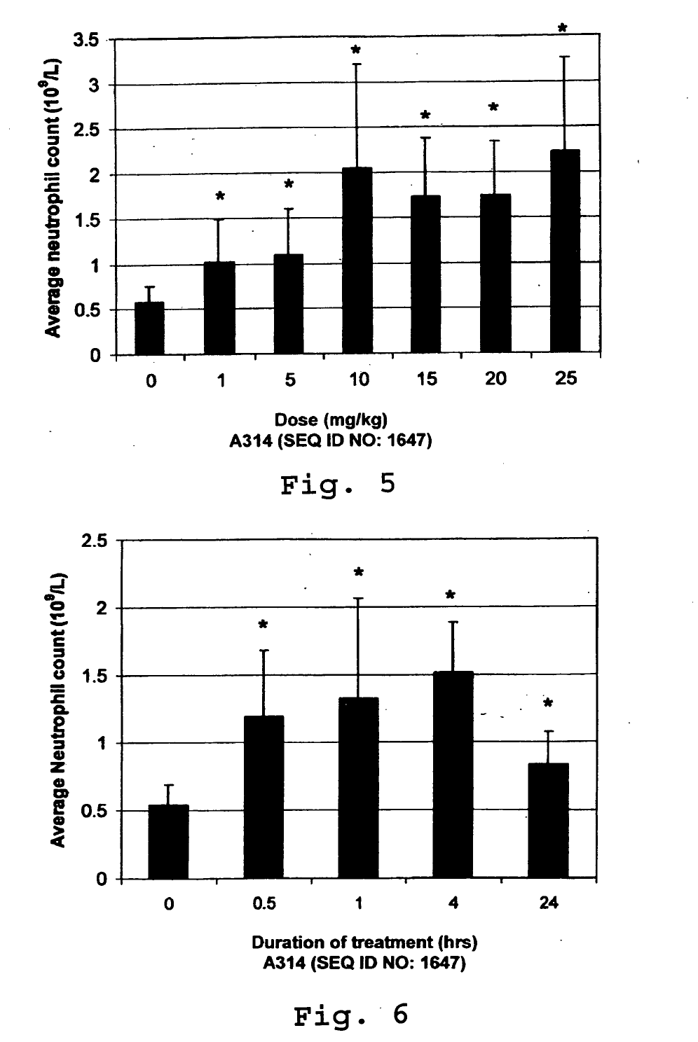

This example illustrates the efficacy of the IL-8 mimetic a161 (SEQ ID NO:1647) (“the test mimetic”) in increasing the number of circulating neutrophils and hematopoietic progenitor/stem cells in a mouse model. The results are shown in FIGS. 5-8. The experiments consisted of the following groups of female Balb/c mice (Charles River Lab's): (1) an untreated control group of 10 mice; and (2) test groups of 10 mice each.

The control and test groups of 20-23 g mice were randomly grouped in appropriately labeled cages and identified by cage markings and ear punch. The test groups were tested one time subcutaneously with the test analog at doses of 1, 5, 10, 15, 20, or 25 mg/kg in volumes of approximately 200 μl. The mice were anesthetized immediately before blood collection. Blood samples were obtained from the mice at 30 minutes, 1 hour, 4 hours, 6 hours, 24 hours and/or 48 hours after administration of the test mimetic. Blood was collected with an EDTA S-Monovette syringe (Sarstedt) and 25 G needle through a cardiac puncture. Blood was mixed gently by 5 inversions then expelled into a microcentrifuge tube. Differential and CBC analyses were performed on a Hemavet 850 FS (Drew Scientific). The end-point evaluations included complete blood counts with differentials and haematopoietic progenitor/stem cells as colony forming units (CFU).

The number of haematopoietic progenitor/stem cells was determined as follows. The volume of blood in each microfuge tube was determined and nine times the volume of ammonium chloride was added. Cells were incubated on ice for 10 minutes to lyse the red blood cells. The cells were washed twice and resuspended in 300 μL of Iscove's Modified Dulbecco's Medium (IMDM) containing 2% fetal bovine serum. The number of nucleated cells per mL of blood was counted, and all cells were plated in duplicate in standard methylcellulose to determine the number of CFUs including the colony-forming unit granulocyte-macrophage (CFU-GM), the burst-forming unit erythroid (BFU-E), and the colony-forming unit granulocyte erythrocyte macrophage megakaryocyte (CFU-GEMM). Plates were incubated for 7-14 days at 37° C. in a fully humidified 5% CO₂-air atmosphere, and colonies containing more than 50 cells were scored using an inverted microscope. The total CFU per mL of blood from the individual mice was determined.

The differentials were used to evaluate the mobilization of neutrophils and were compared to the untreated control group. A time and concentration dependent increase in neutrophils and haematopoietic progenitor/stem cells in the circulation is shown in FIGS. 5-8, indicating the rapid and potent activity of the test analog in vivo.

FIG. 5 shows the response of circulating neutrophil counts to the administration of varying doses of the test mimetic following one hour of treatment according to some embodiments. The test mimetic was administered by subcutaneous injection into female Balb/c mice in amounts of 1, 5, 10, 15, 20, or 25 mg/kg. At 1 hour post-injection, the mice were euthanized and blood was collected by cardiac puncture. Complete blood counts and differentials were determined using a Hemavet. The values represent the mean (+/−) 1 standard deviation of 10 animals per treatment group. Statistically significant elevations as determined using a p value of <0.05 are indicated in FIG. 5 by a “*”.

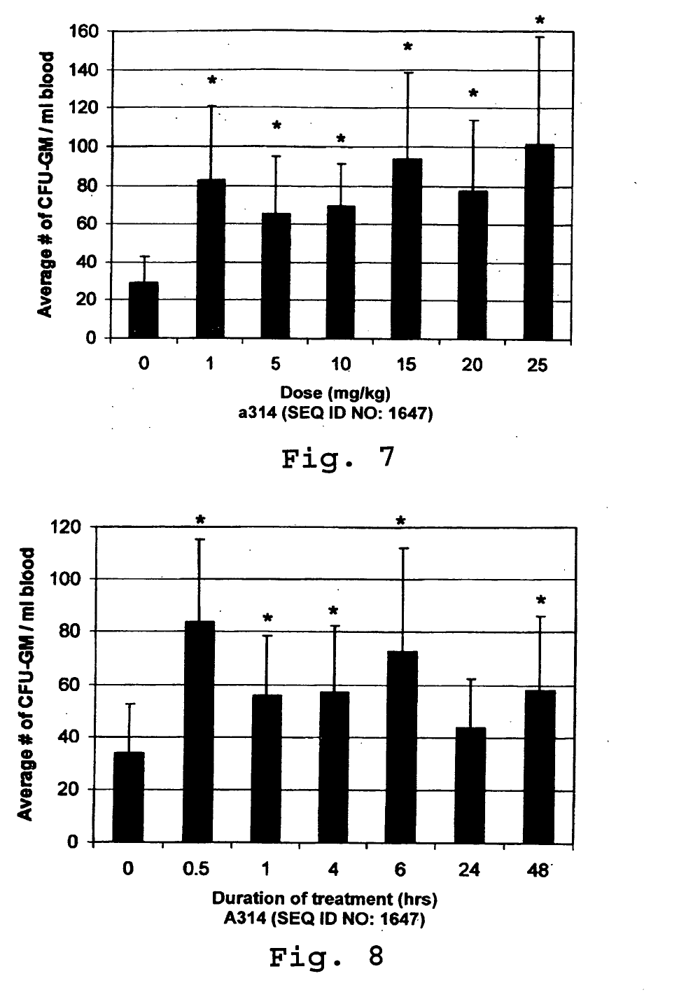

FIG. 6 describes the kinetics of the rise in circulating neutrophil counts in response to the administration of the test mimetic according to some embodiments. The test mimetic was administered by subcutaneous injection into female Balb/c mice at 25 mg/kg at time intervals of 30 minutes, 1 hour, 4 hours, and 24 hours. The mice were euthanized and blood was collected by cardiac puncture at each time interval. Complete blood counts and differentials were determined using a Hemavet®. The values represent the mean+/−one standard deviation for 10 animals per treatment group. Statistically significant elevations as determined using a p value of <0.05 are indicated in FIG. 6 by a “*”.

FIG. 7 shows the response of circulating haematopoietic progenitor/stem cells to the administration of varying doses of the test mimetic according to some embodiments. The test mimetic was administered by subcutaneous injection into female Balb/c mice in amounts of 1, 5, 10, 15, 20, and 25 mg/kg. At 1 hour post-injection, the mice were euthanized and blood was collected by cardiac puncture. The number of hematopoietic progenitor/stem cells (colony forming unit granulocyte-macrophage (CFU-GM), burst-forming unit erythroid (BFU-E) and colony forming unit granulocyte erythrocyte macrophage megakaryocyte (CFU-GEMM)) was determined by growing the cells in methylcellulose and counting the number of respective colonies. The values represent the mean+/−one standard deviation for 10 animals per treatment group. Statistically significant elevations as determined using a p value of <0.05 are indicated in FIG. 7 by a “*”.

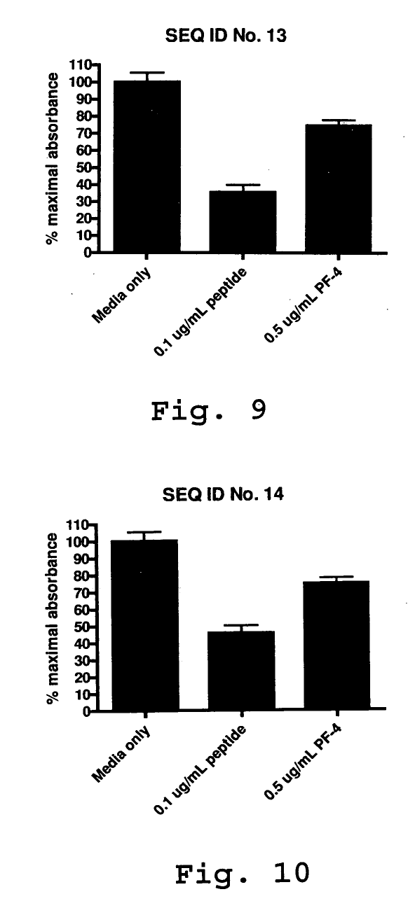

FIG. 8 describes the kinetics of the rise in haematopoietic progenitor/stem cells in response to the administration of the test mimetic according to some embodiments. The test mimetic was administered by subcutaneous injection into female Balb/c mice at 25 mg/kg. At 30 minutes, 1 hour, 4 hours, 6 hours, 24 hours or 48 hours post-injection, mice were euthanized and blood collected by cardiac puncture. The number of haematopoietic progenitor/stem cells as measured by colony forming unit granulocyte-macrophage (CFU-GM), burst-forming unit erythroid (BFU-E), and colony forming unit granulocyte erythrocyte macrophage megakaryocyte (CFU-GEMM) were determined by growing the cells in methylcellulose and counting the number of respective colonies. The values represent the mean (+/−) one standard deviation for 10 animals per treatment group. Statistically significant elevations as determined using a p value of <0.05 are indicated in FIG. 8 by a “*”.

Similar results have been observed in U.S. application Ser. No. 10/243,795 and PCT counterpart PCT/US2003/028745 using other amino acid linkers, such as the four amino acid liner, [Gly]₄ (SEQ ID NO:212).

### Example 6

PF-4s

The PF4 CXC chemokines are the subject of PCT Application No. PCT/CA2006/001848, which claims the benefit of U.S. Provisional Application No. 60/735,186, each of which is hereby incorporated herein by reference in its entirety. The cross-reference SEQ ID NOs from the source application are used in the explanation with regard to any associated figure or table, and the SEQ ID NOs used in the present application are provided to allow for location of the sequences in the formal sequence listing.

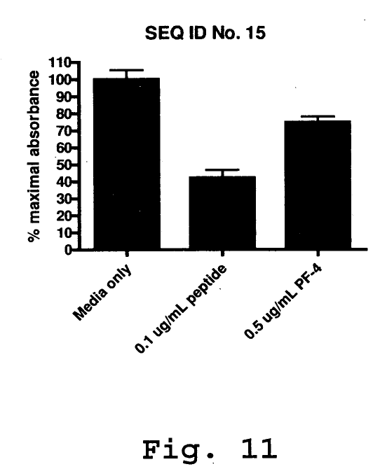

SEQ ID NOs.:13-15 have been prepared to use in the prevention, treatment, and ameliorization of diseases. FIGS. 9-11 illustrate the efficacy of the PF-4 analogs as agonists according to some embodiments. The efficacy is demonstrated through their ability to inhibit growth of human endothelial cells. The inhibition of endothelial cell growth is an important function of angiostatic compounds. The growth and survival of endothelial cells is tightly regulated by growth factors. The present examples illustrate the ability of PF-4 analogs to inhibit the growth stimulating effects of basic Fibroblast Growth Factor (bFGF) on HUVEC cells.

The three analogs tested inhibited the growth of HUVEC cells at a concentration of 0.1 μg/ml as determined using an MTT assay, which is calorimetric and measures cellular proliferation by determining the amount of yellow MTT (3-(4,5-dimethylthiazol-2-yl)-2,5-diphenyltetrazolium bromide) reduced to purple formazan spectrophotometrically. This reduction is indicative of mitochondrial reductase enzyme activity and is therefore related to the number of viable cells (Mosmann, T., Rapid Colorimetrc Assay for Cellular Growth and Survival: Application to Proliferation and Cytoxicity Assays. J. Immunol. Meth.: 55-63, 55 (1983)). The FIGs show that the measured reduction in maximal absorbance attributable to purple formazan is indicative of the degree of inhibition of cellular proliferation caused by the mimetics.

The efficacy of PF-4 analogs can be demonstrated by their ability to block the proliferation of human erythroleukemia cell lines (HEL) with megakaryocyte phenotype. The three analogs tested inhibited the growth of HEL cells at a concentration of 0.1 μg/ml, as determined using the MTT assay and as illustrated in FIGS. 8-10.

Those skilled in the art will recognize, or be able to ascertain using no more than routine experimentation, that there are many equivalents to the specific embodiments described herein that have been described and enabled to the extent that one of skill in the art can practice the invention well-beyond the scope of the specific embodiments taught herein. In addition, there are numerous lists and Markush groups taught and/or claimed herein. One of skill will appreciate that each such list and group contains various species and can be modified by the removal, or addition, of one or more of species, since every list and group taught and claimed herein may not be applicable to every embodiment feasible in the practice of the invention.

The described embodiments are considered in all respects as illustrative and not restrictive. It should also be understood that the invention is not limited to the particular embodiments described herein, but is capable of many equivalents, rearrangements, modifications, and substitutions without departing from the scope of the invention. All publications, patents, and patent applications mentioned in this application are herein incorporated by reference into the specification to the same extent as if each was specifically indicated to be herein incorporated by reference in its entirety.

LISTING OF SEQUENCES

| | | |
|---|---|---|
| Cross- | | |
| reference for | | |
| Sequence | | |
| Listing | | |
| No. of Parent | | |
| Application, | | CXC Chemokine Analog |
| U.S.. PCT, or | | (SEQ ID NO for the parent U.S. Application, |
| Provisional | SEQ ID NO: | where applicable) |

| | |
|---|---|
| 1 | CXCL1 GRO-α Source: Human |
| | Ala Ser Val Ala Thr Glu Leu Arg Cys |
| | Gln Cys Leu Gln Thr Leu Gln Gly Ile |
| | His Pro Lys Asn Ile Gln Ser Val Asn |
| | Val Lys Ser Pro Gly Pro His Cys Ala |
| | Gln Thr Glu Val Ile Ala Thr Leu Lys |
| | Asn Gly Arg Lys Ala Cys Leu Asn Pro |
| | Ala Ser Pro Ile Val Lys Lys Ile Ile |
| | Glu Lys Met Leu Asn Ser Asp Lys Ser |
| | Asn |
| 2 | CXCL2, GRO-β Source: Human |
| | Thr Glu Leu Arg Cys Gln Cys Leu Gln |
| | Thr Leu Gln Gly Ile His Leu Lys Asn |
| | Ile Gln Ser Val Lys Val Lys Ser Pro |
| | Gly Pro His Cys Ala Gln Thr Glu Val |
| | Ile Ala Thr Leu Lys Asn Gly Gln Lys |
| | Ala Cys Leu Asn Pro Ala Ser Pro Met |
| | Val Lys Lys Ile Ile Glu Lys Met Leu |
| | Lys Asn Gly Lys Ser Asn |
| 3 | CXCL3, GRO-γ Source: Human |
| | Thr Glu Leu Arg Cys Gln Cys Leu Gln |
| | Thr Leu Gln Gly Ile His Leu Lys Asn |
| | Ile Gln Ser Val Asn Val Arg Ser Pro |
| | Gly Pro His Cys Ala Gln Thr Glu Val |
| | Ile Ala Thr Leu Lys Asn Gly Lys Lys |
| | Ala Cys Leu Asn Pro Ala Ser Pro Met |
| | Val Gln Lys Ile Ile Glu Lys Ile Leu |
| | Asn Lys Gly Ser Thr Asn |
| 4 | CXCL4, PF-4 Source: Human: |
| | Glu Ala Glu Glu Asp Gly Asp Leu Gln |
| | Cys Leu Cys Val Lys Thr Thr Ser Gln |
| | Val Arg Pro Arg His Ile Thr Ser Leu |
| | Glu Val Ile Lys Ala Gly Pro His Cys |
| | Pro Thr Ala Gln Leu Ile Ala Thr Leu |
| | Lys Asn Gly Arg Lys Ile Cys Leu Asp |
| | Leu Gln Ala Pro Leu Tyr Lys Lys Ile |
| | Ile Lys Lys Leu Leu Glu Ser |
| 5 | CXCL5, ENA-78 Source: Human |
| | Leu Arg Glu Leu Arg Cys Val Cys Leu |
| | Gln Thr Thr Gln Gly Val His Pro Lys |
| | Met Ile Ser Asn Leu Gln Val Phe Ala |
| | Ile Gly Pro Gln Cys Ser Lys Val Glu |
| | Val Val Ala Ser Leu Lys Asn Gly Lys |
| | Glu Ile Cys Leu Asp Pro Glu Ala Pro |
| | Phe Leu Lys Lys Val Ile Gln Lys Ile |
| | Leu Asp Gly Gly Asn Lys Glu Asn |
| 6 | CXCL6, GCP-2 Source: Human |
| | Gly Pro Val Ser Ala Val Leu Thr Glu |
| | Leu Arg Cys Thr Cys Leu Arg Val Thr |
| | Leu Arg Val Asn Pro Lys Thr Ile Gly |
| | Lys Leu Gln Val Phe Pro Ala Gly Pro |
| | Gln Cys Ser Lys Val Glu Val Val Ala |
| | Ser Leu Lys Asn Gly Lys Gln Val Cys |
| | Leu Asp Pro Glu Ala Pro Phe Leu Lys |
| | Lys Val Ile Gln Lys Ile Leu Asp Ser |
| | Gly Asn Lys Lys Asn |
| 7 | CXCL7, NAP-2 Source: Human |
| | Ala Glu Leu Arg Cys Met Cys Ile Lys |
| | Thr Thr Ser Gly Ile His Pro Lys Asn |
| | Ile Gln Ser Leu Glu Val Ile Gly Lys |
| | Gly Thr His Cys Asn Gln Val Glu Val |
| | Ile Ala Thr Leu Lys Asp Gly Arq Lys |
| | Ile Cys Leu Asp Pro Asp Ala Pro Arg |
| | Ile Lys Lys Ile Val Gln Lys Lys Leu |
| | Ala Gly Asp Glu Ser Ala Asp |
| 8 | CXCL8, IL-8 Source: Human |
| | Ser Ala Lys Glu Leu Arg Cys Gln Cys |
| | Ile Lys Thr Tyr Ser Lys Pro Phe His |
| | Pro Lys Phe Ile Lys Glu Leu Arg Val |
| | Ile Glu Ser Gly Pro His Cys Ala Asn |
| | Thr Glu Ile Ile Val Lys Leu Ser Asp |
| | Gly Arg Glu Leu Cys Leu Asp Pro Lys |
| | Glu Asn Trp Val Gln Arg Val Val Glu |
| | Lys Phe Leu Lys Arg Ala Glu Asn Ser |
| 9 | CXCL9, MIG Source: Human |
| | Thr Pro Val Val Arg Lys Gly Arg Cys |
| | Ser Cys Ile Ser Thr Asn Gln Gly Thr |
| | Ile His Leu Gln Ser Leu Lys Asp Leu |
| | Lys Gln Phe Ala Pro Ser Pro Ser Cys |
| | Glu Lys Ile Glu Ile Ile Ala Thr Leu |
| | Lys Asn Gly Val Gln Thr Cys Leu Asn |
| | Pro Asp Ser Ala Asp Val Lys Glu Leu |
| | Ile Lys Lys Trp Glu Lys Gln Val Ser |
| | Gln Lys Lys Lys Gln Lys Asn Gly Lys |
| | Lys His Gln Lys Lys Lys Val Leu Lys |
| | Val Arg Lys Ser Gln Arg Ser Arg Gln |
| | Lys Lys Thr Thr |
| 10 | CXCL10, IP-10 Source: Human |
| | Val Pro Leu Ser Arg Thr Val Arg Cys |
| | Thr Cys Ile Ser Ile Ser Asn Gln Pro |
| | Val Asn Pro Pro Arg Ser Leu Glu Lys |
| | Leu Glu Ile Ile Pro Ala Ser Gln Phe |
| | Cys Pro Arg Val Glu Ile Ile Ala Thr |
| | Met Lys Lys Lys Gly Glu Lys Arg Cys |
| | Leu Asn Pro Glu Ser Lys Ala Ile Lys |
| | Asn Leu Leu Lys Ala Val Ser Lys Glu |
| | Met Ser Lys Arg Ser Pro |
| 11 | CXCL11, I-TAC Source: Human |
| | Phe Pro Met Phe Lys Arg Gly Arg Cys |
| | Leu Cys Ile Gly Pro Gly Val Lys Ala |
| | Val Lys Val Ala Asp Ile Glu Lys Ala |
| | Ser Ile Met Tyr Pro Ser Asn Asn Cys |
| | Asp Lys Ile Glu Val Ile Ile Thr Leu |
| | Lys Glu Asn Lys Gly Gln Arg Cys Leu |
| | Asn Pro Lys Ser Lys Gln Ala Arg Leu |
| | Ile Ile Lys Lys Val Glu Arg Lys Asn |
| | Phe |
| 12 | CXCL12, SDF-1 (Human): |
| | Lys Pro Val Ser Leu Ser Tyr Arg Cys |
| | Pro Cys Arg Phe Phe Glu Ser His Val |
| | Ala Arg Ala Asn Val Lys His Leu Lys |
| | Ile Leu Asn Thr Pro Asn Cys Ala Leu |
| | Gln Ile Val Ala Arg Leu Lys Asn Asn |
| | Asn Arg Gln Val Cys Ile Asp Pro Lys |
| | Leu Lys Trp Ile Gln Glu Tyr Leu Glu |
| | Lys Ala Leu Asn |
| 13 | CXCL13, BCA-I Source: Human |
| | Val Leu Glu Val Tyr Tyr Thr Ser Leu |
| | Arg Cys Arg Cys Val Gln Glu Ser Ser |
| | Val Phe Ile Pro Arg Arg Phe Ile Asp |
| | Arg Ile Gln Ile Leu Pro Arg Gly Asn |
| | Gly Cys Pro Arg Lys Glu Ile Ile Val |
| | Trp Lys Lys Asn Lys Ser Ile Val Cys |
| | Val Asp Pro Gln Ala Glu Trp Ile Gln |
| | Arg Met Met Glu Val Leu Arg Lys Arg |
| | Ser Ser Ser Thr Leu Pro Val Pro Val |
| | Phe Lys Arg Lys Ile Pro |
| 14 | CXCL14, BRAK Source: Human |
| | Ser Lys Cys Lys Cys Ser Arg Lys Gly |
| | Pro Lys Ile Arg Tyr Ser Asp Val Lys |
| | Lys Leu Glu Met Lys Pro Lys Tyr Pro |
| | His Cys Glu Glu Lys Met Val Ile Ile |
| | Thr Thr Lys Ser Val Ser Arg Tyr Arg |
| | Gly Gln Glu His Cys Leu His Pro Lys |
| | Leu Gln Ser Thr Lys Arg Phe Ile Lys |
| | Trp Tyr Asn Ala Trp Asn Glu Lys Arg |
| | Arg Val Tyr Glu Glu |
| 15 | CXCL15, Lungkine Source: Mouse |
| | Gln Glu Leu Arg Cys Leu Cys Ile Gln |
| | Glu His Ser Glu Phe Ile Pro Leu Lys |
| | Leu Ile Lys Asn Ile Met Val Ile Phe |
| | Glu Thr Ile Tyr Cys Asn Arg Lys Glu |
| | Val Ile Ala Val Pro Lys Asn Gly Ser |
| | Met Ile Cys Leu Asp Pro Asp Ala Pro |
| | Trp Val Lys Ala Thr Val Gly Pro Ile |
| | Thr Asn Arg Phe Leu Pro Glu Asp Leu |
| | Lys Gln Lys Glu Phe Pro Pro Ala Met |
| | Lys Leu Leu Tyr Ser Val Glu His Glu |
| | Lys Pro Leu Tyr Leu Ser Phe Gly Arg |
| | Pro Glu Asn Lys Arg Ile Phe Pro Phe |
| | Pro Ile Arg Glu Thr Ser Arg His Phe |
| | Ala Asp Leu Ala His Asn Ser Asp Arg |
| | Asn Phe Leu Arg Asp Ser Ser Glu Val |
| | Ser Leu Thr Gly Ser Asp Ala |
| 16 | CXCL16, SRPSOX Source: Human |
| | Gly Ser Val Thr Gly Ser Cys Tyr Cys |
| | Gly Lys Arg Ile Ser Ser Asp Ser Pro |
| | Pro Ser Val Gln Phe Met Asn Arg Leu |
| | Arg Lys His Leu Arg Ala Tyr His Arg |
| | Cys Leu Tyr Tyr Thr Arg Phe Gln Leu |
| | Leu Ser Trp Ser Val Cys Gly Gly Asn |
| | Lys Asp Pro Trp Val Gln Glu Leu Met |
| | Ser Cys Leu Asp Leu Lys Glu Cys Gly |
| | His Ala Tyr Ser |
| 17 | CXCL17, DMC Source: Human |
| | Met Lys Val Leu Ile Ser Ser Leu Leu |
| | Leu Leu Leu Pro Leu Met Leu Met Ser |
| | Met Val Ser Ser Ser Leu Asn Pro Gly |
| | Val Ala Arg Gly His Arg Asp Arg Gly |
| | Gln Ala Ser Arg Arg Trp Leu Gln Glu |
| | Gly Gly Gln Glu Cys Glu Cys Lys Asp |
| | Trp Phe Leu Arg Ala Pro Arg Arg Lys |
| | Phe Met Thr Val Ser Gly Leu Pro Lys |
| | Lys Gln Cys Pro Cys Asp His Phe Lys |
| | Gly Asn Val Lys Lys Thr Arg His Gln |
| | Arg His His Arg Lys Pro Asn Lys His |
| | Ser Arg Ala Cys Gln Gln Phe Leu Lys |
| | Gln Cys Gln Leu Arg Ser Phe Ala Leu |
| | Pro Leu |

CXCL1 (GRO-α) Analogs Source: Artificial Human

| | |
|---|---|
| 18 | R-X₀₁ X₀₂ X₀₃ X₀₄ X₀₅ X₀₆ X₀₇ X₀₈ X₀₉ X₁₀ X₁₁ |
| | X₁₂ X₁₃ X₁₄ X₁₅ X₁₆ [linker] Y₀₁ Y₀₂ Y₀₃ Y₀₄ |
| | Y₀₅ Y₀₆ Y₀₇ Y₀₈ Y₀₉ Y₁₀ Y₁₁ Y₁₂ Y₁₃ Y₁₄ |
| 19 | R-X₀₄ X₀₅ X₀₆ X₀₇ X₀₈ X₀₉ X₁₀ X₁₁ X₁₂ X₁₃ X₁₄ |
| | X₁₅ X₁₆ [linker] Y₀₁ Y₀₂ Y₀₃ Y₀₄ Y₀₅ Y₀₆ Y₀₇ |
| | Y₀₈ Y₀₉ Y₁₀ Y₁₁ Y₁₂ Y₁₃ Y₁₄ |
| 20 | R-X₀₅ X₀₆ X₀₇ X₀₈ X₀₉ X₁₀ X₁₁ X₁₂ X₁₃ X₁₄ X₁₅ |
| | X₁₆ [linker] Y₀₁ Y₀₂ Y₀₃ Y₀₄ Y₀₅ Y₀₆ Y₀₇ Y₀₈ |
| | Y₀₉ Y₁₀ Y₁₁ Y₁₂ Y₁₃ Y₁₄ |
| 21 | R-X₀₆ X₀₇ X₀₈ X₀₉ X₁₀ X₁₁ X₁₂ X₁₃ X₁₄ X₁₅ X₁₆ |
| | [linker] Y₀₁ Y₀₂ Y₀₃ Y₀₄ Y₀₅ Y₀₆ Y₀₇ Y₀₈ Y₀₉ |
| | Y₁₀ Y₁₁ 12 Y₁₃Y₁₄ |
| 22 | Ala Ser Val Ala Thr Glu Leu Arg Cys |
| | Gln Cys Leu Gln Thr Leu Gln -[linker]- |
| | Ile Val Lys Lys Ile Ile Glu Lys Met |
| | Leu Asn Ser Asp Lys |
| 23 | Ala Ser Val Ala Thr Glu Leu Arg Cys |
| | Gln Cys Leu Gln Thr Leu Gln -[linker]- |
| | Val Lys Lys Ile Ile Glu Lys Met Leu |
| | Asn Ser Asp Lys Ser |
| 24 | Ala Ser Val Ala Thr Glu Leu Arg Cys |
| | Gln Cys Leu Gln Thr Leu Gln -[linker]- |
| | Lys Lys Ile Ile Glu Lys Met Leu Asn |
| | Ser Asp Lys Ser Asn |
| 25 | Ala Thr Glu Leu Arg Cys Gln Cys Leu |
| | Gln Thr Leu Gln Gly Ile His -[linker]- |
| | Ile Val Lys Lys Ile Ile Glu Lys Met |
| | Leu Asn Ser Asp Lys |
| 26 | Thr Glu Leu Arg Cys Gln Cys Leu Gln |
| | Thr Leu Gln Gly Ile His Pro -[linker]- |
| | Ile Val Lys Lys Ile Ile Glu Lys Met |
| | Leu Asn Ser Asp Lys |
| 27 | Glu Leu Arg Cys Gln Cys Leu Gln Thr |
| | Leu Gln Gly Ile His Pro Lys -[linker]- |
| | Ile Val Lys Lys Ile Ile Glu Lys Met |
| | Leu Asn Ser Asp Lys |
| 28 | Glu Leu Arg Cys Gln Cys Leu Gln Thr |
| | Leu Gln Gly Ile His Pro Lys -[linker]- |
| | Ile Val Lys Lys Ile Ile Glu Lys Met |
| | Leu Asn Ser Asp Lys |

CXCL2 (GRO-β) Analogs Source: Artificial Human

| | | |
|---|---|---|
| N/A | 29 | R-X₀₁ X₀₂ X₀₃ X₀₄ X₀₅ X₀₆ X₀₇ X₀₈ X₀₉ X₁₀ X₁₁ |
| | | X₁₂ X₁₃ X₁₄ X₁₅ X₁₆ [linker] Y₀₁ Y₀₂ Y₀₃ Y₀₄ |
| | | Y₀₅ Y₀₆ Y₀₇ Y₀₈ Y₀₉ Y₁₀ Y₁₁ Y₁₂ Y₁₃ Y₁₄ |
| | 30 | Thr Glu Leu Arg Cys Gln Cys Leu Gln |
| | | Thr Leu Gln Gly Ile His Leu -[linker]- |
| | | Met Val Lys Lys Ile Ile Glu Lys Met |
| | | Leu Lys Asn Gly Lys |
| | 31 | Thr Glu Leu Arg Cys Gln Cys Leu Gln |
| | | Thr Leu Gln Gly Ile His Leu -[linker]- |
| | | Val Lys Lys Ile Ile Glu Lys Met Leu |
| | | Lys Asn Gly Lys Ser |
| | 32 | Thr Glu Leu Arg Cys Gln Cys Leu Gln |
| | | Thr Leu Gln Gly Ile His Leu -[linker]- |
| | | Lys Lys Ile Ile Glu Lys Met Leu Lys |
| | | Asn Gly Lys Ser Asn |
| N/A | 33 | Thr Glu Leu Arg Cys Gln Cys Leu Gln |
| | | Thr Leu Gln Gly Ile His Leu -[linker]- |
| | | Lys Lys Ile Ile Glu Lys Met Leu Lys |
| | | Asn Gly Lys Ser Asn |
| | 214 | Thr Glu Leu Arg Cys Gln Cys Leu Gln |
| | | Thr Leu Gln Gly Ile His Leu -[linker]- |
| | | Met Val Gln Lys Ile Ile Glu Lys Ile |
| | | Leu Asn Lys Gly Ser |
| | 34 | Thr Glu Leu Arg Cys Gln Cys Leu Gln |
| | | Thr Leu Gln Gly Ile His Leu -[linker]- |
| | | Val Gln Lys Ile Ile Glu Lys Ile Leu |
| | | Asn Lys Gly Ser Thr |

CXCL3 (GRO-γ) Analogs Source: Artificial Human

| | |
|---|---|
| 35 | R-X₀₁ X₀₂ X₀₃ X₀₄ X₀₅ X₀₆ X₀₇ X₀₈ X₀₉ X₁₀ X₁₁ |
| | X₁₂ X₁₃ X₁₄ X₁₅ X₁₆ [linker] Y₀₁ Y₀₂ Y₀₃ Y₀₄ |
| | Y₀₅ Y₀₆ Y₀₇ Y₀₈ Y₀₉ Y₁₀ Y₁₁ Y₁₂ Y₁₃ Y₁₄ |
| 36 | Thr Glu Leu Arg Cys Gln Cys Leu Gln |
| | Thr Leu Gln Gly Ile His Leu -[linker]- |
| | Gln Lys Ile Ile Glu Lys Ile Leu Asn |
| | Lys Gly Ser Thr Asn |
| 37 | Thr Glu Leu Arg Cys Gln Cys Leu Gln |
| | Thr Leu Gln Gly Ile His Leu [linker]- |
| | Gln Lys Ile Ile Glu Lys Ile Leu Asn |
| | Lys Gly Ser Thr Asn |

CXCL4 (PF-4) Analogs Source: Artificial Human

| | | |
|---|---|---|
| | 38 | R-X₀₁ X₀₂ X₀₃ X₀₄ X₀₅ X₀₆ X₀₇ X₀₈ X₀₉ X₁₀ X₁₁ |
| | | X₁₂ X₁₃ X₁₄ X₁₅ X₁₆ X₁₇ [linker] Y₀₁ Y₀₂ Y₀₃ |
| | | Y₀₄ Y₀₅ Y₀₆ Y₀₇ Y₀₈ Y₀₉ Y₁₀ Y₁₁ Y₁₂ Y₁₃ Y₁₄ |
| PCT/CA2006/ | 39 | Ac Ala Gln Gln Asn Gly Asp Leu Gln Cys |
| 001848 | | Leu Cys Val Lys [11-aminoundecanoic |
| 60/735,186 | | acid] Ala Pro Leu Tyr Lys Lys Ile Ile |
| | | Lys Lys Leu Leu Glu Ser |
| | | (SEQ ID NO:13) |
| | 40 | Ac Ala Gln Gln Asn Gly Asn Leu Gln Cys |
| | | Leu Cys Val [11-aminoundecanoic acid] |
| | | Ala Pro Leu Tyr Lys Lys Ile Ile Lys |
| | | Lys Leu Leu Glu Ser |
| | | (SEQ ID NO:14) |
| | 41 | Ala Glu Glu Asp Gly Asp Leu Gln Cys |
| | | Leu Cys Val Lys [11-aminoundecanoic |
| | | acid] Ala Pro Leu Tyr Lys Lys Ile Ile |
| | | Lys Lys Leu Leu Glu Ser |
| | | (SEQ ID NO:15) |
| | 42 | Ac Ala Glu Glu Asp Gly Asp Leu Gln Cys |
| | | Leu Cys Val Lys Thr Thr [11- |
| | | aminoundecanoic acid] Ala Pro Leu Tyr |
| | | Lys Lys Ile Ile Lys Lys Leu Leu Glu |
| | | Ser |
| | | (SEQ ID NO:17) |
| | 43 | Ac Ala Glu Glu Asp Gly Asp Leu Gln Cys |
| | | Leu Cys Val Lys [11-aminoundecanoic |
| | | acid] Ala Pro Leu Tyr Lys Lys Ile Ile |
| | | Lys Lys Leu Leu Glu Ser |
| | | (SEQ ID NO:18) |
| | | See also PCT Appl. No. |
| | | PCT/CA200E/001848, which is hereby |
| | | incorporated herein by reference in |
| | | its entirety, for additional |
| | | sequences. |

CXCL5 (ENA-78) Analogs Source: Artificial Human

| | | |
|---|---|---|
| | 44 | R-X₀₁ X₀₂ X₀₃ X₀₄ X₀₅ X₀₆ X₀₇ X₀₈ K09 X₁₀ X₁₁ |
| | | X₁₂ X₁₃ X₁₄ X₁₅ X₁₆ [linker] Y₀₁ Y₀₂ Y₀₃ Y₀₄ |
| | | Y₀₅ Y₀₆ Y₀₇ Y₀₈ Y₀₉ Y₁₀ Y₁₁ Y₁₂ Y₁₃ Y₁₄ |
| | 45 | R-X₀₂ X₀₃ X₀₄ X₀₅ X₀₆ X₀₇ X₀₈ X₀₉ X₁₀ X₁₁ X₁₂ |
| | | X₁₃ X₁₄ X₁₅ X₁₆ [linker] Y₀₁ Y₀₂ Y₀₃ Y₀₄ Y₀₅ |
| | | Y₀₆ Y₀₇ Y₀₈ Y₀₉ Y₁₀ Y₁₁ Y₁₂ Y₁₃ Y₁₄ |
| N/A | 46 | Leu Arg Glu Leu Arg Cys Val Cys Leu |
| | | Gln Thr Thr Gln Gly Val His -[linker]- |
| | | Phe Leu Lys Lys Val Ile Gln Lys Ile |
| | | Leu Asp Gly Gly Asn |
| | 47 | Arg Glu Leu Arg Cys Val Cys Leu Gln |
| | | Thr Thr Gln Gly Val His Pro -[linker]- |
| | | Phe Leu Lys Lys Val Ile Gln Lys Ile |
| | | Leu Asp Gly Gly Asn |
| | 48 | Leu Arg Glu Leu Arg Cys Val Cys Leu |
| | | Gln Thr Thr Gln Gly Val His -[linker]- |
| | | Leu Lys Lys Val Ile Gln Lys Ile Leu |
| | | Asp Gly Gly Asn Lys |
| | 49 | Leu Arg Glu Leu Arg Cys Val Cys Leu |
| | | Gln Thr Thr Gln Gly Val His -[linker]- |
| | | Lys Lys Val Ile Gln Lys Ile Leu Asp |
| | | Gly Gly Asn Lys Glu |
| | 50 | Leu Arg Glu Leu Arg Cys Val Cys Leu |
| | | Gln Thr Thr Gln Gly Val His -[linker]- |
| | | Lys Val Ile Gln Lys Ile Leu Asp Gly |
| | | Gly Asn Lys Glu Asn |
| | 51 | Arg Glu Leu Arg Cys Val Cys Leu Gln |
| | | Thr Thr Gln Gly Val His -[linker]-Leu |
| | | Lys Lys Val Ile Gln Lys Ile Leu Asp |
| | | Gly Gly Asn Lys |
| | 52 | Arg Glu Leu Arg Cys Val Cys Leu Gln |
| | | Thr Thr Gln Gly Val His -[linker]-Lys |
| | | Lys Val Ile Gln Lys Ile Leu Asp Gly |
| | | Gly Asn Lys Glu |
| | 53 | Arg Glu Leu Arg Cys Val Cys Leu Gln |
| | | Thr Thr Gln Gly Val His -[linker]-Lys |
| | | Val Ile Gln Lys Ile Leu Asp Gly Gly |
| | | Asn Lys Glu Asn |
| | 54 | Arg Glu Leu Arg Cys Val Cys Leu Gln |
| | | Thr Thr Gln Gly Val His -[linker]-Lys |
| | | Val Ile Gln Lys Ile Leu Asp Gly Gly |
| | | Asn Lys Glu Asn |
| | 55 | Arg Glu Leu Arg Cys Val Cys Leu Gln |
| | | Thr Thr Gln Gly Val His -[linker]-Lys |
| | | Val Ile Gln Lys Ile Leu Asp Gly Gly |
| | | Asn Lys Glu Asn |
| | 56 | Gly Pro Val Ser Ala Val Leu Thr Glu |
| | | Leu Arg Cys Thr Cys Leu Arg -[linker]- |
| | | Phe Leu Lys Lys Val Ile Gln Lys Ile |
| | | Leu Asp Ser Gly Asn |
| | 57 | Val Ser Ala Val Leu Thr Glu Leu Arg |
| | | Cys Thr Cys Leu Arg Val Thr -[linker]- |
| | | Phe Leu Lys Lys Val Ile Gln Lys Ile |
| | | Leu Asp Ser Gly Asn |
| | 58 | Val Leu Thr Glu Leu Arg Cys Thr Cys |
| | | Leu Arg Val Thr Leu Arg Val -[linker]- |
| | | Phe Leu Lys Lys Val Ile Gln Lys Ile |
| | | Leu Asp Ser Gly Asn |
| | 59 | Glu Leu Arg Cys Thr Cys Leu Arg Val |
| | | Thr Leu Arg Val Asn Pro Lys -[linker]- |
| | | Phe Leu Lys Lys Val Ile Gln Lys Ile |
| | | Leu Asp Ser Gly Asn |
| | 60 | Gly Pro Val Ser Ala Val Leu Thr Glu |
| | | Leu Arg Cys Thr Cys Leu Arg -[linker]- |
| | | Leu Lys Lys Val Ile Gln Lys Ile Leu |
| | | Asp Ser Gly Asn Lys |
| | 61 | Val Ser Ala Val Leu Thr Glu Leu Arg |
| | | Cys Thr Cys Leu Arg Val Thr -[linker]- |
| | | Leu Lys Lys Val Ile Gln Lys Ile Leu |
| | | Asp Ser Gly Asn Lys |

CXCL6 (GCP-2) Analogs Source: Artificial Human

| | | |
|---|---|---|
| | 62 | R-X₀₁ X₀₂ X₀₃ X₀₄ X₀₅ X₀₆ X₀₇ X₀₈ X₀₉ X₁₀ X₁₁ |
| | | X₁₂ X₁₃ X₁₄ X₁₅ X₁₆ [linker] Y₀₁ Y₀₂ Y₀₃ Y₀₄ |
| | | Y₀₅ Y₀₆ Y₀₇ Y₀₈ Y₀₉ Y₁₀ Y₁₁ Y₁₂ Y₁₃ Y₁₄ |
| | 63 | R-X₀₆ X₀₇ X₀₈ X₀₉ X₁₀ X₁₁ X₁₂ X₁₃ X₁₄ X₁₅ X₁₆ |
| | | [linker] Y₀₁ Y₀₂ Y₀₃ Y₀₄ Y₀₅ Y₀₆ Y₀₇ Y₀₈ Y₀₉ |
| | | Y₁₀ Y₁₁ Y₁₂ Y₁₃ Y₁₄ |
| | 64 | R-X₀₃ X₀₄ X₀₅ X₀₆ X₀₇ X₀₈ X₀₉ X₁₀ X₁₁ X₁₂ X₁₃ |
| | | X₁₄ X₁₅ X₁₆ [linker] Y₀₁ Y₀₂ Y₀₃ Y₀₄ Y₀₅ Y₀₆ |
| | | Y₀₇ Y₀₈ Y₀₉ Y₁₀ Y₁₁ Y₁₂ Y₁₃ Y₁₄ |
| | 65 | R-X₀₉ X₁₀ X₁₁ X₁₂ X₁₃ X₁₄ X₁₅ X₁₆ [linker] |
| | | Y₀₁ Y₀₂ Y₀₃ Y₀₄ Y₀₅ Y₀₆ Y₀₇ Y₀₈ Y₀₉ Y₁₀ Y₁₁ |
| | | Y₁₂ Y₁₃ Y₁₄ |
| N/A | 66 | Val Leu Thr Glu Leu Arg Cys Thr Cys |
| | | Leu Arg Val Thr Leu Arg Val -[linker]- |
| | | Leu Lys Lys Val Ile Gln Lys Ile Leu |
| | | Asp Ser Gly Asn Lys |
| | 67 | Glu Leu Arg Cys Thr Cys Leu Arg Val |
| | | Thr Leu Arg Val Asn Pro Lys -[linker]- |
| | | Leu Lys Lys Val Ile Gln Lys Ile Leu |
| | | Asp Ser Gly Asn Lys |
| | 68 | Gly Pro Val Ser Ala Val Leu Thr Glu |
| | | Leu Arg Cys Thr Cys Leu Arg -[linker]- |
| | | Lys Lys Val Ile Gln Lys Ile Leu Asp |
| | | Ser Gly Asn Lys Asn |
| | 69 | Val Ser Ala Val Leu Thr Glu Leu Arg |
| | | Cys Thr Cys Leu Arg Val Thr -[linker]- |
| | | Lys Lys Val Ile Gln Lys Ile Leu Asp |
| | | Ser Gly Asn Lys Asn |
| | 70 | Val Leu Thr Glu Leu Arg Cys Thr Cys |
| | | Leu Arg Val Thr Leu Arg Val -[linker]- |
| | | Lys Lys Val Ile Gln Lys Ile Leu Asp |
| | | Ser Gly Asn Lys Asn |
| | 71 | Glu Leu Arg Cys Thr Cys Leu Arg Val |
| | | Thr Leu Arg Val Asn Pro Lys -[linker]- |
| | | Lys Lys Val Ile Gln Lys Ile Leu Asp |
| | | Ser Gly Asn Lys Asn |
| | 72 | Glu Leu Arg Cys Thr Cys Leu Arg Val |
| | | Thr Leu Arg Val Asn Pro Lys -[linker]- |
| | | Phe Leu Lys Lys Val Ile Gln Lys Ile |
| | | Leu Asp Ser Gly Asn |
| | 73 | Glu Leu Arg Cys Thr Cys Leu Arg Val |
| | | Thr Leu Arg Val Asn Pro Lys -[linker]- |
| | | Lys Lys Val Ile Gln Lys Ile Leu Asp |
| | | Ser Gly Asn Lys Asn |

CXCL7 (NAP-2) Analogs Source: Artificial Human

| | |
|---|---|
| 74 | R-X₀₁ X₀₂ X₀₃ X₀₄ X₀₅ X₆ X₀₇ X₀₈ X₀₉ X₁₀ X₁₁ |
| | X₁₂ X₁₃ X₁₄ X₁₅ X₁₆ [linker] Y₀₁ Y₀₂ Y₀₃ Y₀₄ |
| | Y₀₅ Y₀₆ Y₀₇ Y₀₈ Y₀₉ Y₁₀ Y₁₁ Y₁₂ Y₁₃ Y₁₄ |
| 75 | R-X₀₂ X₀₃ X₀₄ X₀₅ X₀₆ X₀₇ X₀₈ X₀₉ X₁₀ X₁₁ X₁₂ |
| | X₁₃ X₁₄ X₁₅ X₁₆ [linker] Y₀₁ Y₀₂ Y₀₃ Y₀₄ Y₀₅ |
| | Y₀₆ Y₀₇ Y₀₈ Y₀₉ Y₁₀ Y₁₁ Y₁₂ Y₁₃ Y₁₄ |
| 76 | Ala Glu Leu Arg Cys Met Cys Ile Lys |
| | Thr Thr Ser Gly Ile His Pro -[linker]- |
| | Arg Ile Lys Lys Ile Val Gln Lys Lys |
| | Leu Ala Gly Asp Glu |
| 77 | Glu Leu Arg Cys Met Cys Ile Lys Thr |
| | Thr Ser Gly Ile His Pro Lys -[linker]- |
| | Arg Ile Lys Lys Ile Val Gln Lys Lys |
| | Leu Ala Gly Asp Glu |
| 78 | Ala Glu Leu Arg Cys Met Cys Ile Lys |
| | Thr Thr Ser Gly Ile His Pro -[linker]- |
| | Pro Asp Pro Asp Ala Pro Arg Ile Lys |
| | Lys Ile Val Gln Lys Lys Leu |
| 79 | Ala Glu Leu Arg Cys Met Cys Ile Lys |
| | Thr Thr Ser Gly Ile His Pro -[linker]- |
| | Ile Lys Lys Ile Val Gln Lys Lys Leu |
| | Ala Gly Asp Glu Ser |
| 80 | Glu Leu Arg Cys Met Cys Ile Lys Thr |
| | Thr Ser Gly Ile His Pro Lys -[linker]- |
| | Lys Lys Ile Val Gln Lys Lys Leu Ala |
| | Gly Asp Glu Ser Ala |
| 81 | Ala Glu Leu Arg Cys Met Cys Ile Lys |
| | Thr Thr Ser Gly Ile His Pro -[linker]- |
| | Lys Ile Val Gln Lys Lys Leu Ala Gly |
| | Asp Glu Ser Ala Asp |
| 82 | Glu Leu Arg Cys Met Cys Ile Lys Thr |
| | Thr Ser Gly Ile His Pro Lys -[linker]- |
| | Lys Ile Val Gln Lys Lys Leu Ala Gly |
| | Asp Glu Ser Ala Asp |
| 83 | Glu Leu Arg Cys Met Cys Ile Lys Thr |
| | Thr Ser Gly Ile His Pro Lys -[linker]- |
| | Lys Ile Val Gln Lys Lys Leu Ala Gly |
| | Asp Glu Ser Ala Asp |

CXCL8 (IL-8) Analogs Source: Artificial Human

| | | |
|---|---|---|
| 10/243,795 | 84 | H₂N-Ser-Ala-Lys-Glu-Leu-Arg-Cys-Gln- |
| | | Cys-Ile-Lys-Thr-Tyr-Ser-Lys-[Gly-Gly- |
| | | Gly-Gly]-Asn-Trp-Val-Gln-Arg-Val-Val- |
| | | Glu-Lys-Phe-Leu-Lys-Arg-Ala-Glu-Asn- |
| | | (OH)NH₂ |
| | | (SEQ ID NO:1632) |
| | 85 | H₂N-Ser-Ala-Lys-Glu-Leu-Arg-Cys-Gln- |
| | | Cys-Ile-Lys-Thr-Tyr-[Gly-Gly-Gly-Gly]- |
| | | Asn-Trp-Val-Gln-Arg-Val-Val-Glu-Lys- |
| | | Phe-Leu-Lys-Arg-Ala-Glu-Asn-(OH)NH₂ |
| | | (SEQ ID NO:1633) |
| 10/932,208 | 86 | H-Asn-Trp-Val-Gln-Arg-Val-Val-Glu-Lys |
| | | Phe-Leu-Lys-Arg-Ala-Glu-Asn-NH₂ |
| | | (SEQ ID NO:1642) |
| | 87 | H-Ser-Ala-Lys-Glu-Leu-Arg-Cys-Gln-Cys- |
| | | Ile-Lys-Thr-Tyr-Ser-Lys-[Gly]₄-Asn- |
| | | Trp-Val-Gln-Arg-Val-Val-Glu-Lys-Phe- |
| | | Leu-Lys-Arg-Ala-Glu-Asn-NH₂ |
| | | (SEQ ID NO:1645) |
| | 88 | H-Ser-Ala-Lys-Glu-Leu-Arg-Cys-Gln-Cys- |
| | | Ile-Lys-Thr-Tyr-Ser-Lys- |
| | | [11-aminoundecanoic acid]-Asn-Trp-Val- |
| | | Gln-Arg-Val-Val-Glu-Lys-Phe-Leu-Lys- |
| | | Arg-Ala-Glu-Asn-NH₂ |
| | | (SEQ ID NO:1646) |
| | 89 | H-Ser-Ala-Lys-Glu-Leu-Arg-Ala-Gln-Phe- |
| | | Ile-Lys-Thr-Tyr-Ser-Lys- |
| | | [11-aminoundecanoic acid]-Asn-Trp-Val- |
| | | Gln-Arg-Val-Val-Glu-Lys-Phe-Leu-Lys- |
| | | Arg-Ala-Glu-Asn-NH₂ |
| | | (SEQ ID NO:1647) |
| | 90 | H-Ser-Ala-Lys-Glu-Leu-Arg-Ser-Gln-Ser- |
| | | Ile-Lys-Thr-Tyr-Ser-Lys- |
| | | [11-aminoundecanoic acid]-Asn-Trp-Val- |
| | | Gln-Arg-Val-Val-Glu-Lys-Phe-Leu-Lys- |
| | | Arg-Ala-Glu-Asn-NH₂ |
| | | (SEQ ID NO:1649) |
| | 91 | H-Ser-Ala-Lys-Glu-Leu-Arg-Ala-Gln-Tyr- |
| | | Ile-Lys-Thr-Tyr-Ser-Lys- |
| | | [11-aminoundecanoic acid]-Asn-Trp-Val- |
| | | Gln-Arg-Val-Val-Glu-Lys-Phe-Leu-Lys- |
| | | Arg-Ala-Glu-Asn-NH₂ |
| | | (SEQ ID NO:1652) |
| | 92 | Ac-Ser-Ala-Lys-Glu-Leu-Arg-Ala-Gln- |
| | | Tyr-Ile-Lys-Thr-Tyr-Ser-Lys- |
| | | [11-aminoundecanoic acid]-Asn-Trp-Val- |
| | | Gln-Arg-Val-Val-Glu-Lys-Phe-Leu-Lys- |
| | | Arg-Ala-Glu-Asn-NH₂ |
| | | (SEQ ID NO:1653) |
| | 93 | H-Ser-Ala-Lys-Glu-Leu-Arg-Tyr-Gln-Phe- |
| | | Ile-Lys-Thr-Tyr-Ser-Lys- |
| | | [11-aminoundecanoic acid]-Asn-Trp-Val- |
| | | Gln-Arg-Val-Val-Glu-Lys-Phe-Leu-Lys |
| | | Arg-Ala-Glu-Asn-NH₂ |
| | | (SEQ ID NO:1654) |
| | 94 | Ac-Ser-Ala-Lys-Glu-Leu-Arg-Tyr-Gln- |
| | | Phe-Ile-Lys-Thr-Tyr-Ser-Lys- |
| | | [11-aminoundecanoic acid]-Asn-Trp-Val- |
| | | Gln-Arg-Val-Val-Glu-Lys-Phe-Leu-Lys- |
| | | Arg-Ala-Glu-Asn-NH₂ |
| | | (SEQ ID NO:1655) |
| | 95 | H-Ser-Ala-Lys-Glu-Leu-Arg-Tyr-Gln-Ala- |
| | | Ile-Lys-Thr-Tyr-Ser-Lys- |
| | | [11-aminoundecanoic acid]-Asn-Trp-Val- |
| | | Gln-Arg-Val-Val-Glu-Lys-Phe-Leu-Lys- |
| | | Arg-Ala-Glu-Asn-NH₂ |
| | | (SEQ ID NO:1656) |
| | 96 | Ac-Ser-Ala-Lys-Glu-Leu-Arg-Tyr-Gln- |
| | | Ala-Ile-Lys-Thr-Tyr-Ser-Lys- |
| | | [11-aminoundecanoic acid]-Asn-Trp-Val- |
| | | Gln-Arg-Val-Val-Glu-Lys-Phe-Leu-Lys- |
| | | Arg-Ala-Glu-Asn-NH₂ |
| | | (SEQ ID NO:1657) |
| | 97 | H-Ser-Ala-Lys-Glu-Leu-Arg-Tyr-Gln-Tyr- |
| | | Ile-Lys-Thr-Tyr-Ser-Lys- |
| | | [11-aminoundecanoic acid]-Asn-Trp-Val- |
| | | Gln-Arg-Val-Val-Glu-Lys-Phe-Leu-Lys- |
| | | Arg-Ala-Glu-Asn-NH₂ |
| | | (SEQ ID NO:1658) |
| | 98 | Ac-Ser-Ala-Lys-Glu-Leu-Arg-Tyr-Gln- |
| | | Tyr-Ile-Lys-Thr-Tyr-Ser-Lys- |
| | | [11-aminoundecanoic acid]-Asn-Trp-Val- |
| | | Gln-Arg-Val-Val-Glu-Lys-Phe-Leu-Lys- |
| | | Arg-Ala-Glu-Asn-NH₂ |
| | | (SEQ ID NO:1659) |
| | 99 | Ac-Ser-Ala-Lys-Glu-Leu-Arg-Tyr-Gln- |
| | | Phe-Ile-Arg-Thr-Tyr-Ser-Lys - |
| | | [11-aminoundecanoic acid]-Asn-Trp-Val- |
| | | Gln-Arg-Val-Val-Glu-Lys-Phe-Leu-Lys |
| | | Arg-Ala-Glu-Asn-NH₂ |
| | | (SEQ ID NO:1661) |
| | 100 | H-Ser-Ala-Lys-Glu-Leu-Arg-Ala-Gln-Phe- |
| | | Ile-Lys-Thr-Tyr-Ser-Lys- |
| | | [11-aminoundecanoic acid]-Asn-Trp-Val- |
| | | Gln-Arg-Val-Val-Glu-Lys-Phe-Leu-Lys- |
| | | Arg-Ala-Glu-Asn-NH₂ |
| | | (SEQ ID NO:1663) |
| | 101 | Ac-Ser-Ala-Lys-Glu-Leu-Arg-Ala-Gln- |
| | | Phe-Ile-Lys-Thr-Tyr-Ser-Lys- |
| | | [11-aminoundecanoic acid]-Asn-Trp-Val- |
| | | Gln-Arg-Val-Val-Glu-Lys-Phe-Leu-Lys- |
| | | Arg-Ala-Glu-Asn-NH₂ |
| | | (SEQ ID NO:1664) |
| | 102 | Ac-Ser-Ala-Lys-Glu-Leu-Arg-Ala-Gln- |
| | | Tyr-Ile-Lys-Thr-Tyr-Ser-Lys - |
| | | [11-aminoundecanoic acid]-Asn-Trp-Val- |
| | | Gln-Arg-Val-Val-Glu-Lys-Phe-Leu-Lys- |
| | | Arg-Ala-Glu-Asn-NH₂ |
| | | (SEQ ID NO:1665) |
| | 103 | Ac-Ser-Ala-Lys-Glu-Leu-Arg-Tyr-Gln- |
| | | Phe-Ile-Lys-Thr-Tyr-Ser-Lys- |
| | | [11-aminoundecanoic acid]-Asn-Trp-VaIL- |
| | | Gln-Arg-Val-Val-Glu-Lys-Phe-Leu-Lys- |
| | | Arg-Ala-Glu-Asn-NH₂ |
| | | (SEQ ID NO:1666) |
| | 104 | Ac-Ser-Ala-Lys-Glu-Leu-Arg-Tyr-Gln- |
| | | Tyr-Ile-Lys-Thr-Tyr-Ser-Lys - |
| | | [11-aminoundecanoic acid]-Asn-Trp-Val- |
| | | Gln-Arg-Val-Val-Glu-Lys-Phe-Leu-Lys- |
| | | Arg-Ala-Glu-Asn-NH₂ |
| | | (SEQ ID NO:1667) |
| | 105 | Ac-Ser-Ala-Lys-Glu-Leu-Arg-His-Gln |
| | | Tyr-Ile-Lys-Thr-Tyr-Ser-Lys- |
| | | [11-aminoundecanoic acid]-Asn-Trp-Val |
| | | Gln-Arg-Val-Val-Glu-Lys-Phe-Leu-Lys |
| | | Arg-Ala-Glu-Asn-NH₂ |
| | | (SEQ ID NO:1668) |
| | 106 | Ac-Ser-Ala-Lys-Glu-Leu-Arg-Tyr-Gln- |
| | | Trp-Ile-Lys-Thr-Tyr-Ser-Lys- |
| | | [11-aminoundecanoic acid]-Asn-Trp-Val- |
| | | Gln-Arg-Val-Val-Glu-Lys-Phe-Leu-Lys- |
| | | Arg-Ala-Glu-Asn-NH₂ |
| | | (SEQ ID NO:1670) |
| | 107 | Ac-Ser-Ala-Lys-Glu-Leu-Arg-Ala-Gln- |
| | | Phe-Ile-Arg-Thr-Tyr-Ser-Lys-[11- |
| | | aminoundecanoic acid]-Asn-Trp-Val-Gln- |
| | | Arg-Val-Val-Glu-Lys-Phe-Leu-Lys-Arg- |
| | | Ala-Glu-Asn-NH₂ |
| | | (SEQ ID NO:1671) |
| | 108 | Ac-Ser-Ala-Lys-Glu-Leu-Arg-Tyr-Gln- |
| | | Trp-Ile-Arg-Thr-Tyr-Ser-Lys- |
| | | [11-aminoundecanoic acid]-Asn-Trp-Val- |
| | | Gln-Arg-Val-Val-Glu-Lys-Phe-Leu-Lys- |
| | | Arg-Ala-Glu-Asn-NH₂ |
| | | (SEQ ID NO:1672) |
| | 109 | Ac-Ser-Ala-Lys-Glu-Leu-Arg-Tyr-Gln- |
| | | Trp-Ile-Arg-Thr-Tyr-Ser-Arg- |
| | | [11-aminoundecanoic acid]-Asn-Trp-Val- |
| | | Gln-Arg-Val-Val-Glu-Lys-Phe-Leu-Lys- |
| | | Arg-Ala-Glu-Asn-NH₂ |
| | | (SEQ ID NO:1673) |
| | 110 | Ac-Ser-Ala-Lys-Glu-Leu-Arg-Trp-Gln- |
| | | Trp-Ile-Arg-Thr-Tyr-Ser-Arg- |
| | | [11-aminoundecanoic acid]-Asn-Trp-Val- |
| | | Gln-Arg-Val-Val-Glu-Lys-Phe-Leu-Lys- |
| | | Arg-Ala-Glu-Asn-NH₂ |
| | | (SEQ ID NO:1674) |
| | 111 | Ac-Ser-Ala-Lys-Glu-Leu-Arg-His-Gln- |
| | | Trp-Ile-Arg-Thr-Tyr-Ser-Arg- |
| | | [11-aminoundecanoic acid]-Asn-Trp-Val- |
| | | Gln-Arg-Val-Val-Glu-Lys-Phe-Leu-Lys |
| | | Arg-Ala-Glu-Asn-NH₂ |
| | | (SEQ ID NO:1675) |
| | | See also U.S. Pat. App. Nos. |
| | | 10/932,208 and 10/243,795, each of |
| | | which is incorporated herein by |
| | | reference in its entirety, for |
| | | additional sequences. |

CXCL9 (MIG) Analogs Source: Artificial Human

| | |
|---|---|
| 112 | R-X₀₁ X₀₂ X₀₃ X₀₄ X₀₅ X₀₆ X₀₇ X₀₈ X₀₉ X₁₀ X₁₁ |
| | X₁₂ X₁₃ X₁₄ X₁₅ X₁₆ [linker] Y₀₁ Y₀₂ Y₀₃ Y₀₄ |
| | Y₀₅ Y₀₆ Y₀₇ Y₀₈ Y₀₉ Y₁₀ Y₁₁ Y₁₂ Y₁₃ Y₁₄ |
| 113 | Thr Pro Val Val Arg Lys Gly Arg Cys |
| | Ser Cys Ile Ser Thr Asn Gln -[linker]- |
| | Asp Ser Ala Asp Val Lys Glu Leu Ile |
| | Lys Lys Trp Glu Lys |
| 114 | Thr Pro Val Val Arg Lys Gly Arg Cys |
| | Ser Cys Ile Ser Thr Asn Gln -[linker]- |
| | Ser Ala Asp Val Lys Glu Leu Ile Lys |
| | Lys Trp Glu Lys Gln |
| 115 | Thr Pro Val Val Arg Lys Gly Arg Cys |
| | Ser Cys Ile Ser Thr Asn Gln -[linker]- |
| | Gln Lys Lys Lys Val Leu Lys Val Arg |
| | Lys Ser Gln Arg Ser |
| 116 | Thr Pro Val Val Arg Lys Gly Arg Cys |
| | Ser Cys Ile Ser Thr Asn Gln -[linker]- |
| | Lys Lys Lys Val Leu Lys Val Arg Lys |
| | Ser Gln Arg Ser Arg |
| 117 | Thr Pro Val Val Arg Lys Gly Arg Cys |
| | Ser Cys Ile Ser Thr Asn Gln -[linker]- |
| | Lys Lys Val Leu Lys Val Arg Lys Ser |
| | Gln Arg Ser Arg Gln |
| 118 | Thr Pro Val Val Arg Lys Gly Arg Cys |
| | Ser Cys Ile Ser Thr Asn Gln -[linker]- |
| | Lys Val Leu Lys Val Arg Lys Ser Gln |
| | Arg Ser Arg Gln Lys |
| 119 | Thr Pro Val Val Arg Lys Gly Arg Cys |
| | Ser Cys Ile Ser Thr Asn Gln -[linker]- |
| | Val Leu Lys Val Arg Lys Ser Gln Arg |
| | Ser Arg Gln Lys Lys |
| 120 | Thr Pro Val Val Arg Lys Gly Arg Cys |
| | Ser Cys Ile Ser Thr Asn Gln -[linker]- |
| | Leu Lys Val Arg Lys Ser Gln Arg Ser |
| | Arg Gln Lys Lys Thr |
| 121 | Thr Pro Val Val Arg Lys Gly Arg Cys |
| | Ser Cys Ile Ser Thr Asn Gln -[linker]- |
| | Lys Val Arg Lys Ser Gln Arg Ser Arg |
| | Gln Lys Lys Thr Thr |
| 122 | Thr Pro Val Val Arg Lys Gly Arg Cys |
| | Ser Cys Ile Ser Thr Asn Gln -[linker]- |
| | Ser Ala Asp Val Lys Glu Leu Ile Lys |
| | Lys Trp Glu Lys Gln |

CXCL10 (IP-10) Analogs Source: Artificial Human

| | | |
|---|---|---|
| 11/590,210 | 123 | Ac-Val-Pro-Leu-Ser-Arg-Thr-Val-Arg- |
| | | Cys-Thr-Cys-Ile-Ser-Ile-Ser¹⁵-Asn-UDA- |
| | | Leu⁶⁶-Lys-Ala-Val-Ser-Lys-Glu-Met-Ser- |
| | | Lys-Arg-Ser-Pro |
| | | (SEQ ID NO:1641) |
| | 124 | Ac-Val-Pro-Leu-Ser-Arg-Thr-Val-Arg |
| | | Ala⁹-Thr-Phe¹¹-Ile-Ser-Ile-Ser¹⁵-Asn- |
| | | UDA-Leu⁶⁶-Lys-Ala-Val-Ser-Lys-Glu-Met- |
| | | Ser-Lys-Arg-Ser-Pro |
| | | (SEQ ID NO:1642) |
| | 125 | Val-Pro-Leu-Ser-Arg-Thr-Pro⁷-Arg-Cys- |
| | | Thr-Cys-Ile-Ser-Ile-Ser¹⁵-UDA-Glu⁵⁸- |
| | | Ser-Lys-Ala-Ile-Lys-Asn-Leu-Leu-Lys |
| | | Ala-Val-Ser-Lys |
| | | (SEQ ID NO:1643) |
| | 126 | Val-Pro-Leu-Ser-Arg-Thr-Val-Arg-Ser⁹- |
| | | Thr-Ser¹¹-Ile-Ser-Ile-Ser¹⁵-UDA-Glu⁵⁸- |
| | | Ser-Lys-Ala-Ile-Glu⁶³-Asn-Leu-Leu-Lys- |
| | | Ala-Val-Ser-Lys |
| | | (SEQ ID NO:1644) |
| | 127 | Val-Pro-Leu-Ser-Arg-Thr-Val-Arg-Ser⁹- |
| | | Thr-Ser¹¹-Ile-Ser-Ile-Ser¹⁵-UDA-Glu⁵⁸- |
| | | Ser-Lys-Ala-Ile-Lys-Asn-Leu-Leu-Glu⁶⁷- |
| | | Ala-Val-Ser-Lys |
| | | (SEQ ID NO:1645) |
| | | wherein, UDA is 11-amino undecanoic |
| | | acid. |
| | | See U.S. Pat. Application No. |
| | | 11/590,210, 11/494,232, and 10/243,795, |
| | | each of which is hereby incorporated |
| | | herein by reference in its entirety, for |
| | | additional sequences. |

CXCL11 (I-TAC) Analogs Source: Artificial Human

| | |
|---|---|
| 128 | R-X₀₁ X₀₂ X₀₃ X₀₄ X₀₅ X₀₆ X₀₇ X₀₈ X₀₉ X₁₀ X₁₁ |
| | X₁₂ X₁₃ X₁₄ X₁₅ X₁₆ [linker] Y₀₁ Y₀₂ Y₀₃ Y₀₄ |
| | Y₀₅ Y₀₆ Y₀₇ Y₀₈ Y₀₉ Y₁₀ Y₁₁ Y₁₂ Y₁₃ Y₁₄ |
| 129 | Phe Pro Met Phe Lys Arg Gly Arg Cys |
| | Leu Cys Ile Gly Pro Gly Val -[linker]- |
| | Lys Ser Lys Gln Ala Arg Leu Ile Ile |
| | Lys Lys Val Glu Arg |
| 130 | Phe Pro Met Phe Lys Arg Gly Arg Cys |
| | Leu Cys Ile Gly Pro Gly Val -[linker]- |
| | Ser Lys Gln Ala Arg Leu Ile Ile Lys |
| | Lys Val Glu Arg Lys |
| 131 | Phe Pro Met Phe Lys Arg Gly Arg Cys |
| | Leu Cys Ile Gly Pro Gly Val -[linker]- |
| | Lys Gln Ala Arg Leu Ile Ile Lys Lys |
| | Val Glu Arg Lys Asn |
| 132 | Phe Pro Met Phe Lys Arg Gly Arg Cys |
| | Leu Cys Ile Gly Pro Gly Val -[linker]- |
| | Gln Ala Arg Leu Ile Ile Lys Lys Val |
| | Glu Arg Lys Asn Phe |
| 133 | Phe Pro Met Phe Lys Arg Gly Arg Cys |
| | Leu Cys Ile Gly Pro Gly Val -[linker]- |
| | Ser Lys Gln Ala Arg Leu Ile Ile Lys |
| | Lys Val Glu Arg Lys |

CXCL12 (SDF-1) Analogs Source: Artificial Human

| | | |
|---|---|---|
| 11/393,769 | 134 | H₂N-Lys-Pro-Val-Ser-Leu-Ser-Tyr-Arg- |
| | | Cys-Pro-Cys-Arg-Phe-Phe- [Gly-Gly-Gly- |
| | | Gly]-Leu-Lys-Trp-Ile-Gln-Glu-Tyr-Leu- |
| | | Glu-Lys-Ala-Leu-Asn-NH₂ |
| | | (SEQ ID NO:809) |
| | 135 | H₂N-Lys-Pro-Val-Ser-Leu-Ser-Tyr-Arg- |
| | | Ala-Pro-Phe-Arg-Phe-Phe-[Gly-Gly-Gly- |
| | | Gly]-Leu-Lys-Trp-Ile-Gln-Glu-Tyr-Leu- |
| | | Glu-Lys-Ala-Leu-Asn-NH₂ |
| | | (SEQ ID NO:810) |
| | 136 | AcHN-Lys-Pro-Val-Ser-Leu-Ser-Tyr-Arg- |
| | | Cys-Pro-Cys-Arg-Phe-Phe-[Gly-Gly-Gly- |
| | | Gly]-Leu-Lys-Trp-Ile-Gln-Glu-Tyr-Leu- |
| | | Glu-Lys-Ala-Leu-Asn-NH₂ |
| | | (SEQ ID NO:811) |
| | 137 | AcHN-Lys-Pro-Val-Ser-Leu-Ser-Tyr-Arg- |
| | | Cys-Pro-Cys-Arg-Phe-Phe- |
| | | [11aminoundecanoic acid]-Leu-Lys-Trp- |
| | | Ile-Gln-Glu-Tyr-Leu-Glu-Lys-Ala-Leu- |
| | | Asn-NH₂ |
| | | (SEQ ID NO:812) |
| | 138 | H₂N-[desNH₂Lys]-Pro-Val-Ser-Leu-Ser- |
| | | Tyr-Arg-Cys-Pro-Cys-Arg-Phe-Phe-[Gly- |
| | | Gly-Gly-Gly]-Leu-Lys-Trp-Ile-Gln-Glu- |
| | | Tyr-Leu-Glu-Lys-Ala-Leu-Asn-NH₂ |
| | | (SEQ ID NO:813) |
| 11/388,542 | 139 | Lys-Pro-Val-Ser-Leu-Ser-Tyr-Arg-Ala- |
| | | Pro-Phe-Arg-Phe-Phe-Lys-Gly-Gly-Gly- |
| | | Leu-Lys-Trp-Ile-Gln-Glu-Tyr-Leu-Glu- |
| | | Lys-Pro-Val-Ser-Leu-Ser-Tyr-Arg-Ala- |
| | | Pro-Phe-Arg-Phe-Phe-Gly Gly-Lys -Gly- |
| | | Leu-Lys-Trp-Ile-Gln-Glu-Tyr-Leu-Glu- |
| | | Lys-Ala-Leu-Asn |
| | | (SEQ ID NO:5) |
| | | Lys-Ala-Leu-Asn |
| | | (SEQ ID NO:3) |
| | 140 | Lys-Pro-Val-Ser-Leu-Ser-Tyr-Arg-Ala- |
| | | Pro-Phe-Arg-Phe-Phe-Gly-Lys-Gly-Gly- |
| | | Leu-Lys-Trp-Ile-Gln-Glu-Tyr-Leu-Glu- |
| | | Lys-Ala-Leu-Asn |
| | | (SEQ ID NO:4) |
| | 141 | R_{N}-Lys-Pro-Val-Ser-Leu-Ser-Tyr-Arg |
| | | Ala-Pro-Phe-Arg-Phe-Phe-Gly Gly-Lys- |
| | | Gly-Leu-Lys-Trp-Ile-Gln-Glu-Tyr-Leu |
| | | Glu-Lys-Ala-Leu-Asn-R_{C} |
| | | (SEQ ID NO:5) |
| | 142 | Lys-Pro-Val-Ser-Leu-Ser-Tyr-Arg-Ala |
| | | Pro-Phe-Arg-Phe-Phe-Gly-Gly-Gly-Lys- |
| | | Leu-Lys-Trp-Ile-Gln-Glu-Tyr-Leu-Glu |
| | | Lys-Ala-Leu-Asn |
| | | (SEQ ID NO:6) |
| | 143 | Lys-Pro-Val-Ser-Leu-Ser-Tyr-Arg-Ala- |
| | | Pro-Phe-Arg-Phe-Phe-Lys-Lys-Gly-Gly- |
| | | Leu-Lys-Trp-Ile-Gln-Glu-Tyr-Leu-Glu- |
| | | Lys-Ala-Leu-Asn |
| | | (SEQ ID NO:7) |
| | 144 | Lys-Pro-Val-Ser-Leu-Ser-Tyr-Arg-Ala- |
| | | Pro-Phe-Arg-Phe-Phe-Gly-Lys-Lys-Gly- |
| | | Leu-Lys-Trp-Ile-Gln-Glu-Tyr-Leu-Glu- |
| | | Lys-Ala-Leu-Asn |
| | | (SEQ ID NO:8) |
| | 145 | Lys-Pro-Val-Ser-Leu-Ser-Tyr-Arg-Ala- |
| | | Pro-Phe-Arg-Phe-Phe-Gly-Gly-Lys-Lys- |
| | | Leu-Lys-Trp-Ile-Gln-Glu-Tyr-Leu-Glu- |
| | | Lys-Ala-Leu-Asn |
| | | (SEQ ID NO:9) |
| | 146 | Lys-Pro-Val-Ser-Leu-Ser-Tyr-Arg-Ala- |
| | | Pro-Phe-Arg-Phe-Phe-Lys-Gly-Gly-Lys- |
| | | Leu-Lys-Trp-Ile-Gln-Glu-Tyr-Leu-Glu- |
| | | Lys-Ala-Leu-Asn |
| | | (SEQ ID NO:10) |
| | 147 | R_{N}-Lys-Pro-Val-Ser-Leu-Ser-Tyr-Arg- |
| | | Ala-Pro-Phe-Arg-Phe-Phe-Lys-Gly-Lys- |
| | | Gly-Leu-Lys-Trp-Ile-Gln-Glu-Tyr-Leu |
| | | Glu-Lys-Ala-Leu-Asn |
| | | (SEQ ID NO:11) |
| | 148 | Lys-Pro-Val-Ser-Leu-Ser-Tyr-Arg-Ala |
| | | Pro-Phe-Arg-Phe-Phe-Gly-Lys-Gly-Lys- |
| | | Leu-Lys-Trp-Ile-Gln-Glu-Tyr-Leu-Glu |
| | | Lys-Ala-Leu-Asn |
| | | (SEQ ID NO:12) |
| | 149 | Lys-Pro-Val-Ser-Leu-Ser-Tyr-Arg-Ala- |
| | | Pro-Phe-Arg-Phe-Phe-Lys-Lys-Lys-Gly- |
| | | Leu-Lys-Trp-Ile-Gln-Glu-Tyr-Leu-Glu- |
| | | Lys-Ala-Leu-Asn |
| | | (SEQ ID NO:13) |
| | 150 | Lys-Pro-Val-Ser-Leu-Ser-Tyr-Arg-Ala |
| | | Pro-Phe-Arg-Phe-Phe-Gly-Lys-Lys-Lys- |
| | | Leu-Lys-Trp-Ile-Gln-Glu-Tyr-Leu-Glu |
| | | Lys-Ala-Leu-Asn |
| | | (SEQ ID NO:14) |
| | 151 | Lys-Pro-Val-Ser-Leu-Ser-Tyr-Arg-Ala- |
| | | Pro-Phe-Arg-Phe-Phe-Lys-Gly-Lys-Lys - |
| | | Leu-Lys-Trp-Ile-Gln-Glu-Tyr-Leu-Glu- |
| | | Lys-Ala-Leu-Asn |
| | | (SEQ ID NO:15) |
| | 152 | Lys-Pro-Val-Ser-Leu-Ser-Tyr-Arg-Ala- |
| | | Pro-Phe-Arg-Phe-Phe-Lys-Lys-Gly-Lys- |
| | | Leu-Lys-Trp-Ile-Gln-Glu-Tyr-Leu-Glu- |
| | | Lys-Ala-Leu-Asn |
| | | (SEQ ID NO:16) |
| | 153 | Lys-Pro-Val-Ser-Leu-Ser-Tyr-Arg-Ala- |
| | | Pro-Phe-Arg-Phe-Phe-Lys-Lys-Lys-Lys- |
| | | Leu-Lys-Trp-Ile-Gln-Glu-Tyr-Leu-Glu- |
| | | Lys-Ala-Leu-Asn |
| | | (SEQ ID NO:17) |
| | 154 | Lys-Pro-Val-Ser-Leu-Ser-Tyr-Arg-Ala- |
| | | Pro-Phe-Arg-Phe-Phe-Arg-Gly-Gly-Gly- |
| | | Leu-Lys-Trp-Ile-Gln-Glu-Tyr-Leu-Glu- |
| | | Lys-Ala-Leu-Asn |
| | | (SEQ ID NO:18) |
| | 155 | Lys-Pro-Val-Ser-Leu-Ser-Tyr-Arg-Ala- |
| | | Pro-Phe-Arg-Phe-Phe-Gly-Arg-Gly-Gly- |
| | | Leu-Lys-Trp-Ile-Gln-Glu-Tyr-Leu-Glu- |
| | | Lys-Ala-Leu-Asn |
| | | (SEQ ID NO:19) |
| | 156 | Lys-Pro-Val-Ser-Leu-Ser-Tyr-Arg-Ala |
| | | Pro-Phe-Arg-Phe-Phe-Gly Gly-Arg-Gly- |
| | | Leu-Lys-Trp-Ile-Gln-Glu-Tyr-Leu-Glu |
| | | Lys-Ala-Leu-Asn |
| | | SEQ ID NO:20) |
| | 157 | Lys-Pro-Val-Ser-Leu-Ser-Tyr-Arg-Ala- |
| | | Pro-Phe-Arg-Phe-Phe-Gly-Gly-Gly-Arg- |
| | | Leu-Lys-Trp-Ile-Gln-Glu-Tyr-Leu-Glu- |
| | | Lys-Ala-Leu-Asn |
| | | (SEQ ID NO:21) |
| | 158 | Lys-Pro-Val-Ser-Leu-Ser-Tyr-Arg-Ala |
| | | Pro-Phe-Arg-Phe-Phe-Arg-Arg-Gly-Gly- |
| | | Leu-Lys-Trp-Ile-Gln-Glu-Tyr-Leu-Glu |
| | | Lys-Ala-Leu-Asn |
| | | (SEQ ID NO:22) |
| | 159 | Lys-Pro-Val-Ser-Leu-Ser-Tyr-Arg-Ala |
| | | Pro-Phe-Arg-Phe-Phe-Gly-Arg-Arg-Gly- |
| | | Leu-Lys-Trp-Ile-Gln-Glu-Tyr-Leu-Glu |
| | | Lys-Ala-Leu-Asn |
| | | (SEQ ID NO:23) |
| | 160 | Lys-Pro-Val-Ser-Leu-Ser-Tyr-Arg-Ala- |
| | | Pro-Phe-Arg-Phe-Phe-Gly-Gly-Arg-Arg- |
| | | Leu-Lys-Trp-Ile-Gln-Glu-Tyr-Leu-Glu- |
| | | Lys-Ala-Leu-Asn |
| | | (SEQ ID NO:24) |
| | 161 | Lys-Pro-Val-Ser-Leu-Ser-Tyr-Arg-Ala- |
| | | Pro-Phe-Arg-Phe-Phe-Arg-Gly-Gly-Arg- |
| | | Leu-Lys-Trp-Ile-Gln-Glu-Tyr-Leu-Glu- |
| | | Lys-Ala-Leu-Asn |
| | | (SEQ ID NO:25) |
| | 162 | Lys-Pro-Val-Ser-Leu-Ser-Tyr-Arg-Ala |
| | | Pro-Phe-Arg-Phe-Phe-Arg-Gly-Arg-Gly- |
| | | Leu-Lys-Trp-Ile-Gln-Glu-Tyr-Leu-Glu |
| | | Lys-Ala-Leu-Asn |
| | | (SEQ ID NO:26) |
| | 163 | Lys-Pro-Val-Ser-Leu-Ser-Tyr-Arg-Ala |
| | | Pro-Phe-Arg-Phe-Phe-Gly-Arg-Gly-Arg- |
| | | Leu-Lys-Trp-Ile-Gln-Glu-Tyr-Leu-Glu- |
| | | Lys-Ala-Leu-Asn |
| | | (SEQ ID NO:27) |
| | 164 | Lys-Pro-Val-Ser-Leu-Ser-Tyr-Arg-Ala |
| | | Pro-Phe-Arg-Phe-Phe-Arg-Arg-Arg-Gly- |
| | | Leu-Lys-Trp-Ile-Gln-Glu-Tyr-Leu-Glu |
| | | Lys-Ala-Leu-Asn |
| | | (SEQ ID NO:28) |
| | 165 | Lys-Pro-Val-Ser-Leu-Ser-Tyr-Arg-Ala- |
| | | Pro-Phe-Arg-Phe-Phe-Gly-Arg-Arg-Arg- |
| | | Leu-Lys-Trp-Ile-Gln-Glu-Tyr-Leu-Glu- |
| | | Lys-Ala-Leu-Asn |
| | | (SEQ ID NO:29) |
| | 166 | Lys-Pro-Val-Ser-Leu-Ser-Tyr-Arg-Ala- |
| | | Pro-Phe-Arg-Phe-Phe-Arg-Gly-Arg-Arg- |
| | | Leu-Lys-Trp-Ile-Gln-Glu-Tyr-Leu-Glu- |
| | | Lys-Ala-Leu-Asn |
| | | (SEQ ID NO:30) |
| | 167 | Lys-Pro-Val-Ser-Leu-Ser-Tyr-Arg-Ala- |
| | | Pro-Phe-Arg-Phe-Phe-Arg-Arg-Gly-Arg- |
| | | Leu-Lys-Trp-Ile-Gln-Glu-Tyr-Leu-Glu- |
| | | Lys-Ala-Leu-Asn |
| | | (SEQ ID NO:31) |
| | 168 | Lys-Pro-Val-Ser-Leu-Ser-Tyr-Arg-Ala- |
| | | Pro-Phe-Arg-Phe-Phe-Arg-Arg-Arg-Arg- |
| | | Leu-Lys-Trp-Ile-Gln-Glu-Tyr-Leu-Glu- |
| | | Lys-Ala-Leu-Asn |
| | | (SEQ ID NO:32) |
| | | See U.S. Pat. Application Nos. |
| | | 11/393,769; 11/388,542; 10/945,674; |
| | | 10/086,177; and 09/835,107, each of |
| | | which is hereby incorporated herein by |
| | | reference in its entirety, for |
| | | additional sequences. |

CXCL13 (BCA-1) Analogs Source: Artificial Human

| | |
|---|---|
| 169 | R-X₀₁ X₀₂ X₀₃ X₀₄ X₀₅ X₀₆ X₀₇ X₀₈ X₀₉ X₁₀ X₁₁ |
| | X₁₂ X₁₃ X₁₄ X₁₅ X₁₆ [linker] Y₀₁ Y₀₂ Y₀₃ Y₀₄ |
| | Y₀₅ Y₀₆ Y₀₇ Y₀₈ Y₀₉ Y₁₀ Y₁₁ Y₁₂ Y₁₃ Y₁₄ |
| 170 | Val Leu Glu Val Tyr Tyr Thr Ser Leu |
| | Arg Cys Arg Cys Val Gln Glu -[linker]- |
| | Glu Val Leu Arg Lys Arg Ser Ser Ser |
| | Thr Leu Pro Val Pro |
| 171 | Val Leu Glu Val Tyr Tyr Thr Ser Leu |
| | Arg Cys Arg Cys Val Gln Glu -[linker]- |
| | Gln Ala Glu Trp Ile Gln Arg Met Met |
| | Glu Val Leu Arg Lys |
| 172 | Val Leu Glu Val Tyr Tyr Thr Ser Leu |
| | Arg Cys Arg Cys Val Gln Glu -[linker]- |
| | Leu Arg Lys Arg Ser Ser Ser Thr Leu |
| | Pro Val Pro Val Pro |
| 173 | Val Leu Glu Val Tyr Tyr Thr Ser Leu |
| | Arg Cys Arg Cys Val Gln Glu -[linker]- |
| | Arg Lys Arg Ser Ser Ser Thr Leu Pro |
| | Val Pro Val Pro Phe |
| 174 | Val Leu Glu Val Tyr Tyr Thr Ser Leu |
| | Arg Cys Arg Cys Val Gln Glu -[linker]- |
| | Lys Arg Ser Ser Ser Thr Leu Pro Val |
| | Pro Val Pro Ile Lys |
| 175 | Val Leu Glu Val Tyr Tyr Thr Ser Leu |
| | Arg Cys Arg Cys Val Gln Glu -[linker]- |
| | Arg Ser Ser Ser Thr Leu Pro Val Pro |
| | Val Pro Ile Lys Arg |
| 176 | Val Leu Glu Val Tyr Tyr Thr Ser Leu |
| | Arg Cys Arg Cys Val Gln Glu -[linker]- |
| | Ser Ser Ser Thr Leu Pro Val Pro Val |
| | Pro Phe Lys Arg Lys |
| 177 | Val Leu Glu Val Tyr Tyr Thr Ser Leu |
| | Arg Cys Arg Cys Val Gln Glu -[linker]- |
| | Ser Ser Thr Leu Pro Val Pro Val Pro |
| | Phe Lys Arg Lys Ile |
| 178 | Val Leu Glu Val Tyr Tyr Thr Ser Leu |
| | Arg Cys Arg Cys Val Gln Glu -[linker]- |
| | Ser Thr Leu Pro Val Pro Val Pro Phe |
| | Lys Arg Lys Ile Pro |
| 179 | Val Leu Glu Val Tyr Tyr Thr Ser Leu |
| | Arg Cys Arg Cys Val Gln Glu -[linker]- |
| | Glu Val Leu Arg Lys Arg Ser Ser Ser |
| | Thr Leu Pro Val Pro |

CXCL14 (BRAK) Analogs Source: Artificial Human

| | |
|---|---|
| 180 | R-X₀₁ X₀₂ X₀₃ X₀₄ X₀₅ X₀₆ X₀₇ X₀₈ X₀₉ X₁₀ X₁₁ |
| | X₁₂ X₁₃ X₁₄ X₁₅ X₁₆ [linker] Y₀₁ Y₀₂ Y₀₃ Y₀₄ |
| | Y₀₅ Y₀₆ Y₀₇ Y₀₈ Y₀₉ Y₁₀ Y₁₁ Y₁₂ Y₁₃ Y₁₄ |
| 181 | Ser Lys Cys Lys Cys Ser Arg Lys Gly |
| | Pro Lys Ile Arg Tyr Ser Asp -[linker]- |
| | Glu Val Leu Arg Lys Arg Ser Ser Ser |
| | Thr Leu Pro Val Pro |
| 182 | Val Leu Glu Val Tyr Tyr Thr Ser Leu |
| | Arg Cys Arg Cys Val Gln Glu -[linker]- |
| | Lys Leu Gln Ser Thr Lys Arg Phe Ile |
| | Lys Trp Tyr Asn Ala |
| 183 | Val Leu Glu Val Tyr Tyr Thr Ser Leu |
| | Arg Cys Arg Cys Val Gln Glu -[linker]- |
| | Leu Gln Ser Thr Lys Arg Phe Ile Lys |
| | Trp Tyr Asn Ala Trp |
| 184 | Val Leu Glu Val Tyr Tyr Thr Ser Leu |
| | Arg Cys Arg Cys Val Gln Glu -[linker]- |
| | Gln Ser Thr Lys Arg Phe Ile Lys Trp |
| | Tyr Asn Ala Trp Asn |
| 185 | Val Leu Glu Val Tyr Tyr Thr Ser Leu |
| | Arg Cys Arg Cys Val Gln Glu -[linker]- |
| | Ser Thr Lys Arg Phe Ile Lys Trp Tyr |
| | Asn Ala Trp Asn Glu |
| 186 | Val Leu Glu Val Tyr Tyr Thr Ser Leu |
| | Arg Cys Arg Cys Val Gln Glu -[linker]- |
| | Thr Lys Arg Phe Ile Lys Trp Tyr Asn |
| | Ala Trp Asn Glu Lys |
| 187 | Val Leu Glu Val Tyr Tyr Thr Ser Leu |
| | Arg Cys Arg Cys Val Gln Glu -[linker]- |
| | Lys Arg Phe Ile Lys Trp Tyr Asn Ala |
| | Trp Asn Glu Lys Arg |
| 188 | Val Leu Glu Val Tyr Tyr Thr Ser Leu |
| | Arg Cys Arg Cys Val Gln Glu -[linker]- |
| | Arg Phe Ile Lys Trp Tyr Asn Ala Trp |
| | Asn Glu Lys Arg Arg |
| 189 | Val Leu Glu Val Tyr Tyr Thr Ser Leu |
| | Arg Cys Arg Cys Val Gln Glu -[linker]- |
| | Phe Ile Lys Trp Tyr Asn Ala Trp Asn |
| | Glu Lys Arg Arg Val |
| 190 | Val Leu Glu Val Tyr Tyr Thr Ser Leu |
| | Arg Cys Arg Cys Val Gln Glu -[linker]- |
| | Ile Lys Trp Tyr Asn Ala Trp Asn Glu |
| | Lys Arg Arg Val Tyr |
| 191 | Val Leu Glu Val Tyr Tyr Thr Ser Leu |
| | Arg Cys Arg Cys Val Gln Glu -[linker]- |
| | Lys Trp Tyr Asn Ala Trp Asn Glu Lys |
| | Arg Arg Val Tyr Glu |
| 192 | Val Leu Glu Val Tyr Tyr Thr Ser Leu |
| | Arg Cys Arg Cys Val Gln Glu -[linker]- |
| | Trp Tyr Asn Ala Trp Asn Glu Lys Arg |
| | Arg Val Tyr Glu Glu |
| 193 | Val Leu Glu Val Tyr Tyr Thr Ser Leu |
| | Arg Cys Arg Cys Val Gln Glu -[linker]- |
| | Trp Tyr Asn Ala Trp Asn Glu Lys Arg |
| | Arg Val Tyr Glu Glu |

CXCL15 (Lungkine) Analog Source: Artificial Mouse

| | |
|---|---|
| 194 | R-X₀₁ X₀₂ X₀₃ X₀₄ X₀₅ X₀₆ X₀₇ X₀₈ X₀₉ X₁₀ X₁₁ |
| | X₁₂ X₁₃ X₁₄ X₁₅ X₁₆ [linker] Y₀₁ Y₀₂ Y₀₃ Y₀₄ |
| | Y₀₅ Y₀₆ Y₀₇ Y₀₈ Y₀₉ Y₁₀ Y₁₁ Y₁₂ Y₁₃ Y₁₄ |
| 195 | Gln Glu Leu Arg Cys Leu Cys Ile Gln |
| | Glu His Ser Glu Phe Ile Pro -[linker]- |
| | Ile Arg Glu Thr Ser Arg His Phe Ala |
| | Asp Leu Ala His Asn |
| | (SEQ ID NO:117) |
| 196 | Gln Glu Leu Arg Cys Leu Cys Ile Gln |
| | Glu His Ser Glu Phe Ile Pro -[linker]- |
| | Asp Arg Asn Phe Leu Arg Asp Ser Ser |
| | Glu Val Ser Leu Thr |
| | (SEQ ID NO:118) |
| 197 | Gln Glu Leu Arg Cys Leu Cys Ile Gln |
| | Glu His Ser Glu Phe Ile Pro -[linker]- |
| | Arg Glu Thr Ser Arg His Phe Ala Asp |
| | Leu Ala His Asn Ser |
| | (SEQ ID NO:119) |
| 198 | Gln Glu Leu Arg Cys Leu Cys Ile Gln |
| | Glu His Ser Glu Phe Ile Pro -[linker]- |
| | Arg Asn Phe Leu Arg Asp Ser Ser Glu |
| | Val Ser Leu Thr Gly |
| | (SEQ ID NO:120) |
| 199 | Gln Glu Leu Arg Cys Leu Cys Ile Gln |
| | Glu His Ser Glu Phe Ile Pro -[linker]- |
| | Asn Phe Leu Arg Asp Ser Ser Glu Val |
| | Ser Leu Thr Gly Ser |
| | (SEQ ID NO:121) |
| 200 | Gln Glu Leu Arg Cys Leu Cys Ile Gln |
| | Glu His Ser Glu Phe Ile Pro -[linker]- |
| | Phe Leu Arg Asp Ser Ser Glu Val Ser |
| | Leu Thr Gly Ser Asp |
| | (SEQ ID NO:122) |
| 201 | Gln Glu Leu Arg Cys Leu Cys Ile Gln |
| | Glu His Ser Glu Phe Ile Pro -[linker]- |
| | Leu Arg Asp Ser Ser Glu Val Ser Leu |
| | Thr Gly Ser Asp Ala |
| | (SEQ ID NO:123) |
| 202 | Gln Glu Leu Arg Cys Leu Cys Ile Gln |
| | Glu His Ser Glu Phe Ile Pro -[linker]- |
| | Ile Arg Glu Thr Ser Lys His Phe Ala |
| | Asp Leu Ala His Asn |
| | (SEQ ID NO:124) |
| 203 | Gln Glu Leu Arg Cys Leu Cys Ile Gln |
| | Glu His Ser Glu Phe Ile Pro -[linker]- |
| | Asp Arg Asn Phe Leu Lys Asp Ser Ser |
| | Glu Val Ser Leu Thr |
| | (SEQ ID NO:i25) |

CXCL16 (SRPSOX) Analogs Source: Artificial Human

| | |
|---|---|
| 204 | R-X₀₁ X₀₂ X₀₃ X₀₄ X₀₅ X₀₆ X₀₇ X₀₈ X₀₉ X₁₀ X₁₁ |
| | X₁₂ X₁₃ X₁₄ X₁₅ X₁₆ [linker] Y₀₁ Y₀₂ Y₀₃ Y₀₄ |
| | Y₀₅ Y₀₆ Y₀₇ Y₀₈ Y₀₉ Y₁₀ Y₁₁ Y₁₂ Y₁₃ Y₁₄ |
| 205 | Gly Ser Val Thr Gly Ser Cys Tyr Cys |
| | Gly Lys Arg Ile Ser Ser Asp -[linker]- |
| | Trp Val Gln Glu Leu Met Ser Cys Leu |
| | Asp Leu Lys Glu Cys |
| | (SEQ ID NO:126) |
| 206 | Gly Ser Val Thr Gly Ser Cys Tyr Cys |
| | Gly Lys Arg Ile Ser Ser Asp -[linker]- |
| | Val Gln Glu Leu Met Ser Cys Leu Asp |
| | Leu Lys Glu Cys Gly |
| | (SEQ ID NO:127) |
| 207 | Gly Ser Val Thr Gly Ser Cys Tyr Cys |
| | Gly Lys Arg Ile Ser Ser Asp -[linker]- |
| | Gln Glu Leu Met Ser Cys Leu Asp Leu |
| | Lys Glu Cys Gly His |
| | (SEQ ID NO:128) |
| 208 | Gly Ser Val Thr Gly Ser Cys Tyr Cys |
| | Gly Lys Arg Ile Ser Ser Asp -[linker]- |
| | Glu Leu Met Ser Cys Leu Asp Leu Lys |
| | Glu Cys Gly His Ala |
| | (SEQ ID NO:129) |
| 209 | Gly Ser Val Thr Gly Ser Cys Tyr Cys |
| | Gly Lys Arg Ile Ser Ser Asp -[linker]- |
| | Leu Met Ser Cys Leu Asp Leu Lys Glu |
| | Cys Gly His Ala Tyr |
| | (SEQ ID NO:130) |
| 210 | Gly Ser Val Thr Gly Ser Cys Tyr Cys |
| | Gly Lys Arg Ile Ser Ser Asp -[linker]- |
| | Met Ser Cys Leu Asp Leu Lys Glu Cys |
| | Gly His Ala Tyr Ser |
| | (SEQ ID NO:131) |
| 211 | Gly Ser Val Thr Gly Ser Cys Tyr Cys |
| | Gly Lys Arg Ile Ser Ser Asp -[linker]- |

We claim:

1. A composition comprising an analog of a native CXC chemokine selected from a group consisting of CXCL1, CXCL2, CXCL3, CXCL5, CXCL6, CXCL7, CXCL9, CXCL11, CXCL13, CXCL14, CXCL15, CXCL16, and CXCL17, wherein the analog has a length ranging from about 20 to about 37 amino acids and comprises:
an N-terminal region comprising a first conserved sequence consisting of about 13 to 17 of the first 17 of the native CXC chemokine N-terminal residues, or conservatively modified variants thereof, or a sequence having at least 90% homology to the first conserved sequence and capable of binding to a cellular receptor that binds to the first conserved sequence;
a C-terminal region comprising a second conserved sequence consisting of about 6 to 16 of the last 16 of the native CXC chemokine C-terminal residues; or
conservatively modified variants thereof, or a sequence having at least 90% homology to the second conserved sequence and capable of binding to a cellular receptor that binds to the second conserved sequence; and,
a linker selected from a group consisting of from 1 to 4 natural or non-natural amino acids having the following structure:
wherein,
R_{L} is selected from a group consisting of saturated and unsaturated aliphatics and heteroaliphatics consisting of 20 or fewer carbon atoms that are optionally substituted with (i) a hydroxyl, carboxyl, amino, amido, or imino group, or (ii) an aromatic group having from 5 to 7 members in the ring; and —(CH₂)ₙ—, wherein n is an integer ranging from 1 to 20;
the analog is optionally modified with a modifier selected from a group consisting of a poly(ethylene glycol) or derivative thereof, a glycosaminoglycan, a diagnostic label, a radioactive group, an acyl group, an acetyl group, a peptide, a modifier capable of reducing the ability of the analog to act as a substrate for aminopeptidases, and a modifier capable of reducing the ability of the analog to act as a substrate for carboxypeptidases.

2. The composition of claim 1, wherein
the analog is a non-ELR-CXC chemokine analog;
the first conserved sequence consists of about 13 to 17 of the first 17 of the native CXC chemokine N-terminal residues, or conservatively modified variants thereof, or a sequence having at least 90% homology to the first conserved sequence and capable of binding to a cellular receptor that binds to the first conserved sequence, wherein the first conserved sequence does not include an ELR motif; and,
the second conserved sequence consisting of about 6 to 16 of the last 16 of the native CXC chemokine C-terminal residues, or conservatively modified variants thereof, or a sequence having at least 90% homology to the second conserved sequence and capable of binding to a cellular receptor that binds to the second conserved sequence.

3. The composition of claim 1, wherein
the analog is an ELR-CXC chemokine analog;
the first conserved sequence consists of about 13 to 17 of the first 17 of the native CXC chemokine N-terminal residues, or conservatively modified variants thereof, or a sequence having at least 90% homology to the first conserved sequence and capable of binding to a cellular receptor that binds to the first conserved sequence, wherein the first conserved sequence includes an ELR motif; and,
the second conserved sequence consisting of about 6 to 16 of the last 16 of the native CXC chemokine C-terminal residues, or conservatively modified variants thereof, or a sequence having at least 90% homology to the second conserved sequence and capable of binding to a cellular receptor that binds to the second conserved sequence.

4. The composition of claim 1, wherein the C-terminal region is cyclized.

5. The composition of claim 2, wherein the C-terminal region is cyclized.

6. The composition of claim 3, wherein the C-terminal region is cyclized.

7. The composition of claim 1, wherein the linker is 11-aminoundecanoic acid.

8. The composition of claim 2, wherein the linker is 11-aminoundecanoic acid.

9. The composition of claim 3, wherein the linker is 11-aminoundecanoic acid.

10. The composition of claim 1, wherein the linker is a combination of 4 natural amino acids, and the linker optionally contains an amino acid having a side chain bearing positive charge.

11. The composition of claim 2, wherein the linker is a combination of 4 natural amino acids, and the linker optionally contains an amino acid having a side chain bearing positive charge.

12. The composition of claim 3, wherein the linker is a combination of 4 natural amino acids, and the linker optionally contains an amino acid having a side chain bearing positive charge.

13. A method of increasing the activity of a cell having a CXC receptor comprising binding the CXC receptor to the analog of claim 1, wherein the increase is relative to the activity of the cell in the absence of the analog.

14. A method of increasing the activity of a cell having a CXC receptor comprising binding the CXC receptor to the analog of claim 2, wherein the increase is relative to the activity of the cell in the absence of the analog.

15. A method of increasing the activity of a cell having a CXC receptor comprising binding the CXC receptor to the analog of claim 3, wherein the increase is relative to the activity of the cell in the absence of the analog.

16. A method of decreasing the activity of a cell having a CXC receptor comprising binding the CXC receptor to the analog of claim 1, wherein the increase is relative to the activity of the cell in the absence of the analog.

17. A method of decreasing the activity of a cell having a CXC receptor comprising binding the CXC receptor to the analog of claim 2, wherein the increase is relative to the activity of the cell in the absence of the analog.

18. A method of decreasing the activity of a cell having a CXC receptor comprising binding the CXC receptor to the analog of claim 3, wherein the increase is relative to the activity of the cell in the absence of the analog.

19. An antibody produced using the analog of claim 1 as the antigen.

20. The antibody of claim 19, wherein the antibody is monoclonal.


SEQUENCE LISTING (214 sequences)

SEQ ID NO: 1 - protein, 73 aa, Homo sapiens
ASVATELRCQCLQTLQGIHPKNIQSVNVKSPGPHCAQTEVIATLKNGRKACLNPASPIVKKIIEKMLNSDKSN

SEQ ID NO: 2 - protein, 69 aa, Homo sapiens
TELRCQCLQTLQGIHLKNIQSVKVKSPGPHCAQTEVIATLKNGQKACLNPASPMVKKIIEKMLKNGKSN

SEQ ID NO: 3 - protein, 69 aa, Homo sapiens
TELRCQCLQTLQGIHLKNIQSVNVRSPGPHCAQTEVIATLKNGKKACLNPASPMVQKIIEKILNKGSTN

SEQ ID NO: 4 - protein, 70 aa, Homo sapiens
EAEEDGDLQCLCVKTTSQVRPRHITSLEVIKAGPHCPTAQLIATLKNGRKICLDLQAPLYKKIIKKLLES

SEQ ID NO: 5 - protein, 71 aa, Homo sapiens
LRELRCVCLQTTQGVHPKMISNLQVFAIGPQCSKVEVVASLKNGKEICLDPEAPFLKKVIQKILDGGNKEN

SEQ ID NO: 6 - protein, 77 aa, Homo sapiens
GPVSAVLTELRCTCLRVTLRVNPKTIGKLQVFPAGPQCSKVEVVASLKNGKQVCLDPEAPFLKKVIQKILDSGNKKN

SEQ ID NO: 7 - protein, 70 aa, Homo sapiens
AELRCMCIKTTSGIHPKNIQSLEVIGKGTHCNQVEVIATLKDGRKICLDPDAPRIKKIVQKKLAGDESAD

SEQ ID NO: 8 - protein, 72 aa, Homo sapiens
SAKELRCQCIKTYSKPFHPKFIKELRVIESGPHCANTEIIVKLSDGRELCLDPKENWVQRVVEKFLKRAENS

SEQ ID NO: 9 - protein, 103 aa, Homo sapiens
TPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 10 - protein, 78 aa, Homo sapiens
VPLSRTVRCTCISISNQPVNPPRSLEKLEIIPASQFCPRVEIIATMKKKGEKRCLNPESKAIKNLLKAVSKEMSKRSP

SEQ ID NO: 11 - protein, 73 aa, Homo sapiens
FPMFKRGRCLCIGPGVKAVKVADIEKASIMYPSNNCDKIEVIITLKENKGQRCLNPKSKQARLIIKKVERKNF

SEQ ID NO: 12 - protein, 67 aa, Homo sapiens
KPVSLSYRCPCRFFESHVARANVKHLKILNTPNCALQIVARLKNNNRQVCIDPKLKWIQEYLEKALN

SEQ ID NO: 13 - protein, 87 aa, Homo sapiens
VLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 14 - protein, 77 aa, Homo sapiens
SKCKCSRKGPKIRYSDVKKLEMKPKYPHCEEKMVIITTKSVSRYRGQEHCLHPKLQSTKRFIKWYNAWNEKRRVYEE

SEQ ID NO: 15 - protein, 142 aa, Mus musculus
QELRCLCIQEHSEFIPLKLIKNIMVIFETIYCNRKEVIAVPKNGSMICLDPDAPWVKATVGPITNRFLPEDLKQKEFPPAMKLLYSVEHEKPLYLSFGRPENKRIFPFPIRETSRHFADLAHNSDRNFLRDSSEVSLTGSDA

SEQ ID NO: 16 - protein, 76 aa, Homo sapiens
GSVTGSCYCGKRISSDSPPSVQFMNRLRKHLRAYHRCLYYTRFQLLSWSVCGGNKDPWVQELMSCLDLKECGHAYS

SEQ ID NO: 17 - protein, 119 aa, Homo sapiens
MKVLISSLLLLLPLMLMSMVSSSLNPGVARGHRDRGQASRRWLQEGGQECECKDWFLRAPRRKFMTVSGLPKKQCPCDHFKGNVKKTRHQRHHRKPNKHSRACQQFLKQCQLRSFALPL

SEQ ID NO: 18 - protein, 57 aa, Artificial Sequence
  Description of Artificial Sequence: Synthetic peptide
  MOD_RES (1): L-or D-Ala, Gly, L- or D-Phe, the preferred amino acid residues is L- or D-Ala
  MOD_RES (2): any natural or non natural amino acid different from L- or D-Cys, such as L- or D-Ser, L- or D-Thr, or L- or D-Tyr
  MOD_RES (3): L- or D-Val, L- or D-Leu, L- or D-Ile, L- or D-Pro, L- or D-Phe, L- or D-Tyr, L- or D-Ser, or L- or D-Thr
  MOD_RES (4): any natural or non natural amino acid residue different from L- or D-Cys residue, such as L- or D-Ala, Gly, or D-L-Phe
  MOD_RES (5): L- or D-Thr, L- or D-Ser, L- or D-Tyr, L- or D-Trp, or L- or D-His
  MOD_RES (6): any natural or non natural amino acid residue different from L- or D-Glx, L- or D-Asx, L- or D-Arg, L- or D-Lys, L- or D-His, L- or D-Tyr, L- or D-Ser, L- or D-Thr, such as L- or D-Glu, or L- or D-Asp
  MOD_RES (7): any natural or non natural amino acid residue different from L- or D-Cys residue, such as L- or D-Leu, L- or D-Ile, L- or D-L- or D-Val, or L- or D-Ala, L- or D-Phe, or L- or D-Tyr
  MOD_RES (8): any natural or non natural amino acid residue different from L- or D-Cys residue, such as L- or D-Arg, L- or D-Lys, L- or D-Orn- or D-Ala
  MOD_RES (9): L- or D-Cys, L- or D-Ala, L- or D-Phe, L- or D-His, L- or D-Trp, L- or D-Ser, L- or D-Thr, L- or D-Lys, L- or D-His, or L- or D-Tyr
  MOD_RES (10): any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, or L- or D-Trp
  MOD_RES (11): L- or D-Cys, L- or D-Ala, L- or D-Phe, L- or D-His, L- or D-Trp, L- or D-Ser, L- or D-Thr, L- or D-Lys, L- or D-His, or L- or D-Tyr
  MOD_RES (12): any natural or non natural amino acid residue different from L- or D-Cys residue, such as L- or D-Leu, L- or D-Ile, L- or D-L- or D-Val, or L- or D-Ala, L- or D-Phe, or L- or D-Tyr
  MOD_RES (13): any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, or L- or D-Trp
  MOD_RES (14): L- or D-Thr, L- or D-Ser, L- or D-Tyr, L- or D-Trp, or L- or D-His
  MOD_RES (15): any natural or non natural amino acid residue different from L- or D-Cys residue, such as L- or D-Leu, L- or D-Ile, L- or D-L- or D-Val, or L- or D-Ala, L- or D-Phe, or L- or D-Tyr
  MOD_RES (16)..(21): one or up to six natural or non natural amino acid residue different from L- or D-Cys residue; see specification as filed for detailed description of substitutions and preferred embodiments
  MOD_RES (22)..(41): this region may encompass 1-20 variable amino acids
  MOD_RES (42): any natural or non natural amino acid residue different from L- or D-Cys residue, such as L- or D-Ile, L- or D-Leu, L- or D-L- or D-Val, or L- or D-Ala, L- or D-Phe, L- or D-Tyr, or L- or D- Lys
  MOD_RES (43): L- or D-Val, L- or D-Leu, L- or D-Ile, L- or D-Pro, L- or D-Phe, L- or D-Tyr, L- or D-Ser, L- or D-Thr, or L- or D-Lys
  MOD_RES (44): any natural or non natural amino acid residue different from L- or D-Cys residue, such as L- or D-Lys, L- or D-Arg, L- or D-Orn- or D-Ala, or L- or D-Ile
  MOD_RES (45): any natural or non natural amino acid residue different from L- or D-Cys residue, such as L- or D-Lys, L- or D-Arg, L- or D-Orn- or D-Ala, or L- or D-Ile
  MOD_RES (46): any natural or non natural amino acid residue different from L- or D-Cys residue, such as L- or D-Ile, L- or D-Leu, L- or D-L- or D-Val, or L- or D-Ala, L- or D-Phe, L- or D-Tyr, or L- or D-Glu
  MOD_RES (47): any natural or non natural amino acid residue different from L- or D-Cys residue, such as L- or D-Ile, L- or D-Leu, L- or D-L- or D-Val, or L- or D-Ala, L- or D-Phe, L- or D-Tyr, L- or D-Glu, or L- or D-Lys
  MOD_RES (48): any natural or non natural amino acid residue different from L- or D-Glx, L- or D-Asx, L- or D-Arg, L- or D-Lys, L- or D-His, L- or D-Tyr, L- or D-Ser, L- or D-Thr, L- or D-Met, or L- or D-Nle
  MOD_RES (49): any natural or non natural amino acid residue different from L- or D-Lys, L- or D-Met, L- or D-Arg, L- or D-Asx, L- or D-Glx, L- or D-His, L- or D-Tyr, L- or D-Ser, L- or D-Thr, L- or D-Leu, or L- or D-Ile
  MOD_RES (50): L- or D-Met, L- or D-Leu, L- or D-Asx, L- or D-Nle, L- or D-Ala, L- or D-Phe, L- or D-Glx, L- or D-Ile, or L- or D-Tyr
  MOD_RES (51): any natural or non natural amino acid residue different from L- or D-Cys residue, such as L- or D-Leu, L- or D-Ile, L- or D-L- or D-Asx, or L- or D-Glx, L- or D-Ala, L- or D-Val, L- or D-Ser, L- or D-Thr, or L- or D-Tyr
  MOD_RES (52): L- or D-Asx, L- or D-Glx, L- or D-Ser, L- or D-Thr, L- or D-Tyr, L- or D-Lys, or L or D-Arg
  MOD_RES (53): any natural or non natural amino acid different from L- or D-Cys, such as L- or D-Ser, L- or D-Asx, L- or D-Glx, or L- or D-Lys
  MOD_RES (54): L-or D-Glx, L- or D-Asx, L- or D-Lys, L- or D- Ser, L- or D-Thr, L- or D-Tyr, or L- or Arg
  MOD_RES (55)..(57): one or up to three natural or non natural amino acid residue different from L- or D-Cys residue, such as L- or D-Lys, L- or D-Arg, L- or D-Ser, L- or D- Asn, L- or D-Orn-, or D-Ala, and L- or D-(Lys-Ser), L- or D-( Lys-Ser-Asn)
XXXXXXXXXXXXXXXXXXXXXXXXXXXXXXXXXXXXXXXXXXXXXXXXXXXXXXXXX

SEQ ID NO: 19 - protein, 54 aa, Artificial Sequence
  Description of Artificial Sequence: Synthetic peptide
  MOD_RES (1): any natural or non natural amino acid residue different from L- or D-Cys residue, such as L- or D-Ala, Gly, or D-L-Phe
  MOD_RES (2): L- or D-Thr, L- or D-Ser, L- or D-Tyr, L- or D-Trp, or L- or D-His
  MOD_RES (3): any natural or non natural amino acid residue different from L- or D-Glx, L- or D-Asx, L- or D-Arg, L- or D-Lys, L- or D-His, L- or D-Tyr, L- or D-Ser, L- or D-Thr, such as L- or D-Glu, or L- or D-Asp
  MOD_RES (4): any natural or non natural amino acid residue different from L- or D-Cys residue, such as L- or D-Leu, L- or D-Ile, L- or D-L- or D-Val, or L- or D-Ala, L- or D-Phe, or L- or D-Tyr
  MOD_RES (5): any natural or non natural amino acid residue different from L- or D-Cys residue, such as L- or D-Arg, L- or D-Lys, L- or D-Orn- or D-Ala
  MOD_RES (6): L- or D-Cys, L- or D-Ala, L- or D-Phe, L- or D-His, L- or D-Trp, L- or D-Ser, L- or D-Thr, L- or D-Lys, L- or D-His, or L- or D-Tyr
  MOD_RES (7): any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, or L- or D-Trp
  MOD_RES (8): L- or D-Cys, L- or D-Ala, L- or D-Phe, L- or D-His, L- or D-Trp, L- or D-Ser, L- or D-Thr, L- or D-Lys, L- or D-His, or L- or D-Tyr
  MOD_RES (9): any natural or non natural amino acid residue different from L- or D-Cys residue, such as L- or D-Leu, L- or D-Ile, L- or D-L- or D-Val, or L- or D-Ala, L- or D-Phe, or L- or D-Tyr
  MOD_RES (10): any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, or L- or D-Trp
  MOD_RES (11): L- or D-Thr, L- or D-Ser, L- or D-Tyr, L- or D-Trp, or L- or D-His
  MOD_RES (12): any natural or non natural amino acid residue different from L- or D-Cys residue, such as L- or D-Leu, L- or D-Ile, L- or D-L- or D-Val, or L- or D-Ala, L- or D-Phe, or L- or D-Tyr
  MOD_RES (13)..(18): one or up to six natural or non natural amino acid residue different from L- or D-Cys residue; see specification as filed for detailed description of substitutions and preferred embodiments
  MOD_RES (19)..(38): region may encompass 1-20 variable amino acids
  MOD_RES (39): any natural or non natural amino acid residue different from L- or D-Cys residue, such as L- or D-Ile, L- or D-Leu, L- or D-L- or D-Val, or L- or D-Ala, L- or D-Phe, L- or D-Tyr, or L- or D- Lys
  MOD_RES (40): L- or D-Val, L- or D-Leu, L- or D-Ile, L- or D-Pro, L- or D-Phe, L- or D-Tyr, L- or D-Ser, L- or D-Thr, or L- or D-Lys
  MOD_RES (41): any natural or non natural amino acid residue different from L- or D-Cys residue, such as L- or D-Lys, L- or D-Arg, L- or D-Orn- or D-Ala, or L- or D-Ile
  MOD_RES (42): any natural or non natural amino acid residue different from L- or D-Cys residue, such as L- or D-Lys, L- or D-Arg, L- or D-Orn- or D-Ala, or L- or D-Ile
  MOD_RES (43): any natural or non natural amino acid residue different from L- or D-Cys residue, such as L- or D-Ile, L- or D-Leu, L- or D-L- or D-Val, or L- or D-Ala, L- or D-Phe, L- or D-Tyr, or L- or D-Glu
  MOD_RES (44): any natural or non natural amino acid residue different from L- or D-Cys residue, such as L- or D-Ile, L- or D-Leu, L- or D-L- or D-Val, or L- or D-Ala, L- or D-Phe, L- or D-Tyr, L- or D-Glu, or L- or D-Lys
  MOD_RES (45): any natural or non natural amino acid residue different from L- or D-Glx, L- or D-Asx, L- or D-Arg, L- or D-Lys, L- or D-His, L- or D-Tyr, L- or D-Ser, L- or D-Thr, L- or D-Met, or L- or D-Nle
  MOD_RES (46): any natural or non natural amino acid residue different from L- or D-Lys, L- or D-Met, L- or D-Arg, L- or D-Asx, L- or D-Glx, L- or D-His, L- or D-Tyr, L- or D-Ser, L- or D-Thr, L- or D-Leu, or L- or D-Ile
  MOD_RES (47): L- or D-Met, L- or D-Leu, L- or D-Asx, L- or D-Nle, L- or D-Ala, L- or D-Phe, L- or D-Glx, L- or D-Ile, or L- or D-Tyr
  MOD_RES (48): any natural or non natural amino acid residue different from L- or D-Cys residue, such as L- or D-Leu, L- or D-Ile, L- or D-L- or D-Asx, or L- or D-Glx, L- or D-Ala, L- or D-Val, L- or D-Ser, L- or D-Thr, or L- or D-Tyr
  MOD_RES (49): L- or D-Asx, L- or D-Glx, L- or D-Ser, L- or D-Thr, L- or D-Tyr, L- or D-Lys, or L or D-Arg
  MOD_RES (50): any natural or non natural amino acid different from L- or D-Cys, such as L- or D-Ser, L- or D-Asx, L- or D-Glx, or L- or D-Lys
  MOD_RES (51): L-or D-Glx, L- or D-Asx, L- or D-Lys, L- or D- Ser, L- or D-Thr, L- or D-Tyr, or L- or Arg
  MOD_RES (52)..(54): one or up to three natural or non natural amino acid residue different from L- or D-Cys residue, such as L- or D-Lys, L- or D-Arg, L- or D-Ser, L- or D- Asn, L- or D-Orn-, or D-Ala, and L- or D-(Lys-Ser), L- or D-(Lys-Ser-Asn)
XXXXXXXXXXXXXXXXXXXXXXXXXXXXXXXXXXXXXXXXXXXXXXXXXXXXXX

SEQ ID NO: 20 - protein, 53 aa, Artificial Sequence
  Description of Artificial Sequence: Synthetic peptide
  MOD_RES (1): L- or D-Thr, L- or D-Ser, L- or D-Tyr, L- or D-Trp, or L- or D-His
  MOD_RES (2): any natural or non natural amino acid residue different from L- or D-Glx, L- or D-Asx, L- or D-Arg, L- or D-Lys, L- or D-His, L- or D-Tyr, L- or D-Ser, L- or D-Thr, such as L- or D-Glu, or L- or D-Asp
  MOD_RES (3): any natural or non natural amino acid residue different from L- or D-Cys residue, such as L- or D-Leu, L- or D-Ile, L- or D-L- or D-Val, or L- or D-Ala, L- or D-Phe, or L- or D-Tyr
  MOD_RES (4): any natural or non natural amino acid residue different from L- or D-Cys residue, such as L- or D-Arg, L- or D-Lys, L- or D-Orn- or D-Ala
  MOD_RES (5): L- or D-Cys, L- or D-Ala, L- or D-Phe, L- or D-His, L- or D-Trp, L- or D-Ser, L- or D-Thr, L- or D-Lys, L- or D-His, or L- or D-Tyr
  MOD_RES (6): any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, or L- or D-Trp
  MOD_RES (7): L- or D-Cys, L- or D-Ala, L- or D-Phe, L- or D-His, L- or D-Trp, L- or D-Ser, L- or D-Thr, L- or D-Lys, L- or D-His, or L- or D-Tyr
  MOD_RES (8): any natural or non natural amino acid residue different from L- or D-Cys residue, such as L- or D-Leu, L- or D-Ile, L- or D-L- or D-Val, or L- or D-Ala, L- or D-Phe, or L- or D-Tyr
  MOD_RES (9): any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, or L- or D-Trp
  MOD_RES (10): L- or D-Thr, L- or D-Ser, L- or D-Tyr, L- or D-Trp, or L- or D-His
  MOD_RES (11): any natural or non natural amino acid residue different from L- or D-Cys residue, such as L- or D-Leu, L- or D-Ile, L- or D-L- or D-Val, or L- or D-Ala, L- or D-Phe, or L- or D-Tyr
  MOD_RES (12)..(17): one or up to six natural or non natural amino acid residue different from L- or D-Cys residue; see specification as filed for detailed description of substitutions and preferred embodiments
  MOD_RES (18)..(37): region may encompass 1-20 variable amino acids
  MOD_RES (38): any natural or non natural amino acid residue different from L- or D-Cys residue, such as L- or D-Ile, L- or D-Leu, L- or D-L- or D-Val, or L- or D-Ala, L- or D-Phe, L- or D-Tyr, or L- or D- Lys
  MOD_RES (39): L- or D-Val, L- or D-Leu, L- or D-Ile, L- or D-Pro, L- or D-Phe, L- or D-Tyr, L- or D-Ser, L- or D-Thr, or L- or D-Lys
  MOD_RES (40): any natural or non natural amino acid residue different from L- or D-Cys residue, such as L- or D-Lys, L- or D-Arg, L- or D-Orn- or D-Ala, or L- or D-Ile
  MOD_RES (41): any natural or non natural amino acid residue different from L- or D-Cys residue, such as L- or D-Lys, L- or D-Arg, L- or D-Orn- or D-Ala, or L- or D-Ile
  MOD_RES (42): any natural or non natural amino acid residue different from L- or D-Cys residue, such as L- or D-Ile, L- or D-Leu, L- or D-L- or D-Val, or L- or D-Ala, L- or D-Phe, L- or D-Tyr, or L- or D-Glu
  MOD_RES (43): any natural or non natural amino acid residue different from L- or D-Cys residue, such as L- or D-Ile, L- or D-Leu, L- or D-L- or D-Val, or L- or D-Ala, L- or D-Phe, L- or D-Tyr, L- or D-Glu, or L- or D-Lys
  MOD_RES (44): any natural or non natural amino acid residue different from L- or D-Glx, L- or D-Asx, L- or D-Arg, L- or D-Lys, L- or D-His, L- or D-Tyr, L- or D-Ser, L- or D-Thr, L- or D-Met, or L- or D-Nle
  MOD_RES (45): any natural or non natural amino acid residue different from L- or D-Lys, L- or D-Met, L- or D-Arg, L- or D-Asx, L- or D-Glx, L- or D-His, L- or D-Tyr, L- or D-Ser, L- or D-Thr, L- or D-Leu, or L- or D-Ile
  MOD_RES (46): L- or D-Met, L- or D-Leu, L- or D-Asx, L- or D-Nle, L- or D-Ala, L- or D-Phe, L- or D-Glx, L- or D-Ile, or L- or D-Tyr
  MOD_RES (47): any natural or non natural amino acid residue different from L- or D-Cys residue, such as L- or D-Leu, L- or D-Ile, L- or D-L- or D-Asx, or L- or D-Glx, L- or D-Ala, L- or D-Val, L- or D-Ser, L- or D-Thr, or L- or D-Tyr
  MOD_RES (48): L- or D-Asx, L- or D-Glx, L- or D-Ser, L- or D-Thr, L- or D-Tyr, L- or D-Lys, or L or D-Arg
  MOD_RES (49): any natural or non natural amino acid different from L- or D-Cys, such as L- or D-Ser, L- or D-Asx, L- or D-Glx, or L- or D-Lys
  MOD_RES (50): L-or D-Glx, L- or D-Asx, L- or D-Lys, L- or D- Ser, L- or D-Thr, L- or D-Tyr, or L- or Arg
  MOD_RES (51)..(53): one or up to three natural or non natural amino acid residue different from L- or D-Cys residue, such as L- or D-Lys, L- or D-Arg, L- or D-Ser, L- or D- Asn, L- or D-Orn-, or D-Ala, and L- or D-(Lys-Ser), L- or D-(Lys-Ser-Asn)
XXXXXXXXXXXXXXXXXXXXXXXXXXXXXXXXXXXXXXXXXXXXXXXXXXXXX

SEQ ID NO: 21 - protein, 52 aa, Artificial Sequence
  Description of Artificial Sequence: Synthetic peptide
  MOD_RES (1): any natural or non natural amino acid residue different from L- or D-Glx, L- or D-Asx, L- or D-Arg, L- or D-Lys, L- or D-His, L- or D-Tyr, L- or D-Ser, L- or D-Thr, such as L- or D-Glu, or L- or D-Asp
  MOD_RES (2): any natural or non natural amino acid residue different from L- or D-Cys residue, such as L- or D-Leu, L- or D-Ile, L- or D-L- or D-Val, or L- or D-Ala, L- or D-Phe, or L- or D-Tyr
  MOD_RES (3): any natural or non natural amino acid residue different from L- or D-Cys residue, such as L- or D-Arg, L- or D-Lys, L- or D-Orn- or D-Ala
  MOD_RES (4): L- or D-Cys, L- or D-Ala, L- or D-Phe, L- or D-His, L- or D-Trp, L- or D-Ser, L- or D-Thr, L- or D-Lys, L- or D-His, or L- or D-Tyr
  MOD_RES (5): any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, or L- or D-Trp
  MOD_RES (6): L- or D-Cys, L- or D-Ala, L- or D-Phe, L- or D-His, L- or D-Trp, L- or D-Ser, L- or D-Thr, L- or D-Lys, L- or D-His, or L- or D-Tyr
  MOD_RES (7): any natural or non natural amino acid residue different from L- or D-Cys residue, such as L- or D-Leu, L- or D-Ile, L- or D-L- or D-Val, or L- or D-Ala, L- or D-Phe, or L- or D-Tyr
  MOD_RES (8): any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, or L- or D-Trp
  MOD_RES (9): L- or D-Thr, L- or D-Ser, L- or D-Tyr, L- or D-Trp, or L- or D-His
  MOD_RES (10): any natural or non natural amino acid residue different from L- or D-Cys residue, such as L- or D-Leu, L- or D-Ile, L- or D-L- or D-Val, or L- or D-Ala, L- or D-Phe, or L- or D-Tyr
  MOD_RES (11)..(16): one or up to six natural or non natural amino acid residue different from L- or D-Cys residue; see specification as filed for detailed description of substitutions and preferred embodiments
  MOD_RES (17)..(36): region may encompass 1-20 variable amino acids
  MOD_RES (37): any natural or non natural amino acid residue different from L- or D-Cys residue, such as L- or D-Ile, L- or D-Leu, L- or D-L- or D-Val, or L- or D-Ala, L- or D-Phe, L- or D-Tyr, or L- or D- Lys
  MOD_RES (38): L- or D-Val, L- or D-Leu, L- or D-Ile, L- or D-Pro, L- or D-Phe, L- or D-Tyr, L- or D-Ser, L- or D-Thr, or L- or D-Lys
  MOD_RES (39): any natural or non natural amino acid residue different from L- or D-Cys residue, such as L- or D-Lys, L- or D-Arg, L- or D-Orn- or D-Ala, or L- or D-Ile
  MOD_RES (40): any natural or non natural amino acid residue different from L- or D-Cys residue, such as L- or D-Lys, L- or D-Arg, L- or D-Orn- or D-Ala, or L- or D-Ile
  MOD_RES (41): any natural or non natural amino acid residue different from L- or D-Cys residue, such as L- or D-Ile, L- or D-Leu, L- or D-L- or D-Val, or L- or D-Ala, L- or D-Phe, L- or D-Tyr, or L- or D-Glu
  MOD_RES (42): any natural or non natural amino acid residue different from L- or D-Cys residue, such as L- or D-Ile, L- or D-Leu, L- or D-L- or D-Val, or L- or D-Ala, L- or D-Phe, L- or D-Tyr, L- or D-Glu, or L- or D-Lys
  MOD_RES (43): any natural or non natural amino acid residue different from L- or D-Glx, L- or D-Asx, L- or D-Arg, L- or D-Lys, L- or D-His, L- or D-Tyr, L- or D-Ser, L- or D-Thr, L- or D-Met, or L- or D-Nle
  MOD_RES (44): any natural or non natural amino acid residue different from L- or D-Lys, L- or D-Met, L- or D-Arg, L- or D-Asx, L- or D-Glx, L- or D-His, L- or D-Tyr, L- or D-Ser, L- or D-Thr, L- or D-Leu, or L- or D-Ile
  MOD_RES (45): L- or D-Met, L- or D-Leu, L- or D-Asx, L- or D-Nle, L- or D-Ala, L- or D-Phe, L- or D-Glx, L- or D-Ile, or L- or D-Tyr
  MOD_RES (46): any natural or non natural amino acid residue different from L- or D-Cys residue, such as L- or D-Leu, L- or D-Ile, L- or D-L- or D-Asx, or L- or D-Glx, L- or D-Ala, L- or D-Val, L- or D-Ser, L- or D-Thr, or L- or D-Tyr
  MOD_RES (47): L- or D-Asx, L- or D-Glx, L- or D-Ser, L- or D-Thr, L- or D-Tyr, L- or D-Lys, or L or D-Arg
  MOD_RES (48): any natural or non natural amino acid different from L- or D-Cys, such as L- or D-Ser, L- or D-Asx, L- or D-Glx, or L- or D-Lys
  MOD_RES (49): L-or D-Glx, L- or D-Asx, L- or D-Lys, L- or D- Ser, L- or D-Thr, L- or D-Tyr, or L- or Arg
  MOD_RES (50)..(52): one or up to three natural or non natural amino acid residue different from L- or D-Cys residue, such as L- or D-Lys, L- or D-Arg, L- or D-Ser, L- or D- Asn, L- or D-Orn-, or D-Ala, and L- or D-(Lys-Ser), L- or D-(Lys-Ser-Asn)
XXXXXXXXXXXXXXXXXXXXXXXXXXXXXXXXXXXXXXXXXXXXXXXXXXXX

SEQ ID NO: 22 - protein, 50 aa, Artificial Sequence
  Description of Artificial Sequence: Synthetic peptide
  MOD_RES (17)..(36): region may encompass 1-20 variable amino acids
ASVATELRCQCLQTLQXXXXXXXXXXXXXXXXXXXXIVKKIIEKMLNSDK

SEQ ID NO: 23 - protein, 50 aa, Artificial Sequence
  Description of Artificial Sequence: Synthetic peptide
  MOD_RES (17)..(36): region may encompass 1-20 variable amino acids
ASVATELRCQCLQTLQXXXXXXXXXXXXXXXXXXXXVKKIIEKMLNSDKS

SEQ ID NO: 24 - protein, 50 aa, Artificial Sequence
  Description of Artificial Sequence: Synthetic peptide
  MOD_RES (17)..(36): region may encompass 1-20 variable amino acids
ASVATELRCQCLQTLQXXXXXXXXXXXXXXXXXXXXKKIIEKMLNSDKSN

SEQ ID NO: 25 - protein, 50 aa, Artificial Sequence
  Description of Artificial Sequence: Synthetic peptide
  MOD_RES (17)..(36): region may encompass 1-20 variable amino acids
ATELRCQCLQTLQGIHXXXXXXXXXXXXXXXXXXXXIVKKIIEKMLNSDK

SEQ ID NO: 26 - protein, 50 aa, Artificial Sequence
  Description of Artificial Sequence: Synthetic peptide
  MOD_RES (17)..(36): region may encompass 1-20 variable amino acids
TELRCQCLQTLQGIHPXXXXXXXXXXXXXXXXXXXXIVKKIIEKMLNSDK

SEQ ID NO: 27 - protein, 50 aa, Artificial Sequence
  Description of Artificial Sequence: Synthetic peptide
  MOD_RES (17)..(36): region may encompass 1-20 variable amino acids
ELRCQCLQTLQGIHPKXXXXXXXXXXXXXXXXXXXXIVKKIIEKMLNSDK

SEQ ID NO: 28 - protein, 50 aa, Artificial Sequence; identical to SEQ ID NO: 27
  Description of Artificial Sequence: Synthetic peptide
  MOD_RES (17)..(36): region may encompass 1-20 variable amino acids

SEQ ID NO: 29 - protein, 52 aa, Artificial Sequence; identical to SEQ ID NO: 21
  Description of Artificial Sequence: Synthetic peptide
  MOD_RES (1): L- or D-Thr, L- or D-Ser, L- or D-Tyr, L- or D-Trp, L- or D-His, L- or D-Glx, L- or D-Asx, L- or D-Leu, L- or D-Ile, or L- or D-Ala
  MOD_RES (2): any natural or non natural amino acid residue different from L- or D-Glx, L- or D-Asx, L- or D-Arg, L- or D-Lys, L- or D-His, L- or D-Arg, L- or D-Ser, or L- or D-Thr
  MOD_RES (3): any natural or non natural amino acid residue different from L- or D-Cys residue, such as L- or D-Leu, L- or D-Ile, L- or D-L- or D-Val, or L- or D-Ala, L- or D-Phe, or L- or D-Tyr
  MOD_RES (4): any natural or non natural amino acid residue different from L- or D-Cys residue, such as L- or D-Arg, L- or D-Lys, L- or D-Orn-, or D-Ala
  MOD_RES (5): L- or D-Cys, L- or D-Ala, L- or D-Phe, L- or D-His, L- or D-Trp, L- or D-Ser, L- or D-Thr, L- or D-Lys, L- or D-His, or L- or D-Tyr
  MOD_RES (6): any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, or L- or D-Trp
  MOD_RES (7): L- or D-Cys, L- or D-Ala, L- or D-Phe, L- or D-His, L- or D-Trp, L- or D-Ser, L- or D-Thr, L- or D-Lys, L- or D-His, or L- or D-Tyr
  MOD_RES (8): any natural or non natural amino acid residue different from L- or D-Cys residue, such as L- or D-Leu, L- or D-Ile, L- or D-L- or D-Val, or L- or D-Ala, L- or D-Phe, or L- or D-Tyr
  MOD_RES (9): any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, or L- or D-Trp
  MOD_RES (10): L- or D-Thr, L- or D-Ser, L- or D-Tyr, L- or D-Trp, or L- or D-His
  MOD_RES (11): any natural or non natural amino acid residue different from L- or D-Cys residue, such as L- or D-Leu, L- or D-Ile, L- or D-L- or D-Val, or L- or D-Ala, L- or D-Phe, or L- or D-Tyr
  MOD_RES (12): any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, or L- or D-Trp
  MOD_RES (13): Gly, L- or D-Ala, L- or D-Val, L- or D-Leu, or L- or D-Ile
  MOD_RES (14): any natural or non natural amino acid residue different from L- or D-Cys residue, such as L- or D-Ile, L- or D-Leu, L- or D-L- or D-Val, or L- or D-Ala, L- or D-Phe, or L- or D-Tyr
  MOD_RES (15): L- or D-His, L- or D-Trp, L- or D-Tyr, L- or D-Arg, L- or D-Lys, or L- or D-Phe
  MOD_RES (16): any natural or non natural amino acid residue different from L- or D-Cys residue, such as L- or D-Leu, L- or D-Ile, L- or D-L- or D-Val, or L- or D-Ala, L- or D-Phe, or L- or D-Tyr
  MOD_RES (17)..(36): region may encompass 1-20 variable amino acids
  MOD_RES (37): L- or D-Val, L- or D-Leu, L- or D-Ile, L- or D-Pro, L- or D-Phe, L- or D-Tyr, L- or D-Ser, L- or D-Thr, L- or D-Met, L- or D-Lys, L- or D-Arg, or L- or D-Orn
  MOD_RES (38): any natural or non natural amino acid residue different from L- or D-Cys residue, such as L- or D-Lys, L- or D-Arg, L- or D-Orn-, or D-Val
  MOD_RES (39): any natural or non natural amino acid residue different from L- or D-Cys residue, such as L- or D-Lys, L- or D-Arg, L- or D-Ile, or L- or D-Leu
  MOD_RES (40): any natural or non natural amino acid residue different from L- or D-Cys residue, such as L- or D-Ile, L- or D-Leu, L- or D-L- or D-Lys, or L- or D-Ala, L- or D-Phe, or L- or D-Tyr
  MOD_RES (41): any natural or non natural amino acid residue different from L- or D-Cys residue, such as L- or D-Ile, L- or D-Ieu, L- or D-L- or D-Glx, or L- or D-Asx, L- or D-Phe, or L- or D-Tyr
  MOD_RES (42): any natural or non natural amino acid residue different from L- or D-Glx, L- or D-Asx, L- or D-Arg, L- or D-Lys, L- or D-His, L- or D-Tyr, L- or D-Ser, L- or D-Thr, or L- or D-Ile
  MOD_RES (43): any natural or non natural amino acid residue different from L- or D-Cys residue, such as L- or D-Lys, L- or D-Arg, L- or D-Glu-, or D-Ile
  MOD_RES (44): L- or D-Met, L- or D-Ile, L- or D-Nle, L- or D-Ala, L- or D-Phe, L- or D-Leu, L- or D-Lys, or L-or D-Arg
  MOD_RES (45): any natural or non natural amino acid residue different from L- or D-Cys residue, such as  L- or D-Leu, L- or D-Ile, L- or D-L- or D-Val, or L- or D-Ala, L- or D-Phe, L- or D-Lys, or L- or D-Arg
  MOD_RES (46): any natural or non natural amino acid residue different from L- or D-Cys residu or D-Leu, L- or D-Ile, such as L- or D-Lys, L- or D-Arg, L- or D-Leu, L- or D-Asx, or L- or D-Glx
  MOD_RES (47): L- or D-Asx, L- or D-Glx, L- or D-Arg, L- or D-Lys, L- or D-Ala, L- or D-Leu, or L or D-Ile
  MOD_RES (48): Gly, L- or D-Asx, L- or D-Glx, L- or D-Leu, L- or D-Ile, or L- or D-Ala
  MOD_RES (49): is any natural or non natural amino acid residue different from L- or D-Cys residue, such as L- or D-Lys, L- or D-Arg, L- or D-Ser-, or L- or D-Gly
  MOD_RES (50)..(53): one or up to three natural or non natural amino acid residue different from L- or D-Cys residue, such as L- or D-Lys, L- or D-Arg, L- or D-Ser, L- or D- Asn, L- or D-Orn-, or D-Ala, and L- or D-Lys, L- or D-(Lys-Ser), or L- or D-( Lys-Ser-Asn)

SEQ ID NO: 30 - protein, 50 aa, Artificial Sequence
  Description of Artificial Sequence: Synthetic peptide
  MOD_RES (17)..(36): region may encompass 1-20 variable amino acids
TELRCQCLQTLQGIHLXXXXXXXXXXXXXXXXXXXXMVKKIIEKMLKNGK

SEQ ID NO: 31 - protein, 50 aa, Artificial Sequence
  Description of Artificial Sequence: Synthetic peptide
  MOD_RES (17)..(36): region may encompass 1-20 variable amino acids
TELRCQCLQTLQGIHLXXXXXXXXXXXXXXXXXXXXVKKIIEKMLKNGKS

SEQ ID NO: 32 - protein, 50 aa, Artificial Sequence
  Description of Artificial Sequence: Synthetic peptide
  MOD_RES (17)..(36): region may encompass 1-20 variable amino acids
TELRCQCLQTLQGIHLXXXXXXXXXXXXXXXXXXXXKKIIEKMLKNGKSN

SEQ ID NO: 33 - protein, 50 aa, Artificial Sequence; identical to SEQ ID NO: 32
  Description of Artificial Sequence: Synthetic peptide
  MOD_RES (17)..(36): region may encompass 1-20 variable amino acids

SEQ ID NO: 34 - protein, 50 aa, Artificial Sequence
  Description of Artificial Sequence: Synthetic peptide
  MOD_RES (17)..(36): region may encompass 1-20 variable amino acids
TELRCQCLQTLQGIHLXXXXXXXXXXXXXXXXXXXXVQKIIEKILNKGST

SEQ ID NO: 35 - protein, 52 aa, Artificial Sequence; identical to SEQ ID NO: 21
  Description of Artificial Sequence: Synthetic peptide
  MOD_RES (1): L- or D-Thr, L- or D-Ser, L- or D-Tyr, L- or D-Trp, L- or D-His, L- or D-Glx, L- or D-Asx, L- or D-Leu, L- or D-Ile, or L- or D-Ala
  MOD_RES (2): any natural or non natural amino acid residue different from L- or D-Glx, L- or D-Asx, L- or D-Arg, L- or D-Lys, L- or D-His, L- or D-Arg, L- or D-Ser, L- or D-Thr
  MOD_RES (3): any natural or non natural amino acid residue different from L- or D-Cys residue, such as  L- or D-Leu, L- or D-Ile, L- or D-L- or D-Val, or L- or D-Ala, L- or D-Phe, L- or D-Tyr
  MOD_RES (4): any natural or non natural amino acid residue different from L- or D-Cys residue, such as L- or D-Arg, L- or D-Lys, L- or D-Orn- or D-Ala
  MOD_RES (5): L- or D-Cys, L- or D-Ala, L- or D-Phe, L- or D-His, L- or D-Trp, L- or D-Ser, L- or D-Thr, L- or D-Lys, L- or D-His, L- or D-Tyr
  MOD_RES (6): any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, L- or D-Trp
  MOD_RES (7): L- or D-Cys, L- or D-Ala, L- or D-Phe, L- or D-His, L- or D-Trp, L- or D-Ser, L- or D-Thr, L- or D-Lys, L- or D-His, L- or D-Tyr
  MOD_RES (8): any natural or non natural amino acid residue different from L- or D-Cys residue, such as  L- or D-Leu, L- or D-Ile, L- or D-L- or D-Val, or L- or D-Ala, L- or D-Phe, L- or D-Tyr
  MOD_RES (9): any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, L- or D-Trp
  MOD_RES (10): L- or D-Thr, L- or D-Ser, L- or D-Tyr, L- or D-Trp, L- or D-His; L- or D-Ser, L- or D-Tyr
  MOD_RES (11): any natural or non natural amino acid residue different from L- or D-Cys residue, such as  L- or D-Leu, L- or D-Ile, L- or D-L- or D-Val, or L- or D-Ala, L- or D-Phe, L- or D-Tyr
  MOD_RES (12): any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, L- or D-Trp
  MOD_RES (13): Gly, L- or D-Ala, L- or D-Val, L- or D-Leu, L- or D-Ile
  MOD_RES (14): any natural or non natural amino acid residue different from L- or D-Cys residue, such as  L- or D-Ile, L- or D-Leu, L- or D-L- or D-Val, or L- or D-Ala, L- or D-Phe, L- or D-Tyr
  MOD_RES (15): L- or D-His, L- or D-Trp, L- or D-Tyr, L- or D-Arg, L- or D-Lys, L- or D-Phe
  MOD_RES (16): any natural or non natural amino acid residue different from L- or D-Cys residue, such as  L- or D-Leu, L- or D-Ile, L- or D-L- or D-Val, or L- or D-Ala, L- or D-Phe, L- or D-Tyr
  MOD_RES (17)..(36): region may encompass 1-20 variable amino acids
  MOD_RES (37): L- or D-Val, L- or D-Leu, L- or D-Ile, L- or D-Pro, L- or D-Phe, L- or D-Tyr, L- or D-Ser, L- or D-Thr, L- or D-Met
  MOD_RES (38): any natural or non natural amino acid residue different from L- or D-Cys residue, such as Ls- or D-Lys, L- or D-Arg, L- or D-Orn- or D-Val
  MOD_RES (39): any natural or non natural amino acid residue different from L- or D-Cys residue, such as L- or D-Glx, L- or D-Asx, L- or D-Lys, L- or D-Ile
  MOD_RES (40): any natural or non natural amino acid residue different from L- or D-Cys residue, such as  L- or D-Ile, L- or D-Leu, L- or D-L- or D-Lys, or L- or D-Ala, L- or D-Phe, L- or D-Tyr
  MOD_RES (41): any natural or non natural amino acid residue different from L- or D-Cys residue, such as  L- or D-Ile, L- or D-Leu, L- or D-L- or D-Glx, or L- or D-Ala, L- or D-Phe, L- or D-Tyr
  MOD_RES (42): any natural or non natural amino acid residue different from L- or D-Glx, L- or D-Asx, L- or D-Arg, L- or D-Lys, L- or D-His, L- or D-Tyr, L- or D-Ser, L- or D-Thr, L- or D-Ile; L- or D-Glu, L- or D-Lys
  MOD_RES (43): any natural or non natural amino acid residue different from L- or D-Cys residue, such as L- or D-Lys, L- or D-Arg, L- or D-Glu- or D-Ile
  MOD_RES (44): L- or D-Lys, L- or D-Ile, L- or D-Leu, L- or D-Ala, L- or D-Phe, L- or D-Tyr, L- or D-Ser, L-or D-Arg, L- or D-Orn
  MOD_RES (45): any natural or non natural amino acid residue different from L- or D-Cys residue, such as  L- or D-Leu, L- or D-Ile, L- or D-L- or D-Asx, or L- or D-Glx, L- or D-Phe, L- or D-Tyr, L- or D-Nle
  MOD_RES (46): any natural or non natural amino acid residue different from L- or D-Cys residu or D-Leu, L- or D-Ile, such as L- or D-Lys, L- or D-Arg, L- or D-Leu- or D-Asx, L-or D-Glx
  MOD_RES (47): L- or D-Asx, L- or D-Glx, L- or D-Arg, L- or D-Lys, L- or D-Ala, L- or D-Orn, Gly
  MOD_RES (48): any natural or non natural amino acid residue different from L- or D-Cys residue or Gly, L- or D-Asx, L- or D-Glx, L- or D-Ser, L- or D-Tyr, L- or D-Thr
  MOD_RES (49): any natural or non natural amino acid residue different from L- or D-Cys residue, such as Gly, L- or D-Ser, L- or D-Thr
  MOD_RES (50)..(52): one or up to three natural or non natural amino acid different from L- or D-Cys, such as L- or D-Asx, L-or D-Glx, L- or D-Ser, L- or D-Thr, L- or D-Tyr, L- or D-Lys, and L- or D-Ser, L- or D-(Ser-Thr), L- or D-(Ser-Thr-Asn)

SEQ ID NO: 36 - protein, 50 aa, Artificial Sequence
  Description of Artificial Sequence: Synthetic peptide
  MOD_RES (17)..(36): region may encompass 1-20 variable amino acids
TELRCQCLQTLQGIHLXXXXXXXXXXXXXXXXXXXXQKIIEKILNKGSTN

SEQ ID NO: 37 - protein, 50 aa, Artificial Sequence; identical to SEQ ID NO: 36
  Description of Artificial Sequence: Synthetic peptide
  MOD_RES (17)..(36): region may encompass 1-20 variable amino acids

SEQ ID NO: 38 - protein, 51 aa, Artificial Sequence
  Description of Artificial Sequence: Synthetic peptide
  MOD_RES (1): optional natural or non natural amino acid residue different from L- or D-Cys residue, such as L- or D-Glx, L- or  D-Asx
  MOD_RES (2): any natural or non natural amino acid residue different from L- or D-Cys, L- or D-Ala, L- or D-Val, L- or D-Ile, L- or D-Leu
  MOD_RES (3): L- or D-Glx, L- or D-Asx, L- or D-Ala, L- or D-Pro, L- or D-Phe, L- or D-Tyr, L- or D-Ser, L- or D-Thr
  MOD_RES (4): any natural or non natural amino acid residue different from L- or D-Cys residue, such as L- or D-Glx, L- or D-Asx, L- or D,L- or D-Ala, L- or D-Val, L- or D-Phe
  MOD_RES (5): L- or D- Asx, L- or D-Glx, L- or D-Ala, L- or D-Phe, L- or D-Tyr
  MOD_RES (6): any natural or non-natural amino acid residue different from L- or D-Cys, such as  L- or D-Ala,  L- or D-Val, L- or D-Leu, L- or D-Ile, L- or D-Phe, L- or D-Tyr, L- or D-Glx
  MOD_RES (7): L- or D- Asx, L- or D-Glx, L- or D-Ala, L- or D-Phe, L- or D-Tyr
  MOD_RES (8): any natural or non-natural amino acid residue different from L- or D-Cys, such as L- or D-Leu, L- or D-Ala, L- or D,L- or D-Val, L- or D-Leu, L- or D-Ile, L- or D-Phe, L- or D-Tyr
  MOD_RES (9): L- or D-Glx, L- or D-Asx, L- or D-Arg, L- or D-Lys, L- or D-His, L- or D-Tyr, L- or D-Ser, L- or D-Thr
  MOD_RES (10): L- or D-Cys, L- or D-Ala, L- or D-Phe, L- or D-His, L- or D-Trp, L- or D-Ser, L- or D-Thr
  MOD_RES (11): any natural or non-natural amino acid residue different from L- or D-Cys, such as L- or D-Glx, L- or D-Asx, L- or D-Gly, L- or D-Val, L- or D-Leu, L- or D-Ile, L- or D-Pro, L- or D-Phe, L- or D-Tyr, L- or D-His, L- or D-Trp
  MOD_RES (12): L- or D-Cys, L- or D-Ala, L- or D-Phe, L- or D-His, L- or D-Trp, L- or D-Ser, L- or D-Thr, L- or D-Lys, L- or D-His, L- or D-Tyr
  MOD_RES (13): L- or D-Val, L- or D-Leu, L- or D-Ile, L- or D-Ala, L- or D-Phe, L- or D-Tyr
  MOD_RES (14): optional, and may be any natural or non-natural amino acid residue different from L- or D-Cys, such as L- or D-Lys, L- or D-Arg, L- or D-His
  MOD_RES (15): optional and may be L- or D-Thr, L- or D-Ser, L- or D-Tyr, L- or D-Ala, L- or D-Phe
  MOD_RES (16): optional and may be any natural or non-natural amino acid residue different from L- or D-Cys, such as L- or D-Thr, L- or D-Ser, L- or D-Tyr, L- or D-Ala, L- or D-Phe
  MOD_RES (17): optional and may be any natural or non-natural amino acid residue different from L- or D-Cys, such as L- or D-Thr, L- or D-Ser, L- or D-Tyr, L- or D-Ala, L- or D-Phe
  MOD_RES (18)..(37): region may encompass 1-20 variable amino acids
  MOD_RES (38): any natural or non natural amino acid residue different from L- or D-Cys residue, such as L- or D-Ala, L- or D-Val, L- or D-Ile, L- or D-Leu
  MOD_RES (39): L- or D-Pro, L- or D-Ala
  MOD_RES (40): any natural or non natural amino acid residue different from L- or D-Cys, such as L- or D-Leu, L- or D-Ala, L- or D,L- or D-Val, L- or D-Leu, L- or D-Ile, L- or D-Phe, L- or D-Tyr, L- or D-Ser, L- or D-Thr, L- or D-Phe
  MOD_RES (41): L- or D-Tyr, L- or D-Ser, L- or D-Thr, L- or D-Phe
  MOD_RES (42): any natural or non natural amino acid residue different from L- or D-Cys, such as L- or D-Lys, L- or D-Arg, L- or D-His
  MOD_RES (43): any natural or non natural amino acid residue different from L- or D-Cys, such as L- or D-Lys, L- or D-Arg, L- or D-His
  MOD_RES (44): any natural or non natural amino acid residue different from L- or D-Cys, such as L- or D-Glx, L- or D-Asx, L- or D-Gly, L- or D-Val, L- or D-Leu, L- or D-Ile, L- or D-Pro, L- or D-Phe, L- or D-Tyr, L- or D-His, L- or D-Trp
  MOD_RES (45): any natural or non natural amino acid residue different from L- or D-Cys, such as L- or D-Glx, L- or D-Asx, L- or D-Gly, L- or D-Val, L- or D-Leu, L- or D-Ile, L- or D-Pro, L- or D-Phe, L- or D-Tyr, L- or D-His, L- or D-Trp
  MOD_RES (46): any natural or non natural amino acid residue different from L- or D-Cys, such as L- or D-Lys, L- or D-Arg, L- or D-His
  MOD_RES (47): any natural or non natural amino acid residue different from L- or D-Cys, such as L- or D-Lys, L- or D-Arg, L- or D-His
  MOD_RES (48): any natural or non natural amino acid residue different from L- or D-Cys, such as L- or D-Leu, L- or D-Ala, L- or D,L- or D-Val, L- or D-Leu, L- or D-Ile, L- or D-Phe, L- or D-Tyr
  MOD_RES (49): any natural or non natural amino acid residue different from L- or D-Cys, such as L- or D-Leu, L- or D-Ala, L- or D,L- or D-Val, L- or D-Leu, L- or D-Ile, L- or D-Phe, L- or D-Tyr
  MOD_RES (50): L- or D- Asx, L- or D-Glx, L- or D-Ala
  MOD_RES (51): any natural or non natural amino acid residue different from L- or D-Cys, such as L- or D-Thr, L- or D-Ser, L- or D-Tyr, L- or D-Ala, L- or D-Phe
XXXXXXXXXXXXXXXXXXXXXXXXXXXXXXXXXXXXXXXXXXXXXXXXXXX

SEQ ID NO: 39 - protein, 28 aa, Artificial Sequence
  Description of Artificial Sequence: Synthetic peptide
  MOD_RES (1): Ac-Ala
  MOD_RES (14): UDA
AQQNGDLQCLCVKXAPLYKKIIKKLLES

SEQ ID NO: 40 - protein, 27 aa, Artificial Sequence
  Description of Artificial Sequence: Synthetic peptide
  MOD_RES (1): Ac-Ala
  MOD_RES (13): UDA
AQQNGNLQCLCVXAPLYKKIIKKLLES

SEQ ID NO: 41 - protein, 28 aa, Artificial Sequence
  Description of Artificial Sequence: Synthetic peptide
  MOD_RES (14): UDA
AEEDGDLQCLCVKXAPLYKKIIKKLLES

SEQ ID NO: 42 - protein, 30 aa, Artificial Sequence
  Description of Artificial Sequence: Synthetic peptide
  MOD_RES (1): Ac-Ala
  MOD_RES (16): UDA
AEEDGDLQCLCVKTTXAPLYKKIIKKLLES

SEQ ID NO: 43 - protein, 28 aa, Artificial Sequence
  Description of Artificial Sequence: Synthetic peptide
  MOD_RES (1): Ac-Ala
  MOD_RES (14): UDA
AEEDGDLQCLCVKXAPLYKKIIKKLLES

SEQ ID NO: 44 - protein, 54 aa, Artificial Sequence; identical to SEQ ID NO: 19
  Description of Artificial Sequence: Synthetic peptide
  MOD_RES (1): any natural or non natural amino acid residue different from L- or D-Cys residue, such as L- or D-Leu, L- or D-Ile, L- or D-L- or D-Arg, or L- or D-Lys, L- or D-Orn, L- or D-Ala
  MOD_RES (2): any natural or non natural amino acid residue different from L- or D-Cys, such as L- or D-Arg, L- or D-Lys, L- or D-Orn- or L- or D-Glx, L- or D-Asx, L- or D-Thr, L- or D-Tyr
  MOD_RES (3): any natural or non natural amino acid residue different from L- or D-Cys, such as L- or D-Glx, L- or D-Asx, L- or D-Leu, L- or D-Ile, L- or D-Arg, L- or D-Ser, L- or D-Thr
  MOD_RES (4): any natural or non natural amino acid residue different from L- or D-Cys residue, such as L- or D-Leu, L- or D-Arg, or L- or D-Ile, L- or D-Lys
  MOD_RES (5): L- or D- Arg, L- or D-Cys, L- or D-Lys, L- or D-Orn, L- or D-Ala residue, such as L- or D-Arg, L- or D-Cys, L- or D-Orn- or D-Ala, L- or D-Lys
  MOD_RES (6): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn
  MOD_RES (7): any natural or non natural amino acid residue different from L- or D-Cys residue, such as  L- or D-Val, L- or D-Ile, L- or D-Ala, L- or D-Phe, L- or D-Tyr
  MOD_RES (8): L- or D-Cys, L- or D-Leu, L- or D-Phe, L- or D-His, L- or D-Trp, L- or D-Ser, L- or D-Thr, L- or D-Lys, L- or D-His, L- or D-Tyr, L- or D-Ile, L- or D-Orn
  MOD_RES (9): any natural or non natural amino acid residue different from L- or D-Cys residue, such as L- or D-Leu, L- or D-Glx, or L- or D-Asx, L- or D-Lys, L-Ile
  MOD_RES (10): any natural or non natural amino acid residue different from L- or D-Cys residue, such as L- or D-Glx, L- or D-Asx, L- or D-Thr, L- or D-Ser, L- or D-Leu, L- or D-Ile, L- or D-Pro, L- or D-Phe, L- or D-Tyr, L- or D-His, L- or D-Trp
  MOD_RES (11): L- or D-Thr, L- or D-Ser, L- or D-Tyr, L- or D-Trp, L- or D-His
  MOD_RES (12): any natural or non natural amino acid residue different from L- or D-Cys residue, such as  L- or D-Thr, L- or D-Glx, L- or D-L- or D-Asx, L- or D-Tyr
  MOD_RES (13): any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, L- or D-Trp
  MOD_RES (14): any natural or non natural amino acid residue different from L- or D-Cys residue, such as Gly, L- or D-Val, L- or D-Ala
  MOD_RES (15): any natural or non natural amino acid residue different from L- or D-Cys residue, such as L- or D-Val, L- or D-His, L- or D-Orn- or D-Arg
  MOD_RES (16)..(17): one or up to two natural or non natural amino acid residue different from L- or D-Cys residue, such as L- or D-His, L- or D-Pro, Gly, L- or D-Val, L- or D-Leu, L- or D-Ile, L- or D-Ala, L- or D-Phe, L- or D-Tyr
  MOD_RES (18)..(37): region may encompass 1-20 variable amino acids
  MOD_RES (38): any natural or non natural amino acid residue different from L- or D-Cys residue, such as L- or D-Phe, L- or D-Leu, L- or D-Lys, L- or D-Tyr, or L- or D-His, L- or D-Trp
  MOD_RES (39): L- or D-Leu, L- or D-Ile, L- or D-Lys, L- or D-Phe, L- or D-Tyr, L- or D-Ser, L- or D-Thr, L- or D-Orn, L- or D-Arg, L- or D-Val
  MOD_RES (40): any natural or non natural amino acid residue different from L- or D-Cys residue, such as L- or D-Lys, L- or D-Arg, L- or D-Orn- or D-Val, L- or D-Ile, L- or D-Leu
  MOD_RES (41): any natural or non natural amino acid residue different from L- or D-Cys residue, such asL- or D-Lys, L- or D-Arg, L- or D-Glx, L- or D-Asx, L- or D-Orn- or D-Ala, lL- or D-Ile, L- or D-Val
  MOD_RES (42): any natural or non natural amino acid residue different from L- or D-Cys residue, such asL- or D-Val, L- or D-Lys, L- or D-Arg, L- or D-Glx, L- or D-Asx, L- or D-Orn- or D-Ala, lL- or D-Ile, L- or D-Val
  MOD_RES (43): any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (44): any natural or non natural amino acid residue different from L- or D-Cys, such asL- or D-Glx, L- or D-Asx, L- or D-Lys, L- or D-Ile, L- or D-Leu, L- or D-Ala
  MOD_RES (45): any natural or non natural amino acid residue different from L- or D-Cys, such asL- or D-Lys, L- or D-Ile, L- or D-Leu, L- or D-Asx, L- or D-Glx, L- or D-His, L- or D-Tyr, L- or D-Ser, L- or D-Thr, L- or D-Leu, L- or D-Ile
  MOD_RES (46): any natural or non natural amino acid residue different from L- or D-Cys residue, such as L- or D-Ile, L- or D-Glx, L- or D-Asx, L- or D-Leu, Gly, L- or D-Phe, L- or D-Tyr, L- or D-Glu, L- or D-Lys
  MOD_RES (47): any natural or non natural amino acid residue different from L- or D-Cys residue, such asL- or D-Leu, L- or D-Glx, or L- or D-Asx, Gly
  MOD_RES (48): L- or D-Asx, L- or D-Glx, L- or D-Ser, L- or D-Thr, L- or D-Tyr, L- or D-Lys, L or D-Arg, Gly
  MOD_RES (49): any natural or non natural amino acid different from L- or D-Cys, such asGly, L- or D-Asx, L- or D-Glx, L- or D-Lys, L- or D-Arg
  MOD_RES (50): any natural or non natural amino acid different from L- or D-Cys, such asGly, L- or D-Asx, L- or D-Glx, L- or  D-Lys, L- or D-Arg; L- or D-Glu
  MOD_RES (51)..(54): one or up to four natural or non natural amino acid residue different from L- or D-Cys residue; see specification as filed for detailed embodiments

SEQ ID NO: 45 - protein, 53 aa, Artificial Sequence; identical to SEQ ID NO: 20
  Description of Artificial Sequence: Synthetic peptide
  MOD_RES (1): any natural or non natural amino acid residue different from L- or D-Cys, such as L- or D-Arg, L- or D-Lys, L- or D-Orn- or L- or D-Glx, L- or D-Asx, L- or D-Thr, L- or D-Tyr
  MOD_RES (2): any natural or non natural amino acid residue different from L- or D-Cys, such as L- or D-Glx, L- or D-Asx, L- or D-Leu, L- or D-Ile, L- or D-Arg, L- or D-Ser, L- or D-Thr
  MOD_RES (3): any natural or non natural amino acid residue different from L- or D-Cys residue, such as L- or D-Leu, L- or D-Arg, or L- or D-Ile, L- or D-Lys
  MOD_RES (4): L- or D- Arg, L- or D-Cys, L- or D-Lys, L- or D-Orn, L- or D-Ala residue, such as L- or D-Arg, L- or D-Cys, L- or D-Orn- or D-Ala, L- or D-Lys
  MOD_RES (5): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn
  MOD_RES (6): any natural or non natural amino acid residue different from L- or D-Cys residue, such as  L- or D-Val, L- or D-Ile, L- or D-Ala, L- or D-Phe, L- or D-Tyr
  MOD_RES (7): L- or D-Cys, L- or D-Leu, L- or D-Phe, L- or D-His, L- or D-Trp, L- or D-Ser, L- or D-Thr, L- or D-Lys, L- or D-His, L- or D-Tyr, L- or D-Ile, L- or D-Orn
  MOD_RES (8): any natural or non natural amino acid residue different from L- or D-Cys residue, such as L- or D-Leu, L- or D-Glx, or L- or D-Asx, L- or D-Lys, L-Ile
  MOD_RES (9): any natural or non natural amino acid residue different from L- or D-Cys residue, such as L- or D-Glx, L- or D-Asx, L- or D-Thr, L- or D-Ser, L- or D-Leu, L- or D-Ile, L- or D-Pro, L- or D-Phe, L- or D-Tyr, L- or D-His, L- or D-Trp
  MOD_RES (10): L- or D-Thr, L- or D-Ser, L- or D-Tyr, L- or D-Trp, L- or D-His
  MOD_RES (11): any natural or non natural amino acid residue different from L- or D-Cys residue, such as  L- or D-Thr, L- or D-Glx, L- or D-L- or D-Asx, L- or D-Tyr
  MOD_RES (12): any natural or non natural amino acid residue different from L- or D-Cys residue, such as L- or D-Glx, L- or D-Asx, Gly, L- or D-Val, L- or D-Leu, L- or D-Ile, L- or D-Pro, L- or D-Phe, L- or D-Tyr, L- or D-His, L- or D-Trp
  MOD_RES (13): any natural or non natural amino acid residue different from L- or D-Cys residue, such as Gly, L- or D-Val, L- or D-Ala
  MOD_RES (14): any natural or non natural amino acid residue different from L- or D-Cys residue, such as L- or D-Val, L- or D-His, L- or D-Orn- or D-Arg
  MOD_RES (15)..(16): one or up to two natural or non natural amino acid residue different from L- or D-Cys residue, such as L- or D-His, L- or D-Pro, Gly, L- or D-Val, L- or D-Leu, L- or D-Ile, L- or D-Ala, L- or D-Phe, L- or D-Tyr
  MOD_RES (17)..(36): region may encompass 1-20 variable amino acids
  MOD_RES (37): any natural or non natural amino acid residue different from L- or D-Cys residue, such as L- or D-Phe, L- or D-Leu, L- or D-Lys, L- or D-Tyr, or L- or D-His, L- or D-Trp
  MOD_RES (38): L- or D-Leu, L- or D-Ile, L- or D-Lys, L- or D-Phe, L- or D-Tyr, L- or D-Ser, L- or D-Thr, L- or D-Orn, L- or D-Arg, L- or D-Val
  MOD_RES (39): any natural or non natural amino acid residue different from L- or D-Cys residue, such as L- or D-Lys, L- or D-Arg, L- or D-Orn- or D-Val, L- or D-Ile, L- or D-Leu
  MOD_RES (40): any natural or non natural amino acid residue different from L- or D-Cys residue, such asL- or D-Lys, L- or D-Arg, L- or D-Glx, L- or D-Asx, L- or D-Orn- or D-Ala, lL- or D-Ile, L- or D-Val
  MOD_RES (41): any natural or non natural amino acid residue different from L- or D-Cys residue, such asL- or D-Val, L- or D-Lys, L- or D-Arg, L- or D-Glx, L- or D-Asx, L- or D-Orn- or D-Ala, lL- or D-Ile, L- or D-Val
  MOD_RES (42): any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (43): any natural or non natural amino acid residue different from L- or D-Cys, such asL- or D-Glx, L- or D-Asx, L- or D-Lys, L- or D-Ile, L- or D-Leu, L- or D-Ala
  MOD_RES (44): any natural or non natural amino acid residue different from L- or D-Cys, such asL- or D-Lys, L- or D-Ile, L- or D-Leu, L- or D-Asx, L- or D-Glx, L- or D-His, L- or D-Tyr, L- or D-Ser, L- or D-Thr, L- or D-Leu, L- or D-Ile
  MOD_RES (45): any natural or non natural amino acid residue different from L- or D-Cys residue, such as L- or D-Ile, L- or D-Glx, L- or D-Asx, L- or D-Leu, Gly, L- or D-Phe, L- or D-Tyr, L- or D-Glu, L- or D-Lys
  MOD_RES (46): any natural or non natural amino acid residue different from L- or D-Cys residue, such asL- or D-Leu, L- or D-Glx, or L- or D-Asx, Gly
  MOD_RES (47): L- or D-Asx, L- or D-Glx, L- or D-Ser, L- or D-Thr, L- or D-Tyr, L- or D-Lys, L or D-Arg, Gly
  MOD_RES (48): any natural or non natural amino acid different from L- or D-Cys, such asGly, L- or D-Asx, L- or D-Glx, L- or D-Lys, L- or D-Arg
  MOD_RES (49): any natural or non natural amino acid different from L- or D-Cys, such asGly, L- or D-Asx, L- or D-Glx, L- or D-Lys, L- or D-Arg; L- or D-Glu
  MOD_RES (50)..(53): one or up to four natural or non natural amino acid residue different from L- or D-Cys residue; see specification as filed for detailed embodiments

SEQ ID NO: 46 - protein, 50 aa, Artificial Sequence
  Description of Artificial Sequence: Synthetic peptide
  MOD_RES (17)..(36): region may encompass 1-20 variable amino acids
LRELRCVCLQTTQGVHXXXXXXXXXXXXXXXXXXXXFLKKVIQKILDGGN

SEQ ID NO: 47 - protein, 50 aa, Artificial Sequence
  Description of Artificial Sequence: Synthetic peptide
  MOD_RES (17)..(36): region may encompass 1-20 variable amino acids
RELRCVCLQTTQGVHPXXXXXXXXXXXXXXXXXXXXFLKKVIQKILDGGN

SEQ ID NO: 48 - protein, 50 aa, Artificial Sequence
  Description of Artificial Sequence: Synthetic peptide
  MOD_RES (17)..(36): region may encompass 1-20 variable amino acids
LRELRCVCLQTTQGVHXXXXXXXXXXXXXXXXXXXXLKKVIQKILDGGNK

SEQ ID NO: 49 - protein, 50 aa, Artificial Sequence
  Description of Artificial Sequence: Synthetic peptide
  MOD_RES (17)..(36): region may encompass 1-20 variable amino acids
LRELRCVCLQTTQGVHXXXXXXXXXXXXXXXXXXXXKKVIQKILDGGNKE

SEQ ID NO: 50 - protein, 50 aa, Artificial Sequence
  Description of Artificial Sequence: Synthetic peptide
  MOD_RES (17)..(36): region may encompass 1-20 variable amino acids
LRELRCVCLQTTQGVHXXXXXXXXXXXXXXXXXXXXKVIQKILDGGNKEN

SEQ ID NO: 51 - protein, 49 aa, Artificial Sequence
  Description of Artificial Sequence: Synthetic peptide
  MOD_RES (17)..(36): region may encompass 1-20 variable amino acids
RELRCVCLQTTQGVHXXXXXXXXXXXXXXXXXXXXLKKVIQKILDGGNK

SEQ ID NO: 52 - protein, 49 aa, Artificial Sequence
  Description of Artificial Sequence: Synthetic peptide
  MOD_RES (16)..(35): region may encompass 1-20 variable amino acids
RELRCVCLQTTQGVHXXXXXXXXXXXXXXXXXXXXKKVIQKILDGGNKE

SEQ ID NO: 53 - protein, 49 aa, Artificial Sequence
  Description of Artificial Sequence: Synthetic peptide
  MOD_RES (16)..(35): region may encompass 1-20 variable amino acids
RELRCVCLQTTQGVHXXXXXXXXXXXXXXXXXXXXKVIQKILDGGNKEN

SEQ ID NO: 54 - protein, 49 aa, Artificial Sequence; identical to SEQ ID NO: 53
  Description of Artificial Sequence: Synthetic peptide
  MOD_RES (16)..(35): region may encompass 1-20 variable amino acids

SEQ ID NO: 55 - protein, 49 aa, Artificial Sequence; identical to SEQ ID NO: 53
  Description of Artificial Sequence: Synthetic peptide
  MOD_RES (16)..(35): region may encompass 1-20 variable amino acids

SEQ ID NO: 56 - protein, 50 aa, Artificial Sequence
  Description of Artificial Sequence: Synthetic peptide
  MOD_RES (17)..(36): region may encompass 1-20 variable amino acids
GPVSAVLTELRCTCLRXXXXXXXXXXXXXXXXXXXXFLKKVIQKILDSGN

SEQ ID NO: 57 - protein, 50 aa, Artificial Sequence
  Description of Artificial Sequence: Synthetic peptide
  MOD_RES (17)..(36): region may encompass 1-20 variable amino acids
VSAVLTELRCTCLRVTXXXXXXXXXXXXXXXXXXXXFLKKVIQKILDSGN

SEQ ID NO: 58 - protein, 50 aa, Artificial Sequence
  Description of Artificial Sequence: Synthetic peptide
  MOD_RES (17)..(36): region may encompass 1-20 variable amino acids
VLTELRCTCLRVTLRVXXXXXXXXXXXXXXXXXXXXFLKKVIQKILDSGN

SEQ ID NO: 59 - protein, 50 aa, Artificial Sequence
  Description of Artificial Sequence: Synthetic peptide
  MOD_RES (17)..(36): region may encompass 1-20 variable amino acids
ELRCTCLRVTLRVNPKXXXXXXXXXXXXXXXXXXXXFLKKVIQKILDSGN

SEQ ID NO: 60 - protein, 50 aa, Artificial Sequence
  Description of Artificial Sequence: Synthetic peptide
  MOD_RES (17)..(36): region may encompass 1-20 variable amino acids
GPVSAVLTELRCTCLRXXXXXXXXXXXXXXXXXXXXLKKVIQKILDSGNK

SEQ ID NO: 61 - protein, 50 aa, Artificial Sequence
  Description of Artificial Sequence: Synthetic peptide
  MOD_RES (17)..(36): region may encompass 1-20 variable amino acids
VSAVLTELRCTCLRVTXXXXXXXXXXXXXXXXXXXXLKKVIQKILDSGNK

SEQ ID NO: 62 - protein, 62 aa, Artificial Sequence
  Description of Artificial Sequence: Synthetic peptide
  MOD_RES (1): any natural or non natural amino acid residue different from L- or D-Cys residue, such as  Gly, L- or D-Pro, L- or D-L- or D-Val, or L- or D-Ala
  MOD_RES (2): any natural or non natural amino acid residue residue different from L- or D-Cys, such as L- or D-Pro, L- or D-Ala, L- or D-Leu- or L- or D-Ile, L- or D-Phe, L- or D-Thr, L- or D-Tyr
  MOD_RES (3): any natural or non natural amino acid residue different from L- or D-Cys residue, such as  L- or D-Val, L- or D-Ile, L- or D-Ala, L- or D-Phe, L- or D-Tyr
  MOD_RES (4): any natural or non natural amino acid different from L- or D-Cys, such as L- or D-Asx, L-or D-Glx, L- or D-Ser, L- or D-Thr, L- or D-Tyr, L- or D-Lys
  MOD_RES (5): any natural or non natural amino acid residue different from L- or D-Cys residue, such as L- or D-Ala, Gly, or D-L-Phe
  MOD_RES (6): L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn
  MOD_RES (7): any natural or non natural amino acid residue different from L- or D-Cys residue, such as L- or D-Leu, L- or D-Arg, or L- or D-Ile, L- or D-Lys
  MOD_RES (8): L- or D-Thr, L- or D-Ser, L- or D-Tyr, L- or D-Trp, L- or D-His
  MOD_RES (9): any natural or non natural amino acid residue different from L- or D-Cys, such as L- or D-Glx, L- or D-Asx, L- or D-Leu, L- or D-Ile, L- or D-Arg, L- or D-Ser, L- or D-Thr
  MOD_RES (10): any natural or non natural amino acid residue different from L- or D-Cys residue, such as L- or D-Leu, L- or D-Arg, or L- or D-Ile, L- or D-Lys
  MOD_RES (11): L- or D-Arg, L- or D-Lys, L- or D-Orn- or L- or D-Glx, L- or D-Asx, L- or D-Thr, L- or D-Tyr
  MOD_RES (12): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (13): any natural or non natural amino acid residue different from L- or D-Cys residue, such as  L- or D-Thr, L- or D-Glx, L- or D-L- or D-Asx, L- or D-Tyr
  MOD_RES (14): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (15): any natural or non natural amino acid residue different from L- or D-Cys residue, such as L- or D-Leu, L- or D-Arg, or L- or D-Ile, L- or D-Lys
  MOD_RES (16)..(25): one up to ten natural or non natural amino acid residues different from L- or D-Cys; see specification as filed for detailed embodiments
  MOD_RES (26)..(45): region may encompass 1-20 variable amino acid residues
  MOD_RES (46): any natural or non natural amino acid residue different from L- or D-Cys residue, such as L- or D-Phe, L- or D-Leu, L- or D-Lys, L- or D-Tyr, or L- or D-His, L- or D-Trp
  MOD_RES (47): L- or D-Leu, L- or D-Ile, L- or D-Lys, L- or D-Phe, L- or D-Tyr, L- or D-Ser, L- or D-Thr, L- or D-Orn, L- or D-Arg, L- or D-Val
  MOD_RES (48): any natural or non natural amino acid residue different from L- or D-Cys residue, such as L- or D-Lys, L- or D-Arg, L- or D-Orn- or D-Val, L- or D-Ile, L- or D-Leu
  MOD_RES (49): any natural or non natural amino acid residue different from L- or D-Cys residue, such as L- or D-Lys, L- or D-Arg, L- or D-Orn- or D-Val, L- or D-Ile, L- or D-Leu
  MOD_RES (50): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile, L- or D-Val
  MOD_RES (51): any natural or non natural amino acid residue different from L- or D-Cys residue, such as  L- or D-Ile, L- or D-Glx, L- or D-Asx, L- or D-Leu, or L- or D-Ala, L- or D-Phe, L- or  D-Tyr, L- or D-Glu, L- or D-Lys
  MOD_RES (52): any natural or non natural amino acid residue different from L- or D-Cys, such as L- or D-Glx, L- or D-Asx, L- or D-Lys, L- or D-Ile, L- or D-Leu, L- or D-Ala
  MOD_RES (53): any natural or non natural amino acid residue different from L- or D-Cys residue, such as L- or D-Lys, L- or D-Arg, L- or D-Orn- or D-Val, L- or D-Ile, L- or D-Leu
  MOD_RES (54): any natural or non natural amino acid residue different from L- or D-Cys residue, such as  L- or D-Ile, L- or D-Glx, L- or D-Asx, L- or D-Leu, Gly, L- or D-Phe, L- or D-Tyr, L- or D-Glu, L- or D-Lys; L- or D-Leu, L- or D-Asp, Gly
  MOD_RES (55): any natural or non natural amino acid residue different from L- or D-Cys, such as L- or D-Glx, L- or D-Asx, L- or D-Lys, L- or D-Ile, L- or D-Leu, L- or D-Ala
  MOD_RES (56): L- or D-Asx, L- or D-Glx, L- or D-Ser, L- or D-Thr, L- or D-Tyr, L- or D-Lys, L or D-Arg, Gly
  MOD_RES (57): any natural or non natural amino acid different from L- or D-Cys, such as L- or D-Asx, L-or D-Glx, L- or D-Ser, L- or D-Thr, L- or D-Tyr, L- or D-Lys
  MOD_RES (58): any natural or non natural amino acid residue different from L- or D-Cys residue, such as  Gly, L- or D-Pro, L- or D-L- or D-Val, or L- or D-Ala
  MOD_RES (59)..(62): one or up to four natural or non natural amino acid residue different from L- or D-Cys; see specification as filed for detailed embodiments
XXXXXXXXXXXXXXXXXXXXXXXXXXXXXXXXXXXXXXXXXXXXXXXXXXXXXXXXXXXXXX

SEQ ID NO: 63 - protein, 57 aa, Artificial Sequence; identical to SEQ ID NO: 18
  Description of Artificial Sequence: Synthetic peptide
  MOD_RES (1): L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn
  MOD_RES (2): any natural or non natural amino acid residue different from L- or D-Cys residue, such as L- or D-Leu, L- or D-Arg, or L- or D-Ile, L- or D-Lys
  MOD_RES (3): L- or D-Thr, L- or D-Ser, L- or D-Tyr, L- or D-Trp, L- or D-His
  MOD_RES (4): any natural or non natural amino acid residue different from L- or D-Cys, such as L- or D-Glx, L- or D-Asx, L- or D-Leu, L- or D-Ile, L- or D-Arg, L- or D-Ser, L- or D-Thr
  MOD_RES (5): any natural or non natural amino acid residue different from L- or D-Cys residue, such as L- or D-Leu, L- or D-Arg, or L- or D-Ile, L- or D-Lys
  MOD_RES (6): L- or D-Arg, L- or D-Lys, L- or D-Orn- or L- or D-Glx, L- or D-Asx, L- or D-Thr, L- or D-Tyr
  MOD_RES (7): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (8): any natural or non natural amino acid residue different from L- or D-Cys residue, such as  L- or D-Thr, L- or D-Glx, L- or D-L- or D-Asx, L- or D-Tyr
  MOD_RES (9): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (10): any natural or non natural amino acid residue different from L- or D-Cys residue, such as L- or D-Leu, L- or D-Arg, or L- or D-Ile, L- or D-Lys
  MOD_RES (11)..(20): one up to ten natural or non natural amino acid residues different from L- or D-Cys; see specification as filed for detailed embodiments
  MOD_RES (21)..(40): region may encompass 1-20 variable amino acid residues
  MOD_RES (41): any natural or non natural amino acid residue different from L- or D-Cys residue, such as L- or D-Phe, L- or D-Leu, L- or D-Lys, L- or D-Tyr, or L- or D-His, L- or D-Trp
  MOD_RES (42): L- or D-Leu, L- or D-Ile, L- or D-Lys, L- or D-Phe, L- or D-Tyr, L- or D-Ser, L- or D-Thr, L- or D-Orn, L- or D-Arg, L- or D-Val
  MOD_RES (43): any natural or non natural amino acid residue different from L- or D-Cys residue, such as L- or D-Lys, L- or D-Arg, L- or D-Orn- or D-Val, L- or D-Ile, L- or D-Leu
  MOD_RES (44): any natural or non natural amino acid residue different from L- or D-Cys residue, such as L- or D-Lys, L- or D-Arg, L- or D-Orn- or D-Val, L- or D-Ile, L- or D-Leu
  MOD_RES (45): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile, L- or D-Val
  MOD_RES (46): any natural or non natural amino acid residue different from L- or D-Cys residue, such as  L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (47): any natural or non natural amino acid residue different from L- or D-Cys, such as L- or D-Glx, L- or D-Asx, L- or D-Lys, L- or D-Ile, L- or D-Leu, L- or D-Ala
  MOD_RES (48): any natural or non natural amino acid residue different from L- or D-Cys residue, such as L- or D-Lys, L- or D-Arg, L- or D-Orn- or D-Val, L- or D-Ile, L- or D-Leu
  MOD_RES (49): any natural or non natural amino acid residue different from L- or D-Cys residue, such as  L- or D-Ile, L- or D-Glx, L- or D-Asx, L- or D-Leu, Gly, L- or D-Phe, L- or D-Tyr, L- or D-Glu, L- or D-Lys; L- or D-Leu, L- or D-Asp, Gly
  MOD_RES (50): any natural or non natural amino acid residue different from L- or D-Cys, such as L- or D-Glx, L- or D-Asx, L- or D-Lys, L- or D-Ile, L- or D-Leu, L- or D-Ala
  MOD_RES (51): L- or D-Asx, L- or D-Glx, L- or D-Ser, L- or D-Thr, L- or D-Tyr, L- or D-Lys, L or D-Arg, Gly
  MOD_RES (52): any natural or non natural amino acid different from L- or D-Cys, such as L- or D-Asx, L-or D-Glx, L- or D-Ser, L- or D-Thr, L- or D-Tyr, L- or D-Lys
  MOD_RES (53): any natural or non natural amino acid residue different from L- or D-Cys residue, such as  Gly, L- or D-Pro, L- or D-L- or D-Val, or L- or D-Ala
  MOD_RES (54)..(57): one or up to four natural or non natural amino acid residue different from L- or D-Cys; see specification as filed for detailed embodiments

SEQ ID NO: 64 - protein, 60 aa, Artificial Sequence
  Description of Artificial Sequence: Synthetic peptide
  MOD_RES (1): any natural or non natural amino acid residue different from L- or D-Cys residue, such as  L- or D-Val, L- or D-Ile, L- or D-Ala, L- or D-Phe, L- or D-Tyr
  MOD_RES (2): any natural or non natural amino acid different from L- or D-Cys, such as L- or D-Asx, L-or D-Glx, L- or D-Ser, L- or D-Thr, L- or D-Tyr, L- or D-Lys
  MOD_RES (3): any natural or non natural amino acid residue different from L- or D-Cys residue, such as L- or D-Ala, Gly, or D-L-Phe
  MOD_RES (4): L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn
  MOD_RES (5): any natural or non natural amino acid residue different from L- or D-Cys residue, such as L- or D-Leu, L- or D-Arg, or L- or D-Ile, L- or D-Lys
  MOD_RES (6): L- or D-Thr, L- or D-Ser, L- or D-Tyr, L- or D-Trp, L- or D-His
  MOD_RES (7): any natural or non natural amino acid residue different from L- or D-Cys, such as L- or D-Glx, L- or D-Asx, L- or D-Leu, L- or D-Ile, L- or D-Arg, L- or D-Ser, L- or D-Thr
  MOD_RES (8): any natural or non natural amino acid residue different from L- or D-Cys residue, such as L- or D-Leu, L- or D-Arg, or L- or D-Ile, L- or D-Lys
  MOD_RES (9): L- or D-Arg, L- or D-Lys, L- or D-Orn- or L- or D-Glx, L- or D-Asx, L- or D-Thr, L- or D-Tyr
  MOD_RES (10): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (11): any natural or non natural amino acid residue different from L- or D-Cys residue, such as  L- or D-Thr, L- or D-Glx, L- or D-L- or D-Asx, L- or D-Tyr
  MOD_RES (12): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (13): any natural or non natural amino acid residue different from L- or D-Cys residue, such as L- or D-Leu, L- or D-Arg, or L- or D-Ile, L- or D-Lys
  MOD_RES (14)..(23): one up to ten natural or non natural amino acid residues different from L- or D-Cys; see specification as filed for detailed embodiments
  MOD_RES (24)..(43): region may encompass 1-20 variable amino acid residues
  MOD_RES (44): any natural or non natural amino acid residue different from L- or D-Cys residue, such as L- or D-Phe, L- or D-Leu, L- or D-Lys, L- or D-Tyr, or L- or D-His, L- or D-Trp
  MOD_RES (45): L- or D-Leu, L- or D-Ile, L- or D-Lys, L- or D-Phe, L- or D-Tyr, L- or D-Ser, L- or D-Thr, L- or D-Orn, L- or D-Arg, L- or D-Val
  MOD_RES (46): any natural or non natural amino acid residue different from L- or D-Cys residue, such as L- or D-Lys, L- or D-Arg, L- or D-Orn- or D-Val, L- or D-Ile, L- or D-Leu
  MOD_RES (47): any natural or non natural amino acid residue different from L- or D-Cys residue, such as L- or D-Lys, L- or D-Arg, L- or D-Orn- or D-Val, L- or D-Ile, L- or D-Leu
  MOD_RES (48): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile, L- or D-Val
  MOD_RES (49): any natural or non natural amino acid residue different from L- or D-Cys residue, such as  L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (50): any natural or non natural amino acid residue different from L- or D-Cys, such as L- or D-Glx, L- or D-Asx, L- or D-Lys, L- or D-Ile, L- or D-Leu, L- or D-Ala
  MOD_RES (51): any natural or non natural amino acid residue different from L- or D-Cys residue, such as L- or D-Lys, L- or D-Arg, L- or D-Orn- or D-Val, L- or D-Ile, L- or D-Leu
  MOD_RES (52): any natural or non natural amino acid residue different from L- or D-Cys residue, such as  L- or D-Ile, L- or D-Glx, L- or D-Asx, L- or D-Leu, Gly, L- or D-Phe, L- or D-Tyr, L- or D-Glu, L- or D-Lys; L- or D-Leu, L- or D-Asp, Gly
  MOD_RES (53): any natural or non natural amino acid residue different from L- or D-Cys, such as L- or D-Glx, L- or D-Asx, L- or D-Lys, L- or D-Ile, L- or D-Leu, L- or D-Ala
  MOD_RES (54): L- or D-Asx, L- or D-Glx, L- or D-Ser, L- or D-Thr, L- or D-Tyr, L- or D-Lys, L or D-Arg, Gly
  MOD_RES (55): any natural or non natural amino acid different from L- or D-Cys, such as L- or D-Asx, L-or D-Glx, L- or D-Ser, L- or D-Thr, L- or D-Tyr, L- or D-Lys
  MOD_RES (56): any natural or non natural amino acid residue different from L- or D-Cys residue, such as  Gly, L- or D-Pro, L- or D-L- or D-Val, or L- or D-Ala
  MOD_RES (57)..(60): one or up to four natural or non natural amino acid residue different from L- or D-Cys; see specification as filed for detailed embodiments
XXXXXXXXXXXXXXXXXXXXXXXXXXXXXXXXXXXXXXXXXXXXXXXXXXXXXXXXXXXX

SEQ ID NO: 65 - protein, 54 aa, Artificial Sequence; identical to SEQ ID NO: 19
  Description of Artificial Sequence: Synthetic peptide
  MOD_RES (1): any natural or non natural amino acid residue different from L- or D-Cys, such as L- or D-Glx, L- or D-Asx, L- or D-Leu, L- or D-Ile, L- or D-Arg, L- or D-Ser, L- or D-Thr
  MOD_RES (2): any natural or non natural amino acid residue different from L- or D-Cys residue, such as L- or D-Leu, L- or D-Arg, or L- or D-Ile, L- or D-Lys
  MOD_RES (3): L- or D-Arg, L- or D-Lys, L- or D-Orn- or L- or D-Glx, L- or D-Asx, L- or D-Thr, L- or D-Tyr
  MOD_RES (4): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (5): any natural or non natural amino acid residue different from L- or D-Cys residue, such as  L- or D-Thr, L- or D-Glx, L- or D-L- or D-Asx, L- or D-Tyr
  MOD_RES (6): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (7): any natural or non natural amino acid residue different from L- or D-Cys residue, such as L- or D-Leu, L- or D-Arg, or L- or D-Ile, L- or D-Lys
  MOD_RES (8)..(17): one up to ten natural or non natural amino acid residues different from L- or D-Cys; see specification as filed for detailed embodiments
  MOD_RES (18)..(37): region may encompass 1-20 variable amino acid residues
  MOD_RES (38): any natural or non natural amino acid residue different from L- or D-Cys residue, such as L- or D-Phe, L- or D-Leu, L- or D-Lys, L- or D-Tyr, or L- or D-His, L- or D-Trp
  MOD_RES (39): L- or D-Leu, L- or D-Ile, L- or D-Lys, L- or D-Phe, L- or D-Tyr, L- or D-Ser, L- or D-Thr, L- or D-Orn, L- or D-Arg, L- or D-Val
  MOD_RES (40): any natural or non natural amino acid residue different from L- or D-Cys residue, such as L- or D-Lys, L- or D-Arg, L- or D-Orn- or D-Val, L- or D-Ile, L- or D-Leu
  MOD_RES (41): any natural or non natural amino acid residue different from L- or D-Cys residue, such as L- or D-Lys, L- or D-Arg, L- or D-Orn- or D-Val, L- or D-Ile, L- or D-Leu
  MOD_RES (42): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile, L- or D-Val
  MOD_RES (43): any natural or non natural amino acid residue different from L- or D-Cys residue, such as  L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (44): any natural or non natural amino acid residue different from L- or D-Cys, such as L- or D-Glx, L- or D-Asx, L- or D-Lys, L- or D-Ile, L- or D-Leu, L- or D-Ala
  MOD_RES (45): any natural or non natural amino acid residue different from L- or D-Cys residue, such as L- or D-Lys, L- or D-Arg, L- or D-Orn- or D-Val, L- or D-Ile, L- or D-Leu
  MOD_RES (46): any natural or non natural amino acid residue different from L- or D-Cys residue, such as  L- or D-Ile, L- or D-Glx, L- or D-Asx, L- or D-Leu, Gly, L- or D-Phe, L- or D-Tyr, L- or D-Glu, L- or D-Lys; L- or D-Leu, L- or D-Asp, Gly
  MOD_RES (47): any natural or non natural amino acid residue different from L- or D-Cys, such as L- or D-Glx, L- or D-Asx, L- or D-Lys, L- or D-Ile, L- or D-Leu, L- or D-Ala
  MOD_RES (48): L- or D-Asx, L- or D-Glx, L- or D-Ser, L- or D-Thr, L- or D-Tyr, L- or D-Lys, L or D-Arg, Gly
  MOD_RES (49): any natural or non natural amino acid different from L- or D-Cys, such as L- or D-Asx, L-or D-Glx, L- or D-Ser, L- or D-Thr, L- or D-Tyr, L- or D-Lys
  MOD_RES (50): any natural or non natural amino acid residue different from L- or D-Cys residue, such as  Gly, L- or D-Pro, L- or D-L- or D-Val, or L- or D-Ala
  MOD_RES (51)..(54): one or up to four natural or non natural amino acid residue different from L- or D-Cys; see specification as filed for detailed embodiments

SEQ ID NO: 66 - protein, 50 aa, Artificial Sequence
  Description of Artificial Sequence: Synthetic peptide
  MOD_RES (17)..(36): region may encompass 1-20 variable amino acids
VLTELRCTCLRVTLRVXXXXXXXXXXXXXXXXXXXXLKKVIQKILDSGNK

SEQ ID NO: 67 - protein, 50 aa, Artificial Sequence
  Description of Artificial Sequence: Synthetic peptide
  MOD_RES (17)..(36): region may encompass 1-20 variable amino acids
ELRCTCLRVTLRVNPKXXXXXXXXXXXXXXXXXXXXLKKVIQKILDSGNK

SEQ ID NO: 68 - protein, 50 aa, Artificial Sequence
  Description of Artificial Sequence: Synthetic peptide
  MOD_RES (17)..(36): region may encompass 1-20 variable amino acids
GPVSAVLTELRCTCLRXXXXXXXXXXXXXXXXXXXXKKVIQKILDSGNKN

SEQ ID NO: 69 - protein, 50 aa, Artificial Sequence
  Description of Artificial Sequence: Synthetic peptide
  MOD_RES (17)..(36): region may encompass 1-20 variable amino acids
VSAVLTELRCTCLRVTXXXXXXXXXXXXXXXXXXXXKKVIQKILDSGNKN

SEQ ID NO: 70 - protein, 50 aa, Artificial Sequence
  Description of Artificial Sequence: Synthetic peptide
  MOD_RES (17)..(36): region may encompass 1-20 variable amino acids
VLTELRCTCLRVTLRVXXXXXXXXXXXXXXXXXXXXKKVIQKILDSGNKN

SEQ ID NO: 71 - protein, 50 aa, Artificial Sequence
  Description of Artificial Sequence: Synthetic peptide
  MOD_RES (17)..(36): region may encompass 1-20 variable amino acids
ELRCTCLRVTLRVNPKXXXXXXXXXXXXXXXXXXXXKKVIQKILDSGNKN

SEQ ID NO: 72 - protein, 50 aa, Artificial Sequence; identical to SEQ ID NO: 59
  Description of Artificial Sequence: Synthetic peptide
  MOD_RES (17)..(36): region may encompass 1-20 variable amino acids

SEQ ID NO: 73 - protein, 50 aa, Artificial Sequence; identical to SEQ ID NO: 71
  Description of Artificial Sequence: Synthetic peptide
  MOD_RES (17)..(36): region may encompass 1-20 variable amino acids

SEQ ID NO: 74 - protein, 57 aa, Artificial Sequence; identical to SEQ ID NO: 18
  Description of Artificial Sequence: Synthetic peptide
  MOD_RES (1): any natural or non natural amino acid residue different from L- or D-Cys residue, such as L- or D-Ala, Gly, or D-L-Phe
  MOD_RES (2): any natural or non natural amino acid residue different from L- or D-Cys, such as L- or D-Glx, L- or D-Asx, L- or D-Leu, L- or D-Ile, L- or D-Arg, L- or D-Ser, L- or D-Thr
  MOD_RES (3): any natural or non natural amino acid residue different from L- or D-Cys, such as L- or D-Glx, L- or D-Asx, L- or D-Lys, L- or D-Ile, L- or D-Leu, L- or D-Ala
  MOD_RES (4): L- or D-Arg, L- or D-Lys, L- or D-Orn- or L- or D-Glx, L- or D-Asx, L- or D-Thr, L- or D-Tyr
  MOD_RES (5): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (6): L- or D-Met, L- or D-Ieu, L- or D-Asx, L- or D-Nle, L- or D-Ala, L- or D-Phe, L- or D-Glx, L- or D-Ile, L- or D-Tyr
  MOD_RES (7): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (8): any natural or non natural amino acid residue different from L- or D-Cys residue, such as  L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (9): any natural or non natural amino acid residue different from L- or D-Cys residue, such as L- or D-Lys, L- or D-Arg, L- or D-Orn- or D-Val, L- or D-Ile, L- or D-Leu
  MOD_RES (10): any natural or non natural amino acid residue different from L- or D-Cys residue, such as L- or D-Thr, L- or D-Glx, L- or D-L- or D-Asx, L- or D-Tyr
  MOD_RES (11): any natural or non natural amino acid residue different from L- or D-Cys residue, such as L- or D-Thr, L- or D-Glx, L- or D-L- or D-Asx, L- or D-Tyr
  MOD_RES (12): any natural or non natural amino acid different from L- or D-Cys, such as L- or D-Asx, L-or D-Glx, L- or D-Ser, L- or D-Thr, L- or D-Tyr, L- or D-Lys
  MOD_RES (13): any natural or non natural amino acid residue different from L- or D-Cys residue, such as Gly, L- or D-Pro, L- or D-L- or D-Val, or L- or D-Ala
  MOD_RES (14): any natural or non natural amino acid residue different from L- or D-Cys residue, such as  L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (15): L- or D-His, L- or D-Lys, L- or D-Arg, L- or D-Orn, L- or D-Trp, L- or D-Ala, L- or D-Phe, L- or D-Pro
  MOD_RES (16): any natural or non natural amino acid residue residue different from L- or D-Cys, such as L- or D-Pro, L- or D-Ala, L- or D-Leu- or L- or D-Ile, L- or D-Phe, L- or D-Thr, L- or D-Tyr
  MOD_RES (17)..(36): region may encompass 1-20 variable amino acids
  MOD_RES (37)..(41): one or up to five natural or non natural amino acid residue different from L- or D-Cys; see specification as filed for specific embodiments
  MOD_RES (42): L- or D-Leu, L- or D-Ile, L- or D-Lys, L- or D-Phe, L- or D-Tyr, L- or D-Ser, L- or D-Thr, L- or D-Orn, L- or D-Arg, L- or D-Val
  MOD_RES (43): any natural or non natural amino acid residue different from L- or D-Cys residue, such as L- or D-Lys, L- or D-Arg, L- or D-Orn- or D-Val, L- or D-Ile, L- or D-Leu
  MOD_RES (44): any natural or non natural amino acid residue different from L- or D-Cys residue, such as L- or D-Lys, L- or D-Arg, L- or D-Orn- or D-Val, L- or D-Ile, L- or D-Leu
  MOD_RES (45): L- or D-Leu, L- or D-Ile, L- or D-Lys, L- or D-Phe, L- or D-Tyr, L- or D-Ser, L- or D-Thr, L- or D-Orn, L- or D-Arg, L- or D-Val
  MOD_RES (46): any natural or non natural amino acid residue different from L- or D-Cys residue, such as L- or D-Val, L- or D-Lys, L- or D-Arg, L- or D-Glx, L- or D-Asx, L- or D-Orn- or D-Ala, L- or D-Ile, L- or D-Val
  MOD_RES (47): any natural or non natural amino acid residue different from L- or D-Cys, such as L- or D-Glx, L- or D-Asx, L- or D-Lys, L- or D-Ile, L- or D-Leu, L- or D-Ala
  MOD_RES (48): any natural or non natural amino acid residue different from L- or D-Cys residue, such as L- or D-Lys, L- or D-Arg, L- or D-Orn- or D-Val, L- or D-Ile, L- or D-Leu
  MOD_RES (49): any natural or non natural amino acid residue different from L- or D-Cys residue, such as L- or D-Lys, L- or D-Arg, L- or D-Orn- or D-Val, L- or D-Ile, L- or D-Leu
  MOD_RES (50): no residue, or is any natural or non natural amino acid residue different from L- or D-Cys, such as L- or D-Glx, L- or D-Asx, L- or D-Lys, L- or D-Ile, L- or D-Leu, L- or D-Ala
  MOD_RES (51): no residue, or is any natural or non natural amino acid residue different from L- or D-Cys residue, such as L- or D-Ala, Gly, or D-L-Phe
  MOD_RES (52): no residue, or is any natural or non natural amino acid residue different from L- or D-Cys residue, such as Gly, L- or D-Pro, L- or D-L- or D-Val, or L- or D-Ala
  MOD_RES (53): no residue, or is L- or D-Asx, L- or D-Glx, L- or D-Ser, L- or D-Thr, L- or D-Tyr, L- or D-Lys, L or D-Arg, Gly
  MOD_RES (54)..(57): no residue, or is one to four natural or non natural amino acid residues different from L- or D-Cys; see specification as filed for specific embodiments

SEQ ID NO: 75 - protein, 56 aa, Artificial Sequence
  Description of Artificial Sequence: Synthetic peptide
  MOD_RES (1): any natural or non natural amino acid residue different from L- or D-Cys, such as L- or D-Glx, L- or D-Asx, L- or D-Leu, L- or D-Ile, L- or D-Arg, L- or D-Ser, L- or D-Thr
  MOD_RES (2): any natural or non natural amino acid residue different from L- or D-Cys, such as L- or D-Glx, L- or D-Asx, L- or D-Lys, L- or D-Ile, L- or D-Leu, L- or D-Ala
  MOD_RES (3): L- or D-Arg, L- or D-Lys, L- or D-Orn- or L- or D-Glx, L- or D-Asx, L- or D-Thr, L- or D-Tyr
  MOD_RES (4): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (5): L- or D-Met, L- or D-Ieu, L- or D-Asx, L- or D-Nle, L- or  D-Ala, L- or D-Phe, L- or D-Glx, L- or D-Ile, L- or D-Tyr
  MOD_RES (6): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (7): any natural or non natural amino acid residue different from L- or D-Cys residue, such as  L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (8): any natural or non natural amino acid residue different from L- or D-Cys residue, such as L- or D-Lys, L- or D-Arg, L- or D-Orn- or D-Val, L- or D-Ile, L- or D-Leu
  MOD_RES (9): any natural or non natural amino acid residue different from L- or D-Cys residue, such as L- or D-Thr, L- or D-Glx, L- or D-L- or D-Asx, L- or D-Tyr
  MOD_RES (10): any natural or non natural amino acid residue different from L- or D-Cys residue, such as L- or D-Thr, L- or D-Glx, L- or D-L- or D-Asx, L- or D-Tyr
  MOD_RES (11): any natural or non natural amino acid different from L- or D-Cys, such as L- or D-Asx, L-or D-Glx, L- or D-Ser, L- or D-Thr, L- or D-Tyr, L- or D-Lys
  MOD_RES (12): any natural or non natural amino acid residue different from L- or D-Cys residue, such as Gly, L- or D-Pro, L- or D-L- or D-Val, or L- or D-Ala
  MOD_RES (13): any natural or non natural amino acid residue different from L- or D-Cys residue, such as  L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (14): L- or D-His, L- or D-Lys, L- or D-Arg, L- or D-Orn, L- or D-Trp, L- or D-Ala, L- or D-Phe, L- or D-Pro
  MOD_RES (15): any natural or non natural amino acid residue residue different from L- or D-Cys, such as L- or D-Pro, L- or D-Ala, L- or D-Leu- or L- or D-Ile, L- or D-Phe, L- or D-Thr, L- or D-Tyr
  MOD_RES (16)..(35): region may encompass 1-20 variable amino acids
  MOD_RES (36)..(40): one or up to five natural or non natural amino acid residue different from L- or D-Cys; see specification as filed for specific embodiments
  MOD_RES (41): L- or D-Leu, L- or D-Ile, L- or D-Lys, L- or D-Phe, L- or D-Tyr, L- or D-Ser, L- or D-Thr, L- or D-Orn, L- or D-Arg, L- or D-Val
  MOD_RES (42): any natural or non natural amino acid residue different from L- or D-Cys residue, such as L- or D-Lys, L- or D-Arg, L- or D-Orn- or D-Val, L- or D-Ile, L- or D-Leu
  MOD_RES (43): any natural or non natural amino acid residue different from L- or D-Cys residue, such as L- or D-Lys, L- or D-Arg, L- or D-Orn- or D-Val, L- or D-Ile, L- or D-Leu
  MOD_RES (44): L- or D-Leu, L- or D-Ile, L- or D-Lys, L- or D-Phe, L- or D-Tyr, L- or D-Ser, L- or D-Thr, L- or D-Orn, L- or D-Arg, L- or D-Val
  MOD_RES (45): any natural or non natural amino acid residue different from L- or D-Cys residue, such as L- or D-Val, L- or D-Lys, L- or D-Arg, L- or D-Glx, L- or D-Asx, L- or D-Orn- or D-Ala, L- or D-Ile, L- or D-Val
  MOD_RES (46): any natural or non natural amino acid residue different from L- or D-Cys, such as L- or D-Glx, L- or D-Asx, L- or D-Lys, L- or D-Ile, L- or D-Leu, L- or D-Ala
  MOD_RES (47): any natural or non natural amino acid residue different from  L- or D-Cys residue, such as L- or D-Lys, L- or D-Arg, L- or D-Orn- or D-Val, L- or D-Ile, L- or D-Leu
  MOD_RES (48): any natural or non natural amino acid residue different from L- or D-Cys residue, such as L- or D-Lys, L- or D-Arg, L- or D-Orn- or D-Val, L- or D-Ile, L- or D-Leu
  MOD_RES (49): no residue, or is any natural or non natural amino acid residue different from L- or D-Cys, such as L- or D-Glx, L- or D-Asx, L- or D-Lys, L- or D-Ile, L- or D-Leu, L- or D-Ala
  MOD_RES (50): no residue, or is any natural or non natural amino acid residue different from L- or D-Cys residue, such as L- or D-Ala, Gly, or D-L-Phe
  MOD_RES (51): no residue, or is any natural or non natural amino acid residue different from L- or D-Cys residue, such as Gly, L- or D-Pro, L- or D-L- or D-Val, or L- or D-Ala
  MOD_RES (52): no residue, or is L- or D-Asx, L- or D-Glx, L- or D-Ser, L- or D-Thr, L- or D-Tyr, L- or D-Lys, L or D-Arg, Gly
  MOD_RES (53)..(56): no residue, or is one to four natural or non natural amino acid residues different from L- or D-Cys; see specification as filed for specific embodiments
XXXXXXXXXXXXXXXXXXXXXXXXXXXXXXXXXXXXXXXXXXXXXXXXXXXXXXXX

SEQ ID NO: 76 - protein, 50 aa, Artificial Sequence
  Description of Artificial Sequence: Synthetic peptide
  MOD_RES (17)..(36): region may encompass 1-20 variable amino acids
AELRCMCIKTTSGIHPXXXXXXXXXXXXXXXXXXXXRIKKIVQKKLAGDE

SEQ ID NO: 77 - protein, 50 aa, Artificial Sequence
  Description of Artificial Sequence: Synthetic peptide
  MOD_RES (17)..(36): region may encompass 1-20 variable amino acids
ELRCMCIKTTSGIHPKXXXXXXXXXXXXXXXXXXXXRIKKIVQKKLAGDE

SEQ ID NO: 78 - protein, 52 aa, Artificial Sequence
  Description of Artificial Sequence: Synthetic peptide
  MOD_RES (17)..(36): region may encompass 1-20 variable amino acids
AELRCMCIKTTSGIHPXXXXXXXXXXXXXXXXXXXXPDPDAPRIKKIVQKKL

SEQ ID NO: 79 - protein, 50 aa, Artificial Sequence
  Description of Artificial Sequence: Synthetic peptide
  MOD_RES (17)..(36): region may encompass 1-20 variable amino acids
AELRCMCIKTTSGIHPXXXXXXXXXXXXXXXXXXXXIKKIVQKKLAGDES

SEQ ID NO: 80 - protein, 50 aa, Artificial Sequence
  Description of Artificial Sequence: Synthetic peptide
  MOD_RES (17)..(36): region may encompass 1-20 variable amino acids
ELRCMCIKTTSGIHPKXXXXXXXXXXXXXXXXXXXXKKIVQKKLAGDESA

SEQ ID NO: 81 - protein, 50 aa, Artificial Sequence
  Description of Artificial Sequence: Synthetic peptide
  MOD_RES (17)..(36): region may encompass 1-20 variable amino acids
AELRCMCIKTTSGIHPXXXXXXXXXXXXXXXXXXXXKIVQKKLAGDESAD

SEQ ID NO: 82 - protein, 50 aa, Artificial Sequence
  Description of Artificial Sequence: Synthetic peptide
  MOD_RES (17)..(36): region may encompass 1-20 variable amino acids
ELRCMCIKTTSGIHPKXXXXXXXXXXXXXXXXXXXXKIVQKKLAGDESAD

SEQ ID NO: 83 - protein, 50 aa, Artificial Sequence; identical to SEQ ID NO: 82
  Description of Artificial Sequence: Synthetic peptide
  MOD_RES (17)..(36): region may encompass 1-20 variable amino acids

SEQ ID NO: 84 - protein, 35 aa, Artificial Sequence
  Description of Artificial Sequence: Synthetic peptide
SAKELRCQCIKTYSKGGGGNWVQRVVEKFLKRAEN

SEQ ID NO: 85 - protein, 33 aa, Artificial Sequence
  Description of Artificial Sequence: Synthetic peptide
SAKELRCQCIKTYGGGGNWVQRVVEKFLKRAEN

SEQ ID NO: 86 - protein, 16 aa, Artificial Sequence
  Description of Artificial Sequence: Synthetic peptide
NWVQRVVEKFLKRAEN

SEQ ID NO: 87 - protein, 35 aa, Artificial Sequence; identical to SEQ ID NO: 84
  Description of Artificial Sequence: Synthetic peptide

SEQ ID NO: 88 - protein, 32 aa, Artificial Sequence
  Description of Artificial Sequence: Synthetic peptide
  MOD_RES (16): UDA
SAKELRCQCIKTYSKXNWVQRVVEKFLKRAEN

SEQ ID NO: 89 - protein, 32 aa, Artificial Sequence
  Description of Artificial Sequence: Synthetic peptide
  MOD_RES (16): UDA
SAKELRAQFIKTYSKXNWVQRVVEKFLKRAEN

SEQ ID NO: 90 - protein, 32 aa, Artificial Sequence
  Description of Artificial Sequence: Synthetic peptide
  MOD_RES (16): UDA
SAKELRSQSIKTYSKXNWVQRVVEKFLKRAEN

SEQ ID NO: 91 - protein, 32 aa, Artificial Sequence
  Description of Artificial Sequence: Synthetic peptide
  MOD_RES (16): UDA
SAKELRAQYIKTYSKXNWVQRVVEKFLKRAEN

SEQ ID NO: 92 - protein, 32 aa, Artificial Sequence; identical to SEQ ID NO: 91
  Description of Artificial Sequence: Synthetic peptide
  MOD_RES (16): UDA

SEQ ID NO: 93 - protein, 32 aa, Artificial Sequence
  Description of Artificial Sequence: Synthetic peptide
  MOD_RES (16): UDA
SAKELRYQFIKTYSKXNWVQRVVEKFLKRAEN

SEQ ID NO: 94 - protein, 32 aa, Artificial Sequence; identical to SEQ ID NO: 93
  Description of Artificial Sequence: Synthetic peptide
  MOD_RES (16): UDA

SEQ ID NO: 95 - protein, 32 aa, Artificial Sequence
  Description of Artificial Sequence: Synthetic peptide
  MOD_RES (16): UDA
SAKELRYQAIKTYSKXNWVQRVVEKFLKRAEN

SEQ ID NO: 96 - protein, 32 aa, Artificial Sequence; identical to SEQ ID NO: 95
  Description of Artificial Sequence: Synthetic peptide
  MOD_RES (16): UDA

SEQ ID NO: 97 - protein, 32 aa, Artificial Sequence
  Description of Artificial Sequence: Synthetic peptide
  MOD_RES (16): UDA
SAKELRYQYIKTYSKXNWVQRVVEKFLKRAEN

SEQ ID NO: 98 - protein, 32 aa, Artificial Sequence; identical to SEQ ID NO: 97
  Description of Artificial Sequence: Synthetic peptide
  MOD_RES (16): UDA

SEQ ID NO: 99 - protein, 32 aa, Artificial Sequence
  Description of Artificial Sequence: Synthetic peptide
  MOD_RES (16): UDA
SAKELRYQFIRTYSKXNWVQRVVEKFLKRAEN

SEQ ID NO: 100 - protein, 32 aa, Artificial Sequence; identical to SEQ ID NO: 89
  Description of Artificial Sequence: Synthetic peptide
  MOD_RES (16): UDA

SEQ ID NO: 101 - protein, 32 aa, Artificial Sequence; identical to SEQ ID NO: 89
  Description of Artificial Sequence: Synthetic peptide
  MOD_RES (16): UDA

SEQ ID NO: 102 - protein, 32 aa, Artificial Sequence; identical to SEQ ID NO: 91
  Description of Artificial Sequence: Synthetic peptide
  MOD_RES (16): UDA

SEQ ID NO: 103 - protein, 32 aa, Artificial Sequence; identical to SEQ ID NO: 93
  Description of Artificial Sequence: Synthetic peptide
  MOD_RES (16): UDA

SEQ ID NO: 104 - protein, 32 aa, Artificial Sequence; identical to SEQ ID NO: 97
  Description of Artificial Sequence: Synthetic peptide
  MOD_RES (16): UDA

SEQ ID NO: 105 - protein, 32 aa, Artificial Sequence
  Description of Artificial Sequence: Synthetic peptide
  MOD_RES (16): UDA
SAKELRHQYIKTYSKXNWVQRVVEKFLKRAEN

SEQ ID NO: 106 - protein, 32 aa, Artificial Sequence
  Description of Artificial Sequence: Synthetic peptide
  MOD_RES (16): UDA
SAKELRYQWIKTYSKXNWVQRVVEKFLKRAEN

SEQ ID NO: 107 - protein, 32 aa, Artificial Sequence
  Description of Artificial Sequence: Synthetic peptide
  MOD_RES (16): UDA
SAKELRAQFIRTYSKXNWVQRVVEKFLKRAEN

SEQ ID NO: 108 - protein, 32 aa, Artificial Sequence
  Description of Artificial Sequence: Synthetic peptide
  MOD_RES (16): UDA
SAKELRYQWIRTYSKXNWVQRVVEKFLKRAEN

SEQ ID NO: 109 - protein, 32 aa, Artificial Sequence
  Description of Artificial Sequence: Synthetic peptide
  MOD_RES (16): UDA
SAKELRYQWIRTYSRXNWVQRVVEKFLKRAEN

SEQ ID NO: 110 - protein, 32 aa, Artificial Sequence
  Description of Artificial Sequence: Synthetic peptide
  MOD_RES (16): UDA
SAKELRWQWIRTYSRXNWVQRVVEKFLKRAEN

SEQ ID NO: 111 - protein, 32 aa, Artificial Sequence
  Description of Artificial Sequence: Synthetic peptide
  MOD_RES (16): UDA
SAKELRHQWIRTYSRXNWVQRVVEKFLKRAEN

SEQ ID NO: 112 - protein, 56 aa, Artificial Sequence; identical to SEQ ID NO: 75
  Description of Artificial Sequence: Synthetic peptide
  MOD_RES (1): any natural or non natural amino acid residue different from L- or D-Cys residue, such as  L- or D-Thr, L- or D-Glx, L- or D-L- or D-Asx, L- or D-Tyr
  MOD_RES (2): any natural or non natural amino acid residue residue different from L- or D-Cys, such as L- or D-Pro, L- or D-Ala, L- or D-Leu- or L- or D-Ile, L- or D-Phe, L- or D-Thr, L- or D-Tyr
  MOD_RES (3): any natural or non natural amino acid residue different from L- or D-Cys residue, such as L- or D-Val, L- or D-Lys, L- or D-Arg, L- or D-Glx, L- or D-Asx, L- or D-Orn- or D-Ala, L- or D-Ile, L- or D-Val
  MOD_RES (4): any natural or non natural amino acid residue different from L- or D-Cys residue, such as L- or D-Val, L- or D-Lys, L- or D-Arg, L- or D-Glx, L- or D-Asx, L- or D-Orn- or D-Ala, L- or D-Ile, L- or D-Val
  MOD_RES (5): L- or D-Arg, L- or D-Lys, L- or D-Orn- or L- or D-Glx, L- or D-Asx, L- or D-Thr, L- or D-Tyr
  MOD_RES (6): any natural or non natural amino acid residue different from L- or D-Cys residue, such as L- or D-Lys, L- or D-Arg, L- or D-Orn- or D-Val, L- or D-Ile, L- or D-Leu
  MOD_RES (7): any natural or non natural amino acid residue different from L- or D-Cys residue, such as Gly, L- or D-Pro, L- or D-L- or D-Val, or L- or D-Ala
  MOD_RES (8): L- or D-Arg, L- or D-Lys, L- or D-Orn- or L- or D-Glx, L- or D-Asx, L- or D-Thr, L- or D-Tyr
  MOD_RES (9): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (10): any natural or non natural amino acid different from L- or D-Cys, such as L- or D-Asx, L-or D-Glx, L- or D-Ser, L- or D-Thr, L- or D-Tyr, L- or D-Lys
  MOD_RES (11): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (12): any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (13): any natural or non natural amino acid different from L- or D-Cys, such as L- or D-Asx, L-or D-Glx, L- or D-Ser, L- or D-Thr, L- or D-Tyr, L- or D-Lys
  MOD_RES (14): any natural or non natural amino acid residue different from L- or D-Cys residue, such as L- or D-Thr, L- or D-Glx, L- or D-L- or D-Asx, L- or D-Tyr
  MOD_RES (15): L- or D-Asx, L- or D-Glx, L- or D-Arg, L- or D-Lys, L- or D-Ala, L- or D-Orn, Gly
  MOD_RES (16): any natural or non natural amino acid residue different from L- or D-Cys, such as L- or D-Glx, L- or D-Asx, L- or D-Lys, L- or D-Ile, L- or D-Leu, L- or D-Ala
  MOD_RES (17)..(36): region may encompass 1-20 variable amino acids
  MOD_RES (37): any natural or non natural amino acid residue different from L- or D-Cys residue, such as L- or D-Glx, L- or D-Asx, L- or D-Ala, L- or D-Arg, or L- or D-Phe, L- or D-Tyr
  MOD_RES (38): L- or D-Ser, L- or D-Thr, L- or D-Lys, L- or D-Phe, L- or D-Tyr, L- or D-Arg, L- or D-Ala, L- or D-His, L- or D-Trp, L- or D-Val
  MOD_RES (39): L- or D-Ser, L- or D-Thr, L- or D-Lys, L- or D-Phe, L- or D-Tyr, L- or D-Arg, L- or D-Ala, L- or D-His, L- or D-Trp, L- or D-Val
  MOD_RES (40): any natural or non natural amino acid residue different from L- or D-Cys residue, such as  L- or D-Glx, L- or D-Asx, L- or D-Lys, L- or D-Arg, or L- or D-Phe, L- or D-Tyr
  MOD_RES (41): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile, L- or D-Val
  MOD_RES (42): L- or D-Ser, L- or D-Thr, L- or D-Lys, L- or D-Arg, L- or D-Tyr, L- or D-Leu, L- or D-Ile, L- or D-Nle, L- or D-Ala, L- or D-Val
  MOD_RES (43): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile, L- or D-Val; L- or D-Lys
  MOD_RES (44): L- or D-Leu, L- or D-Thr, L- or D-Lys, L- or D-Arg, L- or D-Tyr, L- or D-Ser, L- or D-Ile, L- or D-Nle, L- or D-Ala, L- or D-Val
  MOD_RES (45): any natural or non natural amino acid residue different from L- or D-Cys residue, such as L- or D-Lys, L- or D-Arg, L- or D-Orn- or D-Val, L- or D-Ile, L- or D-Leu
  MOD_RES (46): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile, L- or D-Val
  MOD_RES (47): any natural or non natural amino acid residue different from L- or D-Cys residue, such as L- or D-Glx, L- or D-Asx, L- or D-Lys, L- or D-Arg, or L- or D-Ser, L- or D-Tyr, L- or D-Thr
  MOD_RES (48): any natural or non natural amino acid residue different from L- or D-Cys residue, such as L- or D-Glx, L- or D-Asx, L- or D-Trp, L- or D-His, or L- or D-Phe, L- or D-Tyr, L- or D-Thr, L- or D-Ser
  MOD_RES (49): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile, L- or D-Val; L- or D-Arg
  MOD_RES (50)..(56): one or up to seven natural or non natural amino acid residues different from L- or D-Cys; see specification as filed for further embodiments

SEQ ID NO: 113 - protein, 50 aa, Artificial Sequence
  Description of Artificial Sequence: Synthetic peptide
  MOD_RES (17)..(36): region may encompass 1-20 variable amino acids
TPVVRKGRCSCISTNQXXXXXXXXXXXXXXXXXXXXDSADVKELIKKWEK

SEQ ID NO: 114 - protein, 50 aa, Artificial Sequence
  Description of Artificial Sequence: Synthetic peptide
  MOD_RES (17)..(36): region may encompass 1-20 variable amino acids
TPVVRKGRCSCISTNQXXXXXXXXXXXXXXXXXXXXSADVKELIKKWEKQ

SEQ ID NO: 115 - protein, 50 aa, Artificial Sequence
  Description of Artificial Sequence: Synthetic peptide
  MOD_RES (17)..(36): region may encompass 1-20 variable amino acids
TPVVRKGRCSCISTNQXXXXXXXXXXXXXXXXXXXXQKKKVLKVRKSQRS

SEQ ID NO: 116 - protein, 50 aa, Artificial Sequence
  Description of Artificial Sequence: Synthetic peptide
  MOD_RES (17)..(36): region may encompass 1-20 variable amino acids
TPVVRKGRCSCISTNQXXXXXXXXXXXXXXXXXXXXKKKVLKVRKSQRSR

SEQ ID NO: 117 - protein, 50 aa, Artificial Sequence
  Description of Artificial Sequence: Synthetic peptide
  MOD_RES (17)..(36): region may encompass 1-20 variable amino acids
TPVVRKGRCSCISTNQXXXXXXXXXXXXXXXXXXXXKKVLKVRKSQRSRQ

SEQ ID NO: 118 - protein, 50 aa, Artificial Sequence
  Description of Artificial Sequence: Synthetic peptide
  MOD_RES (17)..(36): region may encompass 1-20 variable amino acids
TPVVRKGRCSCISTNQXXXXXXXXXXXXXXXXXXXXKVLKVRKSQRSRQK

SEQ ID NO: 119 - protein, 50 aa, Artificial Sequence
  Description of Artificial Sequence: Synthetic peptide
  MOD_RES (17)..(36): region may encompass 1-20 variable amino acids
TPVVRKGRCSCISTNQXXXXXXXXXXXXXXXXXXXXVLKVRKSQRSRQKK

SEQ ID NO: 120 - protein, 50 aa, Artificial Sequence
  Description of Artificial Sequence: Synthetic peptide
  MOD_RES (17)..(36): region may encompass 1-20 variable amino acids
TPVVRKGRCSCISTNQXXXXXXXXXXXXXXXXXXXXLKVRKSQRSRQKKT

SEQ ID NO: 121 - protein, 50 aa, Artificial Sequence
  Description of Artificial Sequence: Synthetic peptide
  MOD_RES (17)..(36): region may encompass 1-20 variable amino acids
TPVVRKGRCSCISTNQXXXXXXXXXXXXXXXXXXXXKVRKSQRSRQKKTT

SEQ ID NO: 122 - protein, 50 aa, Artificial Sequence; identical to SEQ ID NO: 114
  Description of Artificial Sequence: Synthetic peptide
  MOD_RES (17)..(36): region may encompass 1-20 variable amino acids

SEQ ID NO: 123 - protein, 30 aa, Artificial Sequence
  Description of Artificial Sequence: Synthetic peptide
  MOD_RES (17): UDA
VPLSRTVRCTCISISNXLKAVSKEMSKRSP

SEQ ID NO: 124 - protein, 30 aa, Artificial Sequence
  Description of Artificial Sequence: Synthetic peptide
  MOD_RES (17): UDA
VPLSRTVRATFISISNXLKAVSKEMSKRSP

SEQ ID NO: 125 - protein, 30 aa, Artificial Sequence
  Description of Artificial Sequence: Synthetic peptide
  MOD_RES (16): UDA
VPLSRTPRCTCISISXESKAIKNLLKAVSK

SEQ ID NO: 126 - protein, 30 aa, Artificial Sequence
  Description of Artificial Sequence: Synthetic peptide
  MOD_RES (16): UDA
VPLSRTVRSTSISISXESKAIENLLKAVSK

SEQ ID NO: 127 - protein, 30 aa, Artificial Sequence
  Description of Artificial Sequence: Synthetic peptide
  MOD_RES (16): UDA
VPLSRTVRSTSISISXESKAIKNLLEAVSK

SEQ ID NO: 128 - protein, 56 aa, Artificial Sequence; identical to SEQ ID NO: 75
  Description of Artificial Sequence: Synthetic peptide
  MOD_RES (1): any natural or non natural amino acid residue different from L- or D-Cys residue, such as L- or D-Phe, L- or D-His, L- or D-L- or D-Trp, L- or D-Tyr
  MOD_RES (2): any natural or non natural amino acid residue residue different from L- or D-Cys, such as L- or D-Pro, L- or D-Ala, L- or D-Leu- or L- or D-Ile, L- or D-Phe, L- or D-Thr, L- or D-Tyr
  MOD_RES (3): any natural or non natural amino acid residue different from L- or D-Cys residue, such as L- or D-Met, L- or D-Nle, L- or D-Leu, L- or D-Glx, L- or D-Asx, L- or D-Orn- or D-Ala, L- or D-Ile, L- or D-Val
  MOD_RES (4): any natural or non natural amino acid residue different from L- or D-Cys residue, such as L- or D-Phe, L- or D-His, L- or D-L- or D-Trp, L- or D-Tyr
  MOD_RES (5): any natural or non natural amino acid residue different from L- or D-Cys residue, such as L- or D-Lys, L- or D-Arg, L- or D-Orn- or D-Val, L- or D-Ile, L- or D-Leu
  MOD_RES (6): L- or D-Arg, L- or D-Lys, L- or D-Orn- or L- or D-Glx, L- or D-Asx, L- or D-Thr, L- or D-Tyr
  MOD_RES (7): any natural or non natural amino acid residue different from L- or D-Cys residue, such as Gly, L- or D-Pro, L- or D-L- or D-Val, or L- or D-Ala
  MOD_RES (8): L- or D-Arg, L- or D-Lys, L- or D-Orn- or L- or D-Glx, L- or D-Asx, L- or D-Thr, L- or D-Tyr
  MOD_RES (9): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (10): any natural or non natural amino acid different from L- or D-Cys, such as L- or D-Leu, L-or D-Ile, L- or D-Ala, L- or D-Thr, L- or D-Tyr, L- or D-Nle, L- or D-Ser
  MOD_RES (11): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (12): any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (13): any natural or non natural amino acid residue different from L- or D-Cys residue, such as  Gly, L- or D-Pro, L- or D-L- or D-Val, or L- or D-Ala
  MOD_RES (14): any natural or non natural amino acid residue residue different from L- or D-Cys, such as L- or D-Pro, L- or D-Ala, L- or D-Leu- or L- or D-Ile, L- or D-Phe, L- or D-Thr, L- or D-Tyr
  MOD_RES (15): any natural or non natural amino acid residue different from L- or D-Cys residue, such as  Gly, L- or D-Pro, L- or D-L- or D-Val, or L- or D-Ala
  MOD_RES (16): any natural or non natural amino acid residue different from L- or D-Cys residue, such as L- or D-Val, L- or D-Lys, L- or D-Arg, L- or D-Glx, L- or D-Asx, L- or D-Orn- or D-Ala, L- or D-Ile, L- or D-Val
  MOD_RES (17)..(36): region may encompass 1-20 variable amino acids
  MOD_RES (37)..(40): one or four natural or non natural amino acid residue different from L- or D-Cys; see specification as filed for further embodiments
  MOD_RES (41): L- or D-Ser, L- or D-Thr, L- or D-Lys, L- or D-Phe, L- or D-Tyr, L- or D-Arg, L- or D-Ala, L- or D-His, L- or D-Trp, L- or D-Val
  MOD_RES (42): L- or D-Ser, L- or D-Thr, L- or D-Lys, L- or D-Phe, L- or D-Tyr, L- or D-Arg, L- or D-Ala, L- or D-His, L- or D-Trp, L- or D-Val
  MOD_RES (43): any natural or non natural amino acid residue different from L- or D-Cys residue, such as  L- or D-Glx, L- or D-Asx, L- or D-Lys, L- or D-Arg, or L- or D-Phe, L- or D-Tyr
  MOD_RES (44): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile, L- or D-Val
  MOD_RES (45): any natural or non natural amino acid residue different from L- or D-Cys residue, such as  L- or D-Ser, L- or D-Thr, L- or D-Lys, L- or D-Arg, L- or D-Tyr, L- or D-Leu, L- or D-Ile, L- or D-Nle, L- or D-Ala, L- or D-Val, L- or D-Orn
  MOD_RES (46): any natural or non natural amino acid residue different from L- or D-Cys residue, such as L- or D-Leu, L- or D-Ile, L- or D-Ala, L- or D-Nle, L- or D-Val
  MOD_RES (47): any natural or non natural amino acid residue different from L- or D-Cys residue, such as L- or D-Leu, L- or D-Ile, L- or D-Ala, L- or D-Nle, L- or D-Val
  MOD_RES (48): any natural or non natural amino acid residue different from L- or D-Cys residue, such as L- or D-Leu, L- or D-Ile, L- or D-Ala, L- or D-Nle, L- or D-Val
  MOD_RES (49): any natural or non natural amino acid residue different from L- or D-Cys residue, such as L- or D-Val, L- or D-Lys, L- or D-Arg, L- or D-Glx, L- or D-Asx, L- or D-Orn- or D-Ala, L- or D-Ile, L- or D-Val
  MOD_RES (50): any natural or non natural amino acid residue different from L- or D-Cys residue, such as L- or D-Val, L- or D-Lys, L- or D-Arg, L- or D-Glx, L- or D-Asx, L- or D-Orn- or D-Ala, L- or D-Ile, L- or D-Val
  MOD_RES (51): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile, L- or D-Val; L- or D-Ala
  MOD_RES (52): any natural or non natural amino acid residue different from L- or D-Cys residue, such as L- or D-Val, L- or D-Lys, L- or D-Arg, L- or D-Glx, L- or D-Asx, L- or D-Orn- or D-Ala, L- or D-Ile, L- or D-Val
  MOD_RES (53)..(56): one or up to four natural or non natural amino acid residues different from L- or D-Cys; see specification as filed for further embodiments

SEQ ID NO: 129 - protein, 50 aa, Artificial Sequence
  Description of Artificial Sequence: Synthetic peptide
  MOD_RES (17)..(36): region may encompass 1-20 variable amino acids
FPMFKRGRCLCIGPGVXXXXXXXXXXXXXXXXXXXXKSKQARLIIKKVER

SEQ ID NO: 130 - protein, 50 aa, Artificial Sequence
  Description of Artificial Sequence: Synthetic peptide
  MOD_RES (17)..(36): region may encompass 1-20 variable amino acids
FPMFKRGRCLCIGPGVXXXXXXXXXXXXXXXXXXXXSKQARLIIKKVERK

SEQ ID NO: 131 - protein, 50 aa, Artificial Sequence
  Description of Artificial Sequence: Synthetic peptide
  MOD_RES (17)..(36): region may encompass 1-20 variable amino acids
FPMFKRGRCLCIGPGVXXXXXXXXXXXXXXXXXXXXKQARLIIKKVERKN

SEQ ID NO: 132 - protein, 50 aa, Artificial Sequence
  Description of Artificial Sequence: Synthetic peptide
  MOD_RES (17)..(36): region may encompass 1-20 variable amino acids
FPMFKRGRCLCIGPGVXXXXXXXXXXXXXXXXXXXXQARLIIKKVERKNF

SEQ ID NO: 133 - protein, 50 aa, Artificial Sequence; identical to SEQ ID NO: 130
  Description of Artificial Sequence: Synthetic peptide
  MOD_RES (17)..(36): region may encompass 1-20 variable amino acids

SEQ ID NO: 134 - protein, 31 aa, Artificial Sequence
  Description of Artificial Sequence: Synthetic peptide
KPVSLSYRCPCRFFGGGGLKWIQEYLEKALN

SEQ ID NO: 135 - protein, 31 aa, Artificial Sequence
  Description of Artificial Sequence: Synthetic peptide
KPVSLSYRAPFRFFGGGGLKWIQEYLEKALN

SEQ ID NO: 136 - protein, 31 aa, Artificial Sequence; identical to SEQ ID NO: 134
  Description of Artificial Sequence: Synthetic peptide

SEQ ID NO: 137 - protein, 28 aa, Artificial Sequence
  Description of Artificial Sequence: Synthetic peptide
  MOD_RES (15): UDA
KPVSLSYRCPCRFFXLKWIQEYLEKALN

SEQ ID NO: 138 - protein, 31 aa, Artificial Sequence; identical to SEQ ID NO: 134
  Description of Artificial Sequence: Synthetic peptide

SEQ ID NO: 139 - protein, 62 aa, Artificial Sequence
  Description of Artificial Sequence: Synthetic peptide
KPVSLSYRAPFRFFKGGGLKWIQEYLEKPVSLSYRAPFRFFGGKGLKWIQEYLEKALNKALN

SEQ ID NO: 140 - protein, 31 aa, Artificial Sequence
  Description of Artificial Sequence: Synthetic peptide
KPVSLSYRAPFRFFGKGGLKWIQEYLEKALN

SEQ ID NO: 141 - protein, 31 aa, Artificial Sequence
  Description of Artificial Sequence: Synthetic peptide
KPVSLSYRAPFRFFGGKGLKWIQEYLEKALN

SEQ ID NO: 142 - protein, 31 aa, Artificial Sequence
  Description of Artificial Sequence: Synthetic peptide
KPVSLSYRAPFRFFGGGKLKWIQEYLEKALN

SEQ ID NO: 143 - protein, 31 aa, Artificial Sequence
  Description of Artificial Sequence: Synthetic peptide
KPVSLSYRAPFRFFKKGGLKWIQEYLEKALN

SEQ ID NO: 144 - protein, 31 aa, Artificial Sequence
  Description of Artificial Sequence: Synthetic peptide
KPVSLSYRAPFRFFGKKGLKWIQEYLEKALN

SEQ ID NO: 145 - protein, 31 aa, Artificial Sequence
  Description of Artificial Sequence: Synthetic peptide
KPVSLSYRAPFRFFGGKKLKWIQEYLEKALN

SEQ ID NO: 146 - protein, 31 aa, Artificial Sequence
  Description of Artificial Sequence: Synthetic peptide
KPVSLSYRAPFRFFKGGKLKWIQEYLEKALN

SEQ ID NO: 147 - protein, 31 aa, Artificial Sequence
  Description of Artificial Sequence: Synthetic peptide
KPVSLSYRAPFRFFKGKGLKWIQEYLEKALN

SEQ ID NO: 148 - protein, 31 aa, Artificial Sequence
  Description of Artificial Sequence: Synthetic peptide
KPVSLSYRAPFRFFGKGKLKWIQEYLEKALN

SEQ ID NO: 149 - protein, 31 aa, Artificial Sequence
  Description of Artificial Sequence: Synthetic peptide
KPVSLSYRAPFRFFKKKGLKWIQEYLEKALN

SEQ ID NO: 150 - protein, 31 aa, Artificial Sequence
  Description of Artificial Sequence: Synthetic peptide
KPVSLSYRAPFRFFGKKKLKWIQEYLEKALN

SEQ ID NO: 151 - protein, 31 aa, Artificial Sequence
  Description of Artificial Sequence: Synthetic peptide
KPVSLSYRAPFRFFKGKKLKWIQEYLEKALN

SEQ ID NO: 152 - protein, 31 aa, Artificial Sequence
  Description of Artificial Sequence: Synthetic peptide
KPVSLSYRAPFRFFKKGKLKWIQEYLEKALN

SEQ ID NO: 153 - protein, 31 aa, Artificial Sequence
  Description of Artificial Sequence: Synthetic peptide
KPVSLSYRAPFRFFKKKKLKWIQEYLEKALN

SEQ ID NO: 154 - protein, 31 aa, Artificial Sequence
  Description of Artificial Sequence: Synthetic peptide
KPVSLSYRAPFRFFRGGGLKWIQEYLEKALN

SEQ ID NO: 155 - protein, 31 aa, Artificial Sequence
  Description of Artificial Sequence: Synthetic peptide
KPVSLSYRAPFRFFGRGGLKWIQEYLEKALN

SEQ ID NO: 156 - protein, 31 aa, Artificial Sequence
  Description of Artificial Sequence: Synthetic peptide
KPVSLSYRAPFRFFGGRGLKWIQEYLEKALN

SEQ ID NO: 157 - protein, 31 aa, Artificial Sequence
  Description of Artificial Sequence: Synthetic peptide
KPVSLSYRAPFRFFGGGRLKWIQEYLEKALN

SEQ ID NO: 158 - protein, 31 aa, Artificial Sequence
  Description of Artificial Sequence: Synthetic peptide
KPVSLSYRAPFRFFRRGGLKWIQEYLEKALN

SEQ ID NO: 159 - protein, 31 aa, Artificial Sequence
  Description of Artificial Sequence: Synthetic peptide
KPVSLSYRAPFRFFGRRGLKWIQEYLEKALN

SEQ ID NO: 160 - protein, 31 aa, Artificial Sequence
  Description of Artificial Sequence: Synthetic peptide
KPVSLSYRAPFRFFGGRRLKWIQEYLEKALN

SEQ ID NO: 161 - protein, 31 aa, Artificial Sequence
  Description of Artificial Sequence: Synthetic peptide
KPVSLSYRAPFRFFRGGRLKWIQEYLEKALN

SEQ ID NO: 162 - protein, 31 aa, Artificial Sequence
  Description of Artificial Sequence: Synthetic peptide
KPVSLSYRAPFRFFRGRGLKWIQEYLEKALN

SEQ ID NO: 163 - protein, 31 aa, Artificial Sequence
  Description of Artificial Sequence: Synthetic peptide
KPVSLSYRAPFRFFGRGRLKWIQEYLEKALN

SEQ ID NO: 164 - protein, 31 aa, Artificial Sequence
  Description of Artificial Sequence: Synthetic peptide
KPVSLSYRAPFRFFRRRGLKWIQEYLEKALN

SEQ ID NO: 165 - protein, 31 aa, Artificial Sequence
  Description of Artificial Sequence: Synthetic peptide
KPVSLSYRAPFRFFGRRRLKWIQEYLEKALN

SEQ ID NO: 166 - protein, 31 aa, Artificial Sequence
  Description of Artificial Sequence: Synthetic peptide
KPVSLSYRAPFRFFRGRRLKWIQEYLEKALN

SEQ ID NO: 167 - protein, 31 aa, Artificial Sequence
  Description of Artificial Sequence: Synthetic peptide
KPVSLSYRAPFRFFRRGRLKWIQEYLEKALN

SEQ ID NO: 168 - protein, 31 aa, Artificial Sequence
  Description of Artificial Sequence: Synthetic peptide
KPVSLSYRAPFRFFRRRRLKWIQEYLEKALN

SEQ ID NO: 169 - protein, 57 aa, Artificial Sequence; identical to SEQ ID NO: 18
  Description of Artificial Sequence: Synthetic peptide
  MOD_RES (1): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile, L- or D-Val
  MOD_RES (2): any natural or non natural amino acid residue different from L- or D-Cys residue, such as L- or D-Leu, L- or D-Ile, L- or D-Ala, L- or D-Nle, L- or D-Val
  MOD_RES (3): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile, L- or D-Val
  MOD_RES (4): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile, L- or D-Val
  MOD_RES (5): L- or D-Tyr, L- or D-Phe, L- or D-His- or L- or D-Glx, L- or D-Asx, L- or D-Thr, L- or D-Trp, L- or D-Ser
  MOD_RES (6): L- or D-Tyr, L- or D-Phe, L- or D-His- or L- or D-Glx, L- or D-Asx, L- or D-Thr, L- or D-Trp, L- or D-Ser
  MOD_RES (7): any natural or non natural amino acid residue different from L- or D-Cys residue, such as L- or D-Thr, L- or D-Ser, L- or D-Tyr, or L- or D-Ala
  MOD_RES (8): any natural or non natural amino acid residue different from L- or D-Cys residue, such as L- or D-Thr, L- or D-Ser, L- or D-Tyr, or L- or D-Ala
  MOD_RES (9): any natural or non natural amino acid different from L- or D-Cys, such as L- or D-Leu, L-or D-Ile, L- or D-Ala, L- or D-Thr, L- or D-Tyr, L- or D-Nle, L- or D-Ser
  MOD_RES (10): L- or D-Arg, L- or D-Lys, L- or D-Orn- or L- or D-Glx, L- or D-Asx, L- or D-Thr, L- or D-Tyr
  MOD_RES (11): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (12): L- or D-Arg, L- or D-Lys, L- or D-Orn- or L- or D-Glx, L- or D-Asx, L- or D-Thr, L- or D-Tyr
  MOD_RES (13): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (14): any natural or non natural amino acid residue different from L- or D-Cys residue, such as L- or D-Val, L- or D-Lys, L- or D-Arg, L- or D-Glx, L- or D-Asx, L- or D-Orn- or D-Ala, L- or D-Ile, L- or D-Val
  MOD_RES (15): any natural or non natural amino acid residue different from L- or D-Cys residue, such as L- or D-Val, L- or D-Lys, L- or D-Arg, L- or D-Glx, L- or D-Asx, L- or D-Orn- or D-Ala, L- or D-Ile, L- or D-Val
  MOD_RES (16): any natural or non natural amino acid residue different from L- or D-Cys residue, such as L- or D-Val, L- or D-Lys, L- or D-Arg, L- or D-Glx, L- or D-Asx, L- or D-Orn- or D-Ala, L- or D-Ile, L- or D-Val
  MOD_RES (17)..(36): region may encompass 1-20 variable amino acids
  MOD_RES (37): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile, L- or D-Val; L- or D-Gln
  MOD_RES (38): L- or D-Val, L- or D-Thr, L- or D-Lys, L- or D-Phe, L- or D-Tyr, L- or D-Arg, L- or D-Ala, L- or D-Ser, L- or D-Trp, L- or D-Leu, L- or D-Ile
  MOD_RES (39): any natural or non natural amino acid different from L- or D-Cys, such as L- or D-Leu, L-or D-Ile, L- or D-Ala, L- or D-Thr, L- or D-Tyr, L- or D-Nle, L- or D-Ser, L- or D-Glx, L- or D-Asx
  MOD_RES (40): L- or D-Arg, L- or D-Lys, L- or D-Orn- or L- or D-Glx, L- or D-Asx, L- or D-Thr, L- or D-Tyr,  L- or D- Trp, L- or D-His
  MOD_RES (41): any natural or non natural amino acid residue different from L- or D-Cys residue, such as L- or D-Val, L- or D-Lys, L- or D-Arg, L- or D-Glx, L- or D-Asx, L- or D-Orn- or D-Ala, L- or D-Ile, L- or D-Val
  MOD_RES (42): any natural or non natural amino acid residue different from L- or D-Cys residue, such as  L- or D-Ser, L- or D-Thr, L- or D-Lys, L- or D-Arg, L- or D-Tyr, L- or D-Leu, L- or D-Ile, L- or D-Nle, L- or D-Ala, L- or D-Glx, L- or D-Asx
  MOD_RES (43): any natural or non natural amino acid residue different from L- or D-Cys residue, such as L- or D-Thr, L- or D-Ser, L- or D-Tyr, or L- or D-Ala, L- or D-Arg
  MOD_RES (44): any natural or non natural amino acid residue different from L- or D-Cys residue, such as L- or D-Thr, L- or D-Ser, L- or D-Tyr, or L- or D-Ala, L- or D- Met, L- or D-Nle
  MOD_RES (45): any natural or non natural amino acid residue different from L- or D-Cys residue, such as L- or D-Thr, L- or D-Ser, L- or D-Tyr, or L- or D-Ala, L- or D-Met, L- or D-Nle
  MOD_RES (46): any natural or non natural amino acid residue different from L- or D-Cys residue, such as L- or D-Thr, L- or D-Ser, L- or D-Tyr, or L- or D-Ala, L- or D-Glx, L- or D-Asx; L- or D-Glu
  MOD_RES (47): any natural or non natural amino acid different from L- or D-Cys, such as L- or D-Leu, L-or D-Ile, L- or D-Ala, L- or D-Thr, L- or D-Tyr, L- or D-Nle, L- or D-Ser, L- or D-Val
  MOD_RES (48): any natural or non natural amino acid residue different from L- or D-Cys residue, such as L- or D-Pro, L- or D-Ala, L- or D-Leu, L- or D-Glx, L- or D-Asx, L- or D-Orn- or, L- or D-Val
  MOD_RES (49): L- or D-Val, L- or D-Thr, L- or D-Lys, L- or D-Phe, L- or D-Tyr, L- or D-Arg, L- or D-Ala, L- or D-Ser, L- or D-Trp, L- or D-Leu, L- or D-Ile
  MOD_RES (50)..(57): one or up to eight natural or non natural amino acid residues different from L- or D-Cys; see specification as filed for further embodiments

SEQ ID NO: 170 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXEVLRKRSSSTLPVP

SEQ ID NO: 171 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXQAEWIQRMMEVLRK

SEQ ID NO: 172 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXLRKRSSSTLPVPVP

SEQ ID NO: 173 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXRKRSSSTLPVPVPF

SEQ ID NO: 174 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXKRSSSTLPVPVPFK

SEQ ID NO: 175 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXRSSSTLPVPVPFKR

SEQ ID NO: 176 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXSSSTLPVPVPFKRK

SEQ ID NO: 177 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXSSTLPVPVPFKRKI

SEQ ID NO: 178 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXSTLPVPVPFKRKIP

SEQ ID NO: 179 - protein, 50 aa, Artificial Sequence; identical to SEQ ID NO: 170
  Description of Artificial Sequence: Synthetic peptide
  MOD_RES (17)..(36): region may encompass 1-20 variable amino acids

SEQ ID NO: 180 - protein, 50 aa, Artificial Sequence
  Description of Artificial Sequence: Synthetic peptide
  MOD_RES (1): any natural or non natural amino acid residue different from L- or D-Cys residue, such as L- or D-Thr, L- or D-Ser, L- or D-Tyr, or L- or D-Ala
  MOD_RES (2): any natural or non natural amino acid residue different from L- or D-Cys residue, such as L- or D-Lys, L- or D-Arg, L- or D-Orn- or D-Val, L- or D-Ile, L- or D-Leu
  MOD_RES (3): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (4): any natural or non natural amino acid residue different from L- or D-Cys residue, such as L- or D-Lys, L- or D-Arg, L- or D-Orn- or D-Val, L- or D-Ile, L- or D-Leu
  MOD_RES (5): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (6): any natural or non natural amino acid residue different from L- or D-Cys residue, such as L- or D-Thr, L- or D-Ser, L- or D-Tyr, or L- or D-Ala
  MOD_RES (7): L- or D-Arg, L- or D-Lys, L- or D-Orn- or L- or D-Glx, L- or D-Asx, L- or D-Thr, L- or D-Tyr
  MOD_RES (8): any natural or non natural amino acid residue different from L- or D-Cys residue, such as L- or D-Lys, L- or D-Arg, L- or D-Orn- or D-Val, L- or D-Ile, L- or D-Leu
  MOD_RES (9): any natural or non natural amino acid residue different from L- or D-Cys residue, such as  Gly, L- or D-Pro, L- or D-L- or D-Val, or L- or D-Ala
  MOD_RES (10): any natural or non natural amino acid residues different from L- or D-Cys, such as L- or D-Pro, L- or D-Val, L- or D-Phe, L- or D-Glx, L- or D-Asx, L- or D-Lys, L- or D-Arg, L- or D-Ile, L- or D-Ser
  MOD_RES (11): any natural or non natural amino acid residue different from L- or D-Cys residue, such as L- or D-Lys, L- or D-Arg, L- or D-Orn- or D-Val, L- or D-Ile, L- or D-Leu
  MOD_RES (12): any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (13): L- or D-Arg, L- or D-Lys, L- or D-Orn- or L- or D-Glx, L- or D-Asx, L- or D-Thr, L- or D-Tyr
  MOD_RES (14): L- or D-Tyr, L- or D-Phe, L- or D-His- or L- or D-Glx, L- or D-Asx, L- or D-Thr, L- or D-Trp, L- or D-Ser
  MOD_RES (15): any natural or non natural amino acid residue different from L- or D-Cys residue, such as L- or D-Thr, L- or D-Ser, L- or D-Tyr, or L- or D-Ala
  MOD_RES (16): any natural or non natural amino acid residue different from L- or D-Cys residue, such as L- or D-Val, L- or D-Lys, L- or D-Arg, L- or D-Glx, L- or D-Asx, L- or D-Orn- or D-Ala, L- or D-Ile, L- or D-Val
  MOD_RES (17)..(36): region may encompass 1-20 variable amino acids
  MOD_RES (37): any natural or non natural amino acid residue different from L- or D-Cys residue, such as  L- or D-Lys, L- or D-Trp, L- or D-Arg, L- or D-Phe, L- or D-Tyr, L- or D-His, L- or D-Ala, L- or D-Glx, L- or D-Asx, L- or D-Val
  MOD_RES (38): L- or D-Ser, L- or D-Thr, L- or D-Lys, L- or D-Phe, L- or D-Tyr, L- or D-Arg, L- or D-Ala, L- or D-His, L- or D-Trp, L- or D-Val, L- or D-Leu, L- or D-Ile
  MOD_RES (39): any natural or non natural amino acid residue different from L- or D-Cys residue, such as  L- or D-Glx, L- or D-Asx, L- or D-Lys, L- or D-Arg, or L- or D-Phe, L- or D-Tyr
  MOD_RES (40): any natural or non natural amino acid residue different from L- or D-Cys residue, such as L- or D-Val, L- or D-Lys, L- or D-Arg, L- or D-Glx, L- or D-Asx, L- or D-Ser- or D-Ala, L- or D-Thr, L- or D-Tyr
  MOD_RES (41): any natural or non natural amino acid residue different from L- or D-Cys residue, such as L- or D-Thr, L- or D-Ser, L- or D-Tyr, or L- or D-Trp
  MOD_RES (42): any natural or non natural amino acid residue different from L- or D-Cys residue, such as  L- or D-Lys, L- or D-Trp, L- or D-Arg, L- or D-Phe, L- or D-Tyr, L- or D-His, L- or D-Ala, L- or D-Glx, L- or D-Asx, L- or D-Val
  MOD_RES (43): any natural or non natural amino acid residue different from L- or D-Cys residue, such as L- or D-Val, L- or D-Lys, L- or D-Arg, L- or D-Glx, L- or D-Asx, L- or D-Orn- or D-Ala, L- or D-Ile, L- or D-Val
  MOD_RES (44): any natural or non natural amino acid residue different from L- or D-Cys residue, such as L- or D-Phe, L- or D-Lys, L- or D-Ala, L- or D-Arg
  MOD_RES (45): any natural or non natural amino acid residue different from L- or D-Cys residue, such as L- or D-Leu, L- or D-Ile, L- or D-Ala, L- or D-Nle, L- or D-Arg, L- or D-Lys; L- or D-Arg
  MOD_RES (46): any natural or non natural amino acid residue different from L- or D-Cys residue, such as L- or D-Val, L- or D-Lys, L- or D-Arg, L- or D-Glx, L- or D-Asx, L- or D-Orn- or D-Ala,  L- or D-Ile, L- or D-Val
  MOD_RES (47): any natural or non natural amino acid residue different from L- or D-Cys residue, such as L- or D-Val, L- or D-Lys, L- or D-Arg, L- or D-Glx, L- or D-Asx, L- or D-Trp- or D-Ala, L- or D-Phe, L- or D-His
  MOD_RES (48): L- or D-Tyr, L- or D-Phe, L- or D-His- or L- or D-Glx, L- or D-Asx, L- or D-Thr, L- or D-Trp, L- or D-Ser
  MOD_RES (49): any natural or non natural amino acid residue different from L- or D-Cys residue, such as L- or D-Val, L- or D-Lys, L- or D-Arg, L- or D-Glx, L- or D-Asx, L- or D-Orn- or D-Ala, L- or D-Ile, L- or D-Val
  MOD_RES (50): any natural or non natural amino acid residue different from L- or D-Cys residue, such as L- or D-Val, L- or D-Lys, L- or D-Arg, L- or D-Glx, L- or D-Asx, L- or D-Orn- or D-Ala, L- or D-Ile, L- or D-Val
XXXXXXXXXXXXXXXXXXXXXXXXXXXXXXXXXXXXXXXXXXXXXXXXXX

SEQ ID NO: 181 - protein, 50 aa, Artificial Sequence
  Description of Artificial Sequence: Synthetic peptide
  MOD_RES (17)..(36): region may encompass 1-20 variable amino acids
SKCKCSRKGPKIRYSDXXXXXXXXXXXXXXXXXXXXEVLRKRSSSTLPVP

SEQ ID NO: 182 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXKLQSTKRFIKWYNA

SEQ ID NO: 183 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXLQSTKRFIKWYNAW

SEQ ID NO: 184 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXQSTKRFIKWYNAWN

SEQ ID NO: 185 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXSTKRFIKWYNAWNE

SEQ ID NO: 186 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXTKRFIKWYNAWNEK

SEQ ID NO: 187 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXKRFIKWYNAWNEKR

SEQ ID NO: 188 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXRFIKWYNAWNEKRR

SEQ ID NO: 189 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXFIKWYNAWNEKRRV

SEQ ID NO: 190 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXIKWYNAWNEKRRVY

SEQ ID NO: 191 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXKWYNAWNEKRRVYE

SEQ ID NO: 192 - protein, 50 aa, Artificial Sequence
  Description of Artificial Sequence: Synthetic peptide
  MOD_RES (17)..(36): region may encompass 1-20 variable amino acids
VLEVYYTSLRCRCVQEXXXXXXXXXXXXXXXXXXXXWYNAWNEKRRVYEE

SEQ ID NO: 193 - protein, 50 aa, Artificial Sequence; identical to SEQ ID NO: 192
  Description of Artificial Sequence: Synthetic peptide
  MOD_RES (17)..(36): region may encompass 1-20 variable amino acids

SEQ ID NO: 194 - protein, 54 aa, Artificial Sequence; identical to SEQ ID NO: 19
  Description of Artificial Sequence: Synthetic peptide
  MOD_RES (1): any natural or non natural amino acid residue different from L- or D-Cys residue, such as L- or D-Val, L- or D-Lys, L- or D-Arg, L- or D-Glx, L- or D-Asx, L- or D-Orn- or D-Ala, L- or D-Ile
  MOD_RES (2): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile
  MOD_RES (3): any natural or non natural amino acid residue different from L- or D-Cys residue, such as  L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (4): L- or D-Arg, L- or D-Lys, L- or D-Orn- or L- or D-Glx, L- or D-Asx, L- or D-Thr, L- or D-Tyr
  MOD_RES (5): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (6): any natural or non natural amino acid residue different from L- or D-Cys residue, such as  L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (7): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (8): any natural or non natural amino acid residue different from L- or D-Cys residue, such as  L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (9): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile
  MOD_RES (10): any natural or non natural amino acid residue different from L- or D-Cys residue, such as L- or D-Val, L- or D-Lys, L- or D-Arg, L- or D-Glx, L- or D-Asx, L- or D-Orn- or D-Ala, L- or D-Ile
  MOD_RES (11): L- or D-His, L- or D-Lys, L- or D-Orn- or L- or D-Glx, L- or D-Asx, L- or D-Trp, L- or D-Phe, L- or D-Tyr
  MOD_RES (12): any natural or non natural amino acid residue different from L- or D-Cys residue, such as L- or D-Thr, L- or D-Ser, L- or D-Tyr, or L- or D-Ala
  MOD_RES (13): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile
  MOD_RES (14): any natural or non natural amino acid residue different from L- or D-Cys residue, such as L- or D-Phe, L- or D-Lys, L- or D-Ala, L- or D-Arg
  MOD_RES (15): any natural or non natural amino acid residue different from L- or D-Cys residue, such as L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (16): any natural or non natural amino acid residues different from L- or D-Cys, such as L- or D-Pro, L- or D-Val, L- or D-Phe, L- or D-Glx, L- or D-Asx, L- or D-Lys, L- or D-Arg, L- or D-Ile, L- or D-Ser
  MOD_RES (17)..(36): region may encompass 1-20 variable amino acids
  MOD_RES (37): any natural or non natural amino acid residue different from L- or D-Cys residue, such as  L- or D-Ile, L- or D-Glx, L- or D-Asx, L- or D-Leu, or L- or D-Ala, L- or D-Phe, L- or D-Tyr, L- or D-Glu, L- or D-Lys; L- or D-Asp
  MOD_RES (38): L- or D-Arg, L- or D-Lys, L- or D-Orn- or L- or D-Glx, L- or D-Asx, L- or D-Thr, L- or D-Tyr
  MOD_RES (39): any natural or non natural amino acid residue different from L- or D-Cys residue, such as  L- or D-Glx, L- or D-Asx, L- or D-Lys, L- or D-Arg, or L- or D-Phe, L- or D-Tyr; L- or D-Asn
  MOD_RES (40): any natural or non natural amino acid residue different from L- or D-Cys residue, such as L- or D-Phe, L- or D-Tyr, L- or D-His, L- or D-Glx, L- or D-Asx, L- or D-Ser- or D-Ala, lL- or D-Thr, L- or D-Trp; L- or D-Phe
  MOD_RES (41): L- or D-Ser, L- or D-Leu, L- or D-Ile- or L- or D-Glx, L- or D-Asx, L- or D-Thr L- or D-Tyr, L- or Ala, L- or D-Phe, L- or D-His
  MOD_RES (42): L- or D-Arg, L- or D-Lys, L- or D-Orn- or L- or D-Glx, L- or D-Asx, L- or D-Thr, L- or D-Tyr
  MOD_RES (43): any natural or non natural amino acid residue different from L- or D-Cys residue, such as L- or D-His, L- or D-Arg, L- or D-Glx, L- or D-Asx, L- or D-Orn- or D-Phe, L- or D-Ile, L- or D-Trp
  MOD_RES (44): any natural or non natural amino acid residue different from L- or D-Cys residue, such as L- or D-Phe, L- or D-Ser, L- or D-Thr, L- or D-Tyr
  MOD_RES (45): any natural or non natural amino acid residue different from L- or D-Cys residue, such as L- or D-Ser, L- or D-Thr, L- or D-Ala, L- or D-Tyr, L- or D-Arg, L- or D-Lys
  MOD_RES (46): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile, L- or D-Val
  MOD_RES (47): any natural or non natural amino acid residue different from L- or D-Cys residue, such as L- or D-Val, L- or D-Leu, L- or D-Ile, L- or D-Glx, L- or D-Asx, L- or D-Trp- or D-Ala, lL- or D-Phe, L- or D-His
  MOD_RES (48): any natural or non natural amino acid residue different from L- or D-Cys residue, such as L- or D-Ser, L- or D-Thr, L- or D-Ala, L- or D-Tyr, L- or D-Arg, L- or D-Lys
  MOD_RES (49): any natural or non natural amino acid residue different from L- or D-Cys residue, such as L- or D-His, L- or D-Leu, L- or D-Ile, L- or D-Glx, L- or D-Asx, L- or D-Trp- or D-Ala, L- or D-Phe
  MOD_RES (50)..(54): one or up to five natural or non natural amino acid residue different from L- or D-Cys; see specification as filed for specific embodiments

SEQ ID NO: 195 - protein, 50 aa, Artificial Sequence
  Description of Artificial Sequence: Synthetic peptide
  MOD_RES (17)..(36): region may encompass 1-20 variable amino acids
QELRCLCIQEHSEFIPXXXXXXXXXXXXXXXXXXXXIRETSRHFADLAHN

SEQ ID NO: 196 - protein, 50 aa, Artificial Sequence
  Description of Artificial Sequence: Synthetic peptide
  MOD_RES (17)..(36): region may encompass 1-20 variable amino acids
QELRCLCIQEHSEFIPXXXXXXXXXXXXXXXXXXXXDRNFLRDSSEVSLT

SEQ ID NO: 197 - protein, 50 aa, Artificial Sequence
  Description of Artificial Sequence: Synthetic peptide
  MOD_RES (17)..(36): region may encompass 1-20 variable amino acids
QELRCLCIQEHSEFIPXXXXXXXXXXXXXXXXXXXXRETSRHFADLAHNS

SEQ ID NO: 198 - protein, 50 aa, Artificial Sequence
  Description of Artificial Sequence: Synthetic peptide
  MOD_RES (17)..(36): region may encompass 1-20 variable amino acids
QELRCLCIQEHSEFIPXXXXXXXXXXXXXXXXXXXXRNFLRDSSEVSLTG

SEQ ID NO: 199 - protein, 50 aa, Artificial Sequence
  Description of Artificial Sequence: Synthetic peptide
  MOD_RES (17)..(36): region may encompass 1-20 variable amino acids
QELRCLCIQEHSEFIPXXXXXXXXXXXXXXXXXXXXNFLRDSSEVSLTGS

SEQ ID NO: 200 - protein, 50 aa, Artificial Sequence
  Description of Artificial Sequence: Synthetic peptide
  MOD_RES (17)..(36): region may encompass 1-20 variable amino acids
QELRCLCIQEHSEFIPXXXXXXXXXXXXXXXXXXXXFLRDSSEVSLTGSD

SEQ ID NO: 201 - protein, 50 aa, Artificial Sequence
  Description of Artificial Sequence: Synthetic peptide
  MOD_RES (17)..(36): region may encompass 1-20 variable amino acids
QELRCLCIQEHSEFIPXXXXXXXXXXXXXXXXXXXXLRDSSEVSLTGSDA

SEQ ID NO: 202 - protein, 50 aa, Artificial Sequence
  Description of Artificial Sequence: Synthetic peptide
  MOD_RES (17)..(36): region may encompass 1-20 variable amino acids
QELRCLCIQEHSEFIPXXXXXXXXXXXXXXXXXXXXIRETSKHFADLAHN

SEQ ID NO: 203 - protein, 50 aa, Artificial Sequence
  Description of Artificial Sequence: Synthetic peptide
  MOD_RES (17)..(36): region may encompass 1-20 variable amino acids
QELRCLCIQEHSEFIPXXXXXXXXXXXXXXXXXXXXDRNFLKDSSEVSLT

SEQ ID NO: 204 - protein, 55 aa, Artificial Sequence
  Description of Artificial Sequence: Synthetic peptide
  MOD_RES (1): any natural or non natural amino acid residue different from L- or D-Cys residue, such as  Gly, L- or D-Pro, L- or D-L- or D-Val, or L- or D-Ala
  MOD_RES (2): any natural or non natural amino acid residue different from L- or D-Cys residue, such as L- or D-Thr, L- or D-Ser, L- or D-Tyr, or L- or D-Ala
  MOD_RES (3): any natural or non natural amino acid residue different from L- or D-Cys residue, such as L- or D-Val, L- or D-Ala, L- or D-Leu- or D-Ile
  MOD_RES (4): any natural or non natural amino acid residue different from L- or D-Cys residue, such as L- or D-Thr, L- or D-Ser, L- or D-Tyr, or L- or D-Ala
  MOD_RES (5): any natural or non natural amino acid residue different from L- or D-Cys residue, such as  Gly, L- or D-Pro, L- or D-L- or D-Val, or L- or D-Ala
  MOD_RES (6): any natural or non natural amino acid residue different from L- or D-Cys residue, such as L- or D-Thr, L- or D-Ser, L- or D-Tyr, or L- or D-Ala
  MOD_RES (7): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (8): any natural or non natural amino acid residue different from L- or D-Cys residue, such as L- or D-Tyr, L- or D-Phe, L- or D-His- or L- or D-Glx, L- or D-Asx, L- or D-Thr, L- or D-Trp, L- or D-Ser
  MOD_RES (9): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe
  MOD_RES (10): any natural or non natural amino acid residue different from L- or D-Cys residue, such as  Gly, L- or D-Pro, L- or D-L- or D-Val, or L- or D-Ala
  MOD_RES (11): any natural or non natural amino acid residue different from L- or D-Cys residue, such as L- or D-Lys, L- or D-Arg, L- or D-Orn- or D-Val, L- or D-Ile, L- or D-Leu
  MOD_RES (12): L- or D-Arg, L- or D-Lys, L- or D-Orn- or L- or D-Glx, L- or D-Asx, L- or D-Thr, L- or D-Tyr
  MOD_RES (13): any natural or non natural amino acid residue different from L- or D-Cys residue, such as  L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (14): any natural or non natural amino acid residue different from L- or D-Cys residue, such as L- or D-Thr, L- or D-Ser, L- or D-Tyr, or L- or D-Ala
  MOD_RES (15): any natural or non natural amino acid residue different from L- or D-Cys residue, such as L- or D-Thr, L- or D-Ser, L- or D-Tyr, or L- or D-Ala
  MOD_RES (16): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile, L- or D-Val
  MOD_RES (17)..(36): region may encompass 1-20 variable amino acids
  MOD_RES (37): any natural or non natural amino acid residue different from L- or D-Cys residue, such asL- or D-Trp, L- or D-Arg, L- or D-Phe, L- or D-Tyr, L- or D-His, L- or D-Ala, L- or D-Glx, L- or D-Asx, L- or D-Val
  MOD_RES (38): any natural or non natural amino acid residue different from L- or D-Cys residue, such asL- or D-Val, L- or D-Ala, L- or D-Leu- or D-Ile
  MOD_RES (39): any natural or non natural amino acid residue different from L- or D-Cys residue, such as  L- or D-Glx, L- or D-Asx, L- or D-Lys, L- or D-Arg, or L- or D-Phe, L- or D-Tyr
  MOD_RES (40): any natural or non natural amino acid residue different from L- or D-Cys residue, such as L- or D-Val, L- or D-Lys, L- or D-Arg, L- or D-Glx, L- or D-Asx, L- or D-Ser- or D-Ala, lL- or D-Thr, L- or D-Tyr
  MOD_RES (41): any natural or non natural amino acid residue different from L- or D-Cys residue, such as  L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (42): L- or D-Met, L- or D-Nle, L- or D-His- or L- or D-Glx, L- or D-Asx, L- or D-Thr, L- or D-Trp, L- or D-Ser, L- or D-Tyr, L- or D-Lys, L- or D-Orn
  MOD_RES (43): any natural or non natural amino acid residue different from L- or D-Cys residue, such as L- or D-Thr, L- or D-Ser, L- or D-Tyr, or L- or D-Ala
  MOD_RES (44): L- or D-Cys, L- or D-Val, L- or D-Phe, L- or D-His, L- or D-Trp, L- or D-Ser, L- or D-Thr, L- or D-Lys, L- or D-His, L- or D-Tyr, L- or D-Ala, L- or D-Orn, L- or D-Trp, L- or D-His, L- or D-Phe, L-or D-Nle
  MOD_RES (45): any natural or non natural amino acid residue different from L- or D-Cys residue, such as  L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (46): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile, L- or D-Val
  MOD_RES (47): any natural or non natural amino acid residue different from L- or D-Cys residue, such as  L- or D-Ile, L- or D-Glx, L- or D-Asx, L- or D-Leu, or L- or D-Ala, L- or D-Phe, L- or D-Tyr, L- or D-Glu, L- or D-Lys
  MOD_RES (48): any natural or non natural amino acid residue different from L- or D-Cys residue, such as L- or D-Lys, L- or D-Arg, L- or D-Orn- or D-Val, L- or D-Ile, L- or D-Leu
  MOD_RES (49): any natural or non natural amino acid residue different from L- or D-Cys residue, such as L- or D-Val, L- or D-Lys, L- or D-Arg, L- or D-Glx, L- or D-Asx, L- or D-Orn- or D-Ala, lL- or D-Ile, L- or D-Val
  MOD_RES (50)..(55): one or up to six natural or non natural amino acid residues; see specification as filed for specific embodiments
XXXXXXXXXXXXXXXXXXXXXXXXXXXXXXXXXXXXXXXXXXXXXXXXXXXXXXX

SEQ ID NO: 205 - protein, 50 aa, Artificial Sequence
  Description of Artificial Sequence: Synthetic peptide
  MOD_RES (17)..(36): region may encompass 1-20 variable amino acids
GSVTGSCYCGKRISSDXXXXXXXXXXXXXXXXXXXXWVQELMSCLDLKEC

SEQ ID NO: 206 - protein, 50 aa, Artificial Sequence
  Description of Artificial Sequence: Synthetic peptide
  MOD_RES (17)..(36): region may encompass 1-20 variable amino acids
GSVTGSCYCGKRISSDXXXXXXXXXXXXXXXXXXXXVQELMSCLDLKECG

SEQ ID NO: 207 - protein, 50 aa, Artificial Sequence
  Description of Artificial Sequence: Synthetic peptide
  MOD_RES (17)..(36): region may encompass 1-20 variable amino acids
GSVTGSCYCGKRISSDXXXXXXXXXXXXXXXXXXXXQELMSCLDLKECGH

SEQ ID NO: 208 - protein, 50 aa, Artificial Sequence
  Description of Artificial Sequence: Synthetic peptide
  MOD_RES (17)..(36): region may encompass 1-20 variable amino acids
GSVTGSCYCGKRISSDXXXXXXXXXXXXXXXXXXXXELMSCLDLKECGHA

SEQ ID NO: 209 - protein, 50 aa, Artificial Sequence
  Description of Artificial Sequence: Synthetic peptide
  MOD_RES (17)..(36): region may encompass 1-20 variable amino acids
GSVTGSCYCGKRISSDXXXXXXXXXXXXXXXXXXXXLMSCLDLKECGHAY

SEQ ID NO: 210 - protein, 50 aa, Artificial Sequence
  Description of Artificial Sequence: Synthetic peptide
  MOD_RES (17)..(36): region may encompass 1-20 variable amino acids
GSVTGSCYCGKRISSDXXXXXXXXXXXXXXXXXXXXMSCLDLKECGHAYS

SEQ ID NO: 211 - protein, 50 aa, Artificial Sequence
  Description of Artificial Sequence: Synthetic peptide
  MOD_RES (17)..(36): region may encompass 1-20 variable amino acids
GSVTGSCYCGKRISSDXXXXXXXXXXXXXXXXXXXXWVQELKSCLDLKEC

SEQ ID NO: 212 - protein, 4 aa, Artificial Sequence
  Description of Artificial Sequence: Synthetic peptide
GGGG

SEQ ID NO: 213 - protein, 4 aa, Artificial Sequence
  Description of Artificial Sequence: Synthetic peptide
  MOD_RES (1)..(4): variable natural or non natural amino acid
XXXX

SEQ ID NO: 214 - protein, 50 aa, Artificial Sequence
  Description of Artificial Sequence: Synthetic peptide
  MOD_RES (17)..(36): region may encompass 1-20 variable amino acids
TELRCQCLQTLQGIHLXXXXXXXXXXXXXXXXXXXXMVQKIIEKILNKGS